SEQUENCE LISTING (185 sequences)

SEQ ID NO: 1 - DNA, 4141 bp, Nicotiana tabacum
atggaggagccaactcgattagtagaagaagcaacgattaataacatggacggacaacagaatgaagaagaaagagatccagagagcaattcactgcatcagcctcttctcaagagaaacagaacactatcatccagtccatttgccttggttggagctaaggtctcccacatcgaaagtttggattatgagtaagaacaactaataatcttatcatagatcaagtatagcttttctttacttgtgcattaaaagggccaacagaaattggatgtcctaattgtgtgtgtctgttttaggatcaacgagaatgatctcttcaagcatgactggagaaggagatctagagttcaagtattacagtatgtgttcttgaaatggacactggcatttttggtcggcctgcttacaggagttacagccaccctcatcaatcttgcaatcgaaaacatggctggttacaaacttcgagctgttgtgaactatatcgaggatagaaggtaggtgatgttttccctatgatcaacaattcataaatgcttccagaagtcttactactgattcttcaatacgataccactagctaatgactaagaacaagaccaaagatcacttatttgacttgaattatgttattgatttattcataattgagattgtaacaatggttacaggtaccttatgggatttgcatattttgcgggtgctaattttgtgctcactttgatagctgcccttctctgcgtgtgctttgcacctactgctgcagggcctggaattcctgaaatcaaagcttatctcaacggtgtagatactcccaatatgtatggagcaaccacactttttgtcaaggtgcgtcacacacccaattttatcagtgctggcaattcagatagcaggcagattataacgccatcagtatagtattgagattctgtcgaaccagatgtataaatagatagaatagcagcaaataacacatttttatcttagtcgtgatggcacctaatccgacccgctagataagccaaatacaatcaacacatatttatggaattcaatctcatttgggaagtgatctctatctttcagtaatcagataggaagtggtttaagaataaaaagagaattttagaatcgaatgcactcatccagcgaggaagatccatcagtggtatctaatttactcttgaacttccagcagttcaatcctttggtaccgtcactgtaacttgtttttttcaatctttgtgactaacatggaagggaggaaaatcctgactttcagtgattttcctcgcttacagtgaaagtcaggatatagcttcggtgagactcagcttatatgtcttaattgaatatgctatttgttgactaacatggatttgccctatcatgaaaatgaaggaagcgccaaaaatacatatacttaaacaggggcggacccaagtggtgagaagtgggttcaactgaacccgcttcgtcaaaaaaatactgtgtatatgtataaattatggctaaagcaaggtaaattttgtatagaaataagcttatgttagttatggacttctcctgggtccgctactgtacttaaaagcacatacgaagagatacacaaactaagggcaaaggttcataatttaaggcagttgtgtccagaagaacaaattttgcttgcatgttgcagtgtgaatttaacaataaaagaattatgatcgcaaatttccacttgtaattgtactataagattctaaattttgagagatttgacatgtttgctttccctttgactgaatcgtaaaagtgaaagtgaagttcatcagaagtagattatgatacttaccaacccctttttcccttaaacaatctttaatctgttcactcacagatcattggaagcattgcagcagtttctgctagcttagaccttggaaaagaagggccattggttcacattggcgcttgctttgcttccttactaggtcaaggtggtccagataattaccggctcaggtggcgttggctccgttacttcaacaacgatcgggacaggcgagatcttatcacatgtgggtcatcatcaggtgtgtgtgctgctttccgttctccagtaggtggtgtcctatttgctttagaggaagtggcaacatggtggagaagtgcactcctctggagaactttcttcagcacggcagttgtggtggtgatactgagggccttcattgaatactgcaaatctggcaactgtggactttttggaagaggagggcttatcatgtttgatgtgagtggtgtcagtgttagctaccatgttgtggacatcatccctgttgtagtgattggaatcataggcggacttttgggaagcctctacaatcatgtcctccacaaaattctgaggctctacaatctgatcaacgagtaagcacctactcttccacattcccaactggatcatcaaacattcagttggttctctatattttaaaggcaatgcatatccacacaaaaatgagcttacttggattagaatcatcttgagacattgatccaactgtcttgcatctttttaagtttaaatcctaattcctatccaaacatggccttcttatcacatttaactgccaaaaaaaaagggaaaactatagatgcaaaatcctgactttcaatctttgatccttttttatcttgcaggaagggaaaactacataaggttcttctcgctctgagtgtctcccttttcacctccatttgcatgtatggacttccttttttggccaaatgcaagccttgtgatccatcacttcccgggtcttgtcctggtactggagggacaggaaacttcaagcagttcaactgcccagacggctattacaatgatcttgctactcttctccttacaaccaacgatgatgcagtccgaaacattttctccataaacactcccggtgaattccaagttatgtctcttattatctacttcgttctgtattgcatattgggactcatcacttttgggattgctgtgccatctggtctcttccttccaatcatcctcatgggttcagcttatggtcgcttgcttgccattgccatgggatcttatacaaaaattgatccagggctgtatgcggttctcggagcagcttcccttatggctggttcaatgagaatgactgtttctctttgcgtcatatttcttgagctaacaaacaatcttctccttctgccaataacaatgctggttcttctaattgccaaaagtgtaggagactgcttcaacctaagtatttatgaaataatattggagctgaaaggtctacctttcctggatgccaacccggagccatggatgagaaatatcactgctggtgagcttgctgatgtaaagccaccagtagttacactctgtggagttgagaaggtgggacgtatcgtagaggccttgaagaacaccacatataacggattccctgtcgtcgatgaaggagtagtgccaccggtgggtctgccagttggggcaactgaattgcacggtcttgtcctaagaactcaccttcttttggttctcaagaaaaagtggttccttcatgaaagacggaggacagaggagtgggaagtgagagagaaattcacctggattgatttagctgagaggggcggtaagatcgaagatgtgttagttacaaaggatgaaatggagatgtatgtcgatttgcatcccctgactaacacaaccccttatactgtggtagaaagcttgtcagtggctaaggcaatggtgcttttcaggcaggtggggctccgccacatgctcattgtacccaaataccaagcagcaggggtgagattataagcaaatttcagttatttttcttatgcaaatatctccctcctatcatagtataaagatgcacagaaatagtcatatggtaatataagcacttgtttagaataattataggtggcaaagttattttacattagaagtgataaaagcattacttacatcacacttgtgctccttttgtaggtatctcctgtggtgggaatcttgaccaggcaagacttgagagcccacaacattttgagtgtcttccctcatctggagaagtcaaaaagcggtaaaaaggggaactga

SEQ ID NO: 2 - DNA, 3781 bp, Nicotiana tabacum
atggaggagccaactcgattagtagaagaagcaacgattaataacatggacagacaacagaatgaagaagaaagagatccagagagcaattcactgcatcagcctctcctcaagagaaacagaacactatcatccagtccatttgccttggttggagctaaggtctcccatattgaaagtttagactatgagtaagaacaactaataatcttatctttagatcaagtatagcttttctttataaatgggccaacagaaattggatgtcctaattttgtgtatctgctttaggatcaacgagaatgatctcttcaagcatgactggagaagaagatccagagttcaagtattacagtatgtattcttgaaatggacactggcatttttggtcgggcttcttacaggagtgacagcctcccttatcaatcttgcaatcgaaaacattgctggctacaaacttagagctgttgtgaactatatcgaggatagaaggttggtgatgttttccctatgatcagcaattcataaaggctactataattcttcaatatgattccactagctaatgactaagaacaagatcaaagatcacttatttgacttgaattatgttattgatttgttcataattgagattgtaacaatggttacaggtaccttgtgggatttgcatattttgcgggtgctaattttgtgctcactttgatagctgcccttctctgcgtgtgttttgcgcctactgctgcagggcctggaattcctgaaatcaaagcttatctcaacggtgtagatactcccaacatgtacggagcaaccacactttttgtcaaggtgcgtcacgcacccaattttatcagtgctggcaattcaggtagcaggcagattataacgccatcagtatagtattgagatcctgttgacctagatgtataaatagaaagaatagcagcaaataacacatttttagcctacatatttatggaattcaatctcatttgggaagtgatatctatctttcagtaatcagataggaagttgtttaagaataaaaagagaattttatcgaatgcactcatccagcaaggaagatccatcagtggtatctaatctactcttgaacttccagtagttcaatcctttggtactgtcactgtaacttgttttctcatccaccattaaaatacaatagcttccatgagactcagcttatatgtctcaattgaatatgctatttggtgactaacatgaatttgccctatcatgaaaataaatggaagtgacaaaaatacatatacttaaaagcacatatgtagagacacgcagactaagggcaaaggttcacaattttaaggcagttgtgtccagaagaacaaatgaagaattatgatcacaaatttccacttgtaattgtactataaaatttttaattttgagagattctgacatgtttgctttccctttgattgaatcgtaaaagtgaaagtgaagttcatcagaagtagattatgatacttaccaactcctttttccccctaaacaatctttaatctcttcacttacagatcattggaagcattgcagcagtttctgctagcttagaccttggaaaagaagggccgttggttcacattggcgcttgttttgcttccttactaggtcaaggtggtccagataattaccggctcaaatggcgctggctccgttacttcaacaacgatcgggacaggcgagatctcatcacatgtgggtcatcatcaggtgtgtgtgctgctttccgttctccagtaggtggtgtcctatttgctttagaggaagtggcaacatggtggagaagtgcactcctctggagaactttcttcagcacggcagttgtggtggtgatactgagggccttcatagaatactgcaaatctggctactgtggactttttggaagaggagggcttatcatgtttgatgtgagtggtgtcagtgttagctaccatgttgtggacatcatccctgttgttgtgattggaatcataggcggacttttgggaagcctctacaattgtgtcctccacaaagttctgaggctctacaatctcatcaacgagtaagcaccaactcttccacattcccaactggatcatcaaacattcagttggttctctatatttaaaaggcaatgcatatccacacaaaaatgagcttacttggattagaatcatcttgagacattgatccaactgccttgcatctttttaagtttgaatcccaattcctatccaaacatggtctttttatcacatttaactgccaaaaaaagttactctatagatgtaaaatcctgactttcaaactttgatccttttttatcttgcaggaagggaaaactacataaggttcttctcgctctgagcgtctcccttttcacctccatttgcatgtatggacttccttttttggccaaatgcaagccttgtgattcatcacttcaagggtcttgtcctggcactggaggtacaggaaacttcaagcagttcaactgccctgacggctattacaatgatctcgctactcttctccttacaaccaacgatgatgcagtccgaaacattttctccataaacactcccggtgaattccatgttacgtctcttattatctacttcgttctgtattgtatcttgggactcatcacttttgggattgctgtgccatctggtctcttccttccaatcatcctcatgggttcagcttatggtcgcttgcttgccattgccatgggatcttatacaaaaattgatccagggctgtatgccgttctgggagcagcttcccttatggctggttcaatgagaatgactgtttctctttgcgtcatatttcttgagctaacaaacaatcttctccttctgccaataacaatgctggttcttctaattgccaaaagtgtaggagactgctttaacctaagtatttatgaaataatattggaactgaaaggtctacctttcctggatgccaacccggagccatggatgagaaatatcactgctggtgagcttgctgatgtaaagccaccagtagttacactttgtggagttgagaaggtgggacgtatcgtcgaggtcttgaagaacaccacatataacggattccctgtcgtcgatgaaggagtggtgccaccggtgggtctgccagttggggcaactgaattgcacggtcttgtcctaagaactcaccttcttttggttctcaagaaaaagtggttccttaatgaaagacgaaggacagaggagtgggaagtgagagagaaattcacctggattgatttagctgagaggggcggtaagatcgaagatgtggtagttacgaaggatgaaatggagatgtatgtcgatttgcatcccctgactaacacaaccccttatactgtggtagaaagcttgtcagtggctaaggcaatggtgcttttcaggcaggtggggctccgccacatgctcattgtacccaaataccaagcagcaggggtgagattataagcaaatttcagttattattcttatgcaaatatctccctcctatcatagtattaagatgcacagaaatagtcatatcgtggcaaagttattttacgttagtaagtgataaaagcattacttacatcacacttgtgctccttttgtaggtatctccggtggtgggaatcttgaccaggcaagacttgagagcccacaacattttgagtgtcttccctcatctggagaagtcaaaaagcggtaaaaaggggaactga

SEQ ID NO: 3 - DNA, 44278 bp, Nicotiana tabacum
atgaatcacggaagttgttgggtcgtcatccaaattgctggccttgggctcgacgaccatctcttcctccgggacgttcctctgacggaaacattgaaaaagaacaagatatgtgcgacagcagcaaagtcgatagtgatagtggcatccagataggatctctgctcgaggaagttatcccacaaggcaataataccgctataatctcggcttgctttgttggcctcttcaccggtatcagtgtcgtgcttttcaacgctgcggtacgtgcgctataggtctttcatttctcttttcatgtactattcctccttacttacttggcctcagtcaatcagccccctgcctactttaaattattgtacattttatcagaggagtgtcctatacatcaaattcacataacttagtaaaatatgctgatattctgaattttaaacttaccagcttagaacatccaggttagttcagaaacagataatctaaattggtctcatttataagtcattttgttattcaagacatacaatttggctcttgataaaagattatgcagcgcccgatgattacctaatatttatcagcaacccatgtaatttaacaatattgtcaccatataaaagagaactgaagagaatgttcaatttgtggtcatataacggatatctcccttggttaggttcatgaaatacgtgatctttgttgggatggaattccatatcgagctgcctcagaggagcccattggagtacattggcaacgtgtaatcttagtaccagcttgtggcggtttggtagtcagctttttgaatgccttccgagccactctggaggtttcaactgaaggaagttggacatcatctgttaaatctgtattggaaccagttttgaagacaatggccgcttgtgtcacattaggaactgggaattccttaggaccagaaggccctagtgttgaaattggcacatctgttgccaagggagttggagctctgcttgataaaggtggtcgtagaaagctgtcactcaaggctgctggatcagctgctggaatcgcttctggtttgttccccatattattcttggttctgaaccatacatggtacattttccttataattacatgtagcctgttgtatgctttcctctttcccgggaagcctttttgtaaatacaagtgtgtttgcactcaaaccaataaactgtaaaaaaggtgaactccttaagcaagcaaaagcattagaaatgtaaactagacatatttctcagattgagagtctgagagattagaacacgagtgtttccattagagagagaaaagagacttctagatatttctattatctctgtaagagtgaatccgttcctatacaaaaaataggccttcattaaatacaagcttgggctgggtactactgggccaaagtaaaaaataaaaagaatcacccactatcaaatgggcctagtctaacaacccccttcaagctggagggtgacacaacccctagcttgcgaatatgaaaatgatgagcaggcccaagtaacactttggtaagaacatcaaccacttgagaagcactggagttgtgaaatagactgatcaggccattcccaagcttgccacaaacaaaatgacagtccagcttaatgtgtttagtgcgttcatggaaaacttggttttttgcaatgtggacttcctgattatcacaaaataaaggaacaggtaaagaaggagaaactccaatatcagacaataatttggtgagccaagacacctctgcaacagccttactcatggacctatactcagcttcaattgatgatagtgagacaacaggttgcttctttgatttccagctcaccaagctgccccccaagaaaaatacaaaaaccagtgacagacctgcggctgtctgggcaagaagcccaatcactgcacaataaagctgcaaagacaagtctggagagttattgcggaagattccaaagtcaaaagtgcccttgaggtatcttagcaagtgcagggcagcctgcatgttaggaacacagggagactgcataaactgactcagatgctgaacaacaaaactaaggtcaggccttgtgcgtatcaaaaagtttagcttgtgcattagactcctgtactcttcaggcctgggcaaaggagtgccaatcttagcttttaacttcacattcaattcaagggggcaagtgacagaagagcaattcgaggaatgaaaatcagccagcaaatcatgaatgaactttttctgatgaagaagaaccccagaatcagtgtataaaacctcaatgctaaggaagtaattaagagagcccatgtccttaatcttgaactggtcactgagaaaggacttcaaagcagccaattcagctagatcacacctagtcaatatgatatcattcacatagacaaccaagatgaccaaggaatccctagaacccttggtaaaaatagagaaatcattcaaggaacgagagaagccattagagcacaaggcttgagataatttagcatactattgtcttgaagccagtcttaaaccataaagagacttctggagtttgcatactaaaggagcagaagaagagtgaggaacagttaggcccggtggcagcttcatgaatacctcctcatcaaggtccccatgtaagaagacattattcacatctagttgaaagaggggccagtgttgtttaacagctacaacaataagagttttgacaatagacatattgaccacaggagaaaaagtttcattaaagtcaataccctcaacttgagtgacctagctttatatctctcaatactttcattagccctatatttaaccttgtatacccacttacaactagtaggtttcttgccaggaggcaattcaacaatgtcccaagttctgttggcatccaaggcctcaaattcacatctcatggctgcctgccattcaggaacagctgcaacctgagagtaagaataaggctcaggaacatgaagttgactaagagaaggagcattagaaatagatctggagggaggaggagaagaagtggaggtgcagacataactcttgagatagttggttggattgtgtggcacggaagatcttctcaaagcaggaggaggtacaagagagttagaataatgagaaggagaagagatggaagtgggaacagagaagattgagaagcagtagaaggagaaagtgaaggagatgaaggagaggaagaagacggaaaggaacattcatcaaaacaagcagaaaagggaaaggggaagacttgaggtactacatgagaggattgaaagaaaggaaaaatggtgttcataaaaaatgacatcttttgatacaaaacaggtgttattctgaagattaaggcgcttgtagccctttttggcaaaagggtagccaatgaaaacacaaggaagggacctaggatgaaatttgttttgtgaggggtggtgacagttgagtaacagaggcacccaaaagctctaaggtggtgataagtagggtggaagaatgaagcaattcatagggacttttgtgattaagaagaggaaaaggaaatctgttaattaaatatgtggcagttaaaaagcagtcaccccaaaatttaagtggtagatgagactgaaacataagtgacctagcagtctctagtaaatttctgtgttctctttctacaataccattttattggggggtgtgaggacaggaggtttggtgtactatccctttttctgaaaagaaaaggcaaccagaagaactagatcccagttccaaagcattatcactcctaacagtttgaactttagattggaattgggtttcaaccatagcaatgaaaaccttgagcaaatcaaaggcattgcggcacccattaaatgtgtccaagtagccctagagtagtcatctacaatggttaaaaaatacctagaaccattataggtaggagtagaatagggtcaccaagtatttatgtgtattagctgaaaaggctgggtggagtgaatagaactatcagggaaggacaacctggtctgcctcgctaaaggacaaaccggactagtgaatgaccgtttggaagacagtttgcaattaagaccagaaatgcatttcattttatagaagggaatatggccaagtttgtaatgccaaacaacatcatctttattcacattatgcaaagcagtactagtatttacaattggagtatcatcaggtacagaaataggagcagaaactgaattaagcaaacaagaaataaggaaattagaaagaggtaaaggagatgatgttggaggcctggcattctgaaatagtttgtagagtccattgtccaatctaccaagaaccactggcttcctcactgaagggccctgtagggtacaagtagccttggtaaattgtacaatatcatcatcatgggaaagtaatttgtacacaaagatgagattatattgaaaactaggaatatagagcacattataaagaatcaagtcagggaacaaggctaaggaaccaatattagtgaccttaaccttatacccattaggaagggagacaaggtatggtacaggaagtgtttgaacattaaaaaaacaaatgtttaagggaggtcatgtggtcagatgcccagggtctattactcaaactacactatctatcatagtcagcataaatgcaccataagacaacccttgtgaggtaataactcaccagcaaagttggtagaagcaagatagttggttgaagaagtagatgatgctgatgaagacagttgagattgttgaagtaacattagctgagaatattggttcttggtaagaccaggaactggataggactgttcaggagcagaggtaccttcaggaccagctgacattgcagaaccaccagaggtatccacctcagcatgggcaacagaccttctgggaggaagagatctatttgacttgaaatttggaggaaagccattgagcttatagcacttatcaatgctatgtccgggtttcttacaatagtagacatgtgaagctcaaaagatcccttagaggtagtaccggacctttgaggttcaaaatttattttaggagagggaggaggcctggatacaccaacactgaaagaagcagaatttgaggcatattgagttctagcaaaaatttgtctttgcttctcatcagatagcaaaatcccatatacattaccaatggaaggtaagggcttcatcatgatgatgttgcttcttgtttggacataagtatcattcagtcccataaagaactggtagaccttttgttccctgtcttcagcagatttacccccacaagtacacattcaaactctcccggcagacaaagatgcaatatcatcccatagtcgtttaattttgttgaaatatgatgctatgtccatggacccttgggaaatatgagccagttccttctttagctcaaagatcctagtacctctcttctaactcagtccaaatattcttagcaaactcagagtattcaacactcttggatatttccttgtacatagagttagtcaaccaagagaccacaaggtcattgcaacgttaccactgtctggctagaggagaaccttcaggaggtctgtgagaagtaccattaatgaaatctagcttgttacgaatagacaaggcaactaggacattacgtctccaattgccataacagcttccatcaaaaggaccggaaactaaggaagttcccagcacgtctgatggatggacatataaggggcgacagggatgggtataatcatcttcatggaaaattaggcgtaagggagtagaagaagtcgcatcagcactggtgttattatcatttgccatttttttcaacagattgtcaatcaaccaacacaatacagatacacatatatagattgtgagaaagcacgagagaaaaatctatattattgatattctatttaattataatacaatgagccctatttatacaatacatatcatactcctattctatgtgggactaggactaattcatattatgtacataactatctaacactccccctcaagccggtgcatacaaatcatatgtaccgaacttgttacatatgtaactaatacaaggaccagtaaggaacttggtgaaaatatctgcaaactgatcatttgacttcacaaactttgtagcaatatctcatgagagtatcttttctctgacgaaatgacaattaatctcaatgtgtttagttctctcatgaaacaccggatttgatgctatatgaatggcagcttggttatcacacatcagttccatcttgctgacctcaccaaatttcaactaattaagtaaatgtttgatccaaactagctcacaagttgtcacagccattgctcgatattctgcttctgcactagaccgagcaaccacattttgtttcttgctcttccaagacacctaattacctcctactaaaacacaatatccagacgtagaacatctgtcaaaaggtgatcctgcctagccagcatttgagtacccaacaatttgctcatggcctcgatcttcaaacaataatctgttacctggagctgattttatatatcgaagaatgcagacaactgcatcccaatgactatcacaaggagaatccaagaactgacttaccacactcactggaaaggaaatatcaggtctaatcactgtgaggtaatttaatttaccaaccagccgcctatatctagcaggatcgctaagcggctccccctgtcctggtagaagtttagaattccgatccataggagtgtcaataggtctacaacgtgtcattcctgtctcctcaagaatgtctaaggcatacttcctttgtgagataacaatacatgtgctagactaagcgacctcaatacctagaaaatactttaatctgcccagatccttagtctgaaagtgctgaaagagatgttgtttcaacttagtaataccatcttgatcattgccggtaataacaatattatcaacataaaccaccagataaatactaagatttgaagaagaatgccgataaaacacagagtgatcagcttcactacgagtcatgccgaactcttgaataactgtgctgaacttaccaaaccaggctcgaggagactgttttagaccatagagggaccgacgcaaccgacatacaaggccactagactccccctgagcaacaaaaccaggtggttgctccatataaacttcacctcaaggtcaccacgaagaaaagcattcttaatgtccaactgatagagaggccaatggagaacaacaaccatggatagaaaaaggcggactgatgctattttagccacaggagagaaagtatcactgtaatcaagcccaaatatctgagtataccctttggcaacaagacgagccttaagtcgatcaacctggccatctggaccaactttgactgcatacacccaacgacaaccaacaataaatttacccgaaggaagaggaacaaactcccaagtaccactcgtatgtaaagcagacatctcgtcaatcatagcctgtcaccaccctagatgagacagtgcttcacctggatggaaatagaggacaaagatgatacaaatgcacaatagggtgatgacagacgatggtaacttaaaccgacataatggggattagcatttagtgtagaccgttcacctttccggagtgcaatcaattgactaagaggagacaagtccgcagtattagcaggatcaggtgcaggacgtgaatcagctgggcctgatgctgggcgcggacgacgatgataagttaggagtggtagagctgtagaaggttgaactggactaggcagtggaactgaagctatatgtggtggaactggagctataggtggtggagctggagctgtaggtgaagatgaatgggagatagtgactgaatctccaaaagatggaactggtagcacctcagatatatctaagtgattacctggactggtgaagtatgattgggtttcaaagaaggtaacatcagcagacataaggtaccacctgaggtcaggagaatagcatcgatatcccttttgtgttctcgagtaacccaaaaatacgcacttaagagcacgaggagctaatttatcttttcttggagtaaggttatgaacaaaacacgtgctcccaaaggcacggggtggaagagagaacaaaggtaagtggggaaacaagacagagaatggaacttgattctggatagctgaagatggcatacgattaataagatagcaagatgtaagaactgcatccccccaaaaacgcaacggaacgtgagattgtatgagtaaggtacgagcagtttcaataagatgtctattctttctttcagctacccgattttgttgggatgtgtatggacaagatgttttatgaataatcccatgagagttcataaactgttgaaatgggaaagacaaatactctaaggcattatcactacgaaatatgcggatagaaaccccaaattgattttgaatttcagcgtggaaggtctggaaagtagaaaacaactcagatcgattttttatcaaaaatatccaagtgcacctgtaataatcatcaatgaaactgacaaagtagcggaatcccaaggtagaactgacctgactaggaccccaaacatctgaatggactaaagtaaaaggtgactgactctgctcgattatcaagacggcgagggaaatgggagcacgtatgcttaccgagctgacatgactcacactctagagtggacaagtgagataaaccagataccattttttgaagttttgacaaactgggatgtcccaaccgtttatgtaatagatctggtgaatcagtaacaggacaagttgttgaagaaagacaagatgtaagtccatgtgattttgcaagaataaggtagtaaaatccatttaattcacgcccggtaccaatgatccgccctgtactgcgttcctgtataaaaacaaggtcatcaagaaataaaacagagcatttaagtgatttggctaagcgactaacggctatgagattaaaaagactaacgagaacataaagaactgaatctaaaggtaaggaaggaagtggacttacttggcttattccagttgccatggtttgagactcgttatccattgtgactgttgggagtgattgagaatatgaaatagtaatgaaaagagatttgttaccaaaaatatgatcagatgcacctgaatcaatgacccaagactcagaggttgaagattgggagacacaagtcacactactatctgtttgagcaacggaagctatccctgaagatgtttgtttacatgttttgaactgaaggaactcaatataatccggtagagaaaccatccaactcttcgtagtattggattccattttgctacaaccaatttctcaaattcttgattacaacttgtgtggttaaccttggaatgccaaatcagaacaccccttttttttttttggaaaacattgttcactcgctggaaaataaaaaaggttgccggaatttgatgaaacttgaatagaccgactcggaataatgtcctaagaaggctgtccaaaaggagttttgtcagaaactgaccagaaggaggtccacgcaccggcgcgtggacagatctcgccgaaaaaaaaaatcactttggttggcgcgtgatggcgcgtgggtggggtttttccggtcgggttttgtggggtttgctcccccggagatggagaacactgtggtggtgttggtttatgcacaacactggtaaaaagtggttttgatgcgaacagctactcaggtcaccaaaaaattgcacggtgacgactgatttcttcccggatgtcgttggaatgacgcacaacgataattatctcaccaatgctctgataccatgtgagaaagtacgggagaaaaatctatattattgatattctatttaattataatacaatgagccctatttataagactaggattaattcatattatgtacataactatctaacatagatcaaataggcatgcaattcacaataatggtgaataaaatgatacgaagttacccagctcttttcgcgatcgaaaaggagaaaatagccttcaatcacaaacgagaaagaagaatctccggcttgacagtagacgacttcgaaaccctagctcgagatgaaaaccacaaaatccccaaatcacattaccaaccaaacaatttgagatcacaaatgttgaatatgtgagaatccgactaagaaatcaacaaaaaatcaatagaaatggttgaagaataccgacttgaaccctaaatgagtcagacatcacctagaatgaaatacaccttcgaaattgacgaaaacaggaccggttgaaagcggagaacgtgccatagaaggatctacgctctgataccatgtaaacttgacatacttctcagattgagagtctgagagattagaaaacgagtgtttccattagaaagagagaaaagagacttctagatatttcgattatctgtgtaaaaatgaatccgttcctatacaaaaattaggccttcattaaatacaagattcggccgggtattactggcccaaagtaaaatataaaaagaatcacccactatcaaatgggcctagtctaacaagaaaaccaacaaatagtccccccccccccccccaaaagataccactgaaatgacaccgggtgcccaaaaataaagcagcttacttcttgactttgagaggaactgcaatccttatcggtttgagaggaactgcaatcagctataagtagcttattaatttccagtgcctgcattctgccaagtactatgatatatttctgaagctttgtttccccagttcctttttcagacgtttgctgtcaataaagttgagccagccaacttggctcccacaagctactaattttgtccaagcttactctatgggagaagttaaatttcccaaattccttgagcggaaaatgaaaaatggactcaaagtgtcatattatgcaactatctaaagaaaaatactcaattgaagtttagataagaaaagtgaatgtatattgatgtagtctccgttaggtgagaagcgtatcacttacccagcaacatatggacctaacattttactagtgaagttttcacattgtatcaaaagctcaacaaacggaaaggtgactaatcctaaaatgttatttcacatatatgggcacacggtttgtcaaccttctcatacgtgcattatttgttctctatctttctatttcatccgatataaccaatcgttattgtaaattctataatgcctgtggttacttttgtctttagtgacaaatgacatttaggataaccatgtagttattgacttatttcacttgaggtctcttccaattatgtagtagtagagtgttgagatatggatatgttaccttctaaaaaaaagagtgtagagatgcggatagtttgctagctggcttttgtctcccttcaagttgaattagcaaaagcttgtctcataagttggatagctagacaagaaaaactccaaattactttatgtagagtattcttaagcttgagtcgcgagttggaaactggaattatgtaaaaaaacctggaattatttggttgagcctgctttttagttttgtcaatatttccagtatctaacccaacatgtttagagtgattcccggagagcctcagtacaaggcatttgcagagtctttatgagagtccaggaaggggcacacattctgtagaggtatagtcttgtccttattttcagggttgaactagttctttagaagttacctaggcttcctaatttccaaatttctgccaggtccttttttggtgaagtacttgaagtttaataaatcaaattttaatttctaacatatcctgagaaatttattcacaaattcaactggtgacttctgatgcagaaacataagcaactgcttatgggttcatatgttcctgcaattttattgttgacatggattggcttcatatggttttgttcctgcaattttatcgctgacactaatcctttcatatggttttatgtggagtgttaaatagaggttaagagacaagaagaggctgaaaaaggtgggcagttcatttgttagtagactactctatttactaagagatatgatgtcccatacattactcgaattggctccgaatccagattccacttctttgccgagtttccttattgtacatagttcgactcgtcaagggaaattcacttcctttgactgaataatgctagtttgagtagtaccttacattaaatggaccatttagttctatctacttgatagaatagactggtcatcaactagttgcaaatacaatgacaactttgccatgtttgcagagtcacctgatgaagaagtacctcaattagtagaacatttcttgaatgttctacagtattctctatgcctacatgaccacatcacttttccttttgcgttgtgagaacttgaacttggtgagcgggggttccccaggaatggcatcttgatggcagatgaccattctgtccttgtcttagctaatgcttcttgcattgcctcactagatttattatacctttaaaaaatgtttgccattgttctgccataatagaaggatgtacccagctggtgcttcaaaactaatgaaatgctttacaattgtcgagtcctaaaggatgatttgtggaatcagatctcaaacaattctttttgaggaagaaaaataccaaaggttttttctgtttgttggaagattaaaaatcctttaaatggtaaagatttatgaacttaattcagcgtttttgtggccattgctggaaaagagaaaaaacaatggcacttcttcgagtttgcttatccaaaaaaaagaagaagagaatgtcacgtaatgcaatttcatcttaggaaactttgcaggagaaaagcaagagtgataaaacagaactatttgttttttttaacaagttgttgtgacctatttcttgtcattcttatttgctaataagctaatgtactatagttcctgtactatggtttgttttgacttaatacggggatgttcaatgagcattttcttgttttttctgctttcagcatctgctgccttacaggaattcattttctggaaatttacttcttgttctgctaacattttcctgttatatcttgtcagtcattttctctccatggttatactgtttgtgtcactttaaactctccttgttttctactttaaaggatttaatgctgctgtcgggggctgtttctttgctgtggaatctgtgttatggccatcacctgcagagtcctccttgtccttaacaaatacgacttcaatggttattctcagtgctgttatagcttctgtagtctcagaaattggtcttggctctgaacctgcatttgcggtcccaggatatgattttcgtacacctactggtaattttggacttctttctcgagtttgattcttaaatacaattgtacccgtcacttacagcaacaactacatttcaacagctagttggggttggctacacagatcatcactatccatttcaattcatttagtcccatttctttcgaatattgagtactttgggattctataatatcaaggttctttatattttctactttgacgtacaaatctctaaatagattaaagaagactcctagagacactggcctaatgcaaatgtaccaccatgaataaactttaatctgaaatagctggtatcttatataaggacccttagctttaattgtgttctatattgatcttttgggacaacttccttccaatattatgtcttacttatacagttatacttatccttaagccttactctttagagtggttatccctaattcaagcttttgttggcaccatagctagtttggttctaagtaaaaagttactctttagagtggtaactttttgtcaattttcttagtgaaaatataacctctgtgacaaatctaccaagtataaatccaatttggttctatgtcatccttgtagtttatccaagtcaatgctccatcactcttacaaaggttcatcgtatgactaatcttttttggagaaaggtaacagtttgtattgataataagatcagcgccaggttggtcattagtgctaatagctgtacgtacaactccaaaagagcaaaagacaagcacctgatgtaaggtaaattacaagctgcctataaaatctatcaggtgtcctatctcactaaacatttcttgtttacaccaaaaaaataaaacaaggaaagacaatccatcttaatcttctgaatggagtttctttttccttcaaaacatctggagttccttccgttccatgcaatccaccatatacaagctgggatgattttccatttgtctttatccatttcttctaccaattcccttccaattgattagaagttccaatgtggttctagatatgacccaattaactcccaacagataaaagaagatgtgccacggatttgtagtgattctgcaatgtaggaacaagtgagcattactttctacttcctgtccacaaagaaaacatcttgagcaaatctggaaacctcttctttgtaagttatcatgtgttaaacatgcctttttcaccaccaaccagacaaaacatgatactttgggaggagttttaaccctccaaatgtgtttccaaggccacacctcagttgttgaaacattaggatgtagagtccagtatgctcttttactgaaaatgcaccttttctattcagcttttaaactactttatctatggtctgtgatgtacccttgaaaggttcaagagtttggaggaagatagaaactctgtttatctcccaatcatccaaagatcttctaaagttccagctccatccttgtgagctccagactgacttaccaatgcttggctttgaagacttagagagaataagtcaggaaaatatctttcaaccttccttgccctatccggtgatcttcccaaaaagatgtctgcaacccattgccaatattgatcttgatattgctactgaaagatttcttttggtggcaggattactctcattaacaatgtacttgacaatctccatacatactaatgtctctttaccctcttgccattaaggttgtaaagagacttgtcaaattaagaaaaggtttcctatggaactgtttcaaggaaggaacctcctttcctttggtcaagtggagttaagtcatataatctaggaagtggaggcttgggtatgaaatagctgcaaatacagaaaaggagcatcttatttaaatgatcacggaaatgtgcccaaaactttaaatatctgcacagcatatggttgtagcaaaatttgaatcttcctgtcaatggtgctcatgtccagtgaatacccctgatggtgaaagtgtcctgaagggaagcaggaacttattggaagaattggcatctaacactcagcttttcggtgggtcatagcccattgaaaattgagtgcccagatttatatagttttgctctaaactgacgatgcagttgcacaacatacgacaaactaaggtgggacatcatcttcttcggaaggaattttgaggattaagagatagagtggttgattcagttgcaaatgaagcttcaagggttcaatatcatccaggagacaccggattctgatagataaaacaacagaaagatgagcactactttgttaggcttgttacaagttgctatcgtctttcttatctcggtacacaatttagatttgggaacttagttggaaaagcagagtggttgtttttgtgaatagcatcagacaaagcttctgagctggtacgacagaaaactcaacagggagaatagaagactgtggttcacaatttctgcatgcatcttgtaggttatttggtgggtaaattatttaatgttttgaagggaaggtagaacatgttcataggcttagattcaaatgtttgtatttttttggctctttggtgagagatgctgaacgtaaatgacataggcagctgactataatttctcagctccttgctttttaaattgacaggcactgatatgtacatgtgaacatccaacacttttgtggtgccgttccgatgaataaagaacattaatcacttactgatcaggagtaatagtttaggagttctagaatttttgtacataaaatgaaccaaaaagaagatcggaatgagaacatgtttctttttttgttttttctttttcgtgaaaacttcaataacacttctgatagaatagctaggtccatttgaattcctttggagacccttacacaaccaatgaatgacaagtatagcatttctaactccctcccacacgtataacccagattttagggtttagatgtggatctgatttgaccttattgcctttttttgtttttgttctttttgaagtagagagtgaggaggctcaacaattaattcggctcaacgggctaatgattggacttacatgctacgacaatgttaggagagagagagagagagagaagcccagagcagttacatgagttaagaaagagaagtccaaagcgatagaatatgaagagagaaagcggttgtgctaacaggctccctgaagtttggctctgagcatccaactcaaaaccttaaggcaatgagtagagtagcccaggaccatttaaattgctgttgaaaaccttacacaaccaataagggaacaagtgtaacattctcttacaaccctaccgtcttataagtcagtgctctaatttagcataaaatcaaagtgaggcgatctacaatgaaatgaagtaaataactgataaatacaaagaatgttaattctccaatatagcctgaatgttcccagaacaaaataaactagtctcaggatttatcattaacatgatgttcctcttattttgagtgattaggaaggttaatcaaggtataaattctttctaatttgtatcgtctagaattatttatctaacaaattttcagattaccggttcaaaagaggaatatattttgcatacaacgttaccataccttacaaaagggagatgaacatttttttattttattattgtcctttttttcaattagggattatgcagtcttcctccacgtgatattactcttagaatcacgtttttgtcattgctattacttaatgtggtaagtacaaatgtgttttgaactctttttggtatgtaatattgagttaatttttggtttccatttcagagctgccgctttatcttctgctgggcatcttttgtggcttagtttcagtggcattatcaagttgtacatcatttatgctgcaaatagtggaaaatattcaaacgaccagcggcatgccaaaagcagcttttcctgtcctgggtggtcttctggttgggctggtagctttagcatatcctgaaatcctttaccagggttttgagaatgttaatattttgctagaatctcgcccactagtgaaaggcctctccgctgatctgttgctccagcttgtagctgtcaaaatagtaacaacttcattatgtcgagcctctggattggttggaggctactatgcaccatctctattcatcggtgctgctactggaactgcatatgggaaaattgttagctacattatctctcatgctgatccaatctttcatctttccatcttggaagttgcatccccacaagcatatggcctggtatgaatttgtcttttgttagaagtagcattacatatctggataagtgagttttttattattgaaaagtaataacaggagagcaagagaatatagcacccaaatctacttctttcctctcttctattcttctgaaattcaaggtcctttaactcctccacggcctgtctagttattgatcctgtagacttaattcacataggtttaggacattcaagtttatccaaacttcgtgaaaaggtttctaatttttttacattacagtatgagtcgtgtctacttgagaaacatatcactccatgtttctatagagtctgttttctcctcagtttattttgatatatggggtcctattaagacagttcaaccttggattttcattatttttgttgtttcattgataattattcaagatgtacttggattttcttaacaagagatagttctcagttgttttttgtgttcctaagtttttgtgctgcaatacaaaattagtttgatgtctctatttgcatttttcccaatgataatgccttagaatattttcttctcggtttcagtagcttatgatttctttagaaactctctatcagaaatctcaactgagatagatgagaggaagaataagcatatcattgagacggctcgtacccttctcattcagtcccctgtcaagcttagtttcttgggcgatgcagtttcacgtcctttgattagattaattggatgcctcatctgctatccaaaatcagattcaactttcgatattgtttcctcgcttacctttatactctctttccctcgagtctttgggagcacatgttttgttcaataacatagctcctggaaagtgaccagcgcaaccgacaagcaaggccttcttaatatagaaggagggcatatgctattctagccacgagggagaaagtaatattgtaatcaaacccaaatatctgagtataacctttggcaatggcgatcaatttgattatatggaccaactttgcctacatatacccaccgatagatttacggggaggtagagaaataagctcccaagtaccactaatatgtaaagcagacatctctttgatcatagcctgtccttgtggacatagggatagaaattgaggactaagatgacacaaaagcataatgctgtgatgataaacgatgataactcaaatcaatatgatggggatgggaattaagagtggattgaatatctttgcggaatgtgattggtagactaggaggagacaagtccgcaataggtaaaagatccagtacatggaatgaatcttctggacatgatgttggactgacgtcaatgataagtcaagagtggtggagttgcagaacatggaactggagctgtaggtgacataatcgaagttgtagggggtggagctatagaggaaggtgaaggagagatagtgactgaatctccaaaatatgaaaccggtaatacctcaaaaaatgtctaagagatcatttggacctatgaagtatggttgcgttttaaagaaggtaacatcagcagacataaggtaccgcggaaagtcaggtgaataacattgatatccttgttgcgtcctcgagtaacttagaaatacatatttgagagcacggggagctaacttatcttttctggagtaaggttataaaaaaacacatgctcccatagacacgaggtggaagagagaaaggtgagtggggaaacaagacagagtatgaaacttgattcttgatagttgaagatggcatacaattaataagacaataggatgtgagaactgtatccccacgtaaacacaacagaacatgagattgtacgagttgggtatgagcagtctcaatgagatacctattcttcctttcagctatcccattttattgagatgtgtatggacaaaatatttgatgtatgatcctatgagagttcatgaactgctgaaatggagaagacaaatactctggggcattatcactatgaaatgtgcggttagaaaccccaaattgattttggatttcagagtgaaaggtctgaaaaatagagaccaactcagattgatttttcatgagaaatatccaagtggacttggaataatcatcaatgaaactgacaaagtagcagaattccaaggtagaactaactcgacaaggacctcaaacatctgaatggactaaagtgaaaggtgactctattcgattatcaagacaccgaggaaaatgagagcgagtatgccttctgagcggatatgactgacgctctagagtggacaagtgagacaaaccaggtaccattttctgaagttctgataaattgggatgtcctaaccgtttatgtaataaatctggtggatcagtaaaaggacaagctgtaaggggacaaaaataccaaatatttccagaagatggcaaactacaacagaagaagcaactacattaacaggctcaggatatgtgatgaaatgaggacaaagagttgatcaagaaggagattctggaattctaccagaacttatatagtgaaaatgaaccgtggaggcccagtgcaaattttgaaggcatctcctcactaagcatagaagagaagaactagttggaagctccatttgaagaaatagaggtgcttgaagctttgaaatcatgtgcccctgataaagcaccaggtccagacggcttcaccatggctttctttcagaaaaattgggatactcttaaaatggacatcatggccgcacttaatcactttcaccagagctgtcacatggttagggcttgcaatgccaccttcatcgccttaattccaaagaaaaagggtgctatggagctcagagactacagatctattgacaaactagtctcgggggaacaaaatgctttcatcaagaacaggcacatcactgatgcttccttgattgccagtgaagtgctggattggagaatgaaaagtggaaaaccaggcgtgttgtgcaaactggacattgaaaaggcttttgatcaattaagatggtcttacctcatgagtatcttgaggcagatggctttggggagaaatggataagatggataaactattgcatttcaactgtcaagaactctgttttggtgaatagtggcccgaccggttttttctcctgccaaaagggcctaaggcaggggatctcctctcccctttcctattcattttggcgatggaaggactcactaaaatgttggagaaggctaagcaactacaatggatacaaggctttcaggtgggaaggaatcctgccagctcagttacagtatcccatctactctttgcggatgatactcttattttttgtggtactgagagatcacaagcacgaaatctcaacctgacgctgatgatcttcgaggcactatcaggactccacaacaatatgataaagagcatcatataccctgtgaatgcagtccccaacatacaggagctagcagacatcctatgctgcaaaacagatactttcccaacatatcttggacttcccttgggagctaaattcaaatcaaaagaagtttggaatggagtcctagagaagtttgaaaagaggcttgcgacttggcgaatgcaatacctctccatcggtggcaagttaactttaatcaatagtgtactggacagtcttcctacataccacatgtctttgttcccaattccaatctcagtcctaaagcagatggacaaactcagaaggaagttcttacgggaaggatgcagcaaaacacacaaatttccactagtgaaatgactcaaggtaactcaaccaaaattcaaaggaggcttgagcatcagggatctacaagcacacaacaaagctatgctcttaaaatggctctggagatatggacaggaggaatctaggctatggaaggacatcatagttgctaaatatggagcacacaatcactggtgttccaagaaaacaaacactccttatggagttggtctgtggaagaacatcagcaaccactgggatgaattcttccaaaatgtaactttcaaagttgggaatggaactcgtattaagttttggaaggatagatggctcggaaatacacctttgaaagacatgtttcccggtatgtatcagattgccttgaccaaagactccactgttgctcaaaatagagacaatggcacttggtgcccattttcagaagaaatttgcaggattgggaggtcaacagcctactcacaatgttaagctccctagaaggtcataatatcgaagatcaacagcctgacaaacttatttggggaaattctgagagaggcaagtacacagtcaaagaatgatacattcacctctgtgaccagaatccaataatagataactagccatggaaacacatctggagaactgaagtgcctaccaaggtgacttgcttcacatggttgactctaaatggggcatgtctcactcaagacaacttaatcaagaggaatatcatactagttaatagatgctacatgtgccaacaacagtcagaaagtgtaaaccacctattcctccactgctcagttgcaaaagacatttggaacttcttctacactacctttggtctgaaatgggttatgccacaatcaacaaagcaagcttttgaaagttggtatttttggagagttgacaaatccatcaaaaaaatctggaaaacggtgccggctgcatttttttggtgtatttggaaagaaaggaaccgaagatgttttgatgacatattaactccactctactccctcaaggctgcgtgtttagttaacttatttagttttgtggattttattagctccctgatagtagcataggcttttgtaaatggagctaattatcctatctcttttgtactctttgcatcttcttgatgccttttaatgaatctaatttacttcataaaaaataaaaggacaagttgttgaaggaggaaaagatgtgagtccatgtgatttagcaaggataaggtactaaagtccatttgattcacgcccggtaccaatgatccatcccgcattgcattcctgtattaaaacagagtcatcaagaaataaaatagagcaaataagtgattggccaaacgactagtggatatgagattaaaaggactatcgggaacataaagaactgaattcaaaggtaaggaaggaagtggactagcttaacctattccagttgccatggtttgagaatagttggccattgtgactgttggaagtgattgagagtaagaaatagtagtgaaaagagatttgttaccagaaatataatcagatgcaactgaatcaataacctaagagtcggaaaaagaaacacaagtcatgttattacctgtttgaacaatagaagttatctccgaagaggattatttacatgttttgtactgatggaactcaatataagccgataaagaaaccatccggatattcaaagtattggatcaacagcttataagccaaaagcatccgatacgagtgccattataatggatcaagagagatcaaacaacaaatcaccaaatatcataaacaaccaagaatctcgctggaatgtgaacaaagattgaaaaacaacaatgtagctcgccaaaaatgtgcaaagtgatcgaaaaatattgaatcgtgagtggagagaaataggagcttcaatcgacccacacagtaccaaaaaatccaaaaacggttgtcggagctcaagaaagttgtcaaaaagtatattgtatgcttcgaaagtagccgaaaaaggttggaagtgggatgtgtcaactccgaattatgatacgagcaccacagaagatcaatttgtgtcaaaactaccgaaaaaaatacttcacaccccgacgcgtggagtactcgctcgttggaacccttgctgccaacgtcgcatgtaggatcagttttcgaagaatcttattggggtttggtcgccggacgatgtcggatcttgtggtgccgttggaattcgcacaaccctgaaggaaaagaaggttacacaaatcagatctgaaagtcaccgaaaagacacatggcgattgacttttttgtctcagatgtttctcaccgtcgctctgataccagttgttgggctcaactcgtttgaagatactcttaacatagtgtgatattgtcccttttggaatgtgagtcatcttagctcggtaagcatactcgctcttccaactagcccgaagatacttttaacagagtgtaatattatctgctttgagccaagctggcgcggttttcatcaaaagacctcatactattaaaagatccatacaccttatatgtaggcttctaagttgctcggacacgggtgcgagtacccgacacaggtgcaaatctagaggtcagatcctttaaaatgtaaattctaagatttggggatacgaatcctagtacggatacgggtgcgaggatccgattaaaaataattcaaaaaaataagaaaataaaaaagtctctaaattatgtgaaattttgtggaataactacgtatagcttgtaaagtgtggatttattttttattctcaagttgtagataagtaaatgattgatttcctagataaggtatgttattttcttcaaatttaccctagtttggttcgaatttcgggaaattgtatcttgtctcgaatttttccttctgtcctgattaaactactcaaaatcgtctgaccagatccggtacggatcccatacccacatccacactagtgtcgtgtggacaagggtgcggcacctaaacttccgtgtaggagcaatttaggtaggctcctaatcttttcagctattaatgtgggacttttacgcacctctatcaaattccccaataaactaagtttcacgtggtccatcatcgcaatccacgggtctcttcctctagttaagtcccacatggcccattaccatgatccacgggtcaattttcgtgattcatcgtgtgccacccacatcgttagtatttatggtaactaaagtacgcaactagcttttgcttgtgagcgtgtctccaagctcgtaaaggtaagaaaaccgagccgcatattccatcactctatcatcaccatactcgtcccgcgaaacttgtaagataaaggtggctggttggtcagttgaactacctcagagtgacttggtatagtatttcctttcttgtgaatatttaactcaattatggactctctgtgtgatagtcattgagagccattttctatatagccggtgcacacaaatcatatgtaccaagcttgttatatatgtaactaatacgaggaccagtgaaggactcggtgaaaatatctgcaatctggtcattcgacatacaaggccaatagactccccagcaataaaatcagggggttgctgataaatagaattggccgaaatgttgccagaaaaatttgaaaatagtgagactaagccgaattctacactacaaaataggttttaaaacacaaccagaaaacaaaaacttttttggaaattactgttcacatcgaaaaaataaaagttgtcagaatttgatgtaatttatatggataggctcgtaatcactggacgagtaagttgtcctgaagaagttttgtcaaaaggtggccggaatggctcacacatgccggaaaacttattgtagctcgccggaaccctagttctggcggtgcgtagaggcgtgtgactttctgccagactgattgactgtggtttgtcgcctgacttttcctaacaagatggtagtattggttttcgcacaacaattaccgatgaggagataacgcaaatcaatcttgagtcgtcaatcggaaagacgcacggtggctgactttctatttagatgggactggaatttctggagtttaatcgcacaagcgttttggatctgatggtaatactggtatgcacagtaccactgtagcagtgatgaaccctcaaaataagacaaagttgccagaaaattgcacggcgatgagatctttcttccggatgtcaccggaatgacgcacaacgataatttctcactgaagctctgacaccatgtgagaatacacgggagaaaaatctatttttattaacaatgatacaatgagccctatatataatacatattctactctactacatatgggaatagggcatattttactcctactacatatgagactaggactatttacacataactatctaacaagggctatatctcagatttatgagaatatctacccaacgacccagagagacgagcctaatcattttgcagtggcacagactataacaacaaaaaacctactcataatggttaaaccaactgattaagatgcttacaggactatcttgagaaatgtacatattatatagatgcttgagttgcgtcccaatcctaaatagaagcttttattcgtaagcaagaagggaagcagctttacttgagccaatagctttcaaggtgcatgttgtcacaccaaggacatccagaatttgattttatagtgggaatatcgtttaaagataaaaaagatagcgtgcagaagattgcatacattagagatgcaaaatacggaatacccatactcccagataatgcagtatgccttttgcatgacctactggttgaatggaagcacctggtgaatttactaggtgtgttagtgatttctgctgcttccttcccctttctaaactgcatactatctaaaatgttaggggggcagaagcccagtcaatctgactaggtgatgttagtggtttccgcttcttcctcccacttctaaatgcgtactttctcaaatttaggagcatagaaacttaagcagctgcctacctgaggagttgcatgggaacataagagaatagactttacctgtcatattttccataccttagttaattacagtgttatcctgataatgatctgttttctggatctaggctgaatcgagattcaatcgcttttggttgaaaggatgctgctacagatccttagtttacatcattttggttcttattctataagtacttcccctatcaactacttccttcttttttcttaggttatttgcctctttaggttgtttggaaggaaaggaacagtagatgttttgatggaatagcaactccaaaccacttccttaaggctaatatcctgattggccaagtttctccaaagtccaaaacactttttttttccttcaaaaaagtacctttttttttcaaagttgaggtgtttggccaagcttttggaaggaaaaaaagtgtttttgagtagaagcagatgctcttgagaagcagaagaagtagcttcttcccggaagcacttttgagaaaaataaatttagaaacactttttaaaagcttggccaaacactaattgctgcttaaaagtattttcagatttattagacaaacacaaactgcttctcaccaaaaatacttttttgaaaagtacttttcaaacaaagcacttttcaaaataagttttttagaagcttggctaaacaggctataaatgtcttttatttttacagctggagtaccctaacacctgtaaattcccctatacatttttttcgactttggtagctcattaaccctagtataggactctttgttttggagctagcaaactcttttgttttcctatttttgcatcttcttggtgccatttataatatctcttcaccaaaaaaaaaaagttcccaaactatgactaccttgagttggtcaaagcataaccaaagcatgggcacaccagtgtttgcgtgaattttatggatgttccttacctttatccttctgtgcttatgtagcatctgtcttggtcaatcttttctgaagtctatattgtatttctgtgttgcaacatgagtttactgttaatcttactgtttgacctcaattttgggttctttttgattttggaagacatcgtttaacaggttggcatggctgctactcttgctggtgtctgtcaggtgcctctcactgcggttttgcttctctttgaactgacacaggattatcggatagttctgcccctcttgggagctgtggggttgtcttcttgggttacatctggacaaacaaggaaaagtgtagtgaaggatagagaaaaactaaaagatgcaagagcccacatgatgcagcgacaaggaacttctttctccaacatttctagtttaacttattcttcaggttcaccttcacagaaagagagtaacctctgcaaacttgagagttccctctgtctttatgaatctgatgatgaagaaaatgatttggcaaggacaattctagtttcacaggcaatgagaacacgatatgtgacagttctaatgagcaccttgctaatggagaccatatccctcatgctagctgagaagcaatcttgtgcaataatagttgatgaaaataattttctcattggtctgctgacacttggtgatatccagaattacagcaagttgccaagaacagagggcaatttccaggaggtagcttcttggtacatttcaatattcttaactgatgaaaaaataagggaaattgatctagcatgaaatgaagctaattataagttttacacagtagaactggtaaaacagggttggctggatatttctttgttgaatttttaggattatatatattgttttagttttgtaggttgttttctgatgtgctttttgactcggcagaatcttaagatgaaatggaaggttgtatcatcaaatgttaaataagggaatatgtgactttcaaagttaagcacggagtattttggagtcaatagttacttcctgaatcttttaggatggaggagacagtttctataggaataggaaaaggggacctgatttcattatttgtgtgtatatacatttgttatctgaattcgcattactttctaacaaccaacaaaaggaaagtggacattcaatttgagccggagggagaaaatttaactagaaaatgacctggccgtgaaataaaattattgatccgtcctttaactagttttcatggattgcctccttgcggatgatttttccaaccggtagaactactgttagtcgtccaaattctgaccccctactatgaataaaaatgtattagtaagtttagtgggtaatctccttgagaaataaaggaacaggagaaatattttattgatatatgctaagtgttttacaatagccctatttatatacaatgtttacataaacctaaagccttctatataaatgtgggacactatacatgaactaactctaacactatccctcaagctagtgcatataaattatatatatgcttgttacatatataattaatttctctactttttggtatacttcttgtatacgggagttatctcccttttgattaatacaatttaccttatcaaaaaaaaattaatacgaggaccagtgagggacttggtgaaaatatctgcaagttgatcatttgacttctcaaactttgtaacaatatctcctgagaatcttctctctcgtgaagtgacagtcaatctcagtgtgtttggtcctctcatggaacactggatttgatgcaatatgaaggacaacttgattatcacacacaagttccatctgactgattgctccaaattttaattatttgagcaattgtttgatccaaactagctcacatggtgcaagagtcatgactcgatattcggcttctgcgctagatcgagcaactacattctgtttcttgcttttccgagagacaaattacctcctattaaaacacaatatccagatacgtaacgtctatcagaaggtgaccctgcccaattagcatctgtgcgtccaacaatatgctcatggcatcgatcttcgaatattagtcatttgtctggagctgattttatataacgaacaatgcgaacaactgcatcccaatgactatcgcaaggaaattccataaactgacttacaacactcacaggaaataaaatatcaggtctagtaattatgaggtaattcaattttccaaccaggcgcctatattttgcaggattgctaagaggctcccccctatcctggcagaagcttagcattcggattcataagagtatcaatagttctgcagcccattattcatgtctcctcaagaatgtctaaagcatacttcctttgcgaaataacaacctgaactagaccgagcgacctcaatacctacaaagtacttcaatctgctaaggtcgttagtctggaagtgttgaaagtgatgttgtttcaaattagtaataccatcctgatcattgcgagtaataacaatatcatcaacataaaccaccagataaatacagagattaggagcagaatgccgataaaatacagagtgatcagcttcactattagtcatgccaaattcccgaataattgtcctgaacttacgaaactaggctcgacgagattgttttaaaccatagagacttgcataagtgacatacaatacctctagactccccttgagcaacaaaaccaagtggttgctccatattaactttatcctcaagatcaccatggagaaaggcattctttatgtccaactgataaagaggccaatgatgaacaatagccatggacaggaaaaggcgaacagatacgactttagccacgggagaaaagtgtcattattatcaagcccaaatagctgagtatatccttttgcaatcagacgagccttgagccaatcaacctggccatccaggtagactttgactgcataaacccaacgacaaccaacagtagacttacttgaaggaagagaacaaactcccatgtaccactcactcacatgtaaagcaaacatctcgtcaatcatagcctgtcgccatcctggatgagatagtgcctcacctgtaaacttaggaatggaaacagtggacaaagatgatacaaaatcataatagggtgatgagatgcggtgataacttaaaccaacataatggggactaggattaagtttggatcatacaccctttcgaagtgcaatcagtggactaggaggagccaagtccgcactagacgtggatgacaatgataagtcaagagtggtggcctcgtggttggagatgtaggatgagcaactgtagactcctcagaagtcggtataggtaggagtacctgtgatgttgatgtggatttaagaggaggaacaatagattcctcacaagtagatacaggtaagacctcagatatatcaagatgattagatgaagtaaagtaaggttgagactcaaaaaatgtgacatcgactgacataagatatctacgaagatcaggtgagtagcagcgataccccttttgaacccgagaatagccaagaaagacacacctgagaacacaaggagctattttatctttttcaggagctaagttatgaacaaatgtactccttaaaacactaggaggaaagagtataaagatgacctagggaacaatactgagtgtggaaactgattctagatggaagatgaaggcatccgattaattaagtaacaggttgtaagaactgcatcgtcccaaaaacgttgtggaacataggactgaatgagaagtgtgcgagcagttttaatgagatacctattctttctctctactaccctataatgttgaggagtatacagacataggataatattttgagaagtcataaactattgaaactaagagaatacatattttaaggcattatcactacgaaaagcgaataaaaacaccaagcggagttttaatttcagcataaaaactctagaatattgaaaacaactcaaaacgatctttcatttggaaaatccaaatacatcttgagtaatcattaatgaaactaacaaaatccaaatcttaaggttgtgactctactaagaccccatatatcataatgaactaaagacaaaacagactctacacgactcttagcacgacgtgaaaatgtagctcgaatatatttcccaagttgacacgaatcacaatctaatgtggacaaaccagacaccatcttctgaagcttggataaactcggatgtcctaaacgtttgtgaattaggtctagaggatctgtagttggacatgttgtagagggattgagtgagttaagatagtcaaggtcttgtgattcacgccatgtgccaatcgtctgtaccgtactgcggtcctgcatagtaaaagaatcatcaataaaatatatatcacaatggaattcacgagtcaaatgactaacagatgcgagattaaaggacaaccggggacataaaaaatagaatctaaagtgacagaggacatgtgattagcttgtccaactccttttgcttttgtttagacttcatttgctaaagtatcattgggaagagattgtgaataaacaattatttgacaaaagtgacatattaccactggggtatcaagttgcttagtcatactaagaatgtttgggagagggtggtggaagtgagggtaaggaggacagtgtctctatccgagaaccagttcggattcatgcatgatcgttcaactgcggaagctatccgtcttattaggaggctggtggaacagtacaaggataggaagaaggatttgcacatgatgtttacctagagtaagcgtatgacaaggtccctaaggaggttccttggagatgtcagaaggttaaaggtgttccggtagcatatactagggtgatgaaggacatgtatgatggagctaagactcgggttaggacaatggaaagagactctaagcattgtttggttgttatggggttacagtaaggatctacgctcaaaccgttcttatttgccttggcgatggacgcattaacgtaccatattcagggagatgtgccatggtgtatgttattcgcggatgatatagttctgattgatgagacgcgaggcggtgttaacgagaggttgggggtttggagacagacccttgaatttaaaggtttcaagttgagcaggactaagacagaatacttggaatgtaagttcagcgacgtgacggaggaagctgacatggacgcgaggcttgattcataagtcatccccaagagaggaagtttcaagtatcttgagtcagttatacagggagaagatggggagattgacaaggatgtcacgcaccgtattaagggcggggtggatgaaatggaggttagcattcggtatcttttgtcacaagaatgtgccaccaaaacttaaaggtaagttctatagagcggtggttagaccaaccatgttgtatggggcagagtgttggccagtcaagaattctcatatctagaagatgaaagtagcagaaatgagaatgttgagacggatatgcgggcatactacgttggaagattaagaatgaaaatatttgggtgaaggtgggcgtggccccatggaagttgtgcccaccattaaagactgctatctgaaaactaattctttgggcccaaacattctggcccaaagtacctcgtgaataataatattgagctcatgtctgacatgttggaagaggagttactagcaaacacttatacacctatgttggtaacacaattgaagaactacgaaaaacactcttctgcaaaggaaaatgagaagaagaagaagaagaagacgaagaagaaggatgatgcaatgatcattgaagaaaaaggagagcaggaggacccatctaaacttacaaagtctagaggaagaggaggacccagagtttgatgcttccctctgggtacaccaaaacatcgtcaaacttaggcaaggagtttggggtaaacattcaggggtgtgagaaggaagctttggagcttttcgtaaaattacaactagaggcataaaaaaaaaaaaggcaatccaggcatggaggtgacaaccttcgaaaagaaagggattcaaagaactgaaagggctggatttttggagtaacttcaagagtaatagaacaagaagtagggggttgcattattatcaaagatcaatgaagattaacattgaagaagtgggaaatccaaaaagactccaccgagaaggatgatgcaatgatcattgaagaaaaaggagagcatgagaaaaaacccgtagaaattgacagcactcacacacaataagacgagataataaagtagtgagttggccaattgaagaagctttacctcttaacttacaaagtctagaggaagaggaggacccagagtttgatgcttccctctgggtacaccaaaacatcgtcaaacttaggcaaggagtttggggtaaactttcaggggtgtgagaaggatgttttggagcttttcataaaattataacaagaggcatgggaaaaaaaaggaaatccaggcatgcaggtgacaaaaccttccaaaagaaagggactggaagaactgaaagggctggatttttggcgtaacttcaagagtaataggacaagaagtacgggattgcattattatcaaagatcaatgaagattaacattgtatcatggaatgtcagggggttaaatcgacatagaaaaagaatgttgattaggagtttaattcataggtggaaagcagatgttttctgtttccaagattcaaaattaaaaggggacattagggagtttataagagaactatgggcaaataggtggtttaaatatgcacagttggaggctagtgggcctagagggggtattattgtcttatgggatagtaaaattggggagggggagatcagcagcctgagctcctattctgttacttgtaaatttataggtaaaactcaggagtatacttggaatttatccactgtatacgctccaaatgatagggaggaaaggaaagaagtatggtgggaattagcaggtgccaggggaatttttatggaccttgggtaatttctggggatttcaatactgtgaggtacccaccagagaaaaagaattacagcaaaatcactagagcaataaatgaattctcataatttattgaagatatggaactggtggatctacaacttgcaggaggaagttacacttggaggacaggagatagacatgtgataacagctagactggataggttcttggtttttatggattggaatgagagcatcagaaacaccaagcaatcagttctccattgaattacctctgaccattcccctgtgatgcttcaatgtggtaaccggtaccctgtcaaatcctattacaagtttgagaattggtggctggaaacagagggcttcaaagaaaggattaaagtctggtggagctcttttgcttgtgaaggaagacgtgactttattctggctttcaaacttaaagcatcgaaggaaaaaattgaagaaatggagtaaatctattcaaggaaacttggagatgcagaaattgagtattcttagtcaacttgcagaactagaagagacacatgatcaaaggagccttactgaagaagaaatacacactaaatatgcagtctatggagtttggggagattgcaaaacatgaggaggtggcttggagacaaagatctagggctctttggttgaaagaagggacaaaaacatcaattttttcctcaaaattgcaagtgcacataggaaatacaataacatagaccaactgttacttgaaggaaaatttgtggcgaatccaacatacataacaaataatattggtacattttatcaaaaactatatataaagattgctagaggacaatcttatgttgcaaagtcttttcgaagcttaggaaatttgggatagtgtcaggcatgtgaaagggataaagcacctggacctgagaactgggaggtgataaacacggatatgatagctgcagttctttgttcatggaatgtttgaggaaagctttaatgttacctttgtggtattgattcctaagaagatggaagctaaggaatagaaggactttaggcctattatgataggcaatgtgtacaagatcttgatagaaagacttaagaaattggtgaacaagttggtgaagggtcaacggatgacttttattaaaggtagacagataatggatgttgttctaattgccaaatgaatgtgtagatgcaagaacaaaggcgagaaacctacaatactatgcaaactagatattgagaaggcatatgaccatctaaattggaactttctattggaatcgctgatgaggatgggctttggtgtaagatgggtcagctggatcaaattctgcatcagcacaatgaaattctcaattttgataaatgtttcaccagtaggtttcttcccttctcagagggatttgagacagggtgatccactatctccttttattattcattagtgctatgggaggcttaaatgatatgttaaagactactcaagataacaactgcatacggggttttaaggtgaagtccagggcagacagtactattgagatttttcatcttcgatatgcagatgacgcacttatgttctgtgaggttgacaatgaacaattgaaagtgctgaaggtgatcttcattctgtttgaagccacatctgtattacaaattaactggaatgaaagctttatctatctagttaatgaggtaactaagatccactttttggttggaatcctagaaggtaaaattggggaattgcctacagttatttggggatgccatgggggccaagagcaattttaaggggatttggactagggtcgtagagatatgtgaaaaaattttaacaaactggaagagttagtatttatccttaagggacaaactaatactaatcaattctatacttgatgattttcctacttacatgatgttcctcttctcaatccatgtgaatgttgtgaagagaatatatacccttagaaggaacttcctatggggaggaaactatgacaaggaaagatctatttggtcaaatggaagtctctcacagtcagcaagaagtaagagtgttttggaatcaagaattggagaattcagaaccaaagtttgatgatgaagtggctatggagatttactacagaagaacattgtttgtggaaagaggtgatcatggagaagtatggcatagaagataaacggataacaaagtctgtaaatagatcttatggagttagtcgatggaaatccatcagggacctatagcttcagctcttgaataagtccaaattctgaataggaaatggattgaaaatatctttttggaaggataattggctaaccaaggaactttgaaacaactctttcttgacatttacattccaaatcaacagcataaagcaataatagtagaattatgggctaatcaaggttggaatctcacatacagaagactatcaaaagacccggagattggcaggtcaacagagttcaaaggcactttggaacaatttaaagaggtctatacttctatagactatttgacttggcaagggaagtttattgttaattcagcctataaggaattcaacttctcagctaactggattggttgttggccatagaagttgatttggaaagttaaaattccttatagagttgcttgtttctcttggcttttggctaaagaggcagttctgacgcatgataatctaaccaagagagattaccatttatgttcaagatgttatttatgtgaagagcaggcagagacaaccaatccacttttttttgcattgtaagttcactgcagttatggaggattttcattagtttaaagggtatcatgtgggctatgcgtagaagtatacctgaagttctagcatactggaaaaaagaaagaaatctttccaattataaaaagagatggaggattatcctagcttgcatctggtggaccatttgggaagaaagaaatcaaagatgcttcaaagataaatcagtcatattcagataattaaaatgaagtggctagtcttgttttatttttggtgttaagtgttagatagttatgtattatgtataagttgtctagtcccacattggaacgggagtaatatgtactatgtagagtatagctataaataggacttcttgtactttattgtagagaatatattaataatatatttttcccgtgttgtctcacatggtatcagagaaaccgtgagatatcagtcgttgtgaaaaataccagcggcttcgggaagaaaaaaatcaatcaactgctaggtatattagtcttcggcgaccgatccattaaatttctctggcaaagaaccactcatgggccctcacgcgcccaccgaaagaaatatttccggcgaggttccaatttcatgcgcccgcgcgtgaggcagtttccggtcaaattttgacaaaggtcctttttgacagtttgttcaccctgtaattcccagtctatccatcattttttttatttcgatcacttcgcaatttctcgggcagctacagtgatttttccggcagaagcggtgtttcctttgcctgcttcagcgagatacagttgattatttctattatttgtttctagacctctctccaatccaacgatgtctttggaatttgatgtatttggttctgaaaacacgagttctagaaagtcaagcttcatgattactttagagccattaatggggagttcaaactatttagcttgggtttcctctgttgaattgtggtgtaaaggtcaaggtgttcgagatcacttaatcaaaaaggctagtgagggctgtgaaaaggtcaatttaagcagtttatgacgtctgtataccactcagcagaataggatagcaaagaaagaatatgcacatcattgagactgctcgcacacttctcattgagtctcacgttctgctacattttctgagcgatgcagttctaacggcttgttatttgattaatcggatgcctttatcttccatccagaatcagattctgcagttagtattgttttctcagtcacccttatacttttttcgtcctcgtgcttttgggagcatgtgtttgttcataacttagctcccgaaaaaaataagttagctcctcgtgctctcaagtgtgtcttccttggatattcccgagtttaaaagtgatattgttgctactcacctgatcgtaggtaccttatgtcagttgatgttgcattttttgagtctagaccttactttacctcttctgaccaccttgatatatatatgaggtcttacctataccgactcttgaggggtttactatagctcctcctctacatactgagccacagaaatcttactcatacctaccattggggaatctagtgttgctcctcctagatccccagctacaggaacacttttaacttatcgtcgtcgtccgcgcccagcatcatgtccagctgattcacgttctgcacctgctcctactgcggactagtctcatcctaatctaccaattgcacttcggaaaggtatatagtccacacttaatcctaatccatattatgtcggtttgagttatcatcgtgtcatcacctcattatgcttttataacttctttgtccactgtttcaattcataagtttacaggtgaagcactgtcacatccaggatggcaacatgctatgattgacgagatgtctgctttacatacgagtagtacttgtgaacttgttcctcttccttcaggcaaatctactgttggttatcgttgggtttatgccgtcaaagttggtccagatgaccagattgccaaagggtatagtcaaatatttggggcttggttacagtgatattttctctcccgtggctaaaataccatcagttcatctctttatatccatggttgttgttcgtcattggcatctctatcagtttgacattaagaatgtttttcttcacagtgagattgaggatgaagtttatatgaattaaccacctaattttgttgcttagggggagtctagtggctttgtatgttggttgcctcagacgctctatggtctaaagtaatctcctcgagccttgtttagtaagttgagcacagttattcgggaatttggccaactcgtagtgaagcttatcactttgtgctttattggcattttacttcaaatctctgtatttatttggtggtttatgttgacgatattgttattaccggcaatgaacaggatggtattactgagttgaagcaacatctctttcagcacttttagactaaggatctgagtagattgaagtattttttaggtattgtgattgctcagtctagcttaggttttgttatttcacattggaagtagaaaaacttcaatcatttttctttatttgaaaggaagaaaaaaaaggtaatatctagacctaaatattaatctgaagacaagtgaggcttgctcagttggtaaaagcacctccacctacgatcgttaggtcctgggttcgagtcaccatggaggggaagtgtggaaacactatagatcctcctaatttgggagggggaaaaaaatattaatctgaattgacatgaatctcaatgacaatgaccaacgatttcctgcaattcttttcagtatggaatgaataaaaaatcaagctacaagtctctattaaacgaaatgcactaacagggatcactctcaagaaaggaagtggttttggttgttgttattccaggttggataaatcactttctttataaatatcataaaagacaagggctttcttgcttcagcacatgtgggaaatgccggggggcttggctggtaccaagctcgagcggtctttctatctttttggattgcatgcccaaggcaatgctttttgtagattgggatggattgatcttcgcagaagtatgctttagacattcttgaggagacaggaatgacggattgtagacccattgacacacctatggatccaaatgccacacttctaccaggatagggggagcctcttagtgatcctgcaagatataggcggctggttggcaagttgaattacctcacagtaactagaccttatatatcctttcctgtgagtgttgtaagtcagtttatggactctccttgtgatagtcattgggatgtggttttccgaattcttcgatataaaatcagctccaagcaaagaactgttgttcgaggatcgaggcccatgagcagatgttgattgggcacgatcaccttctaatagacattctatatctggatattgtatgttaataggagttaatttggtgtcttggaagatcaagacgtaaaatgtagttgatcggtctagtgcggaagcaaataatcgagcaattgttatggtaacacgtgagctagtttggatcaaacaactgctcaaagaattgaaatttggagaaattgatggaaccagtgtgtaataatcaagcagctcttcatattgcgtcaaatccggtgttccatgacagaattaaacacattgagattgactctcactttgccggagaaaagatactctcaggagataccgttacaaagattgtgaagtcgaatgatcagcttagagatatttttaccaagtcccttgctggtcctcgtattagttatatttgtagcaaactcggtatatatgatttatatgcaccaacttaagggagagtgtgagatagttatgtacaacaaaatacccggtataatcccacaagtggggtatggagggtagtgtatacgtagagcttacccttaccctgtgaaggtagagaagctgtttccaaataccctcggctccagtacaaatgaaaaggagcagtagcaacaagcagtaacaacaatgatatagtaaaataactgaagaaagaaataacatgtagacatataactccactaacaaacatgcaaggttaatactattgccacgagaatggcaaaggaatgttagatagttatgtattatatgtatattaatagtctagtctcacgttggaataggagtaatatgtactatgtagagtatagctataactaggacttcttgtaatatattgcatagagatatcaataatatatttttcctgtgctttctcacgtaaaggaatgtaatgtacttagaagatcatgaatctatctttgatgttttagacacctcgtgagaacacaaaggtttaggaactttattgtgttctttgtaattatgggtgactgccaatatgttaccttttcataaaaatgattatttggccattggattagtttcaacagcctctctgcccctccgggtaggggtaaggtctgcgtacatattaccctctccagaccccacttgtgggattatactgggttgttgttgttgttgttgtggattagtttcaacaattttgatagttcttttatttgaatcaaactactcattcacatggattttgtatcgtatcattgagttaaaaaaattggttttgctaatttatcctcatgtataacaactacctatttttcaatatattggattcaggagcttgtagtagctggagtttgctcttcaaagggcaataagtgccgggtatcatgcacagtgactccaaatacagatctcctttctgctctaactcttatggagaaacatgatctaagtcagctacctgttatactaggggacgtggaggatgaaggcatccatcctgtgggcattttggacagagaatgcatcaatgtagcttgcaggtttttgacattcaacttttacttcaaagatataatgctttctggaaccattgatgataaaatatgcaagaaacttgtgcagaagtcgcactttactatcgattaccagataaagttacttatcaagaagtcaaatatattgaacatatttctctaaaacactttgactggactgtaagcagaaacttactaaagtaggtcgtaagaaatggtttgatagggaaatcaccatctacacttaaaagagttgtgtgaatttgaattcttaaagcatgtgaaagttataaaaacttgttattatctaagcatctgaagcattttggccatccaaaggatcaaaaataggaaataatttcatttgtacaatgaactccctgcacaaattctcacactaggtgtattctctattcatcactagcactacatgtgtcactacgaatcatatacaataaatctttgtaacataaaagacgacacataatatggaagtaagccgagtatacaagggaagtttcatcattacggtgagctttttataagataatcaagttttactggaaaagggcaaaaactctcccgtatagaagtataccaaaaagtagaataccttacaaaaatatgattttctatgaacaacaccctatcttctatacttgtagggatctcatcggggcaccaaaaagagataaagggataagaggcttttcctcaaatgtacaaaatccttctctattccttcaaaagctctcctatttctctctctgcacactgtccacataagttcaatggagcaacatccacgccctgtgtcttcttttccgtcttctataggtccagctgaacatggcttctttgactgagtgtggcatcaacgttgaagaccaaaccatcccagtacttccaaccacaaacgagacactatatgacaatttagaagaagatgattcacatcttctcccgaacatttacacataaaacaccagctgatacatgtaatcttcctcttcctcaaattatcagccgtcaggatcacccgtctcgtagctaactaggtgaagaagcacacctttctcgaaaacctcaggatccatacagagagatatggaaaagctgattcctccatgcccagaagcttctcataataagacttaacaaagaaacaccactacttccccccccccccaaaaaaaaaaaatctccatacatcgactttcatgtgtaattcttgttcgtgaaacgacccaatcaacctttggcacaaatctcccagtcttgcgagttcctcctaaacttcaaatcacaatgaacttctccaccttgtagcctccgtgtcccttggactggcaactcctttggcatgaaactttgtacatattaggagatgtgatactcaaagtgttgttcctgcaccaattgtacccccaaaaaacttaccatgctcccatcacctaacattgaatgatacgttccaaaatcttcgcactccttcaagaaacttttccgtaggccccacccataagggagtgtgattttttttgctctccatcccctctccaagaatccattccctaaaccactgcaggacactttaacaatcactatgtcactttttctactagttctacattgagtgatatcttgatgtcattgaaatgcctctggaaaatcttcttctcatctaaaagaacacttgtttgccttttgaatccccctctaacattttctatgtttcattcatctttggtggaacagagcattagcaactagagaacagctttgctag

SEQ ID NO: 4 - DNA, 36700 bp, Nicotiana tabacum
atgaatcacgaaagttgttgggtcgtcatccaaattgctggccttgggctcgacgaccatctcttcctccgggacgttcctgtgacggaaacattgaaaaagaacaagatatgtgcgacagcagcaaagacgatagtgatagtgatagtggtatccagataggatctctgctcgaggaagttatcccacaaggcaataataccgctataatctcggcttgctttgttggcctcttcaccggtatcagtgtcgtgcttttcaacgctgcggtaagtgcgctataggtctttcatttctcttttcatctactattctcccttacttacttggcctcagtcaatcagccccctgcctactttaaattattgtacaatttatcagaggagtatcctatacatcaaattcacataacttagtaaaatatgctgacattctgaattttaaccttaccagcttagaacatccaggctagttcagaaacagataatctaaattggcctcatttataagtcattttgttaatcaagacatacaatttggctcttgataaaagattatgcagcgcccgatgataacctaatatttatcagcaacccatatgtcactttcttttgtttaaatgctctcccatgtaatttaacaatattgtcaccatacaaaagagaactgaagtgaatgttccatttgtggtcatataacggatatctcccttggttaggttcatgaaatacgtgatctttgttgggatggaattccatatcgagctgcctcagaggagcccattggagtacattggcaacgtgtaatcttagtaccagcttgtggcggtttggtagtcagctttttgaatgccttccgagccactctggaggtttcaactgaagaaagttggacatcatctgttaaatctgtgttggggccagttttgaagacaatggccgcttgtgtcacattaggaactgggaattccttaggaccagaaggccctagtgttgaaattggtacatctgttgccaagggagttggagctctgcttgataaaggtggtcgtagaaagctgtcactcaaggctgctggatcagctgctggaatcgcttctggtttgttccccatattattcttggttctgaaccatacatggtacattttccttataattacatgtagcctgttgtatgctttcctctttcctgggaagcctttctgtaaatgcaaatgtgtttgcactcaaaccaataaactgtaaaaacagtgaaccccttgagcaagcaaaagcactagaaaaccaacaaatagatcccccccccaagataccagtgaaatgacaccgggtgacccaaaaataaagcagcttacatcttgactttgagaggaactgcaatcagctataagtaggttattaatttccagtgcctgcattctgcccaagtactatgatatatttctgaagctttgtttccccagttcctttttcagacgtttgctgtcaataaagttgagccagccaacttggttcccacaagctactaattttgtccaagcttactctatgggagaagttaaatttcccaaattccttgagcagaaaatgaaaaatgaactcaaagtgtcatattaggcaactatctaaagaaaaatacttaattgaagtttagataagaaaagtgaatatatattgatgtagtctccgttaggtgagaagcgcatcacttacccagcaacatatggacctaaaatttactagtgaacttttcacattgtatcaaaagctcaacaaacagaaagatgactagtcctaaaatgttatttcacatcaaccttatcatacgtgcattatttgttctctatatttctatttcatccgatataaccaatcgtcattgtaaattctataatgcctgtggttacttttgtctttagtgacaaatgacatttaggctaaccatgtagttattgactgatttcgcttgacgtctcttccaattatgtagtagtagagtgttgagatatggatatgttaccttctaaaaaaaaagagtgttgagatgcggatggtttgctagctggcttttgtctcccttcaagttgaattagcaaaagcaatgtctcataagttggatagctagacaagaaaaactccaaattactttatgtagagtattcttaagcttgagtcgcgagttggaaattggaattatgtaaaaaaacctggaattatttggttgagcctgctttttatttttgtcaatatttccagtatctaacccaacatgtttagagcaattcccagagagcctcaatacgaggcatttgcagagtctttatgagagtccaggaaggggcacacactgtagaggtatagtgttgtccttatttttttttttttgataaggtaagattttattaaaaggtaccaagatggtgcaaaattacaaacatccaaactaatacaacaaagcaactacattcctcctagctcctctagaaaattcatatattgttccatatttttcattacatgtcttttacaccagaaatacaagtttaataagcatctgtttttaatcctggatacatgctgcctttccccttcaaagcaaatcctgtttctttccaaccatattgtccagaacacacatagaggaattgttcttcatactatctgttgactctttgccactttttgttgttgccatgtctccaacaaactttacactggcaggcattgcccacttgacatcatatatatttaggaagagctaccaacactgctttgccactttgaaatggatgattagatggttgactgtttctgcctcttcttcacacatgtaacaccggttacatagagcaaaacctctcttctgcaagttctcctgagttagaaaagcttcctttgctccaatccaaccaaaacgggctactttaataagtgcttttgacttccatattgctttccatggccaatttgactgataaagcccttgtagtttttgtaacaagctataacaactgctgactgtgaaaataccatcattacttgctgcccagattaatgagtctctcctgttttcctccaatctaacattattcaataactgcatcaattgggaaaattcatcaacttcccagtcattgaggcccctcttgaagattagctgccagccggtgcttgaatagaagtctaacactcttccatttttgttaatagagcagctatatagaccaggaaactttgatctaagacttccattttccaaccacatatcagaccaaaacagggtattattaccatttccaagtttcagtttcacaaactgactatatttattccaaagattactaattgtgctccaaactcccccttttgaagaagattgaattgaacgaggagcccacatgtccttcataccatacttggcatctatcacctttttccataatctattcccatcataattatatctccatagccatttaaataaaagacttttgttatgcatctttagattcctcactcctaatccccctctttctttttttttcatcacctcttgccatttgaccaagtgaaatttcttgttatcattattaccttcccacaaaaatttattcctcatagtattcaattttttctccactgatgttggcattttaacgagagatattagataagtaggtataccatccatcacactattgaccagtgtaagcctaccaccaagagataaatattgtcttttccatgacaccagtttactgctacatctatccaagaccccctgccacatctttgcatcattcttttttgctccaagtggtaggcccagataggtggatggtagctgctccactttacaacccaaaacatctgccagatcatcaatacaatgctcggcattaatactaaacacattactctttgccaagttcactttcaatcccgagacagcttcaaaagctagtagtactcctatgaggtgtaagagttgctctttttcagcttcacataatatcaatgtatcatcagcatagagtatgtgtgagaaatacagttcttccccctctctttttctaattttcaatcctctaatccaccctaacttttctgcttttaaaagcattctgctaaagatttccatcaccaacaaaaataaatagggggatattggatccccctgtcttaaccccctctgagaattaaagtatctatgtggactcccattaattaaaactgagaagctaattgaggatatgcagaattttatccacccaatccatctttccccaaaattcgtatgtttcatcagatttaacagacatgaccaatttacatgatcataagccttttccacgtcaagtttgcaggccacccctttaatcttcctcttgaatagatattcaagacactcattagctaccatagcagcatcaataaattgccttcctcttacaaaggcattctgattatctaatatcaattttcctatcaccatctttaatctttcagctatcgactttgcaattattttatagacactgcccaacaagctgataggtctaaaatctttcacttccgctgccccctttttcttaggaataagagcaatgaaaattgagtttaggctcttagtcttgtccttattttcagggttgaactagttctttagaagtttcctaggcttcctaatttccaaagttctgccaggtccttttctagtgaagtacttgaagtttaataaatcaaattttaatttctaacatatcccgagaaattcattcacaaattcaactggtgacttctgatgcagaaacataagcaactgcttatgggttcatatgttcctgcaattttattgttgacatggattggcttcatatggttttgttcctgcaattttatcgctgacactaatcctttcatatggttttatgtggggtggtaaatagaggttaagagacaagaagaggctggaaaaggtgggcagttcatttgttagtagactactctatttactaagagatatgatgtcccatacattactcgaattggctccaaatacagattccacttctttgtcgagtttccttattgtacagagttcgactcgtcaagggaaattcacttcctttgactgaataatgctagtttgagtagtaccttaaattaaatggaccatttaattctatctacttgatagaatagactggtcatcaactagttgcaaatataatgacaactccgccatgtttgcagagtcacctgatgaagaagtacctcaattagtagaccatttcttgaatgttctacagtattctctatgcctacatgaccacatcacttttccttttgcgttgtgagaacttgaacttggtgagcgggggttccccaggaatggcatcttggtggcagatgaccattctgtccttatcttagctaatgcttcttggattgcctcactagatttattatacctttaataaatgtttgccattgttctgccataatagagggatgtacctagctggtgcttcacatcacatagtccaaaactaatgaaatgctttacaattgtcgagtactaaaggatgatttgtggaatcagatctcaaacaatttattttgaggaagaaaaataccaaaggttttttctgtttgttggaagattaaaaatcctttaaaaggtaaagatttatgaacttaattcagcatttttgtggccattgctgaaaaagagaaaacaatggcacttattcgagtttgcttatccaaaaaaaaagaagaagagaatgtcacgtaatgcaatttcatcttaggaaactttgcaggagaaaagcaagagtgataaaacagaactatttgtttttttgataagttgttgtgacctatttctttgtcattcttatttgctaataagctaatgtaccctgtactatggttgttttgacttaatccggggatgttcagtgagcattttcttgttttttctgctgtcagcatctgctgccttacaggaattcattttctggaaatttacttcttgttctgctaacattttcctgttatatcttgtcagtcattttctctccatggttatactgtttgtgtcactttgaaactctccttgttttctactttaaaggatttaatgctgctgtcgggggctgtttctttgctgtggaatctgtgttatggccatcacctgcagagtcctccttgtacttgacaaatacgacttcaatggttattctcagtgctgttatagcttctgtagtctcagaaattggtcttggctctgaacctgcatttgcagttccaggatatgatttccgtacacctactggtaattttggacttctttctcgagtttgattcttaaatacaattgtacccgtcacttacagcaacaacaactacatttcaacagctagttggggttggctacacagatcatcactatccatttcaatttctttagtcccatttctttcgaatattcagtactttgggattctctattatcagaggttctctttattttctactttgacgtacaaatctctaaatagattaaagaagactcctagagacactggcctaatgcaaatgtaccaccatgaataaaccttaatctgaaatagctggtatcgtatataagaacctttagctttaattgtgttctatattgatcttttgggacaacttccgtccaataatattatgtcttacttatacagttatacttatccttaaactttactctttagagtggttatccgtagttcaagcttttgttggcaccatagctagtttggttcttagtaaaaagttactctttagagtggtaactttttgtcaattttcttagtgaaaatataacctctgtgacaaatctaccaagtataaatccaatatggttctgtgtcatacttgtagtttatccaagtctatgctccatcactcttacaaaggctcatcgtatgactaattttttttgagaaaggtaacagtttgtattgataataagatcagcgccaggttagtcattagtgctaatagctgtatgtacaactccaaaagagcaaaagacaagcacctggtgtaacgtaaattacaagctgcctataaaatctatcaggtctcctacctcactaaacatttcttgtttacaccaaaaaaataaaacaaggaaagacaatccatcttaatcttctgaatggagtttcttttgccttcaaacatctcgagttcctttcgttccatgcaatccaccatatacaagctgggatgcttttccatttgtctttatccattttttctaccaattcccttccaattgactagaagttccaatgtggttctagatatgacccaattaactcccaacatataaaagaacatgttccacggatttgtagtgattctgcaatgtaggaacaagtgagcattactttctacttcctgtccacaaagaaaacatcttgagcaaatctggaaacctcttctttgtaagttatcatgtgttaaacatgcttttttaccactaaccagacaaaacatgatactttgggaggagttttaaccctccaaatgtgtttccaaggccacacctcagtcattgaaacattatgatttagagtccagtatgcatcttttactgaaaatgcacctttgctattcagcttccaaactattttatctatggtcttgttagtttacagctatgtatatagtgtagtcttgtcccacattggaataggagtagtatgtccttgtatagtatagctataaataaggacctcttgtattgtattgaacatccaatatcaataacatattttctcccgtgctttctcacatggtatcagagcaattgtgagagatttatcgctgcgcataaattccagcgactccgggaagagaaatcagtcaccggaagtctttttccgacgactctttcaaggttgtttgcgtttgctttataaatccaacactaccacaagagtaatcactgtccggcgaccaaaccccagtaaaaatctccggcagcagcctcctcacgccaccagaagctcacgcgccggcgcgtacgaccacttccgtccattttttgaaaaacttccttcagaacagttgggtcgcctggtaattcctatcctacccctactgttttcatttcattccgaccactttgagttttttccggctgctacagtactattccggcagctatagtactattccgacaactacagtaagattccggctgctacagtatttcattattctgtttttgtgtttccttactctgtttcagtggattacaattgattctttctcttatttggtaataatttgcaacaatgtctatgggatttgatgtttttgggtctagaaacatgagttctggaagctctagtgttattattacctcagaaccttaaatgggaggttcaaactacttagcttgggcttcatctgtcgagttgtggtgtagaggccaaggtgttcaagatcatctaatcaaaccgtctagcgaaggagatgaaaaggcaataacactttggacaaaaatcgatgctcagttatgtagcatcttgtggcgatctattgattccaagttgatgcccttgtttcgtccattcctgacatgttatttggtttgggcaaaggcacacaccttatacactaatgacatatctcgcttctatgatgtgatatcgcggatgacaaactgaaagaagcaagaattagatatgtctacttacttgggtcaagtacaagcaatcatgggggaatttgagaagttgatgccagtttctgctagtgttgaaaaacaacaagagcagcgacaaaagatgtttctcgctcttaccctcgctgaacttcctaatgatcttgattcagtacgcgaccatattttagctagtccgactgtcccgacagttgatgaattattctctcgattactccgccttgctgtagcaccaagtcacccagtgatctcatcacagatacttgattcctctgttcttgcatcccagacaatggatgttcgggcatctcaaactatggagcatagacgaggaggaggtcgttttggaagatctagacccaagtgttcttattgtcacaaacttggacacactcgtgaaatgtgttattccttacatggtcgtccacccaaaaatgcttacattgctcagaccgagactccaggtaaccagggattttctttatctaaagaagaatataatgaactccttcagtatcgaacaagtaagcagacatctccacaagtagcctcagttgcttagactgatacttcttttactggtaatttttttgcttgtgtttcccagtctagcactcttggcccatgggtcatggactcaggcgcttctgatcacatctctggtaatatatcacttttgttaaatattgtatattcatagtctcttcccattgttactttagccaatggatgtcaaattacggcaaaaggagttggacaagctaatcccttgtcttctatcaccctagattctgttctttatgtccctggctgtctttttcgtcttgcatctgttagtcgtttgactcgtgccctccattgtggtatatattttattgacgattcttttattatgcaggactgcagtacgggacagacaattggtggaggacgtgaatcagaaggcctttactaccttaactcacccagtccttccacaacatgtctggttacagatcctccagatctaatccacagacgtttaggacatccgagtttatccaaacttcagaagatggtgcctagtttatctagtttgtctacattagattgtgagtcgtgtcagcttgggaaacatacccgagcctccttttcgcgtagtgttgagagtcttgcatagtctgccttctccttagttcattctgatatatggggtcctagtagagtaagttcaaccttgggatttcgttattttgttagtttcattgatgattattcaagatgtacttggcttttcttaatgaaagaccgttctgagttattttctatattccagagtttctgtgctgaaatgaaaaaccaatttggtgtttctattcgcatttttcgcagtgataatgccttagaatatttatcttttcaatttcagcagtttatgacttctcaaggaattattcatcagacatcttgtccttatacccctcaacaaaatggggttgctgagagaaagaataggcaccttattgagattgctcgcacacttctaattgaatctcgtgttccgttgcgtttttggggcgatgcagtgctcacaacttgttatttgattaatcggatgccttcatctcccatcaaggatcagattccacattcagtattgtttccccagtcacccttatactctcttccaccccgtatttttggaagcacgtgttttgttcataacttagcccctgggaaagataagttagctcttcgtgctctcaagtgtgtcttccttggttattctcgtgttcagaagggatatcgttattattctccagatcttcgtaggtaccttatgtcagctgacgtcacattttttgagtctaaacctttctttacttttgctgaccaccatgatatatctgaggtcttacctataccgacctttgaggagtttactatagctcctcctccaccttcgaccacagaggtttcatccataccagccgttgaggagtctagtgttgttcctcgtagttccccagccacaggaacaccactcttgacttatcatcatcgttcgcgccctacatcgggcccaactggttctcgtcctgcacctgacccttctcctgctgcggaccctgctcctagtacactgattgcacttcggaaaggtatacgaaccatacttaaccctaatcctcattatgtcggtttgagttatcatcgtctgtcatttccccattatgcttttatatcttctttgaactcggtttccatccctaagtctacaggtgaaacgttgtctcacccaggatggcgacaggctatgagtgacgagatgtctgctttacatacaagtggtacttgggagcttgttcctcttccctcaggtaaatctactgttggttgtcgttgggtttatgcagtcaaagttggtcccgatggccagattgatcgacttaaggcccgtcttgttgccaaaggatatactcagatatttgggctcgattacagtgataccttctctcccgtggctaaagtggcttcagtccgtctttttctatccatggctgcggttcgtcattggcccctctatcagctgaacactaagaatgccttttttcacggtgatcttgaggatgaggtttatatagagcaaccacctggttttgttgctcaggagggggtctcgtggccttgtatgtcgcttgcgtcggtcactttatggtctaaagcagtctcctagagcctggtttggtaagttcagcacggttatccaggagtttggcatgactcgtagtgaagctgatcactctgtgttttatcggcaccctgttgacattccgatggatccgaattctaaacttatgccaggacagggggagccgcttagcgatcctgcaagctataggcggctggttggaaaattaaattatctcacagtgactagacccgatatttcttatcctgtaagtgttgtgagtcgatttatgaattctccctgtgatagtcattgggttgcagttgtccgcattattcggtatataaaatcggctccaggcaaagggttactgtttgaggatcaaggtcatgagcagatcgttggatactcagatgctgattgggcaggatcaccttctgatagacgttctacgtctggatgttgtgttttagtaggaggcaatttggtgtcttggaagagcaagaaacagaatgtagttgctcggtctagtgcagaagcagaatatcgagcaatggctatggcaacatatgagctagtctcgaccaaacaattgctcaaggagttgaaatttggtgaaatcaatcggatggaacttgtgtgcgataatcaagctgcccttcatattgcatcaaatccggtgttccatgagagaactaaacacattgagattgattgtcacttcgtcagagaaaagatactttcaggagagattgctacaaagtttgtgaggtcgaatgatcaacttgcagatattttcaccaagtctctcactggtcctcgtattggttatatatgtaacaagctcggtacatatgatttgtatgcaccggcttgagggggagtgttagtttacagctatgtatatagtgtagtcttgtctcacattggaataggagtagtatgtccttgtatagtatagctataaataagacagtactaacgtcccttttgccgggggttctgcatctttaaatagatgcacgtggttccatagcagaccgtgttgatcacagatcgtgctgcatcctcttcccagcggactcggtgagcccctcttgtattgtattgaacatccaatatcaataacatattttctctcgtgctttctcacaggtctgtgatgtacccttgaaaggttcaagagtttggaggaagatagaaactctgtttatctcccaatcatccaaagatcttctaaagttccagttccatccttgtgagctccagactgacttaccaatgcttggctttgaagacttagagagaataagtcaggaaaaatctttcaaccttccttgccctatccggtgatcttcccaaaaagatgtcttcaacccattgccaacattgatcctgatattgctactgaaagatttcttttggtggcaggattactctcattaacaatgtacttgacaatctccatacatacgaatgtctctttaccctcttgccattaaggttgtaaagagacttgtcaaattaagaagaggtttcctatggaactgtttcaaggaaggaacctcctttcctttggtcaagtggagttaagtcatataatctaggaagtggagacttgggtataaaatagctgcaactacagaaaaggagcatcttatttaaatgatcacgcaaatgtgcccaaaactttaaatatctgcggagcatatggttgtagcaaaatttgaatcttccggtcaatgttgctcatgtccagtgaatacccctgatggtgaaagtgtcctgaagggaagcaggaacttattggaggaattggcatttaacactcagcatttcgttaggtcatagcccgctgaaaattgagtgcccagatttatatagttttgctctaaactgacgatgcagttgcacaacatacgacaaactaaggtgggacatcttcttcggaaggaattttgaggattaagagatagagtggttgattcagttgcaaatgaagcttcaagggttcaatatcatccaggagacaccggattctgatagataaaacaacagaaagatgaacactactttgttaggcttgttacaagttgctatcgtctttcttatctcggcacacaatttagatttgggaacttatttggaaaatagagtggttgtttttgtgaatagcatcagacaaagcttctgagctggtacgacagaaaactcaacagggagaataaaagactgtggttcacgatttctgcatgcatcttgtaggttatttggtgggtaaaatatttaatgttttgaagggaaggtagaacatgttcataggcttagattcaaatgtttgtatttttttggctctttggtgagagatgctgaatgtaaatgacataggcagctgactataatttctcagctccttgctttttaaattggcaggcactgatatgtacatgtgaacatccaacacttttgtggtgccgttccgatgaataaagcacattaatcacttactgatcaggagtaatagtttaggagttctagaatttttgtacataaaatgaaccaaaaagaatatcggaatgagaacatgtttctttttttgtttcttctttttcgtacaaatttcaataacacttctgatagaatagctaggtccatttgaattcctttggagacccttacacaaccaatgaatggcaagtatagcattttctaacaccctcccacatgtataatccagtttttagggtttagatgtggatttgatttgaccttattgcctttttttgtttttgttctttttgaagtagagagtgaggaggctcacaacgacgggctacgtagagcgagattaattcggctcaacgggctaatgattggacttacatgctacaacaatgttaggagaaagagagagagagagagagaagcccagagcagttccacgagttaagaaagagaagtccaaagcgattgaatatgaagagagaaagcggttgtgctaacaggctccctcaagtttggctctgagcatccaactcaaaaccttaaggcaatgagtagagtagcccaggaccatttaaactcctgttgaaaaccttacacaaccaataagggaacaagtgtaacattctcttacaaccctaccgtcttataagtcagggctctaatttagcataaaatcaaagtgaggcgatctactatgaaatgaagaaaataactgataaatataaagaatgttaattctcccatatagcctgaatgttcccagaacaaaataaattagtctcatgatttatcattaacatgatgttcctcttattttgagtgattaggaaggttaatcaaggagtaaattctttctaatttgtatcgtctagaattatttgtctaacaaattttcagattaccggtgatcaaaagaggaaaatattttgcatacaacgttaccataccttacaaaagggcgatgaacatttttttattttattattgtcctttttttcaattaggggttatgcagtcttcctccacgtgatattactcttagaatcacgtttttgtcattgctattacttactgtggtaagtacaaatgtgttttgaactctttttggtatgtattattgagttaatttttcgtttccatttcagagctgccgctttatcttctgctgggcatcttttgtggcttagtttcagtggcattatcaagttgtacatcatttatgctgcaaatagtggaaaatattcaaatgaccagcggcatgccaaaagcagcttttcctgtcctgggcggtcttctggttgggctggtagctttagcatatcctgaaatcctttaccagggttttgagaatgttaatattctgctagaatctcgcccactagtgaaaggcctctccgctgatctgttgctccagcttgtagctgtcaaaatagtaacaacttcattatgccgagcctctggattggttggaggctactatgcgccatctctattcatcggtgctgctactggaactgcatatgggaaaattgttagctacattatctctcatgctgatccaatctttcatctttccatcttggaagttgcatccccacaagcttatggcctggtatgaatttgtcttttgttagaagtagcattacatatctggataagtgagttttttattattgaaaagtaataacaggagaacaagagaatatatcacccaaatctacttctttcctctcttctattcttctgaaattcaaggtcctttaactcctccacagtctgtctagttattgatcctgtagacttaattcacataggtttaggacattcgagtttatccaaacttcatgaaaaggtttctaatttttttacattacattatgagtcgtgtctacttgagaaacatatcactccatgtttctatagtctgttttctccttagtttattctgatatgtggggtcctattaagtcagttcaaccttgtattttcattatttttgcagtatcattgataattattcaagatgtacttggattttctttacaagagatagttctcagttgttttttgtgttcctaagtttttatgctgcaatacaaaattggtttgatgtctctatttgcatttttcccaatgataatgccttagaatattttcttttccgtttcagtagcttattatttctttaggaactctttatcagaaatctcaactgagatagatgagaggaagaataagcatatcattggtctcattcagtcccctgtcaagcttagtttcttgagcgatgcggtttcacgtccttttattagattaattggatgcctcatctgctatccaaaatcagttaactttcgatattgtttcctcgcttacctttatactctctttccctcgagtctttgggagcacatgttttgttcaataacatagctcctggaaagtgaccagcgcaaccgacaaacaaggccttcttaatgtagaaggtggacatatgctattctagccacgggaaagaaagtaatattgtaatcaaacccaaatatctgagtataacctttggcaatggcgatcaatttgattatatggaccaactttgcctgcatatacccaccgacaaccaataatagatttaccgggaggtagagaaacaagctcccaaataccactaatatgtaaagcagatatatctctgatcatagcttgtccttgtggacatagggatagaaattaaggacaaagatgacacaaaagcataatgcggtgatgataaacgatgataactcaaatcaatataatggggatggggattgagagtggatcgaatatctttgcggaatgcgattggtagactaggaggagagaagtctgtggacatgatgttggactgagatcaataataagtcaagaatggtggagctacagaacatggaactggagctgtaggtgacataatcggagctgtaggaggtggagctatagaggaaggtgaaggagagatagcgactgaatctccaaaagatgaaaccggtaatacctcaaaaaatgtctaagagatcatttggacctatgaagtatgattgcgtttttaaaaaggtaacatcataaggtcaggtgaataacattgatatccccgttgcatcctcgagtaacttagaaatatacatttgagagcacggagagctaacttatcttttctggagcaaggttgtaaacaaaacacgtgctcccaaagacacgaggtggaagagagaaaggtgagtggggaaacaagacagaggatgaaacttgactcttgatagttgaagatgacatacaattaataagacaataggatgtgagatccaatgacagttctcatgaactgctgaaatggagaagacaaatactctggggcgttatcactacgaaatgtgcagttagaaaccccaaattgattttggatttcagtgtggaaggtctaaaaaatagagaacaactcagattgatttttcatcaagaatatccaagtggacttggaataatcatcaatgaaactgacaaagtagcggaattccaaggtagaactaacccgacaaggaccccaaacatctgaatggactaaagtgaaaggtaactctacccgattatcaggatgtcgagggaaatgagagtgagtatgccttctgagcggatatgactcacgctctagagtggacaagtgagacaaacgaggtactattttctaaagttctgataaattgggatgtcctaactgtatatgtaataaatctggtggatcagtaaaaggacaagctgtagggggaaaaaaataccaaatatttccagaagatggcaaactacaacagaagatgcaactgcattaacatgctcaggataggtgatgaaatcattgaggacaaagagttgatcaagaaggagattctggaattttaccagaacttatatagtgaaaatgaaccctggaggcgcagtgcaaatttcgaagacatctcctcactaagcatagaagagaagaactggttggaagctccatttgtagaaatagaggtgcttgaagctttgaaatcatgtgccccttataaagcaccaggtccagaaggcttcactatggatttctttcagaaaaattgggatactcttaaaacagacatcatggctgcacttaatcattttcaccagagctgtcacatggttagggcttgcaatgccaccttcattgccctaattccaaagaaaaatggtgctatggagctcagagactacagacctattagcttgacaggtattgtatacaaattggtttcaaagattttagcagagaggctcaagaaggtaattgacaaactagtctcgggggaacaaaatgctttcatcaagaacaggcagatcactgatgcttccttgattgccaatgaagtgctggattggagaatgaaaagtggagaaccaggcgtgttgtgcaaactggacattaaaaaggcttttgatcaattaagctggtcttacctcatgagtatcttgaggcagatgggctttggggagaaatggagaagatggataaactattgcatttcaactgtcaagtactctgttttggtgaatagggacccaatcggttttttctccccccaaaagggcctaaggcagggggatcccctctcccccttcctattcattctggcgatggaaggactcactaaaatgttggagaaggctaagcaactgcaatggatacaaggctttcaggtgggaaggaatcctgccagctcagttacagtatctcatctactctttgcggatgatactcttattttctgtggtactgagagatcacaagcacgaaatctcaacctgacactgatgatcttcgaggcactatcaggactccacatcaatatgataaagagcatcatataccctgtgaatgcagtccccaacatacaagagctagcagacatcctatgccgcaaaacagacactttcccaaccacatatcttggacttcccttgggagctaaattcaaatcaaaagaagtttggaatggagtcctagagaagtttgaaaagaggcttgcgacttggcaaatgcaatacctccccatgggtggcaggttaactttaatcaatagtgtactggacagtcttcccacataccacatatctttgttcccaattccaatctcagtcctaaagcagatggacaaactcagaaggaagttcttatgggaaggatgcagcaaaacacacaaatttccactagtgaaatggctgaaggtaactcaaccaaaattcaaaggagtcttgggaatcagggatgctatgctcttaaaatggctctggagatatggacaggaggaatctaggctatggaaggacatcatatttgctaaatatggagcacacaaccactggtgttccaagaaaacaaactctccttatggagttggtctgtggaagaacatcagcaaccactgggatgaattcttccaaaatgtaactttcaaagttgggaatgtaactcgtataagttttggaaggatagatggcttggaaatacacctttgaaagacatgtttcccagtatgtatcagattgccgtgaccaaagactccactgttgctcataatagaaacaatgacacttggtacccacttttcagaagaaatttgcaggattgggaggtcaacaacctactcacaatgttaagctccctagaatgtcataacattgaagatcaacaacctgacaaacttatttgggaaaattctaagagaggcaagtacacagtcaaagaatgatacattcacctctgtgaccagaatccaatatataactggccatggaaacatatctggagaactaaagtgcctaccaagatgacttgcttcacatgattgtctctaaatggggcctgtctcactcaagacaacttaatcaagaggaacatcatataagttaatagatgctacatgtgccaacaacagtcagaaagtgtaaagcacttattccttcactgctcagttgcaaaagaaatttggaacttcttctacactacctttggtctaaaatgggttatgccacaatcaactaagcaagcttttgaaagttggtatttttggagagttgataaatccattagaaaaatctggaaaatggtgtcggccgcaagtttttggtgtatttggaaagaaaggaactgaagatgttttgatggcatatcaactccactcaaggctgcgtgtttagttaacttattttgctggaactatctcacccctgttaatagtgctgatacttctgtggatttcattagccccctgatagtagcataggcttttgtaaatggagctaattatcctttctcttttgtactctttgcatcttcttgatgccttttaatgaatctaatttacttcatcaaaaagaaaatgacaagttgttgaaggaggaaaagatgtgagtccatgtgatttagcaaggataaggtactaaagtccatttgattcacgtccggtaccaatgatccgtctcgtgctgcattcctgtattaaaacagagtcatcaagaaataaaatagagcaaataagtgattggccaagcgactagtggatatgagattaaaaggactatggggaacataaaaaactgaattcaaaggtaaggaaggaagtggactagcttaacctattctagttgccatggtttgagaatcgttggccattgtgactattggaagtgattgagagtaagaaatagtagtgaaaggagatttgttacccgaaatataattagatgcacctgaatcaatgacccaaaagtcggaagaagaggaaacacaagtcacgctattacctgtttgaacaatagagattagtttggatcaaatagttgtatagagaactgaaatttggagaaatcaatcatatagaacttgtatgtgattattgttgccctttatattgcgtcaaatcctaaaacacattgagattaactgccacttatcacagaaaagatattctctagagacattgttacaatttcatgaagtcaagtaattagcttgaacatatcttcagcaagtccctcgtcagtcctcatattagttacatttgtaacaatgtcggtacataagacttataagcaccagtttgaggaggagtggtagagagttgatgtacatagttaaagtagatatacttacacttagtgttatgtaaagagtggatataaaaagggatcagcataagacaattgtcttcgcgcgtcttaacatttttttcctgtctttatttctctcatggtatcagataacctatctctatcttggtttacccaatggttggcccccatattgtattagccatgctccagttgactaggcttggacgggcagaggtgttaaattatcccatattggttgaaagaatgagctattgtctccttatatggtcttagacaattctccaactcatgagatattttgttttggctgagttagccctaaggtttattttttgtcatattctttaaccttatggcaatgcttgtacacggaaaaaccggagtgcaagacttaaattaggagaaggaaactattgaaggtgaggaacttaaagggttgtgagaatacacgggagaaaaaaatcttaatactatctagtggccttgtatatcaaatgatcagcttgcaaatattttcaccaagtccctcactggtcctcgtattagttacatatgtaacaagttcggtatatatgatttgtatgcaccggcttgaggttatgcatattctattcctcctactatatatgtgactaggaaatattttactcctactgcatatgggactaggactatttacacataactatctaacattcccctcaagccagtgcacacaagtcatatgtaccgagcttgttacatatgtaactaatacgaggaccagtgagggatttagtaaaaatatctgcaagctggtcattcgacatacaaggccactagactccccccgagcaacaaaaccaggtggttgctgataaacagaaactggccgaaaagttgccggaaaaatttgaaaatagtgagactaagccgaattctacactacaaaataggttctaaaacaccaccagaaaacaaaaacttttctagaaattactcttcacaccggaaaaaataaaagttgtcagaatttgatgtaatttatatagataggttcggaatcactggaggagtaagttgtcccgaagaagttttgtcaaaaagtggccggaatggctcacatgcgccggaaaacttactgtagctcgcaggaaccctagttctggcggtgcgtggaggcgcgtgacttaagattaagatgcttacaggactatcttgagaaatatacatattatatagacgcttgagttgcttcccaatcctaaatagaagcttttattcgtaggcaagaagggaagcagctttacttgagccaatagctttcaaggtgcacgttgtcacaccaaggacatccagaatttgattttatagggggtgtgagaaagcacgggagaaaatatgttattgatatttggataataaatacaatacaagaggtccctatttatagctatacactacaaggagatattactcctcttccaatgtgggacaagaatacactatacatatctgtaaactaacactccccctcaagtcggtgcatacacatcatatgtaccgatcttgttacacatgtagctaatacgagaaccaataagagacttagtgaaaatatctgctagttgatcattcgactttacaaactttgtaacaatatctcctgaaagtattttttctctgacaaagtgacagtcgatctcaatgtgtttagtcctctcatggaacaccggatttgacacaatatgaagagtagcttggttatcacacattagttccatcttgctgatttctccgaattttaactccttgagcaactgcttgacccaaaataactcacacgtcgtcatagccatggcccgatattcggcttcggcgctagatcgagcaactacattctgtttcttgctcttccacgagaccaaattacctcctactagaacacaatatccagacatagaacgtctatcaaaaggtgatcttgcccaatcagcatctgtgtacccaacaatctgctcgtggccttgatcctcgaatagtaatcctttgcccggagctgactttatataccgaagaatgcgaacaactgcatcccagtgactatcacagggagaatccataaactgacttacaacactcaccggaaaagaaatgtcaggtctagtcactgtgaggtaattcaatttgccaaccaacctcctatatctcgtagggtctctaagaggctccccctgtccaggcagaagcttagcattcagatccataggagagtcaataggtctgcaacccatcattccagtctcctcaagaatgtctaagacatacttccgctgtgaaataacaatacctgagctagactgagcgacctcaatacctaaaaaatacttcaatctgcccagatccttagtctggaagtgctgaaagagatgttgcttcagattagtaataccatcctgatcattgccagtaataacaatatcatcaacataaatcactagataaatacacagattaggagcagaatgccgataaaacacagagtgatcagcctcactacgagtcataccgaactcctgaataattgtgctgaacttaccaaaccaagctcgaggggactgtttcaaaccatatagtgacctgcgcaatctgcacacacaaccattaaactcccctaagcaacaaaaccaggtggttgctccatataaacttcttcctcaagatcactgtggagaaaagcattcttaatgtctaactgataaagaggccaatgacgtacaacagccatggacaaaaagagacgaacagatgctactttagccacgggagagaacatatcactataatcaagcccaaaaatctgagtatatccttttgcaacaagacgagccttaaaccgatcaacctggccatccggaccgactttgactgcataaacccaacgacaaccaacagtagacttacctgcaggaagaggaacaagctcccaagtgcaactcgcatgtaaagcagacatctcgtcaatcatagcatgtcgccatcctggatgagatagtgcctcacctgtagacttagggatagaaacagtggacaaagaagatataaaagcataatgaggtgacgacagacgatgataacttaaaccgacatagtggggattaggattaagtgtggatcatacacctttgcggagtgcaattggttgactaagaggagacaagtccgcagtaggtgcagaatctgatgcggggcgtgaatcacctgggcctgatgctggatatggacgacgatgataagtcaagagtggtggagctgccgaaggttgaactggattatgtggaggaactggagctataggtggtggagctacaactggagctgtaggtggtggaactagagtaactgaatctccaaaagatgaaactggtagtacctcagaaatatctaagtgatgacctgaacctgtgaagtatgattgggtttcaaagaaggtaacatcagcagacataaggtactgctggaggttaggagagtagcatcgataccccttttgtgttctcgagaaacctagaaatacgcacttaagagcacgaggagctaacttatccgttcctggaataaggttatgcacaaaacaagtgcttccaaagatacgaggtggaagagagaacaaaggtaagtggtaaaacatgacagagaatggaacttggttctggatagctgatgatgtcatacgattaataagatagcaagatgtaagaactgtatcccccaaaaacgcaacggagcatgagattgtatgagtagggtacgagcagtttcaataaaatgtctattctttctttcagctaccccattttgttgagatgtgtacagacaagatgtttgatgaataatcccatgagatttcataaactgctgaaatggggaagacaaatactctcgggcattatcactacgaaatgtgcgaatagaaaccccaaattgattttgaatttcagcgtggaaggtctggaaaatagaaaacagctcagatcgattttttatcaaaaatatccaagtgcacctggaataatcatcaatgaaactgacaaaatagcagaatcccaaggtggaactgacccgactaggaccccaaacatctgaatggactaaagtaaaaggtgactctgctcgattatcaagacgcctaaggaaatgggagcgagtatgcttaccgagctgacatgactcacactctagagctgacaagtgagataaaccagataccattttctgaagttttgacaaactgggatgtcccaaccgtttatgtaataaatctggtgaatcagtaacaggacatattgtagatggaagacaagatgcgagtccatgtatttagcaaggataaggtaataaagtccgtttgattcacgcccggtaccaatgatccgccccgtactgcgttcttgtataaaaacatggtcatcaagaaataaaataacgcatttaagtgatttggctaagcgactaacaactatgagattaaaaggactattgcgaacataaaggactgaatctaaaggtaaggaagaaagtgggcttgcttgacctattgcagttgccatggtttgagacccattggctattgtgacttttggaaaagattgagaatacgaaatagtagtgaaaagagatttgttaccagaaatatgatctgatgcacctgaatcaatgacccaagactcagaggatgaagattgggaaaaacaagtcacgctattacctgtttgaacaacagaagctatctcagaagatgtctgcttacatgctttgtactaaaggaactcaatataatctgctaaagaaaccatccgactattcaaagcatcggttcccatgtcgctacaatttgtagtagtagggttaacttgaaatagtggaaataagtaactccggtgagaaaactgaagaaatagcttgaaaacactgtttacaacagtaaaaacagaacactgttctgcgccggaatctactgtagctgacggaaaaactcaaagtagtcggaatgaaacgaaaaacagtaggggtaggatcggaattaccaggcgacccaactattctgaaggaagtttttcaaaaaatggccggaagtggtcgtacgtgtcggcgcgtgagctcacgcgcgtgagcttctggtggcgcgtggaggcgcgtgaggaggctgctgccggagattttcactggggtttggtcgccggacagtgactactcttgtggtagtgttggattttgcacaacactgacggagataaagcagacgcaaacagccttgaaaaagtcgccggaaaagacttccggtgactgatttctcttcctggaatcgctggaatttatgcacagcgataaatctctcacaattgctctgataccatgtgagaaagcatgggagaaaatatgttattgatatttggataataaatacaatacaagaggtccctatttatagctatacactacaaggagatattacttctcttccaatgtgggacaaaaatacactatacatatctgtaaactaacaaggggaatatcgtttaaagataaaaaagatagcgtgcagaagattgcatacattagagatgcaaaatacagaatacccatactcccagataatgcagtatgccttttgcatgacccactggttgaatggaagcacctggtcaatttactaggtgtgttagtgatttttgctgcttccttcccctttctaaactacatactatctaaaatgttagggggacagaagcccagtcaatctgactaggtgatgttagtggtttccgcttctttctcccacttctaaatgcgtactttctcaaatttaggagcatagaaacttaagcagctgcctacctgaggaggtgcatgggaacataagagaatagactttacctgtcatattttccataccttagttaattacagtgttatcctgataatgatctgttttctgtatctaggctgaatcgagattcaatcgcttttggctgaaaggatgctgctacagatccttagtttacatcattgtggttcttattctataagtacttcccctatcaactacttccttcttttttcttaggttatttgcctcttaggttgtttgcaaggaaaggaacaatagatgttttgatggaatagcaactccaaaccacttccttaaggctaatatactgtttggccaagcttcttcaaagtccaaagcccttttttgtcttcaaaaaagtatctttttttcccaaagttgaggtgtttggccaaacttttggaaggaaaaaaaagtgcttttgagtaaagcagaagctcttgagaagtagaaaaagtagttttttcccggaagcatttttttgaaaagcacttttgagaaaaataaacttagaaacactttttaaaagtttggccaaacactaattgctgcttaaaagtgtttttcagatttattagccaaacacaaactgcttctcaccaaaagtacttttttgaaaaatacttttttgaaaagtgattttcaaacaaagcacttttcaaaataagtttattttagaagcttgtcaaccggctataaatgtcttttatttttacagctagagtaccctaacacctgtaaattcccctagacatttttttcgactttgttagctcattaaccctagtataggactctttgttttggagctagcaaactcttttgttttcctatttttgcatcttcttggtgccatttataatatctcttacttcaccaaaaaaaataagttcccaaaatatgactaccttgagttggccaaagcataaccaaagcttgggcacaccagtgtttgcgtgaattttatggatgttccttacctttatccttctgtgcttatgtagcatctgtcttggttaatcttttctgaagtctatagtgtatttctgtgttgcaacatgagtttactgtcaatcttactgtttgacctcaattttgggttctttttgattttgaaagacatcgtttaacaggttggcatggctgctactcttgctggtgtctgtcaggtgcctctcactgctgttttgcttctctttgaactgacacagaattatcggatagttctgcccctcttgggagctgtggggttgtcttcttgggttacatctggacaaacaaggaaaagtgtagtgaaggatagagaaagactaaaagatgcaagagcccacatgatgcagcgacaaggaacttctttctccaacatttctagtttaacttattcttcaggtgtgaaaccttcacagaaagagagtaacctatgcaaacttgagagttccctctgtctttatgaatctgatgatgaagaaaatgatttggcaaggacaattctagtttcacaggcaatgagaacacgatatgtgacagttctaatgagcaccttgctaacggagaccatatccctcatgctagctgagaagcaatcttgtgcaataatagttgatgaaaataattttctcattggtctgctgacacttagtgatatccagaattacagcaagttgccaagagcagagggcaatttccaggaggtagcttcttggtacatttcaatattcttaactgatgaaaaaataagggaaattgatctagcatgaaattaagctaattataagttttacactgtagaactggtaaaacagggttggctggatatttctttgttgaatttttaggattatatgtattgttttagttttgtaggttgttttctgatgtgctttttgacttggcagaatcttaagatgaaatggaaggtgtttaaccaaaaaatagaattttcagtcaaagcctatatttagaagaaaacgggttattgataaccaagttttactttacttccccaacaatctatttggtaaatagcaaaagtaatgcgtatgtgagaaagcacgggagaaaatatattattgatattagatattcaatataatacaagaggtcctacacatcatatagctatagtctacaaactacatattactctcattccaatgtgggactacacataactaacactccccctcaagccggtgcatacatatcatatgtaccgagcttgttacacatgtaactaatacgagaaccagtaagagacttagtgaaaatatctgctagttgatcatttgactttacaaactttgtaaaaatatctcctgaaagtattttttctctgacaaagtaacagtcgatctcaatgtgtttagtcctctcatggaatagcggatttgacgcaatatgaagagcagcttggttatcacacaccagttccatcttgctgatttctccaaactttaactccttgagcaactgcttgacccaaactaactctcacgttgccatagccattgcccgatattcgacgtcggcgccagatcgagcaactacattctgtttcttgctcttccacgagaccaaattacctcctactagaacacaatatccaggcgtagaacgtctatcaaaaggtgatcctgcccaatcagcatttgtgtacccaacaatttgctcgtggcctcgatcctcgagtagtaatcctttgcttggagatgactttatataccgaagaatgcgaacaactgcatcccagtgactatcacagggagaatccataaactgacttacaacactcaccggaaaagaaatgtcaggtctagtcactgtgaggtaattcaatttgccaaccaacctcctatatctcgtagggtctctaagaggctccccgtgtctaggcagaagcttagcattcggatccataagagagtcaataggtctgtaacccatcattccagtctcctcaaaaatgtctaaggcataattccgctgtgaaataacaatacctgagctagactgaggcactgagcaacctcaatacctagaaaatacttcaatctgcccagatccttagtctggaagtgctgaaagagatgttgcttcagattagtaatatcatcctgatcattgccagtaataacaatatcatcaacataaaccactagataaatacacagattaggagtaaagtgccgataaaacacagagagatcagcctcactacgagtcatggcgaactcctgaataattatgctgaacttaccaaaccaagctcgaggggactgtttcaaaccatataatgacctgcacaatctacacacacaaccattaaactccccctgagcaacaaaaccaggtggttactccatataaacttcttcctcaagatcaccgtggagaaaagcattcttaatgtctaactgataaagaggccaatgacgtacaacagccatggacaaaaagagacgaacaaatgctattttagccacgggagagaaagtatcactataatcaagcccaaaaatctgagtatatccttttgcaacaagacgagccttaagccgatcaacctggccatccgggccgactttgaccgcataaacctaatgacaaccaacattagacttacctgcaggaagaggaacaagctcccaagtgccactcgcatgtaaagcagacatctcgtcaatcatagcatgtcgccatcctggatgagatagtgcctcacctgtagacttagggatagaaacagtggacaaagaagatataaaagcataatgaggtgatgacacacgatgatgacttaaaccgacatagtggggattaggattacgtgtggatcgtacgcctttgcggagtgcaattggttgactaagaggagacaagatcgtagtaggtgcagaatctgatgcagggcgtgaatcacttgggcatgatgttggatgtggacgacgatgataagtcaagagtggtggagctgcagaaggttgaactggattatgtggaggaactggaggtggagctacaactggagctgtaggtggtggaactggagctataagtggtggagctacaactggagctggagatgtagaggaagatgaatgagagatagtgactgaatctccaaaaaataaaattggtagtacctcagaaatatctaagtgatgacatgaacctgtgaagtatgattgagtttcaaagaaggtaacatcagcggacataaggtaccgctgaaggtcaagagagtagcatcgataccccttttgtgttctcgagtaacctagaaatacgcacttaagagcacgaggagctaacttatctgttcctggagtaaggttatggacaaaacaagtgattccaaagatacagggtggaagagagaacaaaggtaagtggggaaacatgacaaagaatggaacttggttttggataactgaagatggcatacgattaataagatagcaagatataagaactgcatccccccaaaaacgaaacggagcatgagattgtatgagtagggtacgagcaatttcaataagatgtctattttttctttcagctaccccattttgttgagatgtgtacagacaagatgtttgatgaataatcccatgagatttcataaactgctgaaatggggaagacaaatactctcgggcattatcactaggaaatgtgcgaatagaaaccccaaattgattttgaatttttagcgtggaaggtctggaaaaatagaaaacaactcagatcgattttttatcaaaaatatccaagtgcaccttgaataatcatcaattattcaataaaactgacaaagtagcagaatcccaaggtggaactgacccgactaggaccccaaacatttgagaatggactaaagtaaaaggtgactctgcttgattatcaagacgccgagggaaatggaagcgagtatgcttatcgaactgacatgactcacactctagagctgacaagtgagataaaccagataccattttatgaagttttgacaaattgggatgtcccgaccgtttatgtaataaatttggtgtattagtaacaggacaagttgttgaaggaagacaagatgtgagtccgtgtgatttagcaaggataaggtaataaagtccgtttgattcacgtccggtaccaataattcgtcccgtactgcgttcctgtataaaaacatggtcatcaagaaataaaacaacgcatttaagtgatttggctaagcgactaatagttatgagattaaaaggactattgggaacataaatgactgaatataaaggtaaggaaggaagtgagcttgcttgacttattgttgttgccattgtttgagacctattggccattgtgactcttgaaagagattgaaaatacgaaatagtagtgaaaagagatttgttaccagaaatatgatctgatgcacctgaatcaatgacccaaaactcagatgatgaagattgggagaaacaagtcacgctattacctgtttaaacaacagaagctatcacagaagatgtctgcttacatgctttgtaccgaaggaactcaatataatctgctaaagaaaccatccgactattcaaagtatcggttcccatgtcgctacaatttgtagtaataggatggatagactcggaaaattgtaaagttatcggaatttgtcgtaaccaggatcgagcaagctgtcttgaagaaatggtttcaaaaaatgtccggaaaggtcacttttacgccggaaaaatataaaaatggtcgaaatttgatttgaattagatgggtaggctcggaattgtgaggagagcagactgtcctgaagaagcttaatgaaaaaatggccggaaagtggccggaaccctcgccgtaaaagttgttaccggcgcgtgaaggcgcgtggcattttttctgccagataaattttcaggggttggtcgtcggagggtgatcccttgtggtggtgttggtttttgcacaataccgacaggccttaggtcacccgaaaatttgcacgatgactaagttctttcttcccggttaacgctggaatgacgcacatcgatcttttctcactaatgctatgataccatgtgagaaagcacgggagaaaatatattattgatattagatactcaatataatacaagaggtcatatttatagctatagtctacaaagtacatattactctcattcaaatgtgggactacacataactaacaacgtaaattaacaaagagaaataaggaatgtaacaacagtcaatccctaaaatcaaggtagaaaactttgataaagcagagaattatagaatgtatttcagtagtacttggaacttgtccttacaaataaaattctttatccttatataggggcgtacaatcataacatttttcgcacttaattcgaattcattatgagcattaattgtattgattgcccgttatcatagataaccataactgacgtatttgtaactataaatgccttataacggctctgattccccttccttatttacttctggtttgtgtatctttccttctttttagcctttattcattcagttctcgcctcttctttgacaactgtcaagcccgatcctctgttctgtactgtctcgtgggtgtttcccccgtaccttccttatattcttaattctgttaattgagagtgtcacttgtcactatgccattgttccacgcgtcatgtttcatccacgtgtaatatcttttttccaccaatacagataatcccccactttctgaatattctcaactgaatattcgggtaagtttttatggcgggaattctttgccgtcgtttttcgagtatcatcgtgtcatcttcagaaccgatgtgacgtacgtcacgtctatttaatgcctatgccaggtggcttctatcgattggctctgcagttttttagcgctttttagggtttttcagcggctgcgtcagtcacgaagtgacggttccattatgacgcttcataatgactaactttaatgatggtcgtgtcttcttattaatacttcattcctttttgatctcttggagtcttccttcttcagtatccaccacattacttctttgtatttctgcatcttctctttgatattcctttggacaatcatgtcttcttctacaccagacccccgtaaggttgtgattgttgacgaacttgatctttctactgctcctactagaagtaggagaggtggtagacttcgtagtcttggttcactatctaatcgtggttcttcttcccagggtagtgctgctaagccatcttcttctagacctagggctcctttaacccctagatcttcttctaggaatagagatttaaatgatccagtgcgcgaacctacagttgcagagattgttcctcaagaattttcttttgtaactgaccgtgaaaccataaggaatcaaatttcttctatagcctccctcaataccgctaacctttatccaagtttaatcagtaatggtcttctctcccgggttcgaagagaatattactgaaaccagatttcccaattttagtccctggtgccaaccagagaattactccataccatgttggtttttcctttgtttacacctacccttttactttagggttcaaaccacctattgaaccagtaatcattgaattctgtcgttatttcaacgtgtgtcttggccagattgaccacatagtatggagggctgttcatgccttcgttatttatcagatttggtttccatgcctttcacttttcagcacttgcttcatctctactcccctaaattgtttcgtgaagtagtttttactctcgtggctagaagtaagagagtgttggttagccttgaagacgattgggaccgtggctggtacgctcgttttgttgctgctcccactagtgcattagtgggtgaagaaaatatgcctttcccggagaaatggaactttgcacgtaagctttcttctcctctttttttttgtcttaaaaaaactccatgtaatcatatacccacttcttcagcaactatggaagttttttatgcttgggtagaaaagatgttaactgctgcgcctatggagaaaagatcctggaaatacttttctcaaagatttggttggaaagtgaagacgcacggtactttttaccttcattgtttttccttttctcttccttgtttgttcaatgatttctcatccttccctttttttttactagggtttccgattcgtggtattagtcccgcgtctgttccatcaactaggctttccgtgattcttgttcaggaaagaattttaagtgcttcttcttcaaaaaggaaaactgacggagcccgtggctctgatgacgaagaagaaacagaggagggttctttggtgcgaaggtcacgcgtcaggagacgcgtggtttctgatgatgaaactactccttctcatgaccctctatctagttcaatcccttttagactcacggatgagctagagagtacccctttagtgatttcttatgatgatgctgttgatccccctccaagttctgttgatagattgtttgctcatggcttcgagggtgatgaagttttgggcctgtttctgaagaattgccccttgcttcccttccagtttcagttttcattaacccttccgtgtccttacctgatgatactcctgttgttattctcgtggctgcttctactccgtcatctattcccgtgactgcttctcatgcagaggccaaaccttctagcagcagaagggcaatgaaaagagttgttgttgaggttcctgaaggtgagaacttattaagaaaatccggtcaagccgacgtgtagttgaaacctatgctcggccccgtagagaagaagaagttagaaagccatagctcactcactttaatgaatgatatcgttcattcttccttgaaagtacaagcttaattatatttcctttcttttctctttcttattcataactcttcctccttttttgcagatcaacttgattggcacagagcttatgaaaagagtttctcaggcggaccggcaagttatagatttgcgcaccgaggctgataactggaaggaacaattcgaaggtcttcaattggaaaaagaggttccggcggaagagaagaatgctttggaacaacagatgagagtgattgcctctgaattagcagttgaaaaagcttcctcgagccaggttggaaaggataagtatatacttgaatcctcctttgctgaacaactttccaaggcaactgaagaaataaggagtttgaaggaactccttaatcaaaaagaggtttatgcgagagaattggttcaaacacttactcaagttcaggaagatctccgtgcctctacttataagattcagttcttggaaagttctctcgcttctttgaagacagcttacgatgcctctgaagcagaaaaagaagagctgagagctgagatttaccagtgggagaaggattatgagattctcgaggataatctatcgttggatgtaagttgggctttcttaaacactcgtctcgagactctagttgaagccaaccatgagggttttgaccttaatgctgagattgctaaggctaaagaagcaattgataaaactcagcaacgtcaaatcttttcctcacctgaagacgaaggtcccgaaggtgatggagattga

SEQ ID NO: 5 - protein, 786 aa, Nicotiana tabacum
MEEPTRLVEEATINNMDGQQNEEERDPESNSLHQPLLKRNRTLSSSPFALVGAKVSHIESLDYEINENDLFKHDWRRRSRVQVLQYVFLKWTLAFLVGLLTGVTATLINLAIENMAGYKLRAVVNYIEDRRYLMGFAYFAGANFVLTLIAALLCVCFAPTAAGPGIPEIKAYLNGVDTPNMYGATTLFVKIIGSIAAVSASLDLGKEGPLVHIGACFASLLGQGGPDNYRLRWRWLRYFNNDRDRRDLITCGSSSGVCAAFRSPVGGVLFALEEVATWWRSALLWRTFFSTAVVVVILRAFIEYCKSGNCGLFGRGGLIMFDVSGVSVSYHVVDIIPVVVIGIIGGLLGSLYNHVLHKILRLYNLINEKGKLHKVLLALSVSLFTSICMYGLPFLAKCKPCDPSLPGSCPGTGGTGNFKQFNCPDGYYNDLATLLLTTNDDAVRNIFSINTPGEFQVMSLIIYFVLYCILGLITFGIAVPSGLFLPIILMGSAYGRLLAIAMGSYTKIDPGLYAVLGAASLMAGSMRMTVSLCVIFLELTNNLLLLPITMLVLLIAKSVGDCFNLSIYEIILELKGLPFLDANPEPWMRNITAGELADVKPPVVTLCGVEKVGRIVEALKNTTYNGFPVVDEGVVPPVGLPVGATELHGLVLRTHLLLVLKKKWFLHERRRTEEWEVREKFTWIDLAERGGKIEDVLVTKDEMEMYVDLHPLTNTTPYTVVESLSVAKAMVLFRQVGLRHMLIVPKYQAAGVSPVVGILTRQDLRAHNILSVFPHLEKSKSGKKGN

SEQ ID NO: 6 - protein, 786 aa, Nicotiana tabacum
MEEPTRLVEEATINNMDRQQNEEERDPESNSLHQPLLKRNRTLSSSPFALVGAKVSHIESLDYEINENDLFKHDWRRRSRVQVLQYVFLKWTLAFLVGLLTGVTASLINLAIENIAGYKLRAVVNYIEDRRYLVGFAYFAGANFVLTLIAALLCVCFAPTAAGPGIPEIKAYLNGVDTPNMYGATTLFVKIIGSIAAVSASLDLGKEGPLVHIGACFASLLGQGGPDNYRLKWRWLRYFNNDRDRRDLITCGSSSGVCAAFRSPVGGVLFALEEVATWWRSALLWRTFFSTAVVVVILRAFIEYCKSGYCGLFGRGGLIMFDVSGVSVSYHVVDIIPVVVIGIIGGLLGSLYNCVLHKVLRLYNLINEKGKLHKVLLALSVSLFTSICMYGLPFLAKCKPCDSSLQGSCPGTGGTGNFKQFNCPDGYYNDLATLLLTTNDDAVRNIFSINTPGEFHVTSLIIYFVLYCILGLITFGIAVPSGLFLPIILMGSAYGRLLAIAMGSYTKIDPGLYAVLGAASLMAGSMRMTVSLCVIFLELTNNLLLLPITMLVLLIAKSVGDCFNLSIYEIILELKGLPFLDANPEPWMRNITAGELADVKPPVVTLCGVEKVGRIVEVLKNTTYNGFPVVDEGVVPPVGLPVGATELHGLVLRTHLLLVLKKKWFLNERRRTEEWEVREKFTWIDLAERGGKIEDVVVTKDEMEMYVDLHPLTNTTPYTVVESLSVAKAMVLFRQVGLRHMLIVPKYQAAGVSPVVGILTRQDLRAHNILSVFPHLEKSKSGKKGN

SEQ ID NO: 7 - protein, 665 aa, Nicotiana tabacum
MCDSSKVDSDSGIQIGSLLEEVIPQGNNTAIISACFVGLFTGISVVLFNAAVHEIRDLCWDGIPYRAASEEPIGVHWQRVILVPACGGLVVSFLNAFRATLEVSTEGSWTSSVKSVLEPVLKTMAACVTLGTGNSLGPEGPSVEIGTSVAKGVGALLDKGGRRKLSLKAAGSAAGIASGFNAAVGGCFFAVESVLWPSPAESSLSLTNTTSMVILSAVIASVVSEIGLGSEPAFAVPGYDFRTPTELPLYLLLGIFCGLVSVALSSCTSFMLQIVENIQTTSGMPKAAFPVLGGLLVGLVALAYPEILYQGFENVNILLESRPLVKGLSADLLLQLVAVKIVTTSLCRASGLVGGYYAPSLFIGAATGTAYGKIVSYIISHADPIFHLSILEVASPQAYGLVGMAATLAGVCQVPLTAVLLLFELTQDYRIVLPLLGAVGLSSWVTSGQTRKSVVKDREKLKDARAHMMQRQGTSFSNISSLTYSSGSPSQKESNLCKLESSLCLYESDDEENDLARTILVSQAMRTRYVTVLMSTLLMETISLMLAEKQSCAIIVDENNFLIGLLTLGDIQNYSKLPRTEGNFQEELVVAGVCSSKGNKCRVSCTVTPNTDLLSALTLMEKHDLSQLPVILGDVEDEGIHPVGILDRECINVACRALATREQLC

SEQ ID NO: 8 - DNA, 408 bp, Artificial sequence
  Synthetic sequence RNAi sequence used to silence CLC-Nt2
gtcatcatcaggtgtgtgtgctgctttccgttctccagtaggtggtgtcctatttgctttagaggaagtggcaacatggtggagaagtgcactcctctggagaactttcttcagcacggcagttgtggtggtgatactgagggccttcattgaatactgcaaatctggcaactgtggactttttggaagaggagggcttatcatgtttgatgtgagtggtgtcagtgttagctaccatgttgtggacatcatccctgttgtagtgattggaatcataggcggacttttgggaagcctctacaatcatgtcctccacaaaattctgaggctctacaatctgatcaacgagaagggaaaactacataaggttcttctcgctctgagtgtctcccttttcacctccatttg

SEQ ID NO: 9 - DNA, 282 bp, Artificial sequence
  Synthetic sequence RNAi sequence used to silence CLCe
gaaatcctttaccagggttttgagaatgttaatattctgctagaatctcgcccactagtgaaaggcctctccgctgatctgttgctccagcttgtagctgtcaaaatagtaacaacttcattatgccgagcctctggattggttggaggctactatgcgccatctctattcatcggtgctgctactggaactgcatatgggaaaattgttagctacattatctctcatgctgatccaatctttcatctttccatcttggaagttgcatccccacaagcttat

SEQ ID NO: 10 - DNA, 44432 bp, Nicotiana tabacum
atgattagcggccaaaacactgtgctgcacaatcctcctaattcgctcttcaattccttatctcctcgccatatctgtatatctttctgtaacgacaaagctttaaaaaagtcagtcacgcactccgcccctcggtttgctcgtctgttaaacaatgaatcacggaagttgttgggtcgtcatccaaattgctggccttgggctcgacgaccatctcttcctccgggacgttcctctgacggaaacattgaaaaagaacaagatatgtgcgacagcagcaaagtcgatagtgatagtggcatccagataggatctctgctcgaggaagttatcccacaaggcaataataccgctataatctcggcttgctttgttggcctcttcaccggtatcagtgtcgtgcttttcaacgctgcggtacgtgcgctataggtctttcatttctcttttcatgtactattcctccttacttacttggcctcagtcaatcagccccctgcctactttaaattattgtacattttatcagaggagtgtcctatacatcaaattcacataacttagtaaaatatgctgatattctgaattttaaacttaccagcttagaacatccaggttagttcagaaacagataatctaaattggtctcatttataagtcattttgttattcaagacatacaatttggctcttgataaaagattatgcagcgcccgatgattacctaatatttatcagcaacccatgtaatttaacaatattgtcaccatataaaagagaactgaagagaatgttcaatttgtggtcatataacggatatctcccttggttaggttcatgaaatacgtgatctttgttgggatggaattccatatcgagctgcctcagaggagcccattggagtacattggcaacgtgtaatcttagtaccagcttgtggcggtttggtagtcagctttttgaatgccttccgagccactctggaggtttcaactgaaggaagttggacatcatctgttaaatctgtattggaaccagttttgaagacaatggccgcttgtgtcacattaggaactgggaattccttaggaccagaaggccctagtgttgaaattggcacatctgttgccaagggagttggagctctgcttgataaaggtggtcgtagaaagctgtcactcaaggctgctggatcagctgctggaatcgcttctggtttgttccccatattattcttggttctgaaccatacatggtacattttccttataattacatgtagcctgttgtatgctttcctctttcccgggaagcctttttgtaaatacaagtgtgtttgcactcaaaccaataaactgtaaaaaaggtgaactccttaagcaagcaaaagcattagaaatgtaaactagacatatttctcagattgagagtctgagagattagaacacgagtgtttccattagagagagaaaagagacttctagatatttctattatctctgtaagagtgaatccgttcctatacaaaaaataggccttcattaaatacaagcttgggctgggtactactgggccaaagtaaaaaataaaaagaatcacccactatcaaatgggcctagtctaacaacccccttcaagctggagggtgacacaacccctagcttgcgaatatgaaaatgatgagcaggcccaagtaacactttggtaagaacatcaaccacttgagaagcactggagttgtgaaatagactgatcaggccattcccaagcttgccacaaacaaaatgacagtccagcttaatgtgtttagtgcgttcatggaaaacttggttttttgcaatgtggacttcctgattatcacaaaataaaggaacaggtaaagaaggagaaactccaatatcagacaataatttggtgagccaagacacctctgcaacagccttactcatggacctatactcagcttcaattgatgatagtgagacaacaggttgcttctttgatttccagctcaccaagctgccccccaagaaaaatacaaaaaccagtgacagacctgcggctgtctgggcaagaagcccaatcactgcacaataaagctgcaaagacaagtctggagagttattgcggaagattccaaagtcaaaagtgcccttgaggtatcttagcaagtgcagggcagcctgcatgttaggaacacagggagactgcataaactgactcagatgctgaacaacaaaactaaggtcaggccttgtgcgtatcaaaaagtttagcttgtgcattagactcctgtactcttcaggcctgggcaaaggagtgccaatcttagcttttaacttcacattcaattcaagggggcaagtgacagaagagcaattcgaggaatgaaaatcagccagcaaatcatgaatgaactttttctgatgaagaagaaccccagaatcagtgtataaaacctcaatgctaaggaagtaattaagagagcccatgtccttaatcttgaactggtcactgagaaaggacttcaaagcagccaattcagctagatcacacctagtcaatatgatatcattcacatagacaaccaagatgaccaaggaatccctagaacccttggtaaaaatagagaaatcattcaaggaacgagagaagccattagagcacaaggcttgagataatttagcatactattgtcttgaagccagtcttaaaccataaagagacttctggagtttgcatactaaaggagcagaagaagagtgaggaacagttaggcccggtggcagcttcatgaatacctcctcatcaaggtccccatgtaagaagacattattcacatctagttgaaagaggggccagtgttgtttaacagctacaacaataagagttttgacaatagacatattgaccacaggagaaaaagtttcattaaagtcaataccctcaacttgagtgacctagctttatatctctcaatactttcattagccctatatttaaccttgtatacccacttacaactagtaggtttcttgccaggaggcaattcaacaatgtcccaagttctgttggcatccaaggcctcaaattcacatctcatggctgcctgccattcaggaacagctgcaacctgagagtaagaataaggctcaggaacatgaagttgactaagagaaggagcattagaaatagatctggagggaggaggagaagaagtggaggtgcagacataactcttgagatagttggttggattgtgtggcacggaagatcttctcaaagcaggaggaggtacaagagagttagaataatgagaaggagaagagatggaagtgggaacagagaagattgagaagcagtagaaggagaaagtgaaggagatgaaggagaggaagaagacggaaaggaacattcatcaaaacaagcagaaaagggaaaggggaagacttgaggtactacatgagaggattgaaagaaaggaaaaatggtgttcataaaaaatgacatcttttgatacaaaacaggtgttattctgaagattaaggcgcttgtagccctttttggcaaaagggtagccaatgaaaacacaaggaagggacctaggatgaaatttgttttgtgaggggtggtgacagttgagtaacagaggcacccaaaagctctaaggtggtgataagtagggtggaagaatgaagcaattcatagggacttttgtgattaagaagaggaaaaggaaatctgttaattaaatatgtggcagttaaaaagcagtcaccccaaaatttaagtggtagatgagactgaaacataagtgacctagcagtctctagtaaatttctgtgttctctttctacaataccattttattggggggtgtgaggacaggaggtttggtgtactatccctttttctgaaaagaaaaggcaaccagaagaactagatcccagttccaaagcattatcactcctaacagtttgaactttagattggaattgggtttcaaccatagcaatgaaaaccttgagcaaatcaaaggcattgcggcacccattaaatgtgtccaagtagccctagagtagtcatctacaatggttaaaaaatacctagaaccattataggtaggagtagaatagggtcaccaagtatttatgtgtattagctgaaaaggctgggtggagtgaatagaactatcagggaaggacaacctggtctgcctcgctaaaggacaaaccggactagtgaatgaccgtttggaagacagtttgcaattaagaccagaaatgcatttcattttatagaagggaatatggccaagtttgtaatgccaaacaacatcatctttattcacattatgcaaagcagtactagtatttacaattggagtatcatcaggtacagaaataggagcagaaactgaattaagcaaacaagaaataaggaaattagaaagaggtaaaggagatgatgttggaggcctggcattctgaaatagtttgtagagtccattgtccaatctaccaagaaccactggcttcctcactgaagggccctgtagggtacaagtagccttggtaaattgtacaatatcatcatcatgggaaagtaatttgtacacaaagatgagattatattgaaaactaggaatatagagcacattataaagaatcaagtcagggaacaaggctaaggaaccaatattagtgaccttaaccttatacccattaggaagggagacaaggtatggtacaggaagtgtttgaacattaaaaaaacaaatgtttaagggaggtcatgtggtcagatgcccagggtctattactcaaactacactatctatcatagtcagcataaatgcaccataagacaacccttgtgaggtaataactcaccagcaaagttggtagaagcaagatagttggttgaagaagtagatgatgctgatgaagacagttgagattgttgaagtaacattagctgagaatattggttcttggtaagaccaggaactggataggactgttcaggagcagaggtaccttcaggaccagctgacattgcagaaccaccagaggtatccacctcagcatgggcaacagaccttctgggaggaagagatctatttgacttgaaatttggaggaaagccattgagcttatagcacttatcaatgctatgtccgggtttcttacaatagtagacatgtgaagctcaaaagatcccttagaggtagtaccggacctttgaggttcaaaatttattttaggagagggaggaggcctggatacaccaacactgaaagaagcagaatttgaggcatattgagttctagcaaaaatttgtctttgcttctcatcagatagcaaaatcccatatacattaccaatggaaggtaagggcttcatcatgatgatgttgcttcttgtttggacataagtatcattcagtcccataaagaactggtagaccttttgttccctgtcttcagcagatttacccccacaagtacacattcaaactctcccggcagacaaagatgcaatatcatcccatagtcgtttaattttgttgaaatatgatgctatgtccatggacccttgggaaatatgagccagttccttctttagctcaaagatcctagtacctctcttctaactcagtccaaatattcttagcaaactcagagtattcaacactcttggatatttccttgtacatagagttagtcaaccaagagaccacaaggtcattgcaacgttaccactgtctggctagaggagaaccttcaggaggtctgtgagaagtaccattaatgaaatctagcttgttacgaatagacaaggcaactaggacattacgtctccaattgccataacagcttccatcaaaaggaccggaaactaaggaagttcccagcacgtctgatggatggacatataaggggcgacagggatgggtataatcatcttcatggaaaattaggcgtaagggagtagaagaagtcgcatcagcactggtgttattatcatttgccatttttttcaacagattgtcaatcaaccaacacaatacagatacacatatatagattgtgagaaagcacgagagaaaaatctatattattgatattctatttaattataatacaatgagccctatttatacaatacatatcatactcctattctatgtgggactaggactaattcatattatgtacataactatctaacactccccctcaagccggtgcatacaaatcatatgtaccgaacttgttacatatgtaactaatacaaggaccagtaaggaacttggtgaaaatatctgcaaactgatcatttgacttcacaaactttgtagcaatatctcatgagagtatcttttctctgacgaaatgacaattaatctcaatgtgtttagttctctcatgaaacaccggatttgatgctatatgaatggcagcttggttatcacacatcagttccatcttgctgacctcaccaaatttcaactaattaagtaaatgtttgatccaaactagctcacaagttgtcacagccattgctcgatattctgcttctgcactagaccgagcaaccacattttgtttcttgctcttccaagacacctaattacctcctactaaaacacaatatccagacgtagaacatctgtcaaaaggtgatcctgcctagccagcatttgagtacccaacaatttgctcatggcctcgatcttcaaacaataatctgttacctggagctgattttatatatcgaagaatgcagacaactgcatcccaatgactatcacaaggagaatccaagaactgacttaccacactcactggaaaggaaatatcaggtctaatcactgtgaggtaatttaatttaccaaccagccgcctatatctagcaggatcgctaagcggctccccctgtcctggtagaagtttagaattccgatccataggagtgtcaataggtctacaacgtgtcattcctgtctcctcaagaatgtctaaggcatacttcctttgtgagataacaatacatgtgctagactaagcgacctcaatacctagaaaatactttaatctgcccagatccttagtctgaaagtgctgaaagagatgttgtttcaacttagtaataccatcttgatcattgccggtaataacaatattatcaacataaaccaccagataaatactaagatttgaagaagaatgccgataaaacacagagtgatcagcttcactacgagtcatgccgaactcttgaataactgtgctgaacttaccaaaccaggctcgaggagactgttttagaccatagagggaccgacgcaaccgacatacaaggccactagactccccctgagcaacaaaaccaggtggttgctccatataaacttcacctcaaggtcaccacgaagaaaagcattcttaatgtccaactgatagagaggccaatggagaacaacaaccatggatagaaaaaggcggactgatgctattttagccacaggagagaaagtatcactgtaatcaagcccaaatatctgagtataccctttggcaacaagacgagccttaagtcgatcaacctggccatctggaccaactttgactgcatacacccaacgacaaccaacaataaatttacccgaaggaagaggaacaaactcccaagtaccactcgtatgtaaagcagacatctcgtcaatcatagcctgtcaccaccctagatgagacagtgcttcacctggatggaaatagaggacaaagatgatacaaatgcacaatagggtgatgacagacgatggtaacttaaaccgacataatggggattagcatttagtgtagaccgttcacctttccggagtgcaatcaattgactaagaggagacaagtccgcagtattagcaggatcaggtgcaggacgtgaatcagctgggcctgatgctgggcgcggacgacgatgataagttaggagtggtagagctgtagaaggttgaactggactaggcagtggaactgaagctatatgtggtggaactggagctataggtggtggagctggagctgtaggtgaagatgaatgggagatagtgactgaatctccaaaagatggaactggtagcacctcagatatatctaagtgattacctggactggtgaagtatgattgggtttcaaagaaggtaacatcagcagacataaggtaccacctgaggtcaggagaatagcatcgatatcccttttgtgttctcgagtaacccaaaaatacgcacttaagagcacgaggagctaatttatcttttcttggagtaaggttatgaacaaaacacgtgctcccaaaggcacggggtggaagagagaacaaaggtaagtggggaaacaagacagagaatggaacttgattctggatagctgaagatggcatacgattaataagatagcaagatgtaagaactgcatccccccaaaaacgcaacggaacgtgagattgtatgagtaaggtacgagcagtttcaataagatgtctattctttctttcagctacccgattttgttgggatgtgtatggacaagatgttttatgaataatcccatgagagttcataaactgttgaaatgggaaagacaaatactctaaggcattatcactacgaaatatgcggatagaaaccccaaattgattttgaatttcagcgtggaaggtctggaaagtagaaaacaactcagatcgattttttatcaaaaatatccaagtgcacctgtaataatcatcaatgaaactgacaaagtagcggaatcccaaggtagaactgacctgactaggaccccaaacatctgaatggactaaagtaaaaggtgactgactctgctcgattatcaagacggcgagggaaatgggagcacgtatgcttaccgagctgacatgactcacactctagagtggacaagtgagataaaccagataccattttttgaagttttgacaaactgggatgtcccaaccgtttatgtaatagatctggtgaatcagtaacaggacaagttgttgaagaaagacaagatgtaagtccatgtgattttgcaagaataaggtagtaaaatccatttaattcacgcccggtaccaatgatccgccctgtactgcgttcctgtataaaaacaaggtcatcaagaaataaaacagagcatttaagtgatttggctaagcgactaacggctatgagattaaaaagactaacgagaacataaagaactgaatctaaaggtaaggaaggaagtggacttacttggcttattccagttgccatggtttgagactcgttatccattgtgactgttgggagtgattgagaatatgaaatagtaatgaaaagagatttgttaccaaaaatatgatcagatgcacctgaatcaatgacccaagactcagaggttgaagattgggagacacaagtcacactactatctgtttgagcaacggaagctatccctgaagatgtttgtttacatgttttgaactgaaggaactcaatataatccggtagagaaaccatccaactcttcgtagtattggattccattttgctacaaccaatttctcaaattcttgattacaacttgtgtggttaaccttggaatgccaaatcagaacaccccttttttttttttggaaaacattgttcactcgctggaaaataaaaaaggttgccggaatttgatgaaacttgaatagaccgactcggaataatgtcctaagaaggctgtccaaaaggagttttgtcagaaactgaccagaaggaggtccacgcaccggcgcgtggacagatctcgccgaaaaaaaaaatcactttggttggcgcgtgatggcgcgtgggtggggtttttccggtcgggttttgtggggtttgctcccccggagatggagaacactgtggtggtgttggtttatgcacaacactggtaaaaagtggttttgatgcgaacagctactcaggtcaccaaaaaattgcacggtgacgactgatttcttcccggatgtcgttggaatgacgcacaacgataattatctcaccaatgctctgataccatgtgagaaagtacgggagaaaaatctatattattgatattctatttaattataatacaatgagccctatttataagactaggattaattcatattatgtacataactatctaacatagatcaaataggcatgcaattcacaataatggtgaataaaatgatacgaagttacccagctcttttcgcgatcgaaaaggagaaaatagccttcaatcacaaacgagaaagaagaatctccggcttgacagtagacgacttcgaaaccctagctcgagatgaaaaccacaaaatccccaaatcacattaccaaccaaacaatttgagatcacaaatgttgaatatgtgagaatccgactaagaaatcaacaaaaaatcaatagaaatggttgaagaataccgacttgaaccctaaatgagtcagacatcacctagaatgaaatacaccttcgaaattgacgaaaacaggaccggttgaaagcggagaacgtgccatagaaggatctacgctctgataccatgtaaacttgacatacttctcagattgagagtctgagagattagaaaacgagtgtttccattagaaagagagaaaagagacttctagatatttcgattatctgtgtaaaaatgaatccgttcctatacaaaaattaggccttcattaaatacaagattcggccgggtattactggcccaaagtaaaatataaaaagaatcacccactatcaaatgggcctagtctaacaagaaaaccaacaaatagtccccccccccccccccaaaagataccactgaaatgacaccgggtgcccaaaaataaagcagcttacttcttgactttgagaggaactgcaatccttatcggtttgagaggaactgcaatcagctataagtagcttattaatttccagtgcctgcattctgccaagtactatgatatatttctgaagctttgtttccccagttcctttttcagacgtttgctgtcaataaagttgagccagccaacttggctcccacaagctactaattttgtccaagcttactctatgggagaagttaaatttcccaaattccttgagcggaaaatgaaaaatggactcaaagtgtcatattatgcaactatctaaagaaaaatactcaattgaagtttagataagaaaagtgaatgtatattgatgtagtctccgttaggtgagaagcgtatcacttacccagcaacatatggacctaacattttactagtgaagttttcacattgtatcaaaagctcaacaaacggaaaggtgactaatcctaaaatgttatttcacatatatgggcacacggtttgtcaaccttctcatacgtgcattatttgttctctatctttctatttcatccgatataaccaatcgttattgtaaattctataatgcctgtggttacttttgtctttagtgacaaatgacatttaggataaccatgtagttattgacttatttcacttgaggtctcttccaattatgtagtagtagagtgttgagatatggatatgttaccttctaaaaaaaagagtgtagagatgcggatagtttgctagctggcttttgtctcccttcaagttgaattagcaaaagcttgtctcataagttggatagctagacaagaaaaactccaaattactttatgtagagtattcttaagcttgagtcgcgagttggaaactggaattatgtaaaaaaacctggaattatttggttgagcctgctttttagttttgtcaatatttccagtatctaacccaacatgtttagagtgattcccggagagcctcagtacaaggcatttgcagagtctttatgagagtccaggaaggggcacacattctgtagaggtatagtcttgtccttattttcagggttgaactagttctttagaagttacctaggcttcctaatttccaaatttctgccaggtccttttttggtgaagtacttgaagtttaataaatcaaattttaatttctaacatatcctgagaaatttattcacaaattcaactggtgacttctgatgcagaaacataagcaactgcttatgggttcatatgttcctgcaattttattgttgacatggattggcttcatatggttttgttcctgcaattttatcgctgacactaatcctttcatatggttttatgtggagtgttaaatagaggttaagagacaagaagaggctgaaaaaggtgggcagttcatttgttagtagactactctatttactaagagatatgatgtcccatacattactcgaattggctccgaatccagattccacttctttgccgagtttccttattgtacatagttcgactcgtcaagggaaattcacttcctttgactgaataatgctagtttgagtagtaccttacattaaatggaccatttagttctatctacttgatagaatagactggtcatcaactagttgcaaatacaatgacaactttgccatgtttgcagagtcacctgatgaagaagtacctcaattagtagaacatttcttgaatgttctacagtattctctatgcctacatgaccacatcacttttccttttgcgttgtgagaacttgaacttggtgagcgggggttccccaggaatggcatcttgatggcagatgaccattctgtccttgtcttagctaatgcttcttgcattgcctcactagatttattatacctttaaaaaatgtttgccattgttctgccataatagaaggatgtacccagctggtgcttcaaaactaatgaaatgctttacaattgtcgagtcctaaaggatgatttgtggaatcagatctcaaacaattctttttgaggaagaaaaataccaaaggttttttctgtttgttggaagattaaaaatcctttaaatggtaaagatttatgaacttaattcagcgtttttgtggccattgctggaaaagagaaaaaacaatggcacttcttcgagtttgcttatccaaaaaaaagaagaagagaatgtcacgtaatgcaatttcatcttaggaaactttgcaggagaaaagcaagagtgataaaacagaactatttgttttttttaacaagttgttgtgacctatttcttgtcattcttatttgctaataagctaatgtactatagttcctgtactatggtttgttttgacttaatacggggatgttcaatgagcattttcttgttttttctgctttcagcatctgctgccttacaggaattcattttctggaaatttacttcttgttctgctaacattttcctgttatatcttgtcagtcattttctctccatggttatactgtttgtgtcactttaaactctccttgttttctactttaaaggatttaatgctgctgtcgggggctgtttctttgctgtggaatctgtgttatggccatcacctgcagagtcctccttgtccttaacaaatacgacttcaatggttattctcagtgctgttatagcttctgtagtctcagaaattggtcttggctctgaacctgcatttgcggtcccaggatatgattttcgtacacctactggtaattttggacttctttctcgagtttgattcttaaatacaattgtacccgtcacttacagcaacaactacatttcaacagctagttggggttggctacacagatcatcactatccatttcaattcatttagtcccatttctttcgaatattgagtactttgggattctataatatcaaggttctttatattttctactttgacgtacaaatctctaaatagattaaagaagactcctagagacactggcctaatgcaaatgtaccaccatgaataaactttaatctgaaatagctggtatcttatataaggacccttagctttaattgtgttctatattgatcttttgggacaacttccttccaatattatgtcttacttatacagttatacttatccttaagccttactctttagagtggttatccctaattcaagcttttgttggcaccatagctagtttggttctaagtaaaaagttactctttagagtggtaactttttgtcaattttcttagtgaaaatataacctctgtgacaaatctaccaagtataaatccaatttggttctatgtcatccttgtagtttatccaagtcaatgctccatcactcttacaaaggttcatcgtatgactaatcttttttggagaaaggtaacagtttgtattgataataagatcagcgccaggttggtcattagtgctaatagctgtacgtacaactccaaaagagcaaaagacaagcacctgatgtaaggtaaattacaagctgcctataaaatctatcaggtgtcctatctcactaaacatttcttgtttacaccaaaaaaataaaacaaggaaagacaatccatcttaatcttctgaatggagtttctttttccttcaaaacatctggagttccttccgttccatgcaatccaccatatacaagctgggatgattttccatttgtctttatccatttcttctaccaattcccttccaattgattagaagttccaatgtggttctagatatgacccaattaactcccaacagataaaagaagatgtgccacggatttgtagtgattctgcaatgtaggaacaagtgagcattactttctacttcctgtccacaaagaaaacatcttgagcaaatctggaaacctcttctttgtaagttatcatgtgttaaacatgcctttttcaccaccaaccagacaaaacatgatactttgggaggagttttaaccctccaaatgtgtttccaaggccacacctcagttgttgaaacattaggatgtagagtccagtatgctcttttactgaaaatgcaccttttctattcagcttttaaactactttatctatggtctgtgatgtacccttgaaaggttcaagagtttggaggaagatagaaactctgtttatctcccaatcatccaaagatcttctaaagttccagctccatccttgtgagctccagactgacttaccaatgcttggctttgaagacttagagagaataagtcaggaaaatatctttcaaccttccttgccctatccggtgatcttcccaaaaagatgtctgcaacccattgccaatattgatcttgatattgctactgaaagatttcttttggtggcaggattactctcattaacaatgtacttgacaatctccatacatactaatgtctctttaccctcttgccattaaggttgtaaagagacttgtcaaattaagaaaaggtttcctatggaactgtttcaaggaaggaacctcctttcctttggtcaagtggagttaagtcatataatctaggaagtggaggcttgggtatgaaatagctgcaaatacagaaaaggagcatcttatttaaatgatcacggaaatgtgcccaaaactttaaatatctgcacagcatatggttgtagcaaaatttgaatcttcctgtcaatggtgctcatgtccagtgaatacccctgatggtgaaagtgtcctgaagggaagcaggaacttattggaagaattggcatctaacactcagcttttcggtgggtcatagcccattgaaaattgagtgcccagatttatatagttttgctctaaactgacgatgcagttgcacaacatacgacaaactaaggtgggacatcatcttcttcggaaggaattttgaggattaagagatagagtggttgattcagttgcaaatgaagcttcaagggttcaatatcatccaggagacaccggattctgatagataaaacaacagaaagatgagcactactttgttaggcttgttacaagttgctatcgtctttcttatctcggtacacaatttagatttgggaacttagttggaaaagcagagtggttgtttttgtgaatagcatcagacaaagcttctgagctggtacgacagaaaactcaacagggagaatagaagactgtggttcacaatttctgcatgcatcttgtaggttatttggtgggtaaattatttaatgttttgaagggaaggtagaacatgttcataggcttagattcaaatgtttgtatttttttggctctttggtgagagatgctgaacgtaaatgacataggcagctgactataatttctcagctccttgctttttaaattgacaggcactgatatgtacatgtgaacatccaacacttttgtggtgccgttccgatgaataaagaacattaatcacttactgatcaggagtaatagtttaggagttctagaatttttgtacataaaatgaaccaaaaagaagatcggaatgagaacatgtttctttttttgttttttctttttcgtgaaaacttcaataacacttctgatagaatagctaggtccatttgaattcctttggagacccttacacaaccaatgaatgacaagtatagcatttctaactccctcccacacgtataacccagattttagggtttagatgtggatctgatttgaccttattgcctttttttgtttttgttctttttgaagtagagagtgaggaggctcaacaattaattcggctcaacgggctaatgattggacttacatgctacgacaatgttaggagagagagagagagagagaagcccagagcagttacatgagttaagaaagagaagtccaaagcgatagaatatgaagagagaaagcggttgtgctaacaggctccctgaagtttggctctgagcatccaactcaaaaccttaaggcaatgagtagagtagcccaggaccatttaaattgctgttgaaaaccttacacaaccaataagggaacaagtgtaacattctcttacaaccctaccgtcttataagtcagtgctctaatttagcataaaatcaaagtgaggcgatctacaatgaaatgaagtaaataactgataaatacaaagaatgttaattctccaatatagcctgaatgttcccagaacaaaataaactagtctcaggatttatcattaacatgatgttcctcttattttgagtgattaggaaggttaatcaaggtataaattctttctaatttgtatcgtctagaattatttatctaacaaattttcagattaccggttcaaaagaggaatatattttgcatacaacgttaccataccttacaaaagggagatgaacatttttttattttattattgtcctttttttcaattagggattatgcagtcttcctccacgtgatattactcttagaatcacgtttttgtcattgctattacttaatgtggtaagtacaaatgtgttttgaactctttttggtatgtaatattgagttaatttttggtttccatttcagagctgccgctttatcttctgctgggcatcttttgtggcttagtttcagtggcattatcaagttgtacatcatttatgctgcaaatagtggaaaatattcaaacgaccagcggcatgccaaaagcagcttttcctgtcctgggtggtcttctggttgggctggtagctttagcatatcctgaaatcctttaccagggttttgagaatgttaatattttgctagaatctcgcccactagtgaaaggcctctccgctgatctgttgctccagcttgtagctgtcaaaatagtaacaacttcattatgtcgagcctctggattggttggaggctactatgcaccatctctattcatcggtgctgctactggaactgcatatgggaaaattgttagctacattatctctcatgctgatccaatctttcatctttccatcttggaagttgcatccccacaagcatatggcctggtatgaatttgtcttttgttagaagtagcattacatatctggataagtgagttttttattattgaaaagtaataacaggagagcaagagaatatagcacccaaatctacttctttcctctcttctattcttctgaaattcaaggtcctttaactcctccacggcctgtctagttattgatcctgtagacttaattcacataggtttaggacattcaagtttatccaaacttcgtgaaaaggtttctaatttttttacattacagtatgagtcgtgtctacttgagaaacatatcactccatgtttctatagagtctgttttctcctcagtttattttgatatatggggtcctattaagacagttcaaccttggattttcattatttttgttgtttcattgataattattcaagatgtacttggattttcttaacaagagatagttctcagttgttttttgtgttcctaagtttttgtgctgcaatacaaaattagtttgatgtctctatttgcatttttcccaatgataatgccttagaatattttcttctcggtttcagtagcttatgatttctttagaaactctctatcagaaatctcaactgagatagatgagaggaagaataagcatatcattgagacggctcgtacccttctcattcagtcccctgtcaagcttagtttcttgggcgatgcagtttcacgtcctttgattagattaattggatgcctcatctgctatccaaaatcagattcaactttcgatattgtttcctcgcttacctttatactctctttccctcgagtctttgggagcacatgttttgttcaataacatagctcctggaaagtgaccagcgcaaccgacaagcaaggccttcttaatatagaaggagggcatatgctattctagccacgagggagaaagtaatattgtaatcaaacccaaatatctgagtataacctttggcaatggcgatcaatttgattatatggaccaactttgcctacatatacccaccgatagatttacggggaggtagagaaataagctcccaagtaccactaatatgtaaagcagacatctctttgatcatagcctgtccttgtggacatagggatagaaattgaggactaagatgacacaaaagcataatgctgtgatgataaacgatgataactcaaatcaatatgatggggatgggaattaagagtggattgaatatctttgcggaatgtgattggtagactaggaggagacaagtccgcaataggtaaaagatccagtacatggaatgaatcttctggacatgatgttggactgacgtcaatgataagtcaagagtggtggagttgcagaacatggaactggagctgtaggtgacataatcgaagttgtagggggtggagctatagaggaaggtgaaggagagatagtgactgaatctccaaaatatgaaaccggtaatacctcaaaaaatgtctaagagatcatttggacctatgaagtatggttgcgttttaaagaaggtaacatcagcagacataaggtaccgcggaaagtcaggtgaataacattgatatccttgttgcgtcctcgagtaacttagaaatacatatttgagagcacggggagctaacttatcttttctggagtaaggttataaaaaaacacatgctcccatagacacgaggtggaagagagaaaggtgagtggggaaacaagacagagtatgaaacttgattcttgatagttgaagatggcatacaattaataagacaataggatgtgagaactgtatccccacgtaaacacaacagaacatgagattgtacgagttgggtatgagcagtctcaatgagatacctattcttcctttcagctatcccattttattgagatgtgtatggacaaaatatttgatgtatgatcctatgagagttcatgaactgctgaaatggagaagacaaatactctggggcattatcactatgaaatgtgcggttagaaaccccaaattgattttggatttcagagtgaaaggtctgaaaaatagagaccaactcagattgatttttcatgagaaatatccaagtggacttggaataatcatcaatgaaactgacaaagtagcagaattccaaggtagaactaactcgacaaggacctcaaacatctgaatggactaaagtgaaaggtgactctattcgattatcaagacaccgaggaaaatgagagcgagtatgccttctgagcggatatgactgacgctctagagtggacaagtgagacaaaccaggtaccattttctgaagttctgataaattgggatgtcctaaccgtttatgtaataaatctggtggatcagtaaaaggacaagctgtaaggggacaaaaataccaaatatttccagaagatggcaaactacaacagaagaagcaactacattaacaggctcaggatatgtgatgaaatgaggacaaagagttgatcaagaaggagattctggaattctaccagaacttatatagtgaaaatgaaccgtggaggcccagtgcaaattttgaaggcatctcctcactaagcatagaagagaagaactagttggaagctccatttgaagaaatagaggtgcttgaagctttgaaatcatgtgcccctgataaagcaccaggtccagacggcttcaccatggctttctttcagaaaaattgggatactcttaaaatggacatcatggccgcacttaatcactttcaccagagctgtcacatggttagggcttgcaatgccaccttcatcgccttaattccaaagaaaaagggtgctatggagctcagagactacagatctattgacaaactagtctcgggggaacaaaatgctttcatcaagaacaggcacatcactgatgcttccttgattgccagtgaagtgctggattggagaatgaaaagtggaaaaccaggcgtgttgtgcaaactggacattgaaaaggcttttgatcaattaagatggtcttacctcatgagtatcttgaggcagatggctttggggagaaatggataagatggataaactattgcatttcaactgtcaagaactctgttttggtgaatagtggcccgaccggttttttctcctgccaaaagggcctaaggcaggggatctcctctcccctttcctattcattttggcgatggaaggactcactaaaatgttggagaaggctaagcaactacaatggatacaaggctttcaggtgggaaggaatcctgccagctcagttacagtatcccatctactctttgcggatgatactcttattttttgtggtactgagagatcacaagcacgaaatctcaacctgacgctgatgatcttcgaggcactatcaggactccacaacaatatgataaagagcatcatataccctgtgaatgcagtccccaacatacaggagctagcagacatcctatgctgcaaaacagatactttcccaacatatcttggacttcccttgggagctaaattcaaatcaaaagaagtttggaatggagtcctagagaagtttgaaaagaggcttgcgacttggcgaatgcaatacctctccatcggtggcaagttaactttaatcaatagtgtactggacagtcttcctacataccacatgtctttgttcccaattccaatctcagtcctaaagcagatggacaaactcagaaggaagttcttacgggaaggatgcagcaaaacacacaaatttccactagtgaaatgactcaaggtaactcaaccaaaattcaaaggaggcttgagcatcagggatctacaagcacacaacaaagctatgctcttaaaatggctctggagatatggacaggaggaatctaggctatggaaggacatcatagttgctaaatatggagcacacaatcactggtgttccaagaaaacaaacactccttatggagttggtctgtggaagaacatcagcaaccactgggatgaattcttccaaaatgtaactttcaaagttgggaatggaactcgtattaagttttggaaggatagatggctcggaaatacacctttgaaagacatgtttcccggtatgtatcagattgccttgaccaaagactccactgttgctcaaaatagagacaatggcacttggtgcccattttcagaagaaatttgcaggattgggaggtcaacagcctactcacaatgttaagctccctagaaggtcataatatcgaagatcaacagcctgacaaacttatttggggaaattctgagagaggcaagtacacagtcaaagaatgatacattcacctctgtgaccagaatccaataatagataactagccatggaaacacatctggagaactgaagtgcctaccaaggtgacttgcttcacatggttgactctaaatggggcatgtctcactcaagacaacttaatcaagaggaatatcatactagttaatagatgctacatgtgccaacaacagtcagaaagtgtaaaccacctattcctccactgctcagttgcaaaagacatttggaacttcttctacactacctttggtctgaaatgggttatgccacaatcaacaaagcaagcttttgaaagttggtatttttggagagttgacaaatccatcaaaaaaatctggaaaacggtgccggctgcatttttttggtgtatttggaaagaaaggaaccgaagatgttttgatgacatattaactccactctactccctcaaggctgcgtgtttagttaacttatttagttttgtggattttattagctccctgatagtagcataggcttttgtaaatggagctaattatcctatctcttttgtactctttgcatcttcttgatgccttttaatgaatctaatttacttcataaaaaataaaaggacaagttgttgaaggaggaaaagatgtgagtccatgtgatttagcaaggataaggtactaaagtccatttgattcacgcccggtaccaatgatccatcccgcattgcattcctgtattaaaacagagtcatcaagaaataaaatagagcaaataagtgattggccaaacgactagtggatatgagattaaaaggactatcgggaacataaagaactgaattcaaaggtaaggaaggaagtggactagcttaacctattccagttgccatggtttgagaatagttggccattgtgactgttggaagtgattgagagtaagaaatagtagtgaaaagagatttgttaccagaaatataatcagatgcaactgaatcaataacctaagagtcggaaaaagaaacacaagtcatgttattacctgtttgaacaatagaagttatctccgaagaggattatttacatgttttgtactgatggaactcaatataagccgataaagaaaccatccggatattcaaagtattggatcaacagcttataagccaaaagcatccgatacgagtgccattataatggatcaagagagatcaaacaacaaatcaccaaatatcataaacaaccaagaatctcgctggaatgtgaacaaagattgaaaaacaacaatgtagctcgccaaaaatgtgcaaagtgatcgaaaaatattgaatcgtgagtggagagaaataggagcttcaatcgacccacacagtaccaaaaaatccaaaaacggttgtcggagctcaagaaagttgtcaaaaagtatattgtatgcttcgaaagtagccgaaaaaggttggaagtgggatgtgtcaactccgaattatgatacgagcaccacagaagatcaatttgtgtcaaaactaccgaaaaaaatacttcacaccccgacgcgtggagtactcgctcgttggaacccttgctgccaacgtcgcatgtaggatcagttttcgaagaatcttattggggtttggtcgccggacgatgtcggatcttgtggtgccgttggaattcgcacaaccctgaaggaaaagaaggttacacaaatcagatctgaaagtcaccgaaaagacacatggcgattgacttttttgtctcagatgtttctcaccgtcgctctgataccagttgttgggctcaactcgtttgaagatactcttaacatagtgtgatattgtcccttttggaatgtgagtcatcttagctcggtaagcatactcgctcttccaactagcccgaagatacttttaacagagtgtaatattatctgctttgagccaagctggcgcggttttcatcaaaagacctcatactattaaaagatccatacaccttatatgtaggcttctaagttgctcggacacgggtgcgagtacccgacacaggtgcaaatctagaggtcagatcctttaaaatgtaaattctaagatttggggatacgaatcctagtacggatacgggtgcgaggatccgattaaaaataattcaaaaaaataagaaaataaaaaagtctctaaattatgtgaaattttgtggaataactacgtatagcttgtaaagtgtggatttattttttattctcaagttgtagataagtaaatgattgatttcctagataaggtatgttattttcttcaaatttaccctagtttggttcgaatttcgggaaattgtatcttgtctcgaatttttccttctgtcctgattaaactactcaaaatcgtctgaccagatccggtacggatcccatacccacatccacactagtgtcgtgtggacaagggtgcggcacctaaacttccgtgtaggagcaatttaggtaggctcctaatcttttcagctattaatgtgggacttttacgcacctctatcaaattccccaataaactaagtttcacgtggtccatcatcgcaatccacgggtctcttcctctagttaagtcccacatggcccattaccatgatccacgggtcaattttcgtgattcatcgtgtgccacccacatcgttagtatttatggtaactaaagtacgcaactagcttttgcttgtgagcgtgtctccaagctcgtaaaggtaagaaaaccgagccgcatattccatcactctatcatcaccatactcgtcccgcgaaacttgtaagataaaggtggctggttggtcagttgaactacctcagagtgacttggtatagtatttcctttcttgtgaatatttaactcaattatggactctctgtgtgatagtcattgagagccattttctatatagccggtgcacacaaatcatatgtaccaagcttgttatatatgtaactaatacgaggaccagtgaaggactcggtgaaaatatctgcaatctggtcattcgacatacaaggccaatagactccccagcaataaaatcagggggttgctgataaatagaattggccgaaatgttgccagaaaaatttgaaaatagtgagactaagccgaattctacactacaaaataggttttaaaacacaaccagaaaacaaaaacttttttggaaattactgttcacatcgaaaaaataaaagttgtcagaatttgatgtaatttatatggataggctcgtaatcactggacgagtaagttgtcctgaagaagttttgtcaaaaggtggccggaatggctcacacatgccggaaaacttattgtagctcgccggaaccctagttctggcggtgcgtagaggcgtgtgactttctgccagactgattgactgtggtttgtcgcctgacttttcctaacaagatggtagtattggttttcgcacaacaattaccgatgaggagataacgcaaatcaatcttgagtcgtcaatcggaaagacgcacggtggctgactttctatttagatgggactggaatttctggagtttaatcgcacaagcgttttggatctgatggtaatactggtatgcacagtaccactgtagcagtgatgaaccctcaaaataagacaaagttgccagaaaattgcacggcgatgagatctttcttccggatgtcaccggaatgacgcacaacgataatttctcactgaagctctgacaccatgtgagaatacacgggagaaaaatctatttttattaacaatgatacaatgagccctatatataatacatattctactctactacatatgggaatagggcatattttactcctactacatatgagactaggactatttacacataactatctaacaagggctatatctcagatttatgagaatatctacccaacgacccagagagacgagcctaatcattttgcagtggcacagactataacaacaaaaaacctactcataatggttaaaccaactgattaagatgcttacaggactatcttgagaaatgtacatattatatagatgcttgagttgcgtcccaatcctaaatagaagcttttattcgtaagcaagaagggaagcagctttacttgagccaatagctttcaaggtgcatgttgtcacaccaaggacatccagaatttgattttatagtgggaatatcgtttaaagataaaaaagatagcgtgcagaagattgcatacattagagatgcaaaatacggaatacccatactcccagataatgcagtatgccttttgcatgacctactggttgaatggaagcacctggtgaatttactaggtgtgttagtgatttctgctgcttccttcccctttctaaactgcatactatctaaaatgttaggggggcagaagcccagtcaatctgactaggtgatgttagtggtttccgcttcttcctcccacttctaaatgcgtactttctcaaatttaggagcatagaaacttaagcagctgcctacctgaggagttgcatgggaacataagagaatagactttacctgtcatattttccataccttagttaattacagtgttatcctgataatgatctgttttctggatctaggctgaatcgagattcaatcgcttttggttgaaaggatgctgctacagatccttagtttacatcattttggttcttattctataagtacttcccctatcaactacttccttcttttttcttaggttatttgcctctttaggttgtttggaaggaaaggaacagtagatgttttgatggaatagcaactccaaaccacttccttaaggctaatatcctgattggccaagtttctccaaagtccaaaacactttttttttccttcaaaaaagtacctttttttttcaaagttgaggtgtttggccaagcttttggaaggaaaaaaagtgtttttgagtagaagcagatgctcttgagaagcagaagaagtagcttcttcccggaagcacttttgagaaaaataaatttagaaacactttttaaaagcttggccaaacactaattgctgcttaaaagtattttcagatttattagacaaacacaaactgcttctcaccaaaaatacttttttgaaaagtacttttcaaacaaagcacttttcaaaataagttttttagaagcttggctaaacaggctataaatgtcttttatttttacagctggagtaccctaacacctgtaaattcccctatacatttttttcgactttggtagctcattaaccctagtataggactctttgttttggagctagcaaactcttttgttttcctatttttgcatcttcttggtgccatttataatatctcttcaccaaaaaaaaaaagttcccaaactatgactaccttgagttggtcaaagcataaccaaagcatgggcacaccagtgtttgcgtgaattttatggatgttccttacctttatccttctgtgcttatgtagcatctgtcttggtcaatcttttctgaagtctatattgtatttctgtgttgcaacatgagtttactgttaatcttactgtttgacctcaattttgggttctttttgattttggaagacatcgtttaacaggttggcatggctgctactcttgctggtgtctgtcaggtgcctctcactgcggttttgcttctctttgaactgacacaggattatcggatagttctgcccctcttgggagctgtggggttgtcttcttgggttacatctggacaaacaaggaaaagtgtagtgaaggatagagaaaaactaaaagatgcaagagcccacatgatgcagcgacaaggaacttctttctccaacatttctagtttaacttattcttcaggttcaccttcacagaaagagagtaacctctgcaaacttgagagttccctctgtctttatgaatctgatgatgaagaaaatgatttggcaaggacaattctagtttcacaggcaatgagaacacgatatgtgacagttctaatgagcaccttgctaatggagaccatatccctcatgctagctgagaagcaatcttgtgcaataatagttgatgaaaataattttctcattggtctgctgacacttggtgatatccagaattacagcaagttgccaagaacagagggcaatttccaggaggtagcttcttggtacatttcaatattcttaactgatgaaaaaataagggaaattgatctagcatgaaatgaagctaattataagttttacacagtagaactggtaaaacagggttggctggatatttctttgttgaatttttaggattatatatattgttttagttttgtaggttgttttctgatgtgctttttgactcggcagaatcttaagatgaaatggaaggttgtatcatcaaatgttaaataagggaatatgtgactttcaaagttaagcacggagtattttggagtcaatagttacttcctgaatcttttaggatggaggagacagtttctataggaataggaaaaggggacctgatttcattatttgtgtgtatatacatttgttatctgaattcgcattactttctaacaaccaacaaaaggaaagtggacattcaatttgagccggagggagaaaatttaactagaaaatgacctggccgtgaaataaaattattgatccgtcctttaactagttttcatggattgcctccttgcggatgatttttccaaccggtagaactactgttagtcgtccaaattctgaccccctactatgaataaaaatgtattagtaagtttagtgggtaatctccttgagaaataaaggaacaggagaaatattttattgatatatgctaagtgttttacaatagccctatttatatacaatgtttacataaacctaaagccttctatataaatgtgggacactatacatgaactaactctaacactatccctcaagctagtgcatataaattatatatatgcttgttacatatataattaatttctctactttttggtatacttcttgtatacgggagttatctcccttttgattaatacaatttaccttatcaaaaaaaaattaatacgaggaccagtgagggacttggtgaaaatatctgcaagttgatcatttgacttctcaaactttgtaacaatatctcctgagaatcttctctctcgtgaagtgacagtcaatctcagtgtgtttggtcctctcatggaacactggatttgatgcaatatgaaggacaacttgattatcacacacaagttccatctgactgattgctccaaattttaattatttgagcaattgtttgatccaaactagctcacatggtgcaagagtcatgactcgatattcggcttctgcgctagatcgagcaactacattctgtttcttgcttttccgagagacaaattacctcctattaaaacacaatatccagatacgtaacgtctatcagaaggtgaccctgcccaattagcatctgtgcgtccaacaatatgctcatggcatcgatcttcgaatattagtcatttgtctggagctgattttatataacgaacaatgcgaacaactgcatcccaatgactatcgcaaggaaattccataaactgacttacaacactcacaggaaataaaatatcaggtctagtaattatgaggtaattcaattttccaaccaggcgcctatattttgcaggattgctaagaggctcccccctatcctggcagaagcttagcattcggattcataagagtatcaatagttctgcagcccattattcatgtctcctcaagaatgtctaaagcatacttcctttgcgaaataacaacctgaactagaccgagcgacctcaatacctacaaagtacttcaatctgctaaggtcgttagtctggaagtgttgaaagtgatgttgtttcaaattagtaataccatcctgatcattgcgagtaataacaatatcatcaacataaaccaccagataaatacagagattaggagcagaatgccgataaaatacagagtgatcagcttcactattagtcatgccaaattcccgaataattgtcctgaacttacgaaactaggctcgacgagattgttttaaaccatagagacttgcataagtgacatacaatacctctagactccccttgagcaacaaaaccaagtggttgctccatattaactttatcctcaagatcaccatggagaaaggcattctttatgtccaactgataaagaggccaatgatgaacaatagccatggacaggaaaaggcgaacagatacgactttagccacgggagaaaagtgtcattattatcaagcccaaatagctgagtatatccttttgcaatcagacgagccttgagccaatcaacctggccatccaggtagactttgactgcataaacccaacgacaaccaacagtagacttacttgaaggaagagaacaaactcccatgtaccactcactcacatgtaaagcaaacatctcgtcaatcatagcctgtcgccatcctggatgagatagtgcctcacctgtaaacttaggaatggaaacagtggacaaagatgatacaaaatcataatagggtgatgagatgcggtgataacttaaaccaacataatggggactaggattaagtttggatcatacaccctttcgaagtgcaatcagtggactaggaggagccaagtccgcactagacgtggatgacaatgataagtcaagagtggtggcctcgtggttggagatgtaggatgagcaactgtagactcctcagaagtcggtataggtaggagtacctgtgatgttgatgtggatttaagaggaggaacaatagattcctcacaagtagatacaggtaagacctcagatatatcaagatgattagatgaagtaaagtaaggttgagactcaaaaaatgtgacatcgactgacataagatatctacgaagatcaggtgagtagcagcgataccccttttgaacccgagaatagccaagaaagacacacctgagaacacaaggagctattttatctttttcaggagctaagttatgaacaaatgtactccttaaaacactaggaggaaagagtataaagatgacctagggaacaatactgagtgtggaaactgattctagatggaagatgaaggcatccgattaattaagtaacaggttgtaagaactgcatcgtcccaaaaacgttgtggaacataggactgaatgagaagtgtgcgagcagttttaatgagatacctattctttctctctactaccctataatgttgaggagtatacagacataggataatattttgagaagtcataaactattgaaactaagagaatacatattttaaggcattatcactacgaaaagcgaataaaaacaccaagcggagttttaatttcagcataaaaactctagaatattgaaaacaactcaaaacgatctttcatttggaaaatccaaatacatcttgagtaatcattaatgaaactaacaaaatccaaatcttaaggttgtgactctactaagaccccatatatcataatgaactaaagacaaaacagactctacacgactcttagcacgacgtgaaaatgtagctcgaatatatttcccaagttgacacgaatcacaatctaatgtggacaaaccagacaccatcttctgaagcttggataaactcggatgtcctaaacgtttgtgaattaggtctagaggatctgtagttggacatgttgtagagggattgagtgagttaagatagtcaaggtcttgtgattcacgccatgtgccaatcgtctgtaccgtactgcggtcctgcatagtaaaagaatcatcaataaaatatatatcacaatggaattcacgagtcaaatgactaacagatgcgagattaaaggacaaccggggacataaaaaatagaatctaaagtgacagaggacatgtgattagcttgtccaactccttttgcttttgtttagacttcatttgctaaagtatcattgggaagagattgtgaataaacaattatttgacaaaagtgacatattaccactggggtatcaagttgcttagtcatactaagaatgtttgggagagggtggtggaagtgagggtaaggaggacagtgtctctatccgagaaccagttcggattcatgcatgatcgttcaactgcggaagctatccgtcttattaggaggctggtggaacagtacaaggataggaagaaggatttgcacatgatgtttacctagagtaagcgtatgacaaggtccctaaggaggttccttggagatgtcagaaggttaaaggtgttccggtagcatatactagggtgatgaaggacatgtatgatggagctaagactcgggttaggacaatggaaagagactctaagcattgtttggttgttatggggttacagtaaggatctacgctcaaaccgttcttatttgccttggcgatggacgcattaacgtaccatattcagggagatgtgccatggtgtatgttattcgcggatgatatagttctgattgatgagacgcgaggcggtgttaacgagaggttgggggtttggagacagacccttgaatttaaaggtttcaagttgagcaggactaagacagaatacttggaatgtaagttcagcgacgtgacggaggaagctgacatggacgcgaggcttgattcataagtcatccccaagagaggaagtttcaagtatcttgagtcagttatacagggagaagatggggagattgacaaggatgtcacgcaccgtattaagggcggggtggatgaaatggaggttagcattcggtatcttttgtcacaagaatgtgccaccaaaacttaaaggtaagttctatagagcggtggttagaccaaccatgttgtatggggcagagtgttggccagtcaagaattctcatatctagaagatgaaagtagcagaaatgagaatgttgagacggatatgcgggcatactacgttggaagattaagaatgaaaatatttgggtgaaggtgggcgtggccccatggaagttgtgcccaccattaaagactgctatctgaaaactaattctttgggcccaaacattctggcccaaagtacctcgtgaataataatattgagctcatgtctgacatgttggaagaggagttactagcaaacacttatacacctatgttggtaacacaattgaagaactacgaaaaacactcttctgcaaaggaaaatgagaagaagaagaagaagaagacgaagaagaaggatgatgcaatgatcattgaagaaaaaggagagcaggaggacccatctaaacttacaaagtctagaggaagaggaggacccagagtttgatgcttccctctgggtacaccaaaacatcgtcaaacttaggcaaggagtttggggtaaacattcaggggtgtgagaaggaagctttggagcttttcgtaaaattacaactagaggcataaaaaaaaaaaaggcaatccaggcatggaggtgacaaccttcgaaaagaaagggattcaaagaactgaaagggctggatttttggagtaacttcaagagtaatagaacaagaagtagggggttgcattattatcaaagatcaatgaagattaacattgaagaagtgggaaatccaaaaagactccaccgagaaggatgatgcaatgatcattgaagaaaaaggagagcatgagaaaaaacccgtagaaattgacagcactcacacacaataagacgagataataaagtagtgagttggccaattgaagaagctttacctcttaacttacaaagtctagaggaagaggaggacccagagtttgatgcttccctctgggtacaccaaaacatcgtcaaacttaggcaaggagtttggggtaaactttcaggggtgtgagaaggatgttttggagcttttcataaaattataacaagaggcatgggaaaaaaaaggaaatccaggcatgcaggtgacaaaaccttccaaaagaaagggactggaagaactgaaagggctggatttttggcgtaacttcaagagtaataggacaagaagtacgggattgcattattatcaaagatcaatgaagattaacattgtatcatggaatgtcagggggttaaatcgacatagaaaaagaatgttgattaggagtttaattcataggtggaaagcagatgttttctgtttccaagattcaaaattaaaaggggacattagggagtttataagagaactatgggcaaataggtggtttaaatatgcacagttggaggctagtgggcctagagggggtattattgtcttatgggatagtaaaattggggagggggagatcagcagcctgagctcctattctgttacttgtaaatttataggtaaaactcaggagtatacttggaatttatccactgtatacgctccaaatgatagggaggaaaggaaagaagtatggtgggaattagcaggtgccaggggaatttttatggaccttgggtaatttctggggatttcaatactgtgaggtacccaccagagaaaaagaattacagcaaaatcactagagcaataaatgaattctcataatttattgaagatatggaactggtggatctacaacttgcaggaggaagttacacttggaggacaggagatagacatgtgataacagctagactggataggttcttggtttttatggattggaatgagagcatcagaaacaccaagcaatcagttctccattgaattacctctgaccattcccctgtgatgcttcaatgtggtaaccggtaccctgtcaaatcctattacaagtttgagaattggtggctggaaacagagggcttcaaagaaaggattaaagtctggtggagctcttttgcttgtgaaggaagacgtgactttattctggctttcaaacttaaagcatcgaaggaaaaaattgaagaaatggagtaaatctattcaaggaaacttggagatgcagaaattgagtattcttagtcaacttgcagaactagaagagacacatgatcaaaggagccttactgaagaagaaatacacactaaatatgcagtctatggagtttggggagattgcaaaacatgaggaggtggcttggagacaaagatctagggctctttggttgaaagaagggacaaaaacatcaattttttcctcaaaattgcaagtgcacataggaaatacaataacatagaccaactgttacttgaaggaaaatttgtggcgaatccaacatacataacaaataatattggtacattttatcaaaaactatatataaagattgctagaggacaatcttatgttgcaaagtcttttcgaagcttaggaaatttgggatagtgtcaggcatgtgaaagggataaagcacctggacctgagaactgggaggtgataaacacggatatgatagctgcagttctttgttcatggaatgtttgaggaaagctttaatgttacctttgtggtattgattcctaagaagatggaagctaaggaatagaaggactttaggcctattatgataggcaatgtgtacaagatcttgatagaaagacttaagaaattggtgaacaagttggtgaagggtcaacggatgacttttattaaaggtagacagataatggatgttgttctaattgccaaatgaatgtgtagatgcaagaacaaaggcgagaaacctacaatactatgcaaactagatattgagaaggcatatgaccatctaaattggaactttctattggaatcgctgatgaggatgggctttggtgtaagatgggtcagctggatcaaattctgcatcagcacaatgaaattctcaattttgataaatgtttcaccagtaggtttcttcccttctcagagggatttgagacagggtgatccactatctccttttattattcattagtgctatgggaggcttaaatgatatgttaaagactactcaagataacaactgcatacggggttttaaggtgaagtccagggcagacagtactattgagatttttcatcttcgatatgcagatgacgcacttatgttctgtgaggttgacaatgaacaattgaaagtgctgaaggtgatcttcattctgtttgaagccacatctgtattacaaattaactggaatgaaagctttatctatctagttaatgaggtaactaagatccactttttggttggaatcctagaaggtaaaattggggaattgcctacagttatttggggatgccatgggggccaagagcaattttaaggggatttggactagggtcgtagagatatgtgaaaaaattttaacaaactggaagagttagtatttatccttaagggacaaactaatactaatcaattctatacttgatgattttcctacttacatgatgttcctcttctcaatccatgtgaatgttgtgaagagaatatatacccttagaaggaacttcctatggggaggaaactatgacaaggaaagatctatttggtcaaatggaagtctctcacagtcagcaagaagtaagagtgttttggaatcaagaattggagaattcagaaccaaagtttgatgatgaagtggctatggagatttactacagaagaacattgtttgtggaaagaggtgatcatggagaagtatggcatagaagataaacggataacaaagtctgtaaatagatcttatggagttagtcgatggaaatccatcagggacctatagcttcagctcttgaataagtccaaattctgaataggaaatggattgaaaatatctttttggaaggataattggctaaccaaggaactttgaaacaactctttcttgacatttacattccaaatcaacagcataaagcaataatagtagaattatgggctaatcaaggttggaatctcacatacagaagactatcaaaagacccggagattggcaggtcaacagagttcaaaggcactttggaacaatttaaagaggtctatacttctatagactatttgacttggcaagggaagtttattgttaattcagcctataaggaattcaacttctcagctaactggattggttgttggccatagaagttgatttggaaagttaaaattccttatagagttgcttgtttctcttggcttttggctaaagaggcagttctgacgcatgataatctaaccaagagagattaccatttatgttcaagatgttatttatgtgaagagcaggcagagacaaccaatccacttttttttgcattgtaagttcactgcagttatggaggattttcattagtttaaagggtatcatgtgggctatgcgtagaagtatacctgaagttctagcatactggaaaaaagaaagaaatctttccaattataaaaagagatggaggattatcctagcttgcatctggtggaccatttgggaagaaagaaatcaaagatgcttcaaagataaatcagtcatattcagataattaaaatgaagtggctagtcttgttttatttttggtgttaagtgttagatagttatgtattatgtataagttgtctagtcccacattggaacgggagtaatatgtactatgtagagtatagctataaataggacttcttgtactttattgtagagaatatattaataatatatttttcccgtgttgtctcacatggtatcagagaaaccgtgagatatcagtcgttgtgaaaaataccagcggcttcgggaagaaaaaaatcaatcaactgctaggtatattagtcttcggcgaccgatccattaaatttctctggcaaagaaccactcatgggccctcacgcgcccaccgaaagaaatatttccggcgaggttccaatttcatgcgcccgcgcgtgaggcagtttccggtcaaattttgacaaaggtcctttttgacagtttgttcaccctgtaattcccagtctatccatcattttttttatttcgatcacttcgcaatttctcgggcagctacagtgatttttccggcagaagcggtgtttcctttgcctgcttcagcgagatacagttgattatttctattatttgtttctagacctctctccaatccaacgatgtctttggaatttgatgtatttggttctgaaaacacgagttctagaaagtcaagcttcatgattactttagagccattaatggggagttcaaactatttagcttgggtttcctctgttgaattgtggtgtaaaggtcaaggtgttcgagatcacttaatcaaaaaggctagtgagggctgtgaaaaggtcaatttaagcagtttatgacgtctgtataccactcagcagaataggatagcaaagaaagaatatgcacatcattgagactgctcgcacacttctcattgagtctcacgttctgctacattttctgagcgatgcagttctaacggcttgttatttgattaatcggatgcctttatcttccatccagaatcagattctgcagttagtattgttttctcagtcacccttatacttttttcgtcctcgtgcttttgggagcatgtgtttgttcataacttagctcccgaaaaaaataagttagctcctcgtgctctcaagtgtgtcttccttggatattcccgagtttaaaagtgatattgttgctactcacctgatcgtaggtaccttatgtcagttgatgttgcattttttgagtctagaccttactttacctcttctgaccaccttgatatatatatgaggtcttacctataccgactcttgaggggtttactatagctcctcctctacatactgagccacagaaatcttactcatacctaccattggggaatctagtgttgctcctcctagatccccagctacaggaacacttttaacttatcgtcgtcgtccgcgcccagcatcatgtccagctgattcacgttctgcacctgctcctactgcggactagtctcatcctaatctaccaattgcacttcggaaaggtatatagtccacacttaatcctaatccatattatgtcggtttgagttatcatcgtgtcatcacctcattatgcttttataacttctttgtccactgtttcaattcataagtttacaggtgaagcactgtcacatccaggatggcaacatgctatgattgacgagatgtctgctttacatacgagtagtacttgtgaacttgttcctcttccttcaggcaaatctactgttggttatcgttgggtttatgccgtcaaagttggtccagatgaccagattgccaaagggtatagtcaaatatttggggcttggttacagtgatattttctctcccgtggctaaaataccatcagttcatctctttatatccatggttgttgttcgtcattggcatctctatcagtttgacattaagaatgtttttcttcacagtgagattgaggatgaagtttatatgaattaaccacctaattttgttgcttagggggagtctagtggctttgtatgttggttgcctcagacgctctatggtctaaagtaatctcctcgagccttgtttagtaagttgagcacagttattcgggaatttggccaactcgtagtgaagcttatcactttgtgctttattggcattttacttcaaatctctgtatttatttggtggtttatgttgacgatattgttattaccggcaatgaacaggatggtattactgagttgaagcaacatctctttcagcacttttagactaaggatctgagtagattgaagtattttttaggtattgtgattgctcagtctagcttaggttttgttatttcacattggaagtagaaaaacttcaatcatttttctttatttgaaaggaagaaaaaaaaggtaatatctagacctaaatattaatctgaagacaagtgaggcttgctcagttggtaaaagcacctccacctacgatcgttaggtcctgggttcgagtcaccatggaggggaagtgtggaaacactatagatcctcctaatttgggagggggaaaaaaatattaatctgaattgacatgaatctcaatgacaatgaccaacgatttcctgcaattcttttcagtatggaatgaataaaaaatcaagctacaagtctctattaaacgaaatgcactaacagggatcactctcaagaaaggaagtggttttggttgttgttattccaggttggataaatcactttctttataaatatcataaaagacaagggctttcttgcttcagcacatgtgggaaatgccggggggcttggctggtaccaagctcgagcggtctttctatctttttggattgcatgcccaaggcaatgctttttgtagattgggatggattgatcttcgcagaagtatgctttagacattcttgaggagacaggaatgacggattgtagacccattgacacacctatggatccaaatgccacacttctaccaggatagggggagcctcttagtgatcctgcaagatataggcggctggttggcaagttgaattacctcacagtaactagaccttatatatcctttcctgtgagtgttgtaagtcagtttatggactctccttgtgatagtcattgggatgtggttttccgaattcttcgatataaaatcagctccaagcaaagaactgttgttcgaggatcgaggcccatgagcagatgttgattgggcacgatcaccttctaatagacattctatatctggatattgtatgttaataggagttaatttggtgtcttggaagatcaagacgtaaaatgtagttgatcggtctagtgcggaagcaaataatcgagcaattgttatggtaacacgtgagctagtttggatcaaacaactgctcaaagaattgaaatttggagaaattgatggaaccagtgtgtaataatcaagcagctcttcatattgcgtcaaatccggtgttccatgacagaattaaacacattgagattgactctcactttgccggagaaaagatactctcaggagataccgttacaaagattgtgaagtcgaatgatcagcttagagatatttttaccaagtcccttgctggtcctcgtattagttatatttgtagcaaactcggtatatatgatttatatgcaccaacttaagggagagtgtgagatagttatgtacaacaaaatacccggtataatcccacaagtggggtatggagggtagtgtatacgtagagcttacccttaccctgtgaaggtagagaagctgtttccaaataccctcggctccagtacaaatgaaaaggagcagtagcaacaagcagtaacaacaatgatatagtaaaataactgaagaaagaaataacatgtagacatataactccactaacaaacatgcaaggttaatactattgccacgagaatggcaaaggaatgttagatagttatgtattatatgtatattaatagtctagtctcacgttggaataggagtaatatgtactatgtagagtatagctataactaggacttcttgtaatatattgcatagagatatcaataatatatttttcctgtgctttctcacgtaaaggaatgtaatgtacttagaagatcatgaatctatctttgatgttttagacacctcgtgagaacacaaaggtttaggaactttattgtgttctttgtaattatgggtgactgccaatatgttaccttttcataaaaatgattatttggccattggattagtttcaacagcctctctgcccctccgggtaggggtaaggtctgcgtacatattaccctctccagaccccacttgtgggattatactgggttgttgttgttgttgttgtggattagtttcaacaattttgatagttcttttatttgaatcaaactactcattcacatggattttgtatcgtatcattgagttaaaaaaattggttttgctaatttatcctcatgtataacaactacctatttttcaatatattggattcaggagcttgtagtagctggagtttgctcttcaaagggcaataagtgccgggtatcatgcacagtgactccaaatacagatctcctttctgctctaactcttatggagaaacatgatctaagtcagctacctgttatactaggggacgtggaggatgaaggcatccatcctgtgggcattttggacagagaatgcatcaatgtagcttgcaggtttttgacattcaacttttacttcaaagatataatgctttctggaaccattgatgataaaatatgcaagaaacttgtgcagaagtcgcactttactatcgattaccagataaagttacttatcaagaagtcaaatatattgaacatatttctctaaaacactttgactggactgtaagcagaaacttactaaagtaggtcgtaagaaatggtttgatagggaaatcaccatctacacttaaaagagttgtgtgaatttgaattcttaaagcatgtgaaagttataaaaacttgttattatctaagcatctgaagcattttggccatccaaaggatcaaaaataggaaataatttcatttgtacaatgaactccctgcacaaattctcacactaggtgtattctctattcatcactagcactacatgtgtcactacgaatcatatacaataaatctttgtaacataaaagacgacacataatatggaagtaagccgagtatacaagggaagtttcatcattacggtgagctttttataagataatcaagttttactggaaaagggcaaaaactctcccgtatagaagtataccaaaaagtagaataccttacaaaaatatgattttctatgaacaacaccctatcttctatacttgtagggatctcatcggggcaccaaaaagagataaagggataagaggcttttcctcaaatgtacaaaatccttctctattccttcaaaagctctcctatttctctctctgcacactgtccacataagttcaatggagcaacatccacgccctgtgtcttcttttccgtcttctataggtccagctgaacatggcttctttgactgagtgtggcatcaacgttgaagaccaaaccatcccagtacttccaaccacaaacgagacactatatgacaatttagaagaagatgattcacatcttctcccgaacatttacacataaaacaccagctgatacatgtaatcttcctcttcctcaaattatcagccgtcaggatcacccgtctcgtagctaactaggtgaagaagcacacctttctcgaaaacctcaggatccatacagagagatatggaaaagctgattcctccatgcccagaagcttctcataataagacttaacaaagaaacaccactacttccccccccccccaaaaaaaaaaaatctccatacatcgactttcatgtgtaattcttgttcgtgaaacgacccaatcaacctttggcacaaatctcccagtcttgcgagttcctcctaaacttcaaatcacaatgaacttctccaccttgtagcctccgtgtcccttggactggcaactcctttggcatgaaactttgtacatattaggagatgtgatactcaaagtgttgttcctgcaccaattgtacccccaaaaaacttaccatgctcccatcacctaacattgaatgatacgttccaaaatcttcgcactccttcaagaaacttttccgtaggccccacccataagggagtgtgattttttttgctctccatcccctctccaagaatccattccctaaaccactgcaggacactttaacaatcactatgtcactttttctactagttctacattgagtgatatcttgatgtcattgaaatgcctctggaaaatcttcttctcatctaaaagaacacttgtttgccttttgaatccccctctaacattttctatgtttcattcatctttggtggaacagagcattagcaactagagaacagctttgctag

SEQ ID NO: 11 - DNA, 36854 bp, Nicotiana tabacum
atgattagcggccaaaacaccgtgctgcaccatcctcctaattcgctcttcaattccttatctcctcgccatatctgtgtatctttctgtaacgacaaagctttaaaaaagtcagtcacgcactccgcccctcggtttgctcgtctgttaaacaatgaatcacgaaagttgttgggtcgtcatccaaattgctggccttgggctcgacgaccatctcttcctccgggacgttcctgtgacggaaacattgaaaaagaacaagatatgtgcgacagcagcaaagacgatagtgatagtgatagtggtatccagataggatctctgctcgaggaagttatcccacaaggcaataataccgctataatctcggcttgctttgttggcctcttcaccggtatcagtgtcgtgcttttcaacgctgcggtaagtgcgctataggtctttcatttctcttttcatctactattctcccttacttacttggcctcagtcaatcagccccctgcctactttaaattattgtacaatttatcagaggagtatcctatacatcaaattcacataacttagtaaaatatgctgacattctgaattttaaccttaccagcttagaacatccaggctagttcagaaacagataatctaaattggcctcatttataagtcattttgttaatcaagacatacaatttggctcttgataaaagattatgcagcgcccgatgataacctaatatttatcagcaacccatatgtcactttcttttgtttaaatgctctcccatgtaatttaacaatattgtcaccatacaaaagagaactgaagtgaatgttccatttgtggtcatataacggatatctcccttggttaggttcatgaaatacgtgatctttgttgggatggaattccatatcgagctgcctcagaggagcccattggagtacattggcaacgtgtaatcttagtaccagcttgtggcggtttggtagtcagctttttgaatgccttccgagccactctggaggtttcaactgaagaaagttggacatcatctgttaaatctgtgttggggccagttttgaagacaatggccgcttgtgtcacattaggaactgggaattccttaggaccagaaggccctagtgttgaaattggtacatctgttgccaagggagttggagctctgcttgataaaggtggtcgtagaaagctgtcactcaaggctgctggatcagctgctggaatcgcttctggtttgttccccatattattcttggttctgaaccatacatggtacattttccttataattacatgtagcctgttgtatgctttcctctttcctgggaagcctttctgtaaatgcaaatgtgtttgcactcaaaccaataaactgtaaaaacagtgaaccccttgagcaagcaaaagcactagaaaaccaacaaatagatcccccccccaagataccagtgaaatgacaccgggtgacccaaaaataaagcagcttacatcttgactttgagaggaactgcaatcagctataagtaggttattaatttccagtgcctgcattctgcccaagtactatgatatatttctgaagctttgtttccccagttcctttttcagacgtttgctgtcaataaagttgagccagccaacttggttcccacaagctactaattttgtccaagcttactctatgggagaagttaaatttcccaaattccttgagcagaaaatgaaaaatgaactcaaagtgtcatattaggcaactatctaaagaaaaatacttaattgaagtttagataagaaaagtgaatatatattgatgtagtctccgttaggtgagaagcgcatcacttacccagcaacatatggacctaaaatttactagtgaacttttcacattgtatcaaaagctcaacaaacagaaagatgactagtcctaaaatgttatttcacatcaaccttatcatacgtgcattatttgttctctatatttctatttcatccgatataaccaatcgtcattgtaaattctataatgcctgtggttacttttgtctttagtgacaaatgacatttaggctaaccatgtagttattgactgatttcgcttgacgtctcttccaattatgtagtagtagagtgttgagatatggatatgttaccttctaaaaaaaaagagtgttgagatgcggatggtttgctagctggcttttgtctcccttcaagttgaattagcaaaagcaatgtctcataagttggatagctagacaagaaaaactccaaattactttatgtagagtattcttaagcttgagtcgcgagttggaaattggaattatgtaaaaaaacctggaattatttggttgagcctgctttttatttttgtcaatatttccagtatctaacccaacatgtttagagcaattcccagagagcctcaatacgaggcatttgcagagtctttatgagagtccaggaaggggcacacactgtagaggtatagtgttgtccttatttttttttttttgataaggtaagattttattaaaaggtaccaagatggtgcaaaattacaaacatccaaactaatacaacaaagcaactacattcctcctagctcctctagaaaattcatatattgttccatatttttcattacatgtcttttacaccagaaatacaagtttaataagcatctgtttttaatcctggatacatgctgcctttccccttcaaagcaaatcctgtttctttccaaccatattgtccagaacacacatagaggaattgttcttcatactatctgttgactctttgccactttttgttgttgccatgtctccaacaaactttacactggcaggcattgcccacttgacatcatatatatttaggaagagctaccaacactgctttgccactttgaaatggatgattagatggttgactgtttctgcctcttcttcacacatgtaacaccggttacatagagcaaaacctctcttctgcaagttctcctgagttagaaaagcttcctttgctccaatccaaccaaaacgggctactttaataagtgcttttgacttccatattgctttccatggccaatttgactgataaagcccttgtagtttttgtaacaagctataacaactgctgactgtgaaaataccatcattacttgctgcccagattaatgagtctctcctgttttcctccaatctaacattattcaataactgcatcaattgggaaaattcatcaacttcccagtcattgaggcccctcttgaagattagctgccagccggtgcttgaatagaagtctaacactcttccatttttgttaatagagcagctatatagaccaggaaactttgatctaagacttccattttccaaccacatatcagaccaaaacagggtattattaccatttccaagtttcagtttcacaaactgactatatttattccaaagattactaattgtgctccaaactcccccttttgaagaagattgaattgaacgaggagcccacatgtccttcataccatacttggcatctatcacctttttccataatctattcccatcataattatatctccatagccatttaaataaaagacttttgttatgcatctttagattcctcactcctaatccccctctttctttttttttcatcacctcttgccatttgaccaagtgaaatttcttgttatcattattaccttcccacaaaaatttattcctcatagtattcaattttttctccactgatgttggcattttaacgagagatattagataagtaggtataccatccatcacactattgaccagtgtaagcctaccaccaagagataaatattgtcttttccatgacaccagtttactgctacatctatccaagaccccctgccacatctttgcatcattcttttttgctccaagtggtaggcccagataggtggatggtagctgctccactttacaacccaaaacatctgccagatcatcaatacaatgctcggcattaatactaaacacattactctttgccaagttcactttcaatcccgagacagcttcaaaagctagtagtactcctatgaggtgtaagagttgctctttttcagcttcacataatatcaatgtatcatcagcatagagtatgtgtgagaaatacagttcttccccctctctttttctaattttcaatcctctaatccaccctaacttttctgcttttaaaagcattctgctaaagatttccatcaccaacaaaaataaatagggggatattggatccccctgtcttaaccccctctgagaattaaagtatctatgtggactcccattaattaaaactgagaagctaattgaggatatgcagaattttatccacccaatccatctttccccaaaattcgtatgtttcatcagatttaacagacatgaccaatttacatgatcataagccttttccacgtcaagtttgcaggccacccctttaatcttcctcttgaatagatattcaagacactcattagctaccatagcagcatcaataaattgccttcctcttacaaaggcattctgattatctaatatcaattttcctatcaccatctttaatctttcagctatcgactttgcaattattttatagacactgcccaacaagctgataggtctaaaatctttcacttccgctgccccctttttcttaggaataagagcaatgaaaattgagtttaggctcttagtcttgtccttattttcagggttgaactagttctttagaagtttcctaggcttcctaatttccaaagttctgccaggtccttttctagtgaagtacttgaagtttaataaatcaaattttaatttctaacatatcccgagaaattcattcacaaattcaactggtgacttctgatgcagaaacataagcaactgcttatgggttcatatgttcctgcaattttattgttgacatggattggcttcatatggttttgttcctgcaattttatcgctgacactaatcctttcatatggttttatgtggggtggtaaatagaggttaagagacaagaagaggctggaaaaggtgggcagttcatttgttagtagactactctatttactaagagatatgatgtcccatacattactcgaattggctccaaatacagattccacttctttgtcgagtttccttattgtacagagttcgactcgtcaagggaaattcacttcctttgactgaataatgctagtttgagtagtaccttaaattaaatggaccatttaattctatctacttgatagaatagactggtcatcaactagttgcaaatataatgacaactccgccatgtttgcagagtcacctgatgaagaagtacctcaattagtagaccatttcttgaatgttctacagtattctctatgcctacatgaccacatcacttttccttttgcgttgtgagaacttgaacttggtgagcgggggttccccaggaatggcatcttggtggcagatgaccattctgtccttatcttagctaatgcttcttggattgcctcactagatttattatacctttaataaatgtttgccattgttctgccataatagagggatgtacctagctggtgcttcacatcacatagtccaaaactaatgaaatgctttacaattgtcgagtactaaaggatgatttgtggaatcagatctcaaacaatttattttgaggaagaaaaataccaaaggttttttctgtttgttggaagattaaaaatcctttaaaaggtaaagatttatgaacttaattcagcatttttgtggccattgctgaaaaagagaaaacaatggcacttattcgagtttgcttatccaaaaaaaaagaagaagagaatgtcacgtaatgcaatttcatcttaggaaactttgcaggagaaaagcaagagtgataaaacagaactatttgtttttttgataagttgttgtgacctatttctttgtcattcttatttgctaataagctaatgtaccctgtactatggttgttttgacttaatccggggatgttcagtgagcattttcttgttttttctgctgtcagcatctgctgccttacaggaattcattttctggaaatttacttcttgttctgctaacattttcctgttatatcttgtcagtcattttctctccatggttatactgtttgtgtcactttgaaactctccttgttttctactttaaaggatttaatgctgctgtcgggggctgtttctttgctgtggaatctgtgttatggccatcacctgcagagtcctccttgtacttgacaaatacgacttcaatggttattctcagtgctgttatagcttctgtagtctcagaaattggtcttggctctgaacctgcatttgcagttccaggatatgatttccgtacacctactggtaattttggacttctttctcgagtttgattcttaaatacaattgtacccgtcacttacagcaacaacaactacatttcaacagctagttggggttggctacacagatcatcactatccatttcaatttctttagtcccatttctttcgaatattcagtactttgggattctctattatcagaggttctctttattttctactttgacgtacaaatctctaaatagattaaagaagactcctagagacactggcctaatgcaaatgtaccaccatgaataaaccttaatctgaaatagctggtatcgtatataagaacctttagctttaattgtgttctatattgatcttttgggacaacttccgtccaataatattatgtcttacttatacagttatacttatccttaaactttactctttagagtggttatccgtagttcaagcttttgttggcaccatagctagtttggttcttagtaaaaagttactctttagagtggtaactttttgtcaattttcttagtgaaaatataacctctgtgacaaatctaccaagtataaatccaatatggttctgtgtcatacttgtagtttatccaagtctatgctccatcactcttacaaaggctcatcgtatgactaattttttttgagaaaggtaacagtttgtattgataataagatcagcgccaggttagtcattagtgctaatagctgtatgtacaactccaaaagagcaaaagacaagcacctggtgtaacgtaaattacaagctgcctataaaatctatcaggtctcctacctcactaaacatttcttgtttacaccaaaaaaataaaacaaggaaagacaatccatcttaatcttctgaatggagtttcttttgccttcaaacatctcgagttcctttcgttccatgcaatccaccatatacaagctgggatgcttttccatttgtctttatccattttttctaccaattcccttccaattgactagaagttccaatgtggttctagatatgacccaattaactcccaacatataaaagaacatgttccacggatttgtagtgattctgcaatgtaggaacaagtgagcattactttctacttcctgtccacaaagaaaacatcttgagcaaatctggaaacctcttctttgtaagttatcatgtgttaaacatgcttttttaccactaaccagacaaaacatgatactttgggaggagttttaaccctccaaatgtgtttccaaggccacacctcagtcattgaaacattatgatttagagtccagtatgcatcttttactgaaaatgcacctttgctattcagcttccaaactattttatctatggtcttgttagtttacagctatgtatatagtgtagtcttgtcccacattggaataggagtagtatgtccttgtatagtatagctataaataaggacctcttgtattgtattgaacatccaatatcaataacatattttctcccgtgctttctcacatggtatcagagcaattgtgagagatttatcgctgcgcataaattccagcgactccgggaagagaaatcagtcaccggaagtctttttccgacgactctttcaaggttgtttgcgtttgctttataaatccaacactaccacaagagtaatcactgtccggcgaccaaaccccagtaaaaatctccggcagcagcctcctcacgccaccagaagctcacgcgccggcgcgtacgaccacttccgtccattttttgaaaaacttccttcagaacagttgggtcgcctggtaattcctatcctacccctactgttttcatttcattccgaccactttgagttttttccggctgctacagtactattccggcagctatagtactattccgacaactacagtaagattccggctgctacagtatttcattattctgtttttgtgtttccttactctgtttcagtggattacaattgattctttctcttatttggtaataatttgcaacaatgtctatgggatttgatgtttttgggtctagaaacatgagttctggaagctctagtgttattattacctcagaaccttaaatgggaggttcaaactacttagcttgggcttcatctgtcgagttgtggtgtagaggccaaggtgttcaagatcatctaatcaaaccgtctagcgaaggagatgaaaaggcaataacactttggacaaaaatcgatgctcagttatgtagcatcttgtggcgatctattgattccaagttgatgcccttgtttcgtccattcctgacatgttatttggtttgggcaaaggcacacaccttatacactaatgacatatctcgcttctatgatgtgatatcgcggatgacaaactgaaagaagcaagaattagatatgtctacttacttgggtcaagtacaagcaatcatgggggaatttgagaagttgatgccagtttctgctagtgttgaaaaacaacaagagcagcgacaaaagatgtttctcgctcttaccctcgctgaacttcctaatgatcttgattcagtacgcgaccatattttagctagtccgactgtcccgacagttgatgaattattctctcgattactccgccttgctgtagcaccaagtcacccagtgatctcatcacagatacttgattcctctgttcttgcatcccagacaatggatgttcgggcatctcaaactatggagcatagacgaggaggaggtcgttttggaagatctagacccaagtgttcttattgtcacaaacttggacacactcgtgaaatgtgttattccttacatggtcgtccacccaaaaatgcttacattgctcagaccgagactccaggtaaccagggattttctttatctaaagaagaatataatgaactccttcagtatcgaacaagtaagcagacatctccacaagtagcctcagttgcttagactgatacttcttttactggtaatttttttgcttgtgtttcccagtctagcactcttggcccatgggtcatggactcaggcgcttctgatcacatctctggtaatatatcacttttgttaaatattgtatattcatagtctcttcccattgttactttagccaatggatgtcaaattacggcaaaaggagttggacaagctaatcccttgtcttctatcaccctagattctgttctttatgtccctggctgtctttttcgtcttgcatctgttagtcgtttgactcgtgccctccattgtggtatatattttattgacgattcttttattatgcaggactgcagtacgggacagacaattggtggaggacgtgaatcagaaggcctttactaccttaactcacccagtccttccacaacatgtctggttacagatcctccagatctaatccacagacgtttaggacatccgagtttatccaaacttcagaagatggtgcctagtttatctagtttgtctacattagattgtgagtcgtgtcagcttgggaaacatacccgagcctccttttcgcgtagtgttgagagtcttgcatagtctgccttctccttagttcattctgatatatggggtcctagtagagtaagttcaaccttgggatttcgttattttgttagtttcattgatgattattcaagatgtacttggcttttcttaatgaaagaccgttctgagttattttctatattccagagtttctgtgctgaaatgaaaaaccaatttggtgtttctattcgcatttttcgcagtgataatgccttagaatatttatcttttcaatttcagcagtttatgacttctcaaggaattattcatcagacatcttgtccttatacccctcaacaaaatggggttgctgagagaaagaataggcaccttattgagattgctcgcacacttctaattgaatctcgtgttccgttgcgtttttggggcgatgcagtgctcacaacttgttatttgattaatcggatgccttcatctcccatcaaggatcagattccacattcagtattgtttccccagtcacccttatactctcttccaccccgtatttttggaagcacgtgttttgttcataacttagcccctgggaaagataagttagctcttcgtgctctcaagtgtgtcttccttggttattctcgtgttcagaagggatatcgttattattctccagatcttcgtaggtaccttatgtcagctgacgtcacattttttgagtctaaacctttctttacttttgctgaccaccatgatatatctgaggtcttacctataccgacctttgaggagtttactatagctcctcctccaccttcgaccacagaggtttcatccataccagccgttgaggagtctagtgttgttcctcgtagttccccagccacaggaacaccactcttgacttatcatcatcgttcgcgccctacatcgggcccaactggttctcgtcctgcacctgacccttctcctgctgcggaccctgctcctagtacactgattgcacttcggaaaggtatacgaaccatacttaaccctaatcctcattatgtcggtttgagttatcatcgtctgtcatttccccattatgcttttatatcttctttgaactcggtttccatccctaagtctacaggtgaaacgttgtctcacccaggatggcgacaggctatgagtgacgagatgtctgctttacatacaagtggtacttgggagcttgttcctcttccctcaggtaaatctactgttggttgtcgttgggtttatgcagtcaaagttggtcccgatggccagattgatcgacttaaggcccgtcttgttgccaaaggatatactcagatatttgggctcgattacagtgataccttctctcccgtggctaaagtggcttcagtccgtctttttctatccatggctgcggttcgtcattggcccctctatcagctgaacactaagaatgccttttttcacggtgatcttgaggatgaggtttatatagagcaaccacctggttttgttgctcaggagggggtctcgtggccttgtatgtcgcttgcgtcggtcactttatggtctaaagcagtctcctagagcctggtttggtaagttcagcacggttatccaggagtttggcatgactcgtagtgaagctgatcactctgtgttttatcggcaccctgttgacattccgatggatccgaattctaaacttatgccaggacagggggagccgcttagcgatcctgcaagctataggcggctggttggaaaattaaattatctcacagtgactagacccgatatttcttatcctgtaagtgttgtgagtcgatttatgaattctccctgtgatagtcattgggttgcagttgtccgcattattcggtatataaaatcggctccaggcaaagggttactgtttgaggatcaaggtcatgagcagatcgttggatactcagatgctgattgggcaggatcaccttctgatagacgttctacgtctggatgttgtgttttagtaggaggcaatttggtgtcttggaagagcaagaaacagaatgtagttgctcggtctagtgcagaagcagaatatcgagcaatggctatggcaacatatgagctagtctcgaccaaacaattgctcaaggagttgaaatttggtgaaatcaatcggatggaacttgtgtgcgataatcaagctgcccttcatattgcatcaaatccggtgttccatgagagaactaaacacattgagattgattgtcacttcgtcagagaaaagatactttcaggagagattgctacaaagtttgtgaggtcgaatgatcaacttgcagatattttcaccaagtctctcactggtcctcgtattggttatatatgtaacaagctcggtacatatgatttgtatgcaccggcttgagggggagtgttagtttacagctatgtatatagtgtagtcttgtctcacattggaataggagtagtatgtccttgtatagtatagctataaataagacagtactaacgtcccttttgccgggggttctgcatctttaaatagatgcacgtggttccatagcagaccgtgttgatcacagatcgtgctgcatcctcttcccagcggactcggtgagcccctcttgtattgtattgaacatccaatatcaataacatattttctctcgtgctttctcacaggtctgtgatgtacccttgaaaggttcaagagtttggaggaagatagaaactctgtttatctcccaatcatccaaagatcttctaaagttccagttccatccttgtgagctccagactgacttaccaatgcttggctttgaagacttagagagaataagtcaggaaaaatctttcaaccttccttgccctatccggtgatcttcccaaaaagatgtcttcaacccattgccaacattgatcctgatattgctactgaaagatttcttttggtggcaggattactctcattaacaatgtacttgacaatctccatacatacgaatgtctctttaccctcttgccattaaggttgtaaagagacttgtcaaattaagaagaggtttcctatggaactgtttcaaggaaggaacctcctttcctttggtcaagtggagttaagtcatataatctaggaagtggagacttgggtataaaatagctgcaactacagaaaaggagcatcttatttaaatgatcacgcaaatgtgcccaaaactttaaatatctgcggagcatatggttgtagcaaaatttgaatcttccggtcaatgttgctcatgtccagtgaatacccctgatggtgaaagtgtcctgaagggaagcaggaacttattggaggaattggcatttaacactcagcatttcgttaggtcatagcccgctgaaaattgagtgcccagatttatatagttttgctctaaactgacgatgcagttgcacaacatacgacaaactaaggtgggacatcttcttcggaaggaattttgaggattaagagatagagtggttgattcagttgcaaatgaagcttcaagggttcaatatcatccaggagacaccggattctgatagataaaacaacagaaagatgaacactactttgttaggcttgttacaagttgctatcgtctttcttatctcggcacacaatttagatttgggaacttatttggaaaatagagtggttgtttttgtgaatagcatcagacaaagcttctgagctggtacgacagaaaactcaacagggagaataaaagactgtggttcacgatttctgcatgcatcttgtaggttatttggtgggtaaaatatttaatgttttgaagggaaggtagaacatgttcataggcttagattcaaatgtttgtatttttttggctctttggtgagagatgctgaatgtaaatgacataggcagctgactataatttctcagctccttgctttttaaattggcaggcactgatatgtacatgtgaacatccaacacttttgtggtgccgttccgatgaataaagcacattaatcacttactgatcaggagtaatagtttaggagttctagaatttttgtacataaaatgaaccaaaaagaatatcggaatgagaacatgtttctttttttgtttcttctttttcgtacaaatttcaataacacttctgatagaatagctaggtccatttgaattcctttggagacccttacacaaccaatgaatggcaagtatagcattttctaacaccctcccacatgtataatccagtttttagggtttagatgtggatttgatttgaccttattgcctttttttgtttttgttctttttgaagtagagagtgaggaggctcacaacgacgggctacgtagagcgagattaattcggctcaacgggctaatgattggacttacatgctacaacaatgttaggagaaagagagagagagagagagaagcccagagcagttccacgagttaagaaagagaagtccaaagcgattgaatatgaagagagaaagcggttgtgctaacaggctccctcaagtttggctctgagcatccaactcaaaaccttaaggcaatgagtagagtagcccaggaccatttaaactcctgttgaaaaccttacacaaccaataagggaacaagtgtaacattctcttacaaccctaccgtcttataagtcagggctctaatttagcataaaatcaaagtgaggcgatctactatgaaatgaagaaaataactgataaatataaagaatgttaattctcccatatagcctgaatgttcccagaacaaaataaattagtctcatgatttatcattaacatgatgttcctcttattttgagtgattaggaaggttaatcaaggagtaaattctttctaatttgtatcgtctagaattatttgtctaacaaattttcagattaccggtgatcaaaagaggaaaatattttgcatacaacgttaccataccttacaaaagggcgatgaacatttttttattttattattgtcctttttttcaattaggggttatgcagtcttcctccacgtgatattactcttagaatcacgtttttgtcattgctattacttactgtggtaagtacaaatgtgttttgaactctttttggtatgtattattgagttaatttttcgtttccatttcagagctgccgctttatcttctgctgggcatcttttgtggcttagtttcagtggcattatcaagttgtacatcatttatgctgcaaatagtggaaaatattcaaatgaccagcggcatgccaaaagcagcttttcctgtcctgggcggtcttctggttgggctggtagctttagcatatcctgaaatcctttaccagggttttgagaatgttaatattctgctagaatctcgcccactagtgaaaggcctctccgctgatctgttgctccagcttgtagctgtcaaaatagtaacaacttcattatgccgagcctctggattggttggaggctactatgcgccatctctattcatcggtgctgctactggaactgcatatgggaaaattgttagctacattatctctcatgctgatccaatctttcatctttccatcttggaagttgcatccccacaagcttatggcctggtatgaatttgtcttttgttagaagtagcattacatatctggataagtgagttttttattattgaaaagtaataacaggagaacaagagaatatatcacccaaatctacttctttcctctcttctattcttctgaaattcaaggtcctttaactcctccacagtctgtctagttattgatcctgtagacttaattcacataggtttaggacattcgagtttatccaaacttcatgaaaaggtttctaatttttttacattacattatgagtcgtgtctacttgagaaacatatcactccatgtttctatagtctgttttctccttagtttattctgatatgtggggtcctattaagtcagttcaaccttgtattttcattatttttgcagtatcattgataattattcaagatgtacttggattttctttacaagagatagttctcagttgttttttgtgttcctaagtttttatgctgcaatacaaaattggtttgatgtctctatttgcatttttcccaatgataatgccttagaatattttcttttccgtttcagtagcttattatttctttaggaactctttatcagaaatctcaactgagatagatgagaggaagaataagcatatcattggtctcattcagtcccctgtcaagcttagtttcttgagcgatgcggtttcacgtccttttattagattaattggatgcctcatctgctatccaaaatcagttaactttcgatattgtttcctcgcttacctttatactctctttccctcgagtctttgggagcacatgttttgttcaataacatagctcctggaaagtgaccagcgcaaccgacaaacaaggccttcttaatgtagaaggtggacatatgctattctagccacgggaaagaaagtaatattgtaatcaaacccaaatatctgagtataacctttggcaatggcgatcaatttgattatatggaccaactttgcctgcatatacccaccgacaaccaataatagatttaccgggaggtagagaaacaagctcccaaataccactaatatgtaaagcagatatatctctgatcatagcttgtccttgtggacatagggatagaaattaaggacaaagatgacacaaaagcataatgcggtgatgataaacgatgataactcaaatcaatataatggggatggggattgagagtggatcgaatatctttgcggaatgcgattggtagactaggaggagagaagtctgtggacatgatgttggactgagatcaataataagtcaagaatggtggagctacagaacatggaactggagctgtaggtgacataatcggagctgtaggaggtggagctatagaggaaggtgaaggagagatagcgactgaatctccaaaagatgaaaccggtaatacctcaaaaaatgtctaagagatcatttggacctatgaagtatgattgcgtttttaaaaaggtaacatcataaggtcaggtgaataacattgatatccccgttgcatcctcgagtaacttagaaatatacatttgagagcacggagagctaacttatcttttctggagcaaggttgtaaacaaaacacgtgctcccaaagacacgaggtggaagagagaaaggtgagtggggaaacaagacagaggatgaaacttgactcttgatagttgaagatgacatacaattaataagacaataggatgtgagatccaatgacagttctcatgaactgctgaaatggagaagacaaatactctggggcgttatcactacgaaatgtgcagttagaaaccccaaattgattttggatttcagtgtggaaggtctaaaaaatagagaacaactcagattgatttttcatcaagaatatccaagtggacttggaataatcatcaatgaaactgacaaagtagcggaattccaaggtagaactaacccgacaaggaccccaaacatctgaatggactaaagtgaaaggtaactctacccgattatcaggatgtcgagggaaatgagagtgagtatgccttctgagcggatatgactcacgctctagagtggacaagtgagacaaacgaggtactattttctaaagttctgataaattgggatgtcctaactgtatatgtaataaatctggtggatcagtaaaaggacaagctgtagggggaaaaaaataccaaatatttccagaagatggcaaactacaacagaagatgcaactgcattaacatgctcaggataggtgatgaaatcattgaggacaaagagttgatcaagaaggagattctggaattttaccagaacttatatagtgaaaatgaaccctggaggcgcagtgcaaatttcgaagacatctcctcactaagcatagaagagaagaactggttggaagctccatttgtagaaatagaggtgcttgaagctttgaaatcatgtgccccttataaagcaccaggtccagaaggcttcactatggatttctttcagaaaaattgggatactcttaaaacagacatcatggctgcacttaatcattttcaccagagctgtcacatggttagggcttgcaatgccaccttcattgccctaattccaaagaaaaatggtgctatggagctcagagactacagacctattagcttgacaggtattgtatacaaattggtttcaaagattttagcagagaggctcaagaaggtaattgacaaactagtctcgggggaacaaaatgctttcatcaagaacaggcagatcactgatgcttccttgattgccaatgaagtgctggattggagaatgaaaagtggagaaccaggcgtgttgtgcaaactggacattaaaaaggcttttgatcaattaagctggtcttacctcatgagtatcttgaggcagatgggctttggggagaaatggagaagatggataaactattgcatttcaactgtcaagtactctgttttggtgaatagggacccaatcggttttttctccccccaaaagggcctaaggcagggggatcccctctcccccttcctattcattctggcgatggaaggactcactaaaatgttggagaaggctaagcaactgcaatggatacaaggctttcaggtgggaaggaatcctgccagctcagttacagtatctcatctactctttgcggatgatactcttattttctgtggtactgagagatcacaagcacgaaatctcaacctgacactgatgatcttcgaggcactatcaggactccacatcaatatgataaagagcatcatataccctgtgaatgcagtccccaacatacaagagctagcagacatcctatgccgcaaaacagacactttcccaaccacatatcttggacttcccttgggagctaaattcaaatcaaaagaagtttggaatggagtcctagagaagtttgaaaagaggcttgcgacttggcaaatgcaatacctccccatgggtggcaggttaactttaatcaatagtgtactggacagtcttcccacataccacatatctttgttcccaattccaatctcagtcctaaagcagatggacaaactcagaaggaagttcttatgggaaggatgcagcaaaacacacaaatttccactagtgaaatggctgaaggtaactcaaccaaaattcaaaggagtcttgggaatcagggatgctatgctcttaaaatggctctggagatatggacaggaggaatctaggctatggaaggacatcatatttgctaaatatggagcacacaaccactggtgttccaagaaaacaaactctccttatggagttggtctgtggaagaacatcagcaaccactgggatgaattcttccaaaatgtaactttcaaagttgggaatgtaactcgtataagttttggaaggatagatggcttggaaatacacctttgaaagacatgtttcccagtatgtatcagattgccgtgaccaaagactccactgttgctcataatagaaacaatgacacttggtacccacttttcagaagaaatttgcaggattgggaggtcaacaacctactcacaatgttaagctccctagaatgtcataacattgaagatcaacaacctgacaaacttatttgggaaaattctaagagaggcaagtacacagtcaaagaatgatacattcacctctgtgaccagaatccaatatataactggccatggaaacatatctggagaactaaagtgcctaccaagatgacttgcttcacatgattgtctctaaatggggcctgtctcactcaagacaacttaatcaagaggaacatcatataagttaatagatgctacatgtgccaacaacagtcagaaagtgtaaagcacttattccttcactgctcagttgcaaaagaaatttggaacttcttctacactacctttggtctaaaatgggttatgccacaatcaactaagcaagcttttgaaagttggtatttttggagagttgataaatccattagaaaaatctggaaaatggtgtcggccgcaagtttttggtgtatttggaaagaaaggaactgaagatgttttgatggcatatcaactccactcaaggctgcgtgtttagttaacttattttgctggaactatctcacccctgttaatagtgctgatacttctgtggatttcattagccccctgatagtagcataggcttttgtaaatggagctaattatcctttctcttttgtactctttgcatcttcttgatgccttttaatgaatctaatttacttcatcaaaaagaaaatgacaagttgttgaaggaggaaaagatgtgagtccatgtgatttagcaaggataaggtactaaagtccatttgattcacgtccggtaccaatgatccgtctcgtgctgcattcctgtattaaaacagagtcatcaagaaataaaatagagcaaataagtgattggccaagcgactagtggatatgagattaaaaggactatggggaacataaaaaactgaattcaaaggtaaggaaggaagtggactagcttaacctattctagttgccatggtttgagaatcgttggccattgtgactattggaagtgattgagagtaagaaatagtagtgaaaggagatttgttacccgaaatataattagatgcacctgaatcaatgacccaaaagtcggaagaagaggaaacacaagtcacgctattacctgtttgaacaatagagattagtttggatcaaatagttgtatagagaactgaaatttggagaaatcaatcatatagaacttgtatgtgattattgttgccctttatattgcgtcaaatcctaaaacacattgagattaactgccacttatcacagaaaagatattctctagagacattgttacaatttcatgaagtcaagtaattagcttgaacatatcttcagcaagtccctcgtcagtcctcatattagttacatttgtaacaatgtcggtacataagacttataagcaccagtttgaggaggagtggtagagagttgatgtacatagttaaagtagatatacttacacttagtgttatgtaaagagtggatataaaaagggatcagcataagacaattgtcttcgcgcgtcttaacatttttttcctgtctttatttctctcatggtatcagataacctatctctatcttggtttacccaatggttggcccccatattgtattagccatgctccagttgactaggcttggacgggcagaggtgttaaattatcccatattggttgaaagaatgagctattgtctccttatatggtcttagacaattctccaactcatgagatattttgttttggctgagttagccctaaggtttattttttgtcatattctttaaccttatggcaatgcttgtacacggaaaaaccggagtgcaagacttaaattaggagaaggaaactattgaaggtgaggaacttaaagggttgtgagaatacacgggagaaaaaaatcttaatactatctagtggccttgtatatcaaatgatcagcttgcaaatattttcaccaagtccctcactggtcctcgtattagttacatatgtaacaagttcggtatatatgatttgtatgcaccggcttgaggttatgcatattctattcctcctactatatatgtgactaggaaatattttactcctactgcatatgggactaggactatttacacataactatctaacattcccctcaagccagtgcacacaagtcatatgtaccgagcttgttacatatgtaactaatacgaggaccagtgagggatttagtaaaaatatctgcaagctggtcattcgacatacaaggccactagactccccccgagcaacaaaaccaggtggttgctgataaacagaaactggccgaaaagttgccggaaaaatttgaaaatagtgagactaagccgaattctacactacaaaataggttctaaaacaccaccagaaaacaaaaacttttctagaaattactcttcacaccggaaaaaataaaagttgtcagaatttgatgtaatttatatagataggttcggaatcactggaggagtaagttgtcccgaagaagttttgtcaaaaagtggccggaatggctcacatgcgccggaaaacttactgtagctcgcaggaaccctagttctggcggtgcgtggaggcgcgtgacttaagattaagatgcttacaggactatcttgagaaatatacatattatatagacgcttgagttgcttcccaatcctaaatagaagcttttattcgtaggcaagaagggaagcagctttacttgagccaatagctttcaaggtgcacgttgtcacaccaaggacatccagaatttgattttatagggggtgtgagaaagcacgggagaaaatatgttattgatatttggataataaatacaatacaagaggtccctatttatagctatacactacaaggagatattactcctcttccaatgtgggacaagaatacactatacatatctgtaaactaacactccccctcaagtcggtgcatacacatcatatgtaccgatcttgttacacatgtagctaatacgagaaccaataagagacttagtgaaaatatctgctagttgatcattcgactttacaaactttgtaacaatatctcctgaaagtattttttctctgacaaagtgacagtcgatctcaatgtgtttagtcctctcatggaacaccggatttgacacaatatgaagagtagcttggttatcacacattagttccatcttgctgatttctccgaattttaactccttgagcaactgcttgacccaaaataactcacacgtcgtcatagccatggcccgatattcggcttcggcgctagatcgagcaactacattctgtttcttgctcttccacgagaccaaattacctcctactagaacacaatatccagacatagaacgtctatcaaaaggtgatcttgcccaatcagcatctgtgtacccaacaatctgctcgtggccttgatcctcgaatagtaatcctttgcccggagctgactttatataccgaagaatgcgaacaactgcatcccagtgactatcacagggagaatccataaactgacttacaacactcaccggaaaagaaatgtcaggtctagtcactgtgaggtaattcaatttgccaaccaacctcctatatctcgtagggtctctaagaggctccccctgtccaggcagaagcttagcattcagatccataggagagtcaataggtctgcaacccatcattccagtctcctcaagaatgtctaagacatacttccgctgtgaaataacaatacctgagctagactgagcgacctcaatacctaaaaaatacttcaatctgcccagatccttagtctggaagtgctgaaagagatgttgcttcagattagtaataccatcctgatcattgccagtaataacaatatcatcaacataaatcactagataaatacacagattaggagcagaatgccgataaaacacagagtgatcagcctcactacgagtcataccgaactcctgaataattgtgctgaacttaccaaaccaagctcgaggggactgtttcaaaccatatagtgacctgcgcaatctgcacacacaaccattaaactcccctaagcaacaaaaccaggtggttgctccatataaacttcttcctcaagatcactgtggagaaaagcattcttaatgtctaactgataaagaggccaatgacgtacaacagccatggacaaaaagagacgaacagatgctactttagccacgggagagaacatatcactataatcaagcccaaaaatctgagtatatccttttgcaacaagacgagccttaaaccgatcaacctggccatccggaccgactttgactgcataaacccaacgacaaccaacagtagacttacctgcaggaagaggaacaagctcccaagtgcaactcgcatgtaaagcagacatctcgtcaatcatagcatgtcgccatcctggatgagatagtgcctcacctgtagacttagggatagaaacagtggacaaagaagatataaaagcataatgaggtgacgacagacgatgataacttaaaccgacatagtggggattaggattaagtgtggatcatacacctttgcggagtgcaattggttgactaagaggagacaagtccgcagtaggtgcagaatctgatgcggggcgtgaatcacctgggcctgatgctggatatggacgacgatgataagtcaagagtggtggagctgccgaaggttgaactggattatgtggaggaactggagctataggtggtggagctacaactggagctgtaggtggtggaactagagtaactgaatctccaaaagatgaaactggtagtacctcagaaatatctaagtgatgacctgaacctgtgaagtatgattgggtttcaaagaaggtaacatcagcagacataaggtactgctggaggttaggagagtagcatcgataccccttttgtgttctcgagaaacctagaaatacgcacttaagagcacgaggagctaacttatccgttcctggaataaggttatgcacaaaacaagtgcttccaaagatacgaggtggaagagagaacaaaggtaagtggtaaaacatgacagagaatggaacttggttctggatagctgatgatgtcatacgattaataagatagcaagatgtaagaactgtatcccccaaaaacgcaacggagcatgagattgtatgagtagggtacgagcagtttcaataaaatgtctattctttctttcagctaccccattttgttgagatgtgtacagacaagatgtttgatgaataatcccatgagatttcataaactgctgaaatggggaagacaaatactctcgggcattatcactacgaaatgtgcgaatagaaaccccaaattgattttgaatttcagcgtggaaggtctggaaaatagaaaacagctcagatcgattttttatcaaaaatatccaagtgcacctggaataatcatcaatgaaactgacaaaatagcagaatcccaaggtggaactgacccgactaggaccccaaacatctgaatggactaaagtaaaaggtgactctgctcgattatcaagacgcctaaggaaatgggagcgagtatgcttaccgagctgacatgactcacactctagagctgacaagtgagataaaccagataccattttctgaagttttgacaaactgggatgtcccaaccgtttatgtaataaatctggtgaatcagtaacaggacatattgtagatggaagacaagatgcgagtccatgtatttagcaaggataaggtaataaagtccgtttgattcacgcccggtaccaatgatccgccccgtactgcgttcttgtataaaaacatggtcatcaagaaataaaataacgcatttaagtgatttggctaagcgactaacaactatgagattaaaaggactattgcgaacataaaggactgaatctaaaggtaaggaagaaagtgggcttgcttgacctattgcagttgccatggtttgagacccattggctattgtgacttttggaaaagattgagaatacgaaatagtagtgaaaagagatttgttaccagaaatatgatctgatgcacctgaatcaatgacccaagactcagaggatgaagattgggaaaaacaagtcacgctattacctgtttgaacaacagaagctatctcagaagatgtctgcttacatgctttgtactaaaggaactcaatataatctgctaaagaaaccatccgactattcaaagcatcggttcccatgtcgctacaatttgtagtagtagggttaacttgaaatagtggaaataagtaactccggtgagaaaactgaagaaatagcttgaaaacactgtttacaacagtaaaaacagaacactgttctgcgccggaatctactgtagctgacggaaaaactcaaagtagtcggaatgaaacgaaaaacagtaggggtaggatcggaattaccaggcgacccaactattctgaaggaagtttttcaaaaaatggccggaagtggtcgtacgtgtcggcgcgtgagctcacgcgcgtgagcttctggtggcgcgtggaggcgcgtgaggaggctgctgccggagattttcactggggtttggtcgccggacagtgactactcttgtggtagtgttggattttgcacaacactgacggagataaagcagacgcaaacagccttgaaaaagtcgccggaaaagacttccggtgactgatttctcttcctggaatcgctggaatttatgcacagcgataaatctctcacaattgctctgataccatgtgagaaagcatgggagaaaatatgttattgatatttggataataaatacaatacaagaggtccctatttatagctatacactacaaggagatattacttctcttccaatgtgggacaaaaatacactatacatatctgtaaactaacaaggggaatatcgtttaaagataaaaaagatagcgtgcagaagattgcatacattagagatgcaaaatacagaatacccatactcccagataatgcagtatgccttttgcatgacccactggttgaatggaagcacctggtcaatttactaggtgtgttagtgatttttgctgcttccttcccctttctaaactacatactatctaaaatgttagggggacagaagcccagtcaatctgactaggtgatgttagtggtttccgcttctttctcccacttctaaatgcgtactttctcaaatttaggagcatagaaacttaagcagctgcctacctgaggaggtgcatgggaacataagagaatagactttacctgtcatattttccataccttagttaattacagtgttatcctgataatgatctgttttctgtatctaggctgaatcgagattcaatcgcttttggctgaaaggatgctgctacagatccttagtttacatcattgtggttcttattctataagtacttcccctatcaactacttccttcttttttcttaggttatttgcctcttaggttgtttgcaaggaaaggaacaatagatgttttgatggaatagcaactccaaaccacttccttaaggctaatatactgtttggccaagcttcttcaaagtccaaagcccttttttgtcttcaaaaaagtatctttttttcccaaagttgaggtgtttggccaaacttttggaaggaaaaaaaagtgcttttgagtaaagcagaagctcttgagaagtagaaaaagtagttttttcccggaagcatttttttgaaaagcacttttgagaaaaataaacttagaaacactttttaaaagtttggccaaacactaattgctgcttaaaagtgtttttcagatttattagccaaacacaaactgcttctcaccaaaagtacttttttgaaaaatacttttttgaaaagtgattttcaaacaaagcacttttcaaaataagtttattttagaagcttgtcaaccggctataaatgtcttttatttttacagctagagtaccctaacacctgtaaattcccctagacatttttttcgactttgttagctcattaaccctagtataggactctttgttttggagctagcaaactcttttgttttcctatttttgcatcttcttggtgccatttataatatctcttacttcaccaaaaaaaataagttcccaaaatatgactaccttgagttggccaaagcataaccaaagcttgggcacaccagtgtttgcgtgaattttatggatgttccttacctttatccttctgtgcttatgtagcatctgtcttggttaatcttttctgaagtctatagtgtatttctgtgttgcaacatgagtttactgtcaatcttactgtttgacctcaattttgggttctttttgattttgaaagacatcgtttaacaggttggcatggctgctactcttgctggtgtctgtcaggtgcctctcactgctgttttgcttctctttgaactgacacagaattatcggatagttctgcccctcttgggagctgtggggttgtcttcttgggttacatctggacaaacaaggaaaagtgtagtgaaggatagagaaagactaaaagatgcaagagcccacatgatgcagcgacaaggaacttctttctccaacatttctagtttaacttattcttcaggtgtgaaaccttcacagaaagagagtaacctatgcaaacttgagagttccctctgtctttatgaatctgatgatgaagaaaatgatttggcaaggacaattctagtttcacaggcaatgagaacacgatatgtgacagttctaatgagcaccttgctaacggagaccatatccctcatgctagctgagaagcaatcttgtgcaataatagttgatgaaaataattttctcattggtctgctgacacttagtgatatccagaattacagcaagttgccaagagcagagggcaatttccaggaggtagcttcttggtacatttcaatattcttaactgatgaaaaaataagggaaattgatctagcatgaaattaagctaattataagttttacactgtagaactggtaaaacagggttggctggatatttctttgttgaatttttaggattatatgtattgttttagttttgtaggttgttttctgatgtgctttttgacttggcagaatcttaagatgaaatggaaggtgtttaaccaaaaaatagaattttcagtcaaagcctatatttagaagaaaacgggttattgataaccaagttttactttacttccccaacaatctatttggtaaatagcaaaagtaatgcgtatgtgagaaagcacgggagaaaatatattattgatattagatattcaatataatacaagaggtcctacacatcatatagctatagtctacaaactacatattactctcattccaatgtgggactacacataactaacactccccctcaagccggtgcatacatatcatatgtaccgagcttgttacacatgtaactaatacgagaaccagtaagagacttagtgaaaatatctgctagttgatcatttgactttacaaactttgtaaaaatatctcctgaaagtattttttctctgacaaagtaacagtcgatctcaatgtgtttagtcctctcatggaatagcggatttgacgcaatatgaagagcagcttggttatcacacaccagttccatcttgctgatttctccaaactttaactccttgagcaactgcttgacccaaactaactctcacgttgccatagccattgcccgatattcgacgtcggcgccagatcgagcaactacattctgtttcttgctcttccacgagaccaaattacctcctactagaacacaatatccaggcgtagaacgtctatcaaaaggtgatcctgcccaatcagcatttgtgtacccaacaatttgctcgtggcctcgatcctcgagtagtaatcctttgcttggagatgactttatataccgaagaatgcgaacaactgcatcccagtgactatcacagggagaatccataaactgacttacaacactcaccggaaaagaaatgtcaggtctagtcactgtgaggtaattcaatttgccaaccaacctcctatatctcgtagggtctctaagaggctccccgtgtctaggcagaagcttagcattcggatccataagagagtcaataggtctgtaacccatcattccagtctcctcaaaaatgtctaaggcataattccgctgtgaaataacaatacctgagctagactgaggcactgagcaacctcaatacctagaaaatacttcaatctgcccagatccttagtctggaagtgctgaaagagatgttgcttcagattagtaatatcatcctgatcattgccagtaataacaatatcatcaacataaaccactagataaatacacagattaggagtaaagtgccgataaaacacagagagatcagcctcactacgagtcatggcgaactcctgaataattatgctgaacttaccaaaccaagctcgaggggactgtttcaaaccatataatgacctgcacaatctacacacacaaccattaaactccccctgagcaacaaaaccaggtggttactccatataaacttcttcctcaagatcaccgtggagaaaagcattcttaatgtctaactgataaagaggccaatgacgtacaacagccatggacaaaaagagacgaacaaatgctattttagccacgggagagaaagtatcactataatcaagcccaaaaatctgagtatatccttttgcaacaagacgagccttaagccgatcaacctggccatccgggccgactttgaccgcataaacctaatgacaaccaacattagacttacctgcaggaagaggaacaagctcccaagtgccactcgcatgtaaagcagacatctcgtcaatcatagcatgtcgccatcctggatgagatagtgcctcacctgtagacttagggatagaaacagtggacaaagaagatataaaagcataatgaggtgatgacacacgatgatgacttaaaccgacatagtggggattaggattacgtgtggatcgtacgcctttgcggagtgcaattggttgactaagaggagacaagatcgtagtaggtgcagaatctgatgcagggcgtgaatcacttgggcatgatgttggatgtggacgacgatgataagtcaagagtggtggagctgcagaaggttgaactggattatgtggaggaactggaggtggagctacaactggagctgtaggtggtggaactggagctataagtggtggagctacaactggagctggagatgtagaggaagatgaatgagagatagtgactgaatctccaaaaaataaaattggtagtacctcagaaatatctaagtgatgacatgaacctgtgaagtatgattgagtttcaaagaaggtaacatcagcggacataaggtaccgctgaaggtcaagagagtagcatcgataccccttttgtgttctcgagtaacctagaaatacgcacttaagagcacgaggagctaacttatctgttcctggagtaaggttatggacaaaacaagtgattccaaagatacagggtggaagagagaacaaaggtaagtggggaaacatgacaaagaatggaacttggttttggataactgaagatggcatacgattaataagatagcaagatataagaactgcatccccccaaaaacgaaacggagcatgagattgtatgagtagggtacgagcaatttcaataagatgtctattttttctttcagctaccccattttgttgagatgtgtacagacaagatgtttgatgaataatcccatgagatttcataaactgctgaaatggggaagacaaatactctcgggcattatcactaggaaatgtgcgaatagaaaccccaaattgattttgaatttttagcgtggaaggtctggaaaaatagaaaacaactcagatcgattttttatcaaaaatatccaagtgcaccttgaataatcatcaattattcaataaaactgacaaagtagcagaatcccaaggtggaactgacccgactaggaccccaaacatttgagaatggactaaagtaaaaggtgactctgcttgattatcaagacgccgagggaaatggaagcgagtatgcttatcgaactgacatgactcacactctagagctgacaagtgagataaaccagataccattttatgaagttttgacaaattgggatgtcccgaccgtttatgtaataaatttggtgtattagtaacaggacaagttgttgaaggaagacaagatgtgagtccgtgtgatttagcaaggataaggtaataaagtccgtttgattcacgtccggtaccaataattcgtcccgtactgcgttcctgtataaaaacatggtcatcaagaaataaaacaacgcatttaagtgatttggctaagcgactaatagttatgagattaaaaggactattgggaacataaatgactgaatataaaggtaaggaaggaagtgagcttgcttgacttattgttgttgccattgtttgagacctattggccattgtgactcttgaaagagattgaaaatacgaaatagtagtgaaaagagatttgttaccagaaatatgatctgatgcacctgaatcaatgacccaaaactcagatgatgaagattgggagaaacaagtcacgctattacctgtttaaacaacagaagctatcacagaagatgtctgcttacatgctttgtaccgaaggaactcaatataatctgctaaagaaaccatccgactattcaaagtatcggttcccatgtcgctacaatttgtagtaataggatggatagactcggaaaattgtaaagttatcggaatttgtcgtaaccaggatcgagcaagctgtcttgaagaaatggtttcaaaaaatgtccggaaaggtcacttttacgccggaaaaatataaaaatggtcgaaatttgatttgaattagatgggtaggctcggaattgtgaggagagcagactgtcctgaagaagcttaatgaaaaaatggccggaaagtggccggaaccctcgccgtaaaagttgttaccggcgcgtgaaggcgcgtggcattttttctgccagataaattttcaggggttggtcgtcggagggtgatcccttgtggtggtgttggtttttgcacaataccgacaggccttaggtcacccgaaaatttgcacgatgactaagttctttcttcccggttaacgctggaatgacgcacatcgatcttttctcactaatgctatgataccatgtgagaaagcacgggagaaaatatattattgatattagatactcaatataatacaagaggtcatatttatagctatagtctacaaagtacatattactctcattcaaatgtgggactacacataactaacaacgtaaattaacaaagagaaataaggaatgtaacaacagtcaatccctaaaatcaaggtagaaaactttgataaagcagagaattatagaatgtatttcagtagtacttggaacttgtccttacaaataaaattctttatccttatataggggcgtacaatcataacatttttcgcacttaattcgaattcattatgagcattaattgtattgattgcccgttatcatagataaccataactgacgtatttgtaactataaatgccttataacggctctgattccccttccttatttacttctggtttgtgtatctttccttctttttagcctttattcattcagttctcgcctcttctttgacaactgtcaagcccgatcctctgttctgtactgtctcgtgggtgtttcccccgtaccttccttatattcttaattctgttaattgagagtgtcacttgtcactatgccattgttccacgcgtcatgtttcatccacgtgtaatatcttttttccaccaatacagataatcccccactttctgaatattctcaactgaatattcgggtaagtttttatggcgggaattctttgccgtcgtttttcgagtatcatcgtgtcatcttcagaaccgatgtgacgtacgtcacgtctatttaatgcctatgccaggtggcttctatcgattggctctgcagttttttagcgctttttagggtttttcagcggctgcgtcagtcacgaagtgacggttccattatgacgcttcataatgactaactttaatgatggtcgtgtcttcttattaatacttcattcctttttgatctcttggagtcttccttcttcagtatccaccacattacttctttgtatttctgcatcttctctttgatattcctttggacaatcatgtcttcttctacaccagacccccgtaaggttgtgattgttgacgaacttgatctttctactgctcctactagaagtaggagaggtggtagacttcgtagtcttggttcactatctaatcgtggttcttcttcccagggtagtgctgctaagccatcttcttctagacctagggctcctttaacccctagatcttcttctaggaatagagatttaaatgatccagtgcgcgaacctacagttgcagagattgttcctcaagaattttcttttgtaactgaccgtgaaaccataaggaatcaaatttcttctatagcctccctcaataccgctaacctttatccaagtttaatcagtaatggtcttctctcccgggttcgaagagaatattactgaaaccagatttcccaattttagtccctggtgccaaccagagaattactccataccatgttggtttttcctttgtttacacctacccttttactttagggttcaaaccacctattgaaccagtaatcattgaattctgtcgttatttcaacgtgtgtcttggccagattgaccacatagtatggagggctgttcatgccttcgttatttatcagatttggtttccatgcctttcacttttcagcacttgcttcatctctactcccctaaattgtttcgtgaagtagtttttactctcgtggctagaagtaagagagtgttggttagccttgaagacgattgggaccgtggctggtacgctcgttttgttgctgctcccactagtgcattagtgggtgaagaaaatatgcctttcccggagaaatggaactttgcacgtaagctttcttctcctctttttttttgtcttaaaaaaactccatgtaatcatatacccacttcttcagcaactatggaagttttttatgcttgggtagaaaagatgttaactgctgcgcctatggagaaaagatcctggaaatacttttctcaaagatttggttggaaagtgaagacgcacggtactttttaccttcattgtttttccttttctcttccttgtttgttcaatgatttctcatccttccctttttttttactagggtttccgattcgtggtattagtcccgcgtctgttccatcaactaggctttccgtgattcttgttcaggaaagaattttaagtgcttcttcttcaaaaaggaaaactgacggagcccgtggctctgatgacgaagaagaaacagaggagggttctttggtgcgaaggtcacgcgtcaggagacgcgtggtttctgatgatgaaactactccttctcatgaccctctatctagttcaatcccttttagactcacggatgagctagagagtacccctttagtgatttcttatgatgatgctgttgatccccctccaagttctgttgatagattgtttgctcatggcttcgagggtgatgaagttttgggcctgtttctgaagaattgccccttgcttcccttccagtttcagttttcattaacccttccgtgtccttacctgatgatactcctgttgttattctcgtggctgcttctactccgtcatctattcccgtgactgcttctcatgcagaggccaaaccttctagcagcagaagggcaatgaaaagagttgttgttgaggttcctgaaggtgagaacttattaagaaaatccggtcaagccgacgtgtagttgaaacctatgctcggccccgtagagaagaagaagttagaaagccatagctcactcactttaatgaatgatatcgttcattcttccttgaaagtacaagcttaattatatttcctttcttttctctttcttattcataactcttcctccttttttgcagatcaacttgattggcacagagcttatgaaaagagtttctcaggcggaccggcaagttatagatttgcgcaccgaggctgataactggaaggaacaattcgaaggtcttcaattggaaaaagaggttccggcggaagagaagaatgctttggaacaacagatgagagtgattgcctctgaattagcagttgaaaaagcttcctcgagccaggttggaaaggataagtatatacttgaatcctcctttgctgaacaactttccaaggcaactgaagaaataaggagtttgaaggaactccttaatcaaaaagaggtttatgcgagagaattggttcaaacacttactcaagttcaggaagatctccgtgcctctacttataagattcagttcttggaaagttctctcgcttctttgaagacagcttacgatgcctctgaagcagaaaaagaagagctgagagctgagatttaccagtgggagaaggattatgagattctcgaggataatctatcgttggatgtaagttgggctttcttaaacactcgtctcgagactctagttgaagccaaccatgagggttttgaccttaatgctgagattgctaaggctaaagaagcaattgataaaactcagcaacgtcaaatcttttcctcacctgaagacgaaggtcccgaaggtgatggagattga

SEQ ID NO: 12 - protein, 753 aa, Nicotiana tabacum
MISGQNTVLHNPPNSLFNSLSPRHICISFCNDKALKKSVTHSAPRFARLLNNESRKLLGRHPNCWPWARRPSLPPGRSSDGNIEKEQDMCDSSKVDSDSGIQIGSLLEEVIPQGNNTAIISACFVGLFTGISVVLFNAAVHEIRDLCWDGIPYRAASEEPIGVHWQRVILVPACGGLVVSFLNAFRATLEVSTEGSWTSSVKSVLEPVLKTMAACVTLGTGNSLGPEGPSVEIGTSVAKGVGALLDKGGRRKLSLKAAGSAAGIASGFNAAVGGCFFAVESVLWPSPAESSLSLTNTTSMVILSAVIASVVSEIGLGSEPAFAVPGYDFRTPTELPLYLLLGIFCGLVSVALSSCTSFMLQIVENIQTTSGMPKAAFPVLGGLLVGLVALAYPEILYQGFENVNILLESRPLVKGLSADLLLQLVAVKIVTTSLCRASGLVGGYYAPSLFIGAATGTAYGKIVSYIISHADPIFHLSILEVASPQAYGLVGMAATLAGVCQVPLTAVLLLFELTQDYRIVLPLLGAVGLSSWVTSGQTRKSVVKDREKLKDARAHMMQRQGTSFSNISSLTYSSGSPSQKESNLCKLESSLCLYESDDEENDLARTILVSQAMRTRYVTVLMSTLLMETISLMLAEKQSCAIIVDENNFLIGLLTLGDIQNYSKLPRTEGNFQEELVVAGVCSSKGNKCRVSCTVTPNTDLLSALTLMEKHDLSQLPVILGDVEDEGIHPVGILDRECINVACRALATREQLC

SEQ ID NO: 13 - protein, 818 aa, Nicotiana tabacum
MCDSSKDDSDSDSGIQIGSLLEEVIPQGNNTAIISACFVGLFTGISVVLFNAAVHEIRDLCWDGIPYRAASEEPIGVHWQRVILVPACGGLVVSFLNAFRATLEVSTEESWTSSVKSVLGPVLKTMAACVTLGTGNSLGPEGPSVEIGTSVAKGVGALLDKGGRRKLSLKAAGSAAGIASGFNAAVGGCFFAVESVLWPSPAESSLYLTNTTSMVILSAVIASVVSEIGLGSEPAFAVPGYDFRTPTELPLYLLLGIFCGLVSVALSSCTSFMLQIVENIQMTSGMPKAAFPVLGGLLVGLVALAYPEILYQGFENVNILLESRPLVKGLSADLLLQLVAVKIVTTSLCRASGLVGGYYAPSLFIGAATGTAYGKIVSYIISHADPIFHLSILEVASPQAYGLVGMAATLAGVCQVPLTAVLLLFELTQNYRIVLPLLGAVGLSSWVTSGQTRKSVVKDRERLKDARAHMMQRQGTSFSNISSLTYSSGVKPSQKESNLCKLESSLCLYESDDEENDLARTILVSQAMRTRYVTVLMSTLLTETISLMLAEKQSCAIIVDENNFLIGLLTLSDIQNYSKLPRAEGNFQEINLIGTELMKRVSQADRQVIDLRTEADNWKEQFEGLQLEKEVPAEEKNALEQQMRVIASELAVEKASSSQVGKDKYILESSFAEQLSKATEEIRSLKELLNQKEVYARELVQTLTQVQEDLRASTYKIQFLESSLASLKTAYDASEAEKEELRAEIYQWEKDYEILEDNLSLDVSWAFLNTRLETLVEANHEGFDLNAEIAKAKEAIDKTQQRQIFSSPEDEGPEGDGD

SEQ ID NO: 14 - protein, 906 aa, Nicotiana tabacum
MISGQNTVLHHPPNSLFNSLSPRHICVSFCNDKALKKSVTHSAPRFARLLNNESRKLLGRHPNCWPWARRPSLPPGRSCDGNIEKEQDMCDSSKDDSDSDSGIQIGSLLEEVIPQGNNTAIISACFVGLFTGISVVLFNAAVHEIRDLCWDGIPYRAASEEPIGVHWQRVILVPACGGLVVSFLNAFRATLEVSTEESWTSSVKSVLGPVLKTMAACVTLGTGNSLGPEGPSVEIGTSVAKGVGALLDKGGRRKLSLKAAGSAAGIASGFNAAVGGCFFAVESVLWPSPAESSLYLTNTTSMVILSAVIASVVSEIGLGSEPAFAVPGYDFRTPTELPLYLLLGIFCGLVSVALSSCTSFMLQIVENIQMTSGMPKAAFPVLGGLLVGLVALAYPEILYQGFENVNILLESRPLVKGLSADLLLQLVAVKIVTTSLCRASGLVGGYYAPSLFIGAATGTAYGKIVSYIISHADPIFHLSILEVASPQAYGLVGMAATLAGVCQVPLTAVLLLFELTQNYRIVLPLLGAVGLSSWVTSGQTRKSVVKDRERLKDARAHMMQRQGTSFSNISSLTYSSGVKPSQKESNLCKLESSLCLYESDDEENDLARTILVSQAMRTRYVTVLMSTLLTETISLMLAEKQSCAIIVDENNFLIGLLTLSDIQNYSKLPRAEGNFQEINLIGTELMKRVSQADRQVIDLRTEADNWKEQFEGLQLEKEVPAEEKNALEQQMRVIASELAVEKASSSQVGKDKYILESSFAEQLSKATEEIRSLKELLNQKEVYARELVQTLTQVQEDLRASTYKIQFLESSLASLKTAYDASEAEKEELRAEIYQWEKDYEILEDNLSLDVSWAFLNTRLETLVEANHEGFDLNAEIAKAKEAIDKTQQRQIFSSPEDEGPEGDGD

SEQ ID NO: 15 - protein, 818 aa, Artificial sequence
  Synthetic sequence Protein sequence of NtCLCe from Nicotiana tabacum, including a P184S mutation
MCDSSKDDSDSDSGIQIGSLLEEVIPQGNNTAIISACFVGLFTGISVVLFNAAVHEIRDLCWDGIPYRAASEEPIGVHWQRVILVSACGGLVVSFLNAFRATLEVSTEESWTSSVKSVLGPVLKTMAACVTLGTGNSLGPEGPSVEIGTSVAKGVGALLDKGGRRKLSLKAAGSAAGIASGFNAAVGGCFFAVESVLWPSPAESSLYLTNTTSMVILSAVIASVVSEIGLGSEPAFAVPGYDFRTPTELPLYLLLGIFCGLVSVALSSCTSFMLQIVENIQMTSGMPKAAFPVLGGLLVGLVALAYPEILYQGFENVNILLESRPLVKGLSADLLLQLVAVKIVTTSLCRASGLVGGYYAPSLFIGAATGTAYGKIVSYIISHADPIFHLSILEVASPQAYGLVGMAATLAGVCQVPLTAVLLLFELTQNYRIVLPLLGAVGLSSWVTSGQTRKSVVKDRERLKDARAHMMQRQGTSFSNISSLTYSSGVKPSQKESNLCKLESSLCLYESDDEENDLARTILVSQAMRTRYVTVLMSTLLTETISLMLAEKQSCAIIVDENNFLIGLLTLSDIQNYSKLPRAEGNFQEINLIGTELMKRVSQADRQVIDLRTEADNWKEQFEGLQLEKEVPAEEKNALEQQMRVIASELAVEKASSSQVGKDKYILESSFAEQLSKATEEIRSLKELLNQKEVYARELVQTLTQVQEDLRASTYKIQFLESSLASLKTAYDASEAEKEELRAEIYQWEKDYEILEDNLSLDVSWAFLNTRLETLVEANHEGFDLNAEIAKAKEAIDKTQQRQIFSSPEDEGPEGDGD

SEQ ID NO: 16 - DNA, 11 bp, Nicotiana tabacum
ctctgctcgag

SEQ ID NO: 17 - DNA, 11 bp, Nicotiana tabacum
aagttatccca

SEQ ID NO: 18 - DNA, 10 bp, Nicotiana tabacum
aatacgtgat

SEQ ID NO: 19 - DNA, 10 bp, Nicotiana tabacum
tttgttggga

SEQ ID NO: 20 - DNA, 10 bp, Nicotiana tabacum
accagaaggc

SEQ ID NO: 21 - DNA, 10 bp, Nicotiana tabacum
ctagtgttga

SEQ ID NO: 22 - DNA, 9 bp, Nicotiana tabacum
cagctgctg

SEQ ID NO: 23 - DNA, 10 bp, Nicotiana tabacum
aatcgcttct

SEQ ID NO: 24 - DNA, 10 bp, Nicotiana tabacum
tgcgacagca

SEQ ID NO: 25 - DNA, 11 bp, Nicotiana tabacum
caaagtcgata

SEQ ID NO: 26 - DNA, 11 bp, Nicotiana tabacum
tataatctcgg

SEQ ID NO: 27 - DNA, 10 bp, Nicotiana tabacum
ttgctttgtt

SEQ ID NO: 28 - DNA, 10 bp, Nicotiana tabacum
tgaagacaat

SEQ ID NO: 29 - DNA, 10 bp, Nicotiana tabacum
gccgcttgtg

SEQ ID NO: 30 - DNA, 11 bp, Nicotiana tabacum
gctttgttggc

SEQ ID NO: 31 - DNA, 10 bp, Nicotiana tabacum
tcttcaccgg

SEQ ID NO: 32 - DNA, 10 bp, Nicotiana tabacum
agatatgtgc

SEQ ID NO: 33 - DNA, 10 bp, Nicotiana tabacum
acagcagcaa

SEQ ID NO: 34 - DNA, 11 bp, Nicotiana tabacum
aatctcggctt

SEQ ID NO: 35 - DNA, 11 bp, Nicotiana tabacum
ctttgttggcc

SEQ ID NO: 36 - DNA, 11 bp, Nicotiana tabacum
caataataccg

SEQ ID NO: 37 - DNA, 11 bp, Nicotiana tabacum
tataatctcgg

SEQ ID NO: 38 - DNA, 10 bp, Nicotiana tabacum
gctggaatcg

SEQ ID NO: 39 - DNA, 11 bp, Nicotiana tabacum
ttctggtttgt

SEQ ID NO: 40 - DNA, 10 bp, Nicotiana tabacum
ctcttcaccg

SEQ ID NO: 41 - DNA, 10 bp, Nicotiana tabacum
tatcagtgtc

SEQ ID NO: 42 - DNA, 10 bp, Nicotiana tabacum
ccagcttgtg

SEQ ID NO: 43 - DNA, 11 bp, Nicotiana tabacum
cggtttggtag

SEQ ID NO: 44 - DNA, 11 bp, Nicotiana tabacum
ccaagggagtt

SEQ ID NO: 45 - DNA, 10 bp, Nicotiana tabacum
gagctctgct

SEQ ID NO: 46 - DNA, 10 bp, Nicotiana tabacum
agctctgctt

SEQ ID NO: 47 - DNA, 11 bp, Nicotiana tabacum
ataaaggtggt

SEQ ID NO: 48 - DNA, 10 bp, Nicotiana tabacum
ctcaaggctg

SEQ ID NO: 49 - DNA, 11 bp, Nicotiana tabacum
tggatcagctg

SEQ ID NO: 50 - DNA, 11 bp, Nicotiana tabacum
tggatcagctg

SEQ ID NO: 51 - DNA, 11 bp, Nicotiana tabacum
tggaatcgctt

SEQ ID NO: 52 - DNA, 11 bp, Nicotiana tabacum
gacaatggccg

SEQ ID NO: 53 - DNA, 10 bp, Nicotiana tabacum
ttgtgtcaca

SEQ ID NO: 54 - DNA, 11 bp, Nicotiana tabacum
gtgtcacatta

SEQ ID NO: 55 - DNA, 10 bp, Nicotiana tabacum
gaactgggaa

SEQ ID NO: 56 - DNA, 11 bp, Nicotiana tabacum
ctggtttgttc

SEQ ID NO: 57 - DNA, 11 bp, Nicotiana tabacum
ccatattattc

SEQ ID NO: 58 - DNA, 11 bp, Nicotiana tabacum
accagcttgtg

SEQ ID NO: 59 - DNA, 11 bp, Nicotiana tabacum
cggtttggtag

SEQ ID NO: 60 - DNA, 11 bp, Nicotiana tabacum
ggtcgtagaaa

SEQ ID NO: 61 - DNA, 11 bp, Nicotiana tabacum
ctgtcactcaa

SEQ ID NO: 62 - DNA, 11 bp, Nicotiana tabacum
gtatccagata

SEQ ID NO: 63 - DNA, 10 bp, Nicotiana tabacum
gatctctgct

SEQ ID NO: 64 - DNA, 10 bp, Nicotiana tabacum
ggagcccatt

SEQ ID NO: 65 - DNA, 11 bp, Nicotiana tabacum
gagtacattgg

SEQ ID NO: 66 - DNA, 11 bp, Nicotiana tabacum
tcaaggctgct

SEQ ID NO: 67 - DNA, 10 bp, Nicotiana tabacum
gatcagctgc

SEQ ID NO: 68 - DNA, 11 bp, Nicotiana tabacum
accagaaggcc

SEQ ID NO: 69 - DNA, 12 bp, Nicotiana tabacum
tagtgttgaaat

SEQ ID NO: 70 - DNA, 10 bp, Nicotiana tabacum
aacaagatat

SEQ ID NO: 71 - DNA, 11 bp, Nicotiana tabacum
tgcgacagcag

SEQ ID NO: 72 - DNA, 10 bp, Nicotiana tabacum
atgtgcgaca

SEQ ID NO: 73 - DNA, 12 bp, Nicotiana tabacum
cagcaaagacga

SEQ ID NO: 74 - DNA, 11 bp, Nicotiana tabacum
cccttggttag

SEQ ID NO: 75 - DNA, 11 bp, Nicotiana tabacum
ttcatgaaata

SEQ ID NO: 76 - DNA, 9 bp, Nicotiana tabacum
cagcttgtg

SEQ ID NO: 77 - DNA, 10 bp, Nicotiana tabacum
cggtttggta

SEQ ID NO: 78 - DNA, 11 bp, Nicotiana tabacum
gacaatggccg

SEQ ID NO: 79 - DNA, 11 bp, Nicotiana tabacum
ttgtgtcacat

SEQ ID NO: 80 - DNA, 11 bp, Nicotiana tabacum
aactgggaatt

SEQ ID NO: 81 - DNA, 10 bp, Nicotiana tabacum
cttaggacca

SEQ ID NO: 82 - DNA, 11 bp, Nicotiana tabacum
gaatcgcttct

SEQ ID NO: 83 - DNA, 10 bp, Nicotiana tabacum
gtttgttccc

SEQ ID NO: 84 - DNA, 11 bp, Nicotiana tabacum
cattgccatgg

SEQ ID NO: 85 - DNA, 10 bp, Nicotiana tabacum
atcttataca

SEQ ID NO: 86 - DNA, 11 bp, Nicotiana tabacum
attgcatattg

SEQ ID NO: 87 - DNA, 11 bp, Nicotiana tabacum
gactcatcact

SEQ ID NO: 88 - DNA, 10 bp, Nicotiana tabacum
ccttcttttg

SEQ ID NO: 89 - DNA, 11 bp, Nicotiana tabacum
ttctcaagaaa

SEQ ID NO: 90 - DNA, 11 bp, Nicotiana tabacum
cttcaacctaa

SEQ ID NO: 91 - DNA, 10 bp, Nicotiana tabacum
tatttatgaa

SEQ ID NO: 92 - DNA, 11 bp, Nicotiana tabacum
gagtagtgcca

SEQ ID NO: 93 - DNA, 10 bp, Nicotiana tabacum
cggtgggtct

SEQ ID NO: 94 - DNA, 11 bp, Nicotiana tabacum
ctggtgagctt

SEQ ID NO: 95 - DNA, 11 bp, Nicotiana tabacum
ctgatgtaaag

SEQ ID NO: 96 - DNA, 10 bp, Nicotiana tabacum
gatttgcatc

SEQ ID NO: 97 - DNA, 10 bp, Nicotiana tabacum
cctgactaac

SEQ ID NO: 98 - DNA, 10 bp, Nicotiana tabacum
gtaccttatg

SEQ ID NO: 99 - DNA, 10 bp, Nicotiana tabacum
gatttgcata

SEQ ID NO: 100 - DNA, 11 bp, Nicotiana tabacum
tttgatagctg

SEQ ID NO: 101 - DNA, 11 bp, Nicotiana tabacum
ccttctctgcg

SEQ ID NO: 102 - DNA, 11 bp, Nicotiana tabacum
agctgagaggg

SEQ ID NO: 103 - DNA, 10 bp, Nicotiana tabacum
cggtaagatc

SEQ ID NO: 104 - DNA, 11 bp, Nicotiana tabacum
tcaggcaggtg

SEQ ID NO: 105 - DNA, 10 bp, Nicotiana tabacum
ggctccgcca

SEQ ID NO: 106 - DNA, 10 bp, Nicotiana tabacum
gtaccttatg

SEQ ID NO: 107 - DNA, 10 bp, Nicotiana tabacum
gatttgcata

SEQ ID NO: 108 - DNA, 11 bp, Nicotiana tabacum
ctactgctgca

SEQ ID NO: 109 - DNA, 11 bp, Nicotiana tabacum
ggcctggaatt

SEQ ID NO: 110 - DNA, 10 bp, Nicotiana tabacum
gattgctgtg

SEQ ID NO: 111 - DNA, 11 bp, Nicotiana tabacum
catctggtctc

SEQ ID NO: 112 - DNA, 11 bp, Nicotiana tabacum
cggagcagctt

SEQ ID NO: 113 - DNA, 10 bp, Nicotiana tabacum
ccttatggct

SEQ ID NO: 114 - DNA, 11 bp, Nicotiana tabacum
cagggctgtat

SEQ ID NO: 115 - DNA, 10 bp, Nicotiana tabacum
cggttctcgg

SEQ ID NO: 116 - DNA, 11 bp, Nicotiana tabacum
ggttctcggag

SEQ ID NO: 117 - DNA, 11 bp, Nicotiana tabacum
agcttccctta

SEQ ID NO: 118 - DNA, 11 bp, Nicotiana tabacum
catcacttttg

SEQ ID NO: 119 - DNA, 8 bp, Nicotiana tabacum
gattgctg

SEQ ID NO: 120 - DNA, 10 bp, Nicotiana tabacum
gtggggctcc

SEQ ID NO: 121 - DNA, 10 bp, Nicotiana tabacum
ccacatgctc

SEQ ID NO: 122 - DNA, 11 bp, Nicotiana tabacum
tgcggttctcg

SEQ ID NO: 123 - DNA, 10 bp, Nicotiana tabacum
agcagcttcc

SEQ ID NO: 124 - DNA, 11 bp, Nicotiana tabacum
atgccaacccg

SEQ ID NO: 125 - DNA, 11 bp, Nicotiana tabacum
agccatggatg

SEQ ID NO: 126 - DNA, 11 bp, Nicotiana tabacum
aggagtgggaa

SEQ ID NO: 127 - DNA, 11 bp, Nicotiana tabacum
tgagagagaaa

SEQ ID NO: 128 - DNA, 11 bp, Nicotiana tabacum
cagggctgtat

SEQ ID NO: 129 - DNA, 11 bp, Nicotiana tabacum
ccgttctggga

SEQ ID NO: 130 - DNA, 11 bp, Nicotiana tabacum
gcgtcatattt

SEQ ID NO: 131 - DNA, 11 bp, Nicotiana tabacum
ttgagctaaca

SEQ ID NO: 132 - DNA, 11 bp, Nicotiana tabacum
gccatggatga

SEQ ID NO: 133 - DNA, 10 bp, Nicotiana tabacum
aaatatcact

SEQ ID NO: 134 - DNA, 10 bp, Nicotiana tabacum
caaataccaa

SEQ ID NO: 135 - DNA, 11 bp, Nicotiana tabacum
cagcaggggtg

SEQ ID NO: 136 - DNA, 10 bp, Nicotiana tabacum
cttatggctg

SEQ ID NO: 137 - DNA, 11 bp, Nicotiana tabacum
ttcaatgagaa

SEQ ID NO: 138 - DNA, 12 bp, Nicotiana tabacum
cacttcaagggt

SEQ ID NO: 139 - DNA, 11 bp, Nicotiana tabacum
ttgtcctggca

SEQ ID NO: 140 - DNA, 10 bp, Nicotiana tabacum
cattgccatg

SEQ ID NO: 141 - DNA, 11 bp, Nicotiana tabacum
gatcttataca

SEQ ID NO: 142 - DNA, 11 bp, Nicotiana tabacum
ttctccttctg

SEQ ID NO: 143 - DNA, 12 bp, Nicotiana tabacum
caataacaatgc

SEQ ID NO: 144 - DNA, 10 bp, Nicotiana tabacum
gctgagaggg

SEQ ID NO: 145 - DNA, 12 bp, Nicotiana tabacum
cggtaagatcga

SEQ ID NO: 146 - DNA, 10 bp, Nicotiana tabacum
tttgggattg

SEQ ID NO: 147 - DNA, 11 bp, Nicotiana tabacum
tgtgccatctg

SEQ ID NO: 148 - DNA, 12 bp, Nicotiana tabacum
ctccgccacatg

SEQ ID NO: 149 - DNA, 10 bp, Nicotiana tabacum
tcattgtacc

SEQ ID NO: 150 - DNA, 11 bp, Nicotiana tabacum
gcatcccctga

SEQ ID NO: 151 - DNA, 11 bp, Nicotiana tabacum
taacacaaccc

SEQ ID NO: 152 - DNA, 10 bp, Nicotiana tabacum
caataacaat

SEQ ID NO: 153 - DNA, 10 bp, Nicotiana tabacum
ctggttcttc

SEQ ID NO: 154 - DNA, 12 bp, Nicotiana tabacum
gccaacccggag

SEQ ID NO: 155 - DNA, 11 bp, Nicotiana tabacum
catggatgaga

SEQ ID NO: 156 - DNA, 11 bp, Nicotiana tabacum
ccttaatgaaa

SEQ ID NO: 157 - DNA, 10 bp, Nicotiana tabacum
acgaaggaca

SEQ ID NO: 158 - DNA, 11 bp, Nicotiana tabacum
gtgggaagtga

SEQ ID NO: 159 - DNA, 10 bp, Nicotiana tabacum
agagaaattc

SEQ ID NO: 160 - DNA, 11 bp, Nicotiana tabacum
tccctgtcgtc

SEQ ID NO: 161 - DNA, 11 bp, Nicotiana tabacum
atgaaggagtg

SEQ ID NO: 162 - DNA, 11 bp, Nicotiana tabacum
gaactcacctt

SEQ ID NO: 163 - DNA, 11 bp, Nicotiana tabacum
ttttggttctc

SEQ ID NO: 164 - DNA, 10 bp, Nicotiana tabacum
caggcaggtg

SEQ ID NO: 165 - DNA, 11 bp, Nicotiana tabacum
ggctccgccac

SEQ ID NO: 166 - DNA, 10 bp, Nicotiana tabacum
atggctggtt

SEQ ID NO: 167 - DNA, 11 bp, Nicotiana tabacum
aatgagaatga

SEQ ID NO: 168 - DNA, 10 bp, Nicotiana tabacum
tggtgagctt

SEQ ID NO: 169 - DNA, 11 bp, Nicotiana tabacum
ctgatgtaaag

SEQ ID NO: 170 - DNA, 10 bp, Nicotiana tabacum
aaggacagag

SEQ ID NO: 171 - DNA, 11 bp, Nicotiana tabacum
agtgggaagtg

SEQ ID NO: 172 - DNA, 21 bp, Artificial sequence
  Synthetic sequence Primer
tatctcctcgccatatctgta

SEQ ID NO: 173 - DNA, 22 bp, Artificial sequence
  Synthetic sequence Primer
gtgcaaacacacttgtatttac

SEQ ID NO: 174 - DNA, 19 bp, Artificial sequence
  Synthetic sequence Primer
accatctcttcctccggga

SEQ ID NO: 175 - DNA, 23 bp, Artificial sequence
  Synthetic sequence Primer
tataggatactcctctgataaat

SEQ ID NO: 176 - DNA, 23 bp, Artificial sequence
  Synthetic sequence Primer
ttgtacaatttatcagaggagta

SEQ ID NO: 177 - DNA, 20 bp, Artificial sequence
  Synthetic sequence Primer
ttggtttgagtgcaaacaca

SEQ ID NO: 178 - DNA, 22 bp, Artificial sequence
  Synthetic sequence Primer
actatatcgaggatagaaggta

SEQ ID NO: 179 - DNA, 23 bp, Artificial sequence
  Synthetic sequence Primer
tatctatttatacatctggttcg

SEQ ID NO: 180 - DNA, 20 bp, Artificial sequence
  Synthetic sequence Primer
cttgtgatccatcacttccc

SEQ ID NO: 181 - DNA, 23 bp, Artificial sequence
  Synthetic sequence Primer
tatgactatttctgtgcatcttt

SEQ ID NO: 182 - DNA, 22 bp, Artificial sequence
  Synthetic sequence Primer
gccttgtgattcatcacttcaa

SEQ ID NO: 183 - DNA, 23 bp, Artificial sequence
  Synthetic sequence Primer
tatgactatttctgtgcatctta

SEQ ID NO: 184 - DNA, 19 bp, Artificial sequence
  Synthetic sequence Primer
ggttcttctcgctctgagc

SEQ ID NO: 185 - DNA, 22 bp, Artificial sequence
  Synthetic sequence Primer
aacgtaaaataactttgccacg

(19) United States
(12) Patent Application Publication
(10) Pub. No.: US 2017/0145431 A1
(43) Pub. Date: May 25, 2017

(54) MODULATION OF NITRATE CONTENT IN PLANTS

(71) Applicant: PHILIP MORRIS PRODUCTS S.A., Neuchatel (CH)

(72) Inventors: Prisca Campanoni, Vaud (CH); Lucien Bovet, La Chaux-de-Fonds (CH)

(21) Appl. No.: 15/316,696

(22) PCT Filed: Jun. 24, 2015
(86) PCT No.: PCT/EP2015/064308

§ 371 Date: Dec. 6, 2016

(30) Foreign Application Priority Data
Jun. 25, 2014 (EP) 14173987.0

(51) Int. Cl. C12N 15/82; C07K 14/415

(52) U.S. Cl. CPC C12N 15/8243; C07K 14/415

(57) ABSTRACT

In one aspect, there is provided a mutant, non-naturally occurring or transgenic plant cell comprising: (i) a polynucleotide comprising, consisting or consisting essentially of a sequence encoding a member of the CLC family of chloride channels and having at least 60% sequence identity to SEQ ID NO:1 or SEQ ID NO:2 or SEQ ID NO:3 or SEQ ID NO:4 or SEQ ID NO:10 or SEQ ID NO:11; (ii) a polypeptide encoded by the polynucleotide set forth in (i); (iii) a polypeptide comprising, consisting or consisting essentially of a sequence encoding a member of the CLC family of chloride channels and having at least 60% sequence identity to SEQ ID NO:5 or SEQ ID NO:6 or SEQ ID NO:7 or SEQ ID NO:12 or SEQ ID NO:13 or SEQ ID NO: 14; or (iv) a construct, vector or expression vector comprising the isolated polynucleotide set forth in (i); and wherein the expression or activity of the polynucleotide or the polypeptide is modulated as compared to a control plant and wherein the nitrate levels in the mutant, non-naturally occurring or transgenic plant containing the mutant, non-naturally occurring or transgenic plant cell are modulated as compared to the control plant containing the control plant cell.

### FIELD OF THE INVENTION

The present invention discloses novel polynucleotide sequences of genes encoding members of the CLC family of chloride channels from the genus Nicotiana and variants, homologues, fragments and mutants thereof. The polypeptide sequences and variants, homologues, fragments and mutants thereof are also disclosed. The modification of the expression of one or more of these genes or the activity of the protein encoded thereby to modulate the levels of tobacco specific nitrosamines (TSNAs) in a plant or component part thereof is also disclosed.

### BACKGROUND OF THE INVENTION

Tobacco Specific Nitrosamines (TSNAs) are formed primarily during the curing and processing of tobacco leaves. Tobacco curing is a process of physical and biochemical changes that bring out the aroma and flavor of each variety of tobacco. It is believed that the amount TSNA in cured tobacco leaf is dependent on the accumulation of nitrites, which accumulate during the death of the plant cell and are formed during curing by the reduction of nitrates under conditions approaching an anaerobic (oxygen deficient) environment. The reduction of nitrates to nitrites is believed to occur by the action of bacteria on the surface of the leaf under anaerobic conditions, and this reduction is particularly pronounced under certain conditions. Once nitrites are formed, these compounds are believed to combine with various tobacco alkaloids, including pyridine-containing compounds, to form nitrosamines.

The four principal TSNAs, that is, those typically found to be present in the highest concentrations, are N-nitrosonicotine (NNN), 4-(methylnitrosamino)-1-(3-pyridyl)-1-butanone (NNK), N-nitrosoanabasine (NAB) and N-nitrosoanatabine (NAT). Minor compounds, that is, those typically found at significantly lower levels than the principal TSNAs, include 4-(methylnitrosamino) 4-(3-pyridyl) butanal (NNA), 4-(methylnitrosamino)-1-(3-pyridyl)-1-butanol (NNAL), 4-(methylnitrosamino)4-(3-pyridyl)-1-butanol (iso-NNAL), and 4-(methylnitrosamino)-4-(3-pyridyl)-1-butyric acid (iso-NNAC). At least NNN and NNK have been reported to be carcinogenic when applied to animals in laboratory studies.

Lowering the concentrations of compounds responsible for the nitrosation of alkaloids to TSNAs can result in decreased TSNA levels in cured leaves. A major nitrosating agent in tobacco leaves is nitrite (NO₂⁻), resulting from the reduction of free nitrate (NO₃⁻) through an enzymatic reaction possibly catalyzed by bacteria during curing. Fertilizer studies altering nitrate levels in Burley plants resulted in different TSNA levels in cured leaves and smoke. Nitrate is the major source of nitrogen available in the soil. In plants, it is absorbed by root epidermal cells and transported to the whole plant to be first reduced to nitrite which is further reduced to ammonia and then assimilated into amino acids. Unfortunately, limiting nitrogen during Burley growth results in unfavorable agronomic phenotypes such as poor biomass yield and delay in plant maturation. Limiting nitrogen is therefore not a commercially viable approach to reduce TSNA levels. Trying to manipulate nitrate accumulation in tobacco leaf is thus a major challenge.

WO98/58555 describes the treatment of tobacco leaves before or during flue-curing by microwaving for reducing TSNAs. U.S. Pat. No. 5,810,020 describes a process for removing TSNAs from tobacco by contacting the tobacco material with a trapping sink, wherein the trapping sink comprises a select transition metal complex which is readily nitrosated to form a nitrosyl complex with little kinetic or thermodynamic hindrance. U.S. Pat. No. 6,202,649 describes a method of substantially preventing formation of TSNAs by, among other things, curing tobacco in a controlled environment having a sufficient airflow to substantially prevent an anaerobic condition around the vicinity of the tobacco leaf. The controlled environment is provided by controlling one or more curing parameters, such as airflow, humidity, and temperature. However, methods such as these can add considerable cost and time to the production of tobacco and therefore are less likely to be accepted by the tobacco industry. Thus, a need remains for an effective and relatively inexpensive method for reducing TSNAs.

Molecular based methods for reducing the levels of TSNAs in plants are highly desirable since they do not require expensive, and often complex, methods to achieve the reduced levels of TSNAs. One such molecular based approach is disclosed in WO2011/088180. Compositions and methods are disclosed for inhibiting the expression or function of root-specific nicotine demethylase polypeptides that are involved in the metabolic conversion of nicotine to nornicotine in the roots of tobacco plants. The gene sequence of the CYP82E10 nicotine demethylase gene is disclosed. Reducing the expression of this gene was found to reduce the levels of NNN in cured tobacco leaves. Whilst reduced levels of NNN may be obtained, there is more than one TSNA that has been reported to be carcinogenic which will still remain in the modified plants. Other nicotine demethylase genes include CYP82E4 and CYP82E5 which participate in the conversion of nicotine to nornicotine and are described in WO2006091194, WO2008070274 and WO2009064771.

We have now found that modifying certain CLC family genes can also provide an increase in the biomass yield of the plant.

### SUMMARY OF THE INVENTION

The inventors have cloned novel genes encoding various members of the CLC family of chloride channels from plants belonging to the genus Nicotiana and denoted as CLC-Nt2 and NtCLCe. Two copies of the orthologous gene originating from two ancestors, N. tomentosiformis and N. sylvestris exist in Nicotiana tabacum, and are denoted herein as CLC-Nt2-t and CLC-Nt2-s or NtCLCe-t and NtCLCe-s, respectively. The polynucleotide sequences of these genes are set forth in SEQ ID NOs: 1-4, 10 and 11 and the polypeptide sequences of these genes are set forth in SEQ ID NOs: 5-7 and 12-14. By reducing the expression of these genes in tobacco plants a reduction in nitrate levels in plants is seen. In particular, a reduction in nitrate levels in green leaves is seen. Total TSNA content after curing of leaves is reduced in these plants. This suggests that reduced levels of nitrate can cause the formation of lower levels of TSNAs in cured plant material—such as cured leaves. The inventors unexpectedly found that a reduction in at least NNK is seen in cured plant material from both NtCLCe-RNAi and CLC-Nt2-RNAi plants. A reduction in total TSNA content was also observed. Reducing the expression of NtCLCe and/or CLC-Nt2 therefore contributes to reducing nitrate levels in tobacco leaves. After curing, at least NNK and optionally other TSNAs, which may include NNN or NAB or NAT or a combination of two or more thereof, can be reduced. In addition, the visual appearance of the plants is not substantially altered which is an important criterion for acceptance by the industry and for maximising plant yields and the like. The inventors have moreover unexpectedly found that certain CLC mutations described herein can result in an increased biomass yield in the plant. Furthermore, the inventors have unexpectedly found that certain CLC mutations described herein can result in modulation of more than one property, for example, modulation of nitrate/TSNA levels as well as modulation of biomass production in plants. The present invention may therefore be particularly useful to modulate (eg. increase or decrease) levels of nitrate, total TSNAs and/or biomass production in plants, including at least NNK. In particular, the present invention may be particularly useful when combined with other methods that are able to reduce the levels of TSNAs. Thus, it may be desirable in certain embodiments to reduce the expression of the one or more polynucleotides described herein together with reducing the expression of one or more nicotine demethylase genes in a tobacco plant. This combination would be expected to reduce at least NNK and NNN levels in a cured plant material which would be highly desirable since NNK and NNN have both been reported to be carcinogenic when applied to animals in laboratory studies. The tobacco products derived from the tobacco plants described herein may find use in methods for reducing the carcinogenic potential of these tobacco products, and reducing the exposure of humans to carcinogenic nitrosamines. Mutants of the polypeptide sequences described herein that can modulate nitrate content and/or biomass production in plants are also described. Therefore some mutants described herein may result in modulation of nitrate/TSNA levels only, whereas some mutants described herein may result in modulation of nitrate/TSNA levels and of biomass production.

### ASPECTS AND EMBODIMENTS OF THE INVENTION

Aspects and embodiments of the present invention are set forth in the accompanying claims.

In a first aspect, there is described a mutant, non-naturally occurring or transgenic plant cell comprising: (i) a polynucleotide comprising, consisting or consisting essentially of a sequence encoding a member of the CLC family of chloride channels and having at least 60% sequence identity to SEQ ID NO:1 or SEQ ID NO:2 or SEQ ID NO:3 or SEQ ID NO:4 or SEQ ID NO:10 or SEQ ID NO:11; (ii) a polypeptide encoded by the polynucleotide set forth in (i); (iii) a polypeptide comprising, consisting or consisting essentially of a sequence encoding a member of the CLC family of chloride channels and having at least 60% sequence identity to SEQ ID NO:5 or SEQ ID NO:6 or SEQ ID NO:7 or SEQ ID NO:12 or SEQ ID NO:13 or SEQ ID NO:14; or (iv) a construct, vector or expression vector comprising the isolated polynucleotide set forth in (i), and wherein the expression or activity of the polynucleotide or the polypeptide is modulated as compared to a control plant containing the control plant cell and wherein the nitrate and/or biomass levels in the mutant, non-naturally occurring or transgenic plant containing the mutant, non-naturally occurring or transgenic plant cell are modulated as compared to the control plant containing the control plant cell. By reducing the expression of the one or more genes in tobacco plants, nitrate levels can be reduced. Total TSNA content and/or NNK levels can be reduced in cured plant material.

In one embodiment, said mutant, non-naturally occurring or transgenic plant cell comprises one or more mutations in the disclosed polypeptides and polynucleotides that decreases the level of nitrate in the mutant, non-naturally occurring or transgenic plant containing the mutant, non-naturally occurring or transgenic plant cell as compared to the control plant containing the control plant cell. The mutation(s) can comprise a substitution mutation at position G163 of SEQ ID NO:5. In one embodiment, said mutant, non-naturally occurring or transgenic plant cell comprises one or more mutations in the disclosed polypeptides and polynucleotides that increase the level of nitrate in the mutant, non-naturally occurring or transgenic plant containing the mutant, non-naturally occurring or transgenic plant cell as compared to the control plant containing the control plant cell. The mutation(s) can comprise a substitution mutation at position P143 of SEQ ID NO:13.

In one embodiment, said mutant, non-naturally occurring or transgenic plant cell comprises one or more mutations in the disclosed polypeptides and polynucleotides that increase the level of biomass in the mutant, non-naturally occurring or transgenic plant containing the mutant, non-naturally occurring or transgenic plant cell as compared to the control plant containing the control plant cell. The mutation(s) can comprise a substitution mutation at position P184 of SEQ ID NO:13.

In one embodiment, said mutant, non-naturally occurring or transgenic plant cell comprises one or more mutations in the disclosed polypeptides and polynucleotides that modulate the level of nitrate and increase the level of biomass in the mutant, non-naturally occurring or transgenic plant containing the mutant, non-naturally occurring or transgenic plant cell as compared to the control plant containing the control plant cell. The mutation(s) can comprise a substitution mutation at position P184 of SEQ ID NO:13.

In a further aspect, there is described a mutant, non-naturally occurring or transgenic plant or component thereof comprising the plant cell described herein.

In a further aspect, there is described a method for modulating at least the nitrate (for example, 4-(methylnitrosamino)-1-(3-pyridyl)-1-butanone (NNK)) content of a plant or a component thereof, comprising the steps of: (a) modulating the expression or activity of: (i) a polynucleotide comprising, consisting or consisting essentially of a sequence encoding a member of the CLC family of chloride channels and having at least 60% sequence identity to SEQ ID NO:1 or SEQ ID NO:2 or SEQ ID NO:3 or SEQ ID NO:4 or SEQ ID NO:10 or SEQ ID NO:11; (ii) a polypeptide encoded by the polynucleotide set forth in (i); or (iii) a polypeptide comprising, consisting or consisting essentially of a sequence encoding a member of the CLC family of chloride channels and having at least 60% sequence identity to SEQ ID NO:5 or SEQ ID NO:6 or SEQ ID NO:7 or SEQ ID NO:12 or SEQ ID NO:13 or SEQ ID NO:14; (b) measuring at least the nitrate (for example, NNK) content in at least a part of the mutant, non-naturally occurring or transgenic plant obtained in step (a); and (c) identifying a mutant, non-naturally occurring or transgenic plant in which at least the nitrate (for example, NNK) content therein has changed in comparison to a control plant in which the expression or activity of the polynucleotide or polypeptide set forth in (a) has not been modulated.

In a further aspect, there is described a method for modulating at least the nitrate (for example, 4-(methylnitrosamino)-1-(3-pyridyl)-1-butanone (NNK)) content of a plant or a component thereof, comprising the steps of: (a) modulating the expression or activity of: (i) a polynucleotide comprising, consisting or consisting essentially of a sequence encoding a member of the CLC family of chloride channels and having at least 60% sequence identity to SEQ ID NO:1 or SEQ ID NO:2 or SEQ ID NO:3 or SEQ ID NO:10 or SEQ ID NO:11; (ii) a polypeptide encoded by the polynucleotide set forth in (i); or (iii) a polypeptide comprising, consisting or consisting essentially of a sequence encoding a member of the CLC family of chloride channels and having at least 60% sequence identity to SEQ ID NO:5 or SEQ ID NO:6 or SEQ ID NO:7 or SEQ ID NO:12 or SEQ ID NO:13 or SEQ ID NO:14; (b) measuring at least the nitrate (for example, NNK) content in at least a part of the mutant, non-naturally occurring or transgenic plant obtained in step (a); and (c) identifying a mutant, non-naturally occurring or transgenic plant in which at least the nitrate (for example, NNK) content therein has changed in comparison to a control plant in which the expression or activity of the polynucleotide or polypeptide set forth in (a) has not been modulated. Suitably, the nitrate (for example, NNK) content and/or total TSNA content and/or the nicotine content is modulated in the plant—such as cured plant material.

Suitably, the NNN content is substantially the same as the control plant.

Suitably, the component of the plant is a leaf, suitably, a cured leaf or cured tobacco.

In a further aspect, there is provided a method of modulating the biomass yield of a plant comprising modulating the expression of at least a CLC chloride channel polypeptide in said plant.

In one aspect, there is provided a method of modulating the biomass yield of a plant comprising modulating the expression of at least one of (i) a polynucleotide comprising, consisting or consisting essentially of a sequence encoding a member of the CLC family of chloride channels and having at least 60% sequence identity to SEQ ID NO:1 or SEQ ID NO:2 or SEQ ID NO:3 or SEQ ID NO:4 or SEQ ID NO:10 or SEQ ID NO:11; (ii) a polypeptide encoded by the polynucleotide set forth in (i); (iii) a polypeptide comprising, consisting or consisting essentially of a sequence encoding a member of the CLC family of chloride channels and having at least 60% sequence identity to SEQ ID NO:5 or SEQ ID NO:6 or SEQ ID NO:7 or SEQ ID NO:12 or SEQ ID NO:13 or SEQ ID NO:14; or (iv) a construct, vector or expression vector comprising the isolated polynucleotide set forth in (i), and wherein the expression or activity of the polynucleotide or the polypeptide is modulated as compared to a control plant containing the control plant cell and wherein the biomass levels in the mutant, non-naturally occurring or transgenic plant containing the mutant, non-naturally occurring or transgenic plant cell are modulated as compared to the control plant containing the control plant cell.

In a further aspect, there is provided a method of modulating the biomass yield of a plant comprising modulating the expression of a polypeptide comprising, consisting or consisting essentially of a sequence encoding a member of the CLC family of chloride channels and having at least 60% sequence identity to SEQ ID NO:5 or SEQ ID NO:6 or SEQ ID NO:7 or SEQ ID NO:12 or SEQ ID NO:13 or SEQ ID NO:14; wherein the expression or activity of the polynucleotide or the polypeptide is modulated as compared to a control plant containing the control plant cell and wherein the biomass levels in the mutant, non-naturally occurring or transgenic plant containing the mutant, non-naturally occurring or transgenic plant cell are modulated as compared to the control plant containing the control plant cell.

In a further aspect, there is provided a method of modulating at least the nitrate (for example, NNK) content and the biomass yield of a plant comprising modulating the expression of at least a CLC chloride channel polypeptide in said plant.

In one aspect, there is provided a method of modulating at least the nitrate (for example, NNK) content and the biomass yield of a plant comprising modulating the expression of at least one of (i) a polynucleotide comprising, consisting or consisting essentially of a sequence encoding a member of the CLC family of chloride channels and having at least 60% sequence identity to SEQ ID NO:1 or SEQ ID NO:2 or SEQ ID NO:3 or SEQ ID NO:4 or SEQ ID NO:10 or SEQ ID NO:11; (ii) a polypeptide encoded by the polynucleotide set forth in (i); (iii) a polypeptide comprising, consisting or consisting essentially of a sequence encoding a member of the CLC family of chloride channels and having at least 60% sequence identity to SEQ ID NO:5 or SEQ ID NO:6 or SEQ ID NO:7 or SEQ ID NO:12 or SEQ ID NO:13 or SEQ ID NO:14; or (iv) a construct, vector or expression vector comprising the isolated polynucleotide set forth in (i), and wherein the expression or activity of the polynucleotide or the polypeptide is modulated as compared to a control plant containing the control plant cell and wherein at least the nitrate (for example, NNK) content and the biomass levels in the mutant, non-naturally occurring or transgenic plant containing the mutant, non-naturally occurring or transgenic plant cell are modulated as compared to the control plant containing the control plant cell.

In a further aspect, there is provided a method of modulating at least the nitrate (for example, NNK) content and the biomass yield of a plant comprising modulating the expression of a polypeptide comprising, consisting or consisting essentially of a sequence encoding a member of the CLC family of chloride channels and having at least 60% sequence identity to SEQ ID NO:5 or SEQ ID NO:6 or SEQ ID NO:7 or SEQ ID NO:12 or SEQ ID NO:13 or SEQ ID NO:14; wherein the expression or activity of the polynucleotide or the polypeptide is modulated as compared to a control plant containing the control plant cell and wherein at least the nitrate (for example, NNK) content and the biomass levels in the mutant, non-naturally occurring or transgenic plant containing the mutant, non-naturally occurring or transgenic plant cell are modulated as compared to the control plant containing the control plant cell.

In a further aspect, there is described a plant or a component thereof obtained or obtainable by the methods described herein.

In a further aspect, there is described a mutant, non-naturally occurring or transgenic plant wherein the NNK content is about 110 ng/g or less, optionally, wherein the nitrate content is about 7 mg/g or less. Suitably, the plant is in the form of cured plant material.

In a further aspect, there is described a mutant plant wherein the nitrate content is about 6 mg/g or less and the nicotine content is about 13 mg/g or less.

In one embodiment, the mutant non-naturally occurring or transgenic plant has an increase in biomass yield of at least 1.5× in comparison to a control plant containing the control plant cell.

Suitably, the expression of: (i) a polynucleotide comprising, consisting or consisting essentially of a sequence encoding a member of the CLC family of chloride channels and having at least 60% sequence identity to SEQ ID NO:1 or SEQ ID NO:2 or SEQ ID NO:3 or SEQ ID NO:4 or SEQ ID NO:10 or SEQ ID NO:11; (ii) a polypeptide encoded by the polynucleotide set forth in (i); or (iii) a polypeptide comprising, consisting or consisting essentially of a sequence encoding a member of the CLC family of chloride channels and having at least 60% sequence identity to SEQ ID NO:5 or SEQ ID NO:6 or SEQ ID NO:7 or SEQ ID NO:12 or SEQ ID NO:13 or SEQ ID NO:14 is modulated as compared to a control plant.

In a further aspect, there is described plant material including biomass, seed, stem or leaves from the plant described herein.

In a further aspect, there is described a tobacco product comprising the plant cell, at least a part of the plant or plant material as described herein.

In a further aspect, there is described a method for producing cured plant material—such as leaves—with reduced levels of NNK therein comprising the steps of: (a) providing at least part of a plant or plant material as described herein; (b) optionally harvesting the plant material from the plant; and (c) curing the plant material for a period of time sufficient for at least the levels of NNK therein to be reduced.

In a further aspect, there is described an isolated polynucleotide comprising, consisting or consisting essentially of a sequence encoding a member of the CLC family of chloride channels and having at least 99.1% sequence identity to SEQ ID NO:1 or 97.1% sequence identity to SEQ ID NO:2 or 63% sequence identity to SEQ ID NO:3 or 61% sequence identity to SEQ ID NO:4 or 60% sequence identity to SEQ ID NO:10 or 60% sequence identity to SEQ ID NO:11.

In a further aspect, there is described an isolated polypeptide encoded by the polynucleotide(s) described herein.

In a further aspect, there is described an isolated polypeptide comprising, consisting or consisting essentially of a sequence encoding a member of the CLC family of chloride channels and having at least 99.1% sequence identity to SEQ ID NO:5 or at least 98.1% sequence identity to SEQ ID NO:6 or at least 60% sequence identity to SEQ ID NO:7 or at least 60% sequence identity to SEQ ID NO:12 or at least 60% sequence identity to SEQ ID NO:13 or at least 60% sequence identity to SEQ ID NO:14.

In a further aspect, there is described a construct, vector or expression vector comprising one or more of the isolated polynucleotide(s) described herein.

In a further aspect, there is described a mutant plant cell comprising one or more mutations in: (i) a polynucleotide comprising, consisting or consisting essentially of a sequence encoding a member of the CLC family of chloride channels and having at least 60% sequence identity to SEQ ID NO:1 or SEQ ID NO:2 or SEQ ID NO:3 or SEQ ID NO:4 or SEQ ID NO:10 or SEQ ID NO:11; (ii) a polypeptide encoded by the polynucleotide set forth in (i); or (iii) a polypeptide comprising, consisting or consisting essentially of a sequence encoding a member of the CLC family of chloride channels and having at least 60% sequence identity to SEQ ID NO:5 or SEQ ID NO:6 or SEQ ID NO:7 or SEQ ID NO:12 or SEQ ID NO:13 or SEQ ID NO:14; and wherein said one more mutations modulate the expression or activity of the polynucleotide or the polypeptide as compared to a control plant comprising a control plant cell and wherein the nitrate levels in the mutant plant containing the mutant plant cell are modulated as compared to the control plant.

In a further aspect, there is described a mutant plant cell comprising one or more mutations in: (i) a polynucleotide comprising, consisting or consisting essentially of a sequence encoding a member of the CLC family of chloride channels and having at least 60% sequence identity to SEQ ID NO:1 or SEQ ID NO:2 or SEQ ID NO:3 or SEQ ID NO:4 or SEQ ID NO:10 or SEQ ID NO:11; (ii) a polypeptide encoded by the polynucleotide set forth in (i); or (iii) a polypeptide comprising, consisting or consisting essentially of a sequence encoding a member of the CLC family of chloride channels and having at least 60% sequence identity to SEQ ID NO:5 or SEQ ID NO:6 or SEQ ID NO:7 or SEQ ID NO:12 or SEQ ID NO:13 or SEQ ID NO:14; and wherein said one more mutations modulate the expression or activity of the polynucleotide or the polypeptide as compared to a control plant comprising a control plant cell and wherein the nitrate levels in the mutant plant containing the mutant plant cell are modulated as compared to the control plant.

In a further aspect, there is described a mutant plant cell comprising one or more mutations in: (i) a polynucleotide comprising, consisting or consisting essentially of a sequence encoding a member of the CLC family of chloride channels and having at least 60% sequence identity to SEQ ID NO:1 or SEQ ID NO:2 or SEQ ID NO:3 or SEQ ID NO:4 or SEQ ID NO:10 or SEQ ID NO:11; (ii) a polypeptide encoded by the polynucleotide set forth in (i); or (iii) a polypeptide comprising, consisting or consisting essentially of a sequence encoding a member of the CLC family of chloride channels and having at least 60% sequence identity to SEQ ID NO:5 or SEQ ID NO:6 or SEQ ID NO:7 or SEQ ID NO:12 or SEQ ID NO:13 or SEQ ID NO:14; and wherein said one more mutations modulate the expression or activity of the polynucleotide or the polypeptide as compared to a control plant comprising a control plant cell and wherein the biomass levels in the mutant plant containing the mutant plant cell are modulated as compared to the control plant.

In a further aspect, there is provided a method for reducing a carcinogenic potential of a tobacco product, said method comprising preparing said tobacco product from a tobacco plant, or plant part or progeny thereof as described herein.

In a further aspect, there is described the use of the construct as described herein in a method for making plants having modulated levels of nitrate and/or NNK and/or total TSNA relative to a control plant.

In a further aspect, there is described the use of a construct as described herein in a method for making plants having modulated biomass levels relative to a control plant.

In a further aspect, there is described the use of a construct as described herein in a method for making plants having modulated nitrate (for example, NNK) content and biomass yield.

In a further aspect, there is described the use a polynucleotide or a polypeptide as described herein for modulating levels of nitrate and/or NNK and/or total TSNA in a plant relative to a control plant.

In a further aspect there is described a mutant plant cell comprising one or more mutations that decrease the level of nitrate in the mutant plant containing the mutant plant cell as compared to the control plant containing the control plant cell, wherein said mutation(s) comprises a substitution mutation at position G163 of SEQ ID NO:5.

In a further aspect there is described a mutant plant cell comprising one or more mutations that decrease the level of nitrate in the mutant plant containing the mutant plant cell as compared to the control plant containing the control plant cell, wherein said mutation(s) comprises a substitution mutation at position P143 of SEQ ID NO:13.

In a further aspect there is described a mutant plant cell comprising one or more mutations that decrease the level of nitrate and/or increase the biomass yield in the mutant plant containing the mutant plant cell as compared to the control plant containing the control plant cell, wherein said mutation(s) comprises a substitution mutation at position P184 of SEQ ID NO:13.

In a further aspect, there is disclosed a polypeptide sequence comprising or consisting of the sequence set forth in SEQ ID NO:5 with a substitution mutation at position G163, suitably, G163R.

In a further aspect, there is disclosed a polypeptide sequence comprising or consisting of the sequence set forth in SEQ ID NO:13 with a substitution mutation at position P143, suitably, P143L.

In a further aspect, there is provided a polypeptide sequence comprising or consisting of the sequence set forth in SEQ ID NO:13 with a substitution mutation at position P184, suitably, P184S.

In a further aspect, mutant polypeptides as described herein are disclosed.

Each of the embodiments discussed above are disclosed as embodiments of each of the aspects of the invention. Combinations of one or of the embodiments are contemplated.

### BRIEF DESCRIPTION OF THE DRAWINGS

FIG. 1: Semi-quantitative RT-PCR of three representative NtCLCe-RNAi lines (lanes 1, 2 and 3), wt (lanes 4, 5 and 6) and CLC-Nt2-RNAi lines (lanes 7, 8 and 9) showing the expression of tubulin (house-keeping gene), NtCLCe and CLC-Nt2 transcripts.

FIG. 2: Nicotine and nitrate analyses in green leaves of wt (n=11), NtCLCe-RNAi (n=5) and CLC-Nt2-RNAi (n=5) plants (A); total TSNA content in the corresponding leaves following air-curing process. In this experiment, plants were cultivated in 3 litre pots and the highest total TSNA value corresponds to 200 ng/g.

FIG. 3: Nicotine, nitrate analyses in green leaves (A), leaf weight (B) and leaf number (C) of wt (n=4), NtCLCe-RNAi and CLC-Nt2-RNAi plants (n=8) lacking both CLC-Nt2 and NtCLCe transcripts (CLC-RNAi lines). Leaves were harvested after 10 weeks growth in 10 litre pots under controlled greenhouse conditions. In this experiment, the maximum values for nicotine and nitrate were of 29.6 and 6.4 mg/g, respectively.

FIG. 4: Percentage of NNK in air-cured leaves of wt, NtCLCe-RNAi and CLC-Nt2-RNAi plants, after cultivation in 10 litre pots as shown in FIG. 3. In this experiment, the highest NNK value corresponds to 108 ng/g.

FIG. 5: Time course of nitrate and nicotine levels in green leaves of field grown CLCNt2-s G163R mutant plants. Entire leaves are harvested at mid-stalk position from field grown CLCNt2-s G163R homozygous plants (triangle) and out-segregant wild type (diamond) plants growing under Burley regime. Samples are harvested at three different times during the morning (early, mid and late) and freeze-dried. Powdered lamina material is analyzed for nitrate and nicotine content. N=4 to 8 individual plants. Standard deviation is indicated in the Figure. Early=8:00 am-9:00 am; Mid=9:30 am-10:30 am; Late=11:00 am-12:00 pm.

FIG. 6: Time course of nitrate and nicotine levels in green leaves of field grown NtCLCe-t P143L mutant plants. Entire leaves are harvested at mid-stalk position from field grown NtCLCe-t P143L homozygous (square) and out-segregant wild type (diamond) plants growing under Burley regime. Samples are harvested at three different times during the morning (early, mid and late) and freeze-dried. Powdered lamina material is analyzed for nitrate and nicotine content. N=4 to 8 individual plants. Standard deviation is indicated in the Figure. Early=8:00 am-9:00 am; Mid=9:30 am-10:30 am; Late=11:00 am-12:00 pm.

FIG. 7. Biomass comparison of NtCLCe-T P184S homozygous, heterozygous and out-segregant WT variant lines. Biomass of plots for the variant line NtCLCe-T P184S, indicated as grams of cured leaf material per plant within the plot. Out-segregant wild-type (wt) plots in black, heterozygous dotted columns and homozygous in white. A: biomass of single plots (867, 885, etc. are plot identification numbers/single plants, and reported in abscissa). B: mean of plots with the same genotype. Error bars indicate confidence interval at 95%.

FIG. 8. Biomass comparison for different-type plots in the 2013 LaSota field. Biomass of plots for the variant line NtCLCe-T P184S, indicated as grams of cured leaf material per plant within the plot. Columns corresponding to out-segregant wild-type (wt) plots are colored in black, heterozygous are dotted and homozygous in white. Error bars indicate confidence interval at 95%.

FIG. 9. Average number of leaves per plant at topping time. Number of leaves per plant was recorded at topping time for the variant line NtCLCe-T P184S (outsegregant wild-type plots in black, heterozygous as dotted columns and homozygous in white). Error bars indicate confidence interval at 95%.

### DEFINITIONS

The technical terms and expressions used within the scope of this application are generally to be given the meaning commonly applied to them in the pertinent art of plant and molecular biology. All of the following term definitions apply to the complete content of this application. The word “comprising” does not exclude other elements or steps, and the indefinite article “a” or “an” does not exclude a plurality. A single step may fulfil the functions of several features recited in the claims. The terms “about”, “essentially” and “approximately” in the context of a given numerate value or range refers to a value or range that is within 20%, within 10%, or within 5%, 4%, 3%, 2% or 1% of the given value or range.

The term “isolated” refers to any entity that is taken from its natural milieu, but the term does not connote any degree of purification.

An “expression vector” is a nucleic acid vehicle that comprises a combination of nucleic acid components for enabling the expression of nucleic acid. Suitable expression vectors include episomes capable of extra-chromosomal replication such as circular, double-stranded nucleic acid plasmids; linearized double-stranded nucleic acid plasmids; and other functionally equivalent expression vectors of any origin. An expression vector comprises at least a promoter positioned upstream and operably-linked to a nucleic acid, nucleic acid constructs or nucleic acid conjugate, as defined below.

The term “construct” refers to a double-stranded, recombinant nucleic acid fragment comprising one or more polynucleotides. The construct comprises a “template strand” base-paired with a complementary “sense or coding strand.” A given construct can be inserted into a vector in two possible orientations, either in the same (or sense) orientation or in the reverse (or anti-sense) orientation with respect to the orientation of a promoter positioned within a vector—such as an expression vector.

A “vector” refers to a nucleic acid vehicle that comprises a combination of nucleic acid components for enabling the transport of nucleic acid, nucleic acid constructs and nucleic acid conjugates and the like. Suitable vectors include episomes capable of extra-chromosomal replication such as circular, double-stranded nucleic acid plasmids; linearized double-stranded nucleic acid plasmids; and other vectors of any origin.

A “promoter” refers to a nucleic acid element/sequence, typically positioned upstream and operably-linked to a double-stranded DNA fragment. Promoters can be derived entirely from regions proximate to a native gene of interest, or can be composed of different elements derived from different native promoters or synthetic DNA segments.

The terms “homology, identity or similarity” refer to the degree of sequence similarity between two polypeptides or between two nucleic acid molecules compared by sequence alignment. The degree of homology between two discrete nucleic acid sequences being compared is a function of the number of identical, or matching, nucleotides at comparable positions. The percent identity may be determined by visual inspection and mathematical calculation. Alternatively, the percent identity of two nucleic acid sequences may be determined by comparing sequence information using a computer program such as—ClustalW, BLAST, FASTA or Smith-Waterman. Default parameters for these programs can be used.

The term “plant” refers to any plant at any stage of its life cycle or development, and its progenies. In one embodiment, the plant is a “tobacco plant”, which refers to a plant belonging to the genus Nicotiana. Preferred species of tobacco plant are described herein.

A “plant cell” refers to a structural and physiological unit of a plant. The plant cell may be in the form of a protoplast without a cell wall, an isolated single cell or a cultured cell, or as a part of higher organized unit such as but not limited to, plant tissue, a plant organ, or a whole plant.

The term “plant material” refers to any solid, liquid or gaseous composition, or a combination thereof, obtainable from a plant, including biomass, leaves, stems, roots, flowers or flower parts, fruits, pollen, egg cells, zygotes, seeds, cuttings, secretions, extracts, cell or tissue cultures, or any other parts or products of a plant. In one embodiment, the plant material comprises or consists of biomass, stem, seed or leaves. In another embodiment, the plant material comprises or consists of leaves.

The term “variety” refers to a population of plants that share constant characteristics which separate them from other plants of the same species. While possessing one or more distinctive traits, a variety is further characterized by a very small overall variation between individuals within that variety. A variety is often sold commercially.

The term “line” or “breeding line” as used herein denotes a group of plants that are used during plant breeding. A line is distinguishable from a variety as it displays little variation between individuals for one or more traits of interest, although there may be some variation between individuals for other traits.

The term “modulating” may refer to reducing, inhibiting, increasing or otherwise affecting the expression or activity of a polypeptide. The term may also refer to reducing, inhibiting, increasing or otherwise affecting the activity of a gene encoding a polypeptide which can include, but is not limited to, modulating transcriptional activity.

The term “reduce” or “reduced” as used herein, refers to a reduction of from about 10% to about 99%, or a reduction of at least 10%, at least 20%, at least 25%, at least 30%, at least 40%, at least 50%, at least 60%, at least 70%, at least 75%, at least 80%, at least 90%, at least 95%, at least 98%, at least 99%, or at least 100% or more of a quantity or an activity, such as but not limited to polypeptide activity, transcriptional activity and protein expression.

The term “inhibit” or “inhibited” as used herein, refers to a reduction of from about 98% to about 100%, or a reduction of at least 98%, at least 99%, but particularly of 100%, of a quantity or an activity, such as but not limited to polypeptide activity, transcriptional activity and protein expression.

The term “increase” or “increased” as used herein, refers to an increase of from about 5% to about 99%, or an increase of at least 5%, at least 10%, at least 20%, at least 25%, at least 30%, at least 40%, at least 50%, at least 60%, at least 70%, at least 75%, at least 80%, at least 90%, at least 95%, at least 98%, at least 99%, or at least 100% or more of a quantity or an activity, such as but not limited to polypeptide activity, transcriptional activity and protein expression.

The term “control” in the context of a control plant means a plant or plant cell in which the expression or activity of an enzyme has not been modified (for example, increased or reduced) and so it can provide a comparison with a plant in which the expression or activity of the enzyme has been modified. The control plant may comprise an empty vector. The control plant or plant cell may correspond to a wild-type plant or wild-type plant cell.

### DETAILED DESCRIPTION

In one embodiment, there is provided an isolated polynucleotide comprising, consisting or consisting essentially of a polynucleotide sequence having at least 60% sequence identity to any of the sequences described herein, including any of polynucleotides shown in the sequence listing. Suitably, the isolated polynucleotide comprises, consists or consists essentially of a sequence having at least 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99% or 100% sequence identity thereto.

In another embodiment, there is provided an isolated polynucleotide comprising, consisting or consisting essentially of a polynucleotide sequence having at least 60% sequence identity to SEQ ID NO. 1 or SEQ ID NO:2 or SEQ ID NO. 3 or SEQ ID NO. 4 or SEQ ID NO:10 or SEQ ID NO:11. Suitably, the isolated polynucleotide comprises, consists or consist essentially of a sequence having at least about 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99%, 99.1%, 99.2%, 99.3%, 99.4%, 99.5%, 99.6%, 99.7%, 99.8%, 99.9% or 100% sequence identity to SEQ ID NO. 1 or SEQ ID NO:2 or SEQ ID NO. 3 or SEQ ID NO. 4 or SEQ ID NO:10 or SEQ ID NO:11.

In another embodiment, there is provided polynucleotides comprising, consisting or consisting essentially of polynucleotides with substantial homology (that is, sequence similarity) or substantial identity to SEQ ID NO. 1 or SEQ ID NO:2 or SEQ ID NO. 3 or SEQ ID NO. 4 or SEQ ID NO:10 or SEQ ID NO:11.

In another embodiment, there is provided polynucleotide variants that have at least about 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99%, 99.1%, 99.2%, 99.3%, 99.4%, 99.5%, 99.6%, 99.7%, 99.8% or 99.9% sequence identity to the sequence of SEQ ID NO. 1 or SEQ ID NO:2 or SEQ ID NO. 3 or SEQ ID NO. 4 or SEQ ID NO:10 or SEQ ID NO:11 In another embodiment, there is provided fragments of SEQ ID NO. 1 or SEQ ID NO:2 or SEQ ID NO. 3 or SEQ ID NO. 4 or SEQ ID NO:10 or SEQ ID NO:11 and fragments of SEQ ID NO. 1 or SEQ ID NO:2 or SEQ ID NO. 3 or SEQ ID NO. 4 or SEQ ID NO:10 or SEQ ID NO:11 with substantial homology (that is, sequence similarity) or substantial identity thereto that have at least about 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99%, 99.1%, 99.2%, 99.3%, 99.4%, 99.5%, 99.6%, 99.7%, 99.8%, 99.9% or 100% sequence identity to the corresponding fragments of SEQ ID NO. 1 or SEQ ID NO:2 or SEQ ID NO. 3 or SEQ ID NO. 4 or SEQ ID NO:10 or SEQ ID NO:11.

In another embodiment, there is provided polynucleotides comprising a sufficient or substantial degree of identity or similarity to SEQ ID NO. 1 or SEQ ID NO:2 or SEQ ID NO. 3 or SEQ ID NO. 4 or SEQ ID NO:10 or SEQ ID NO:11 that encode a polypeptide that functions as a member of the CLC family of chloride channels.

In another embodiment, there is provided a polymer of polynucleotides which comprises, consists or consists essentially of a polynucleotide designated herein as SEQ ID NO. 1 or SEQ ID NO:2 or SEQ ID NO. 3 or SEQ ID NO. 4 or SEQ ID NO:10 or SEQ ID NO:11.

Suitably, the polynucleotides described herein encode members of the CLC family of chloride channels. CLCs constitute a family of voltage-gated channels. In plants, chloride channels contribute to a number of plant-specific functions—such as in the regulation of turgor, stomatal movement, nutrient transport and/or metal tolerance and the like. The nitrate/proton antiporter AtCLCa mediates nitrate accumulation in plant vacuoles (see Nature (2006) 442 (7105):939-42). In this publication it is shown that AtClCa functions as a 2NO₃⁻/1H⁺ exchanger that is able to accumulate nitrate into the vacuole by using electrophysiological approaches. A similar approach can be used to test the nitrate transport activity of CLC-Nt2. “Solute transporters in plant thylakoid membranes: Key players during photosynthesis and light stress by Spetea C, Schoefs B. Communicative & Integrative Biology. 2010; 3(2)122-129 and Monachello et al., New Phytol. 2009; 183(1):88-94 disclose that AtClCe is predicted to be involved in nitrite translocation from the stroma into the thylakoid lumen, taking over from the nitrite transporter of the chloroplast envelope. Methods described therein for measuring this activity may be used to measure the activity of NtCLCe.

Combinations of SEQ ID NO. 1 or SEQ ID NO:2 or SEQ ID NO. 3 or SEQ ID NO. 4 or SEQ ID NO:10 or SEQ ID NO:11 are also contemplated. These combinations include various combinations of SEQ ID NO. 1, SEQ ID NO:2, SEQ ID NO. 3, SEQ ID NO. 4, SEQ ID NO:10 and SEQ ID NO:11—including the combination of SEQ ID NO:1 and SEQ ID NO:2; the combination of SEQ ID NO:1 and SEQ ID NO:3; the combination of SEQ ID NO:1 and SEQ ID NO:4; the combination of SEQ ID NO:1 and SEQ ID NO:10; the combination of SEQ ID NO:1 and SEQ ID NO:11; the combination of SEQ ID NO:2 and SEQ ID NO:3; the combination of SEQ ID NO:2 and SEQ ID NO:4; the combination of SEQ ID NO:2 and SEQ ID NO:10; the combination of SEQ ID NO:2 and SEQ ID NO:11; the combination of SEQ ID NO:3 and SEQ ID NO:4, the combination of SEQ ID NO:3 and SEQ ID NO:10; the combination of SEQ ID NO:3 and SEQ ID NO:11; the combination of SEQ ID NO:1, SEQ ID NO:2 and SEQ ID NO:3; the combination of SEQ ID NO:1, SEQ ID NO:2 and SEQ ID NO:4; the combination of SEQ ID NO:1, SEQ ID NO:3 and SEQ ID NO:4; the combination of SEQ ID NO:2, SEQ ID NO:3 and SEQ ID NO:4; the combination of SEQ ID NO:1, SEQ ID NO:2, SEQ ID NO:3 and SEQ ID NO:4; or the combination of SEQ ID NO. 1, SEQ ID NO:2 and SEQ ID NO. 3; the combination of SEQ ID NO:1, SEQ ID NO:2, SEQ ID NO:3, SEQ ID NO:10 and SEQ ID NO:11; the combination of SEQ ID NO:1, SEQ ID NO:2, SEQ ID NO:4, SEQ ID NO:10 and SEQ ID NO:11; the combination of SEQ ID NO:1, SEQ ID NO:3 SEQ ID NO:4, SEQ ID NO:10 and SEQ ID NO:11; the combination of SEQ ID NO:2, SEQ ID NO:3, SEQ ID NO:4, SEQ ID NO:10 and SEQ ID NO:11; the combination of SEQ ID NO:1, SEQ ID NO:2, SEQ ID NO:3 and SEQ ID NO:4; or the combination of SEQ ID NO. 1, SEQ ID NO:2 and SEQ ID NO. 3 etc.

A polynucleotide as described herein can include a polymer of nucleotides, which may be unmodified or modified deoxyribonucleic acid (DNA) or ribonucleic acid (RNA). Accordingly, a polynucleotide can be, without limitation, a genomic DNA, complementary DNA (cDNA), mRNA, or antisense RNA or a fragment(s) thereof. Moreover, a polynucleotide can be single-stranded or double-stranded DNA, DNA that is a mixture of single-stranded and double-stranded regions, a hybrid molecule comprising DNA and RNA, or a hybrid molecule with a mixture of single-stranded and double-stranded regions or a fragment(s) thereof. In addition, the polynucleotide can be composed of triple-stranded regions comprising DNA, RNA, or both or a fragment(s) thereof. A polynucleotide can contain one or more modified bases, such as phosphothioates, and can be a peptide nucleic acid. Generally, polynucleotides can be assembled from isolated or cloned fragments of cDNA, genomic DNA, oligonucleotides, or individual nucleotides, or a combination of the foregoing. Although the polynucleotide sequences described herein are shown as DNA sequences, the sequences include their corresponding RNA sequences, and their complementary (for example, completely complementary) DNA or RNA sequences, including the reverse complements thereof.

A polynucleotide as described herein will generally contain phosphodiester bonds, although in some cases, polynucleotide analogues are included that may have alternate backbones, comprising, for example, phosphoramidate, phosphorothioate, phosphorodithioate, or O-methylphophoroamidite linkages; and peptide polynucleotide backbones and linkages. Other analogue polynucleotides include those with positive backbones; non-ionic backbones, and non-ribose backbones. Modifications of the ribose-phosphate backbone may be done for a variety of reasons, for example, to increase the stability and half-life of such molecules in physiological environments or as probes on a biochip. Mixtures of naturally occurring polynucleotides and analogues can be made; alternatively, mixtures of different polynucleotide analogues, and mixtures of naturally occurring polynucleotides and analogues may be made.

A variety of polynucleotide analogues are known, including, for example, phosphoramidate, phosphorothioate, phosphorodithioate, O-methylphophoroamidite linkages and peptide polynucleotide backbones and linkages. Other analogue polynucleotides include those with positive backbones, non-ionic backbones and non-ribose backbones. Polynucleotides containing one or more carbocyclic sugars are also included.

Other analogues include peptide polynucleotides which are peptide polynucleotide analogues. These backbones are substantially non-ionic under neutral conditions, in contrast to the highly charged phosphodiester backbone of naturally occurring polynucleotides. This may result in advantages. First, the peptide polynucleotide backbone may exhibit improved hybridization kinetics. Peptide polynucleotides have larger changes in the melting temperature for mismatched versus perfectly matched base pairs. DNA and RNA typically exhibit a 2-4° C. drop in melting temperature for an internal mismatch. With the non-ionic peptide polynucleotide backbone, the drop is closer to 7-9° C. Similarly, due to their non-ionic nature, hybridization of the bases attached to these backbones is relatively insensitive to salt concentration. In addition, peptide polynucleotides may not be degraded or degraded to a lesser extent by cellular enzymes, and thus may be more stable.

Among the uses of the disclosed polynucleotides, and fragments thereof, is the use of fragments as probes in nucleic acid hybridisation assays or primers for use in nucleic acid amplification assays. Such fragments generally comprise at least about 10, 11, 12, 13, 14, 15, 16, 17, 18, 19 or 20 or more contiguous nucleotides of a DNA sequence. In other embodiments, a DNA fragment comprises at least about 10, 15, 20, 30, 40, 50 or 60 or more contiguous nucleotides of a DNA sequence. Thus, in one aspect, there is also provided a method for detecting a polynucleotide encoding a member of the CLC family of chloride channels comprising the use of the probes or primers or both.

The basic parameters affecting the choice of hybridization conditions and guidance for devising suitable conditions are described by Sambrook, J., E. F. Fritsch, and T. Maniatis (1989, Molecular Cloning: A Laboratory Manual, Cold Spring Harbor Laboratory Press, Cold Spring Harbor, N.Y.). Using knowledge of the genetic code in combination with the amino acid sequences described herein, sets of degenerate oligonucleotides can be prepared. Such oligonucleotides are useful as primers, for example, in polymerase chain reactions (PCR), whereby DNA fragments are isolated and amplified. In certain embodiments, degenerate primers can be used as probes for genetic libraries. Such libraries would include but are not limited to cDNA libraries, genomic libraries, and even electronic express sequence tag or DNA libraries. Homologous sequences identified by this method would then be used as probes to identify homologues of the sequences identified herein.

Also of potential use are polynucleotides and oligonucleotides (for example, primers or probes) that hybridize under reduced stringency conditions, typically moderately stringent conditions, and commonly highly stringent conditions to the polynucleotide(s) as described herein. The basic parameters affecting the choice of hybridization conditions and guidance for devising suitable conditions are set forth by Sambrook, J., E. F. Fritsch, and T. Maniatis (1989, Molecular Cloning: A Laboratory Manual, Cold Spring Harbor Laboratory Press, Cold Spring Harbor, N.Y. and can be readily determined by those having ordinary skill in the art based on, for example, the length or base composition of the polynucleotide.

One way of achieving moderately stringent conditions involves the use of a prewashing solution containing 5× Standard Sodium Citrate, 0.5% Sodium Dodecyl Sulphate, 1.0 mM Ethylenediaminetetraacetic acid (pH 8.0), hybridization buffer of about 50% formamide, 6× Standard Sodium Citrate, and a hybridization temperature of about 55° C. (or other similar hybridization solutions, such as one containing about 50% formamide, with a hybridization temperature of about 42° C.), and washing conditions of about 60° C., in 0.5× Standard Sodium Citrate, 0.1% Sodium Dodecyl Sulphate. Generally, highly stringent conditions are defined as hybridization conditions as above, but with washing at approximately 68° C., 0.2× Standard Sodium Citrate, 0.1% Sodium Dodecyl Sulphate. SSPE (1×SSPE is 0.15M sodium chloride, 10 mM sodium phosphate, and 1.25 mM Ethylenediaminetetraacetic acid, pH 7.4) can be substituted for Standard Sodium Citrate (1× Standard Sodium Citrate is 0.15M sodium chloride and 15 mM sodium citrate) in the hybridization and wash buffers; washes are performed for 15 minutes after hybridization is complete. It should be understood that the wash temperature and wash salt concentration can be adjusted as necessary to achieve a desired degree of stringency by applying the basic principles that govern hybridization reactions and duplex stability, as known to those skilled in the art and described further below (see, for example, Sambrook, J., E. F. Fritsch, and T. Maniatis (1989, Molecular Cloning: A Laboratory Manual, Cold Spring Harbor Laboratory Press, Cold Spring Harbor, N.Y.). When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. The hybridization temperature for hybrids anticipated to be less than 50 base pairs in length should be 5 to 10° C. less than the melting temperature of the hybrid, where melting temperature is determined according to the following equations. For hybrids less than 18 base pairs in length, melting temperature (° C.)=2(number of A+T bases)+4(number of G+C bases). For hybrids above 18 base pairs in length, melting temperature (° C.)=81.5+16.6(log 10 [Na+])+0.41(% G+C)-(600/N), where N is the number of bases in the hybrid, and [Na+] is the concentration of sodium ions in the hybridization buffer ([Na+] for 1× Standard Sodium Citrate=0.165M). Typically, each such hybridizing polynucleotide has a length that is at least 25% (commonly at least 50%, 60%, or 70%, and most commonly at least 80%) of the length of a polynucleotide to which it hybridizes, and has at least 60% sequence identity (for example, at least 70%, 75%, 80%, 85%, 90%, 95%, 96%, 97%, 98%, 99% or 100%) with a polynucleotide to which it hybridizes.

As will be understood by the person skilled in the art, a linear DNA has two possible orientations: the 5′-to-3′ direction and the 3′-to-5′ direction. For example, if a reference sequence is positioned in the 5′-to-3′ direction, and if a second sequence is positioned in the 5′-to-3′ direction within the same polynucleotide molecule/strand, then the reference sequence and the second sequence are orientated in the same direction, or have the same orientation. Typically, a promoter sequence and a gene of interest under the regulation of the given promoter are positioned in the same orientation. However, with respect to the reference sequence positioned in the 5′-to-3′ direction, if a second sequence is positioned in the 3′-to-5′ direction within the same polynucleotide molecule/strand, then the reference sequence and the second sequence are orientated in anti-sense direction, or have anti-sense orientation. Two sequences having anti-sense orientations with respect to each other can be alternatively described as having the same orientation, if the reference sequence (5′-to-3′ direction) and the reverse complementary sequence of the reference sequence (reference sequence positioned in the 5′-to-3′) are positioned within the same polynucleotide molecule/strand. The sequences set forth herein are shown in the 5′-to-3′ direction. Recombinant constructs provided herein can be used to transform plants or plant cells in order to modulate protein expression or activity levels. A recombinant polynucleotide construct can comprise a polynucleotide encoding one or more polynucleotides as described herein, operably linked to a regulatory region suitable for expressing the polypeptide in the plant or plant cell. Thus, a polynucleotide can comprise a coding sequence that encodes the polypeptide as described herein. Plants in which protein expression or activity levels are modulated can include mutant plants, non-naturally occurring plants, transgenic plants, man-made plants or genetically engineered plants. Suitably, the transgenic plant comprises a genome that has been altered by the stable integration of recombinant DNA. Recombinant DNA includes DNA which has been genetically engineered and constructed outside of a cell and includes DNA containing naturally occurring DNA or cDNA or synthetic DNA. A transgenic plant can include a plant regenerated from an originally-transformed plant cell and progeny transgenic plants from later generations or crosses of a transformed plant. Suitably, the transgenic modification alters the expression or activity of the polynucleotide or the polypeptide described herein as compared to a control plant.

The polypeptide encoded by a recombinant polynucleotide can be a native polypeptide, or can be heterologous to the cell. In some cases, the recombinant construct contains a polynucleotide that modulates expression, operably linked to a regulatory region. Examples of suitable regulatory regions are described herein.

Vectors containing recombinant polynucleotide constructs such as those described herein are also provided. Suitable vector backbones include, for example, those routinely used in the art such as plasmids, viruses, artificial chromosomes, bacterial artificial chromosomes, yeast artificial chromosomes, or bacteriophage artificial chromosomes. Suitable expression vectors include, without limitation, plasmids and viral vectors derived from, for example, bacteriophage, baculoviruses, and retroviruses. Numerous vectors and expression systems are commercially available.

The vectors can also include, for example, origins of replication, scaffold attachment regions or markers. A marker gene can confer a selectable phenotype on a plant cell. For example, a marker can confer biocide resistance, such as resistance to an antibiotic (for example, kanamycin, G418, bleomycin, or hygromycin), or an herbicide (for example, glyphosate, chlorsulfuron or phosphinothricin). In addition, an expression vector can include a tag sequence designed to facilitate manipulation or detection (for example, purification or localization) of the expressed polypeptide. Tag sequences, such as luciferase, beta-glucuronidase, green fluorescent protein, glutathione S-transferase, polyhistidine, c-myc or hemagglutinin sequences typically are expressed as a fusion with the encoded polypeptide. Such tags can be inserted anywhere within the polypeptide, including at either the carboxyl or amino terminus.

A plant or plant cell can be transformed by having the recombinant polynucleotide integrated into its genome to become stably transformed. The plant or plant cell described herein can be stably transformed. Stably transformed cells typically retain the introduced polynucleotide with each cell division. A plant or plant cell may also be transiently transformed such that the recombinant polynucleotide is not integrated into its genome. Transiently transformed cells typically lose all or some portion of the introduced recombinant polynucleotide with each cell division such that the introduced recombinant polynucleotide cannot be detected in daughter cells after a sufficient number of cell divisions.

A number of methods are available in the art for transforming a plant cell which are all encompassed herein, including biolistics, gene gun techniques, Agrobacterium-mediated transformation, viral vector-mediated transformation and electroporation. The Agrobacterium system for integration of foreign DNA into plant chromosomes has been extensively studied, modified, and exploited for plant genetic engineering. Naked recombinant DNA molecules comprising DNA sequences corresponding to the subject purified tobacco protein operably linked, in the sense or antisense orientation, to regulatory sequences are joined to appropriate T-DNA sequences by conventional methods. These are introduced into tobacco protoplasts by polyethylene glycol techniques or by electroporation techniques, both of which are standard.

Alternatively, such vectors comprising recombinant DNA molecules encoding the subject purified tobacco protein are introduced into live Agrobacterium cells, which then transfer the DNA into the tobacco plant cells. Transformation by naked DNA without accompanying T-DNA vector sequences can be accomplished via fusion of tobacco protoplasts with DNA-containing liposomes or via electroporation. Naked DNA unaccompanied by T-DNA vector sequences can also be used to transform tobacco cells via inert, high velocity microprojectiles.

If a cell or cultured tissue is used as the recipient tissue for transformation, plants can be regenerated from transformed cultures if desired, by techniques known to those skilled in the art.

The choice of regulatory regions to be included in a recombinant construct depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning regulatory regions relative to the coding sequence. Transcription of a polynucleotide can be modulated in a similar manner. Some suitable regulatory regions initiate transcription only, or predominantly, in certain cell types. Methods for identifying and characterizing regulatory regions in plant genomic DNA are known in the art.

Suitable promoters include tissue-specific promoters recognized by tissue-specific factors present in different tissues or cell types (for example, root-specific promoters, shoot-specific promoters, xylem-specific promoters), or present during different developmental stages, or present in response to different environmental conditions. Suitable promoters include constitutive promoters that can be activated in most cell types without requiring specific inducers. Examples of suitable promoters for controlling RNAi polypeptide production include the cauliflower mosaic virus 35S (CaMV/35S), SSU, OCS, lib4, usp, STLS1, B33, nos or ubiquitin- or phaseolin-promoters. Persons skilled in the art are capable of generating multiple variations of recombinant promoters.

Tissue-specific promoters are transcriptional control elements that are only active in particular cells or tissues at specific times during plant development, such as in vegetative tissues or reproductive tissues. Tissue-specific expression can be advantageous, for example, when the expression of polynucleotides in certain tissues is preferred. Examples of tissue-specific promoters under developmental control include promoters that can initiate transcription only (or primarily only) in certain tissues, such as vegetative tissues, for example, roots or leaves, or reproductive tissues, such as fruit, ovules, seeds, pollen, pistols, flowers, or any embryonic tissue. Reproductive tissue-specific promoters may be, for example, anther-specific, ovule-specific, embryo-specific, endosperm-specific, integument-specific, seed and seed coat-specific, pollen-specific, petal-specific, sepal-specific, or combinations thereof.

Suitable leaf-specific promoters include pyruvate, orthophosphate dikinase (PPDK) promoter from C4 plant (maize), cab-m1Ca+2 promoter from maize, the Arabidopsis thaliana myb-related gene promoter (Atmyb5), the ribulose biphosphate carboxylase (RBCS) promoters (for example, the tomato RBCS 1, RBCS2 and RBCS3A genes expressed in leaves and light-grown seedlings, RBCS1 and RBCS2 expressed in developing tomato fruits or ribulose bisphosphate carboxylase promoter expressed almost exclusively in mesophyll cells in leaf blades and leaf sheaths at high levels).

Suitable senescence-specific promoters include a tomato promoter active during fruit ripening, senescence and abscission of leaves, a maize promoter of gene encoding a cysteine protease. Suitable anther-specific promoters can be used. Suitable root-preferred promoters known to persons skilled in the art may be selected. Suitable seed-preferred promoters include both seed-specific promoters (those promoters active during seed development such as promoters of seed storage proteins) and seed-germinating promoters (those promoters active during seed germination). Such seed-preferred promoters include, but are not limited to, Cim1 (cytokinin-induced message); cZ19B1 (maize 19 kDa zein); milps (myo-inositol-1-phosphate synthase); mZE40-2, also known as Zm-40; nuclc; and celA (cellulose synthase). Gama-zein is an endosperm-specific promoter. Glob-1 is an embryo-specific promoter. For dicots, seed-specific promoters include, but are not limited to, bean beta-phaseolin, napin, β-conglycinin, soybean lectin, cruciferin, and the like. For monocots, seed-specific promoters include, but are not limited to, a maize 15 kDa zein promoter, a 22 kDa zein promoter, a 27 kDa zein promoter, a g-zein promoter, a 27 kDa gamma-zein promoter (such as gzw64A promoter, see Genbank Accession number S78780), a waxy promoter, a shrunken 1 promoter, a shrunken 2 promoter, a globulin 1 promoter (see Genbank Accession number L22344), an Itp2 promoter, cim1 promoter, maize end1 and end2 promoters, nuc1 promoter, Zm40 promoter, eep1 and eep2; lec1, thioredoxin H promoter; mlip15 promoter, PCNA2 promoter; and the shrunken-2 promoter.

Examples of inducible promoters include promoters responsive to pathogen attack, anaerobic conditions, elevated temperature, light, drought, cold temperature, or high salt concentration. Pathogen-inducible promoters include those from pathogenesis-related proteins (PR proteins), which are induced following infection by a pathogen (for example, PR proteins, SAR proteins, beta-1,3-glucanase, chitinase).

In addition to plant promoters, other suitable promoters may be derived from bacterial origin for example, the octopine synthase promoter, the nopaline synthase promoter and other promoters derived from Ti plasmids), or may be derived from viral promoters (for example, 35S and 19S RNA promoters of cauliflower mosaic virus (CaMV), constitutive promoters of tobacco mosaic virus, cauliflower mosaic virus (CaMV) 19S and 35S promoters, or figwort mosaic virus 35S promoter).

In another aspect, there is provided an isolated polypeptide comprising, consisting or consisting essentially of a polypeptide sequence having at least 60% sequence identity to any of the sequences described herein, including any of the polypeptides shown in the sequence listing. Suitably, the isolated polypeptide comprises, consists or consists essentially of a sequence having at least 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99%, 99.1%, 99.2%, 99.3%, 99.4%, 99.5%, 99.6%, 99.7%, 99.8%, 99.9% or 100% sequence identity thereto.

In one embodiment, there is provided a polypeptide encoded by SEQ ID NO: 1 or SEQ ID NO: 2 or SEQ ID NO: 3 or SEQ ID NO: 4 or SEQ ID NO:10 or SEQ ID NO:11.

In another embodiment, there is provided an isolated polypeptide comprising, consisting or consisting essentially of a sequence having at least 60%, 61%, 62%, 63%, 64%, 65%, 66%, 67%, 68%, 69%, 70%, 75%, 80%, 85%, 87%, 88%, 89%, 90%, 91%, 92%, 93%, 94%, 95% 96%, 97%, 98%, 99%, 99.1%, 99.2%, 99.3%, 99.4%, 99.5%, 99.6%, 99.7%, 99.8%, 99.9% or 100% sequence identity to SEQ ID NO: 5 or SEQ ID NO: 6 or SEQ ID NO: 7 or SEQ ID NO:12 or SEQ ID NO:13 or SEQ ID NO:14.

In another embodiment, there is provided a polypeptide variant comprising, consisting or consisting essentially of an amino acid sequence encoded by a polynucleotide variant with at least about 66%, 67%, 68%, 69%, 70%, 75%, 80%, 85%, 87%, 88%, 89%, 90%, 91%, 92%, 93%, 94%, 95% 96%, 97%, 98%, 99% 99.1%, 99.2%, 99.3%, 99.4%, 99.5%, 99.6%, 99.7%, 99.8% or 99.9% sequence identity to SEQ ID NO: 1 or SEQ ID NO: 2 or SEQ ID NO: 3 or SEQ ID NO: 4 or SEQ ID NO:10 or SEQ ID NO:11.

In another embodiment, there is provided fragments of the polypeptide of SEQ ID NO: 5 or SEQ ID NO: 6 or SEQ ID NO: 7 or SEQ ID NO:12 or SEQ ID NO:13 or SEQ ID NO:14 and fragments of SEQ ID NO: 5 or SEQ ID NO: 6 or SEQ ID NO: 7 or SEQ ID NO:12 or SEQ ID NO:13 or SEQ ID NO:14 that have at least about 60%, 65%, 70%, 75%, 80%, 85%, 87%, 88%, 89%, 90%, 91%, 92%, 93%, 94%, 95% 96%, 97%, 98%, 99%, 99.1%, 99.2%, 99.3%, 99.4%, 99.5%, 99.6%, 99.7%, 99.8%, 99.9% or 100% sequence identity to the corresponding fragments of SEQ ID NO: 5 or SEQ ID NO: 6 or SEQ ID NO: 7 or SEQ ID NO:12 or SEQ ID NO:13 or SEQ ID NO:14, respectively.

The polypeptide also include sequences comprising a sufficient or substantial degree of identity or similarity to SEQ ID NO: 5 or SEQ ID NO: 6 or SEQ ID NO: 7 or SEQ ID NO:12 or SEQ ID NO:13 or SEQ ID NO:14 to function as a member of the CLC family of chloride channels. The fragments of the polypeptide(s) typically retain some or all of the activity of the full length sequence.

The polypeptides also include mutants produced by introducing any type of alterations (for example, insertions, deletions, or substitutions of amino acids; changes in glycosylation states; changes that affect refolding or isomerizations, three-dimensional structures, or self-association states), which can be deliberately engineered or isolated naturally provided that they still some or all of their function or activity as a member of the CLC family of chloride channels.

The polypeptides may be in linear form or cyclized using known methods.

A polypeptide encoded by SEQ ID NO: 5 or SEQ ID NO: 6 or SEQ ID NO: 7 or SEQ ID NO:12 or SEQ ID NO:13 or SEQ ID NO:14 that has 100% sequence identity thereto or a polypeptide comprising, consisting or consisting essentially of the sequence set forth in SEQ ID NO: 5 or SEQ ID NO: 6 or SEQ ID NO: 7 or SEQ ID NO:12 or SEQ ID NO:13 or SEQ ID NO:14 that has 100% sequence identity thereto is also disclosed.

Various combinations of SEQ ID NO. 5 or SEQ ID NO:6 or SEQ ID NO. 7 or SEQ ID NO:12 or SEQ ID NO:13 or SEQ ID NO:14 are also contemplated. These combinations include any combinations of SEQ ID NO. 5, SEQ ID NO:6, SEQ ID NO. 7, SEQ ID NO:12, SEQ ID NO:13 or SEQ ID NO:14—including the combination of SEQ ID NO:5 and SEQ ID NO:6; the combination of SEQ ID NO:5 and SEQ ID NO:7; the combination of SEQ ID NO:6 and SEQ ID NO:7; the combination of SEQ ID NO:5, SEQ ID NO:6 and SEQ ID NO:7; the combination of SEQ ID NO. 5, SEQ ID NO:6 and SEQ ID NO. 7; the combination of SEQ ID NO:5, SEQ ID NO:6, SEQ ID NO:12, SEQ ID NO:13 and SEQ ID NO:14; the combination of SEQ ID NO:5, SEQ ID NO:7, SEQ ID NO:12, SEQ ID NO:13 and SEQ ID NO:14; the combination of SEQ ID NO:6, SEQ ID NO:7 SEQ ID NO:12, SEQ ID NO:13 and SEQ ID NO:14; the combination of SEQ ID NO:5, SEQ ID NO:6, SEQ ID NO:7 SEQ ID NO:12, SEQ ID NO:13 and SEQ ID NO:14; the combination of SEQ ID NO. 5, SEQ ID NO:6 and SEQ ID NO. 7, SEQ ID NO:12, SEQ ID NO:13 and SEQ ID NO:14 etc.

Polypeptides include variants produced by introducing any type of alterations (for example, insertions, deletions, or substitutions of amino acids; changes in glycosylation states; changes that affect refolding or isomerizations, three-dimensional structures, or self-association states), which can be deliberately engineered or isolated naturally. A deletion refers to removal of one or more amino acids from a protein. An insertion refers to one or more amino acid residues being introduced into a predetermined site in a polypeptide. Insertions may comprise intra-sequence insertions of single or multiple amino acids. A substitution refers to the replacement of amino acids of the polypeptide with other amino acids having similar properties (such as similar hydrophobicity, hydrophilicity, antigenicity, propensity to form or break a-helical structures or β-sheet structures). Amino acid substitutions are typically of single residues, but may be clustered depending upon functional constraints placed upon the polypeptide and may range from about 1 to about 10 amino acids. The amino acid substitutions are preferably conservative amino acid substitutions as described below. Amino acid substitutions, deletions and/or insertions can be made using peptide synthetic techniques—such as solid phase peptide synthesis or by recombinant DNA manipulation. Methods for the manipulation of DNA sequences to produce substitution, insertion or deletion variants of a protein are well known in the art. The variant may have alterations which produce a silent change and result in a functionally equivalent protein. Deliberate amino acid substitutions may be made on the basis of similarity in polarity, charge, solubility, hydrophobicity, hydrophilicity and the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine. Conservative substitutions may be made, for example according to the Table below. Amino acids in the same block in the second column and preferably in the same line in the third column may be substituted for each other:

| | | |
|---|---|---|
| ALIPHATIC | Non-polar | Gly Ala Pro |
| | | Ile Leu Val |
| | Polar-uncharged | Cys Ser Thr Met |
| | | Asn Gly |
| | Polar-charged | Asp Glu |
| | | Lys Arg |
| AROMATIC | | His Phe Trp Tyr |

The polypeptide may be a mature protein or an immature protein or a protein derived from an immature protein. Polypeptides may be in linear form or cyclized using known methods. Polypeptides typically comprise at least 10, at least 20, at least 30, or at least 40 contiguous amino acids.

Mutant polypeptide variants can be used to create mutant, non-naturally occurring or transgenic plants (for example, mutant, non-naturally occurring, transgenic, man-made or genetically engineered plants) comprising one or more mutant polypeptide variants. Suitably, mutant polypeptide variants retain the activity of the unmutated polypeptide. The activity of the mutant polypeptide variant may be higher, lower or about the same as the unmutated polypeptide.

Mutations in the nucleotide sequences and polypeptides described herein can include man-made mutations or synthetic mutations or genetically engineered mutations. Mutations in the nucleotide sequences and polypeptides described herein can be mutations that are obtained or obtainable via a process which includes an in vitro or an in vivo manipulation step. Mutations in the nucleotide sequences and polypeptides described herein can be mutations that are obtained or obtainable via a process which includes intervention by man.

Examples of mutations in the polypeptide sequences described herein are shown in Table 1. Accordingly, a further aspect relates to the mutant polypeptides as set forth in Table 1.

The mutation(s) can modulate the activity of the encoded polypeptide. The mutation(s) can modulate the activity of the encoded polypeptide such that the nitrate level in the plant is modulated. The mutation(s) can modulate the activity of the encoded polypeptide such that the nitrate level in the plant is increased or decreased. The mutation(s) can modulate the activity of the encoded polypeptide such that the NNK level in the plant—such as cured plant material—is modulated. The mutation(s) can modulate the activity of the encoded polypeptide such that the NNK level in the plant—such as cured plant material—is increased or decreased. The mutation(s) can modulate the activity of the encoded polypeptide such that the overall TSNA level in the plant—such as cured plant material—is modulated. The mutation(s) can modulate the activity of the encoded polypeptide such that the overall TSNA level in the plant—such as cured plant material—is increased or decreased.

In another embodiment, the mutation(s) can alter the biomass yield of the plant. In one embodiment, the Tobacco NtCLCe-T P184S homozygous mutant has almost double biomass production with respect to cured leaves per plant compared to similar plants including for example tobacco plants heterozygous for the said mutation and tobacco plants homozygous for other CLC-mutations. The NtCLCe-T P184S homozygous mutant plant yields approximately 150 g cured leaves per plant compared to approximately 80 g of cured leaves for normal comparative tobacco plants.

Accordingly, there is provided a plant comprising a mutated CLC polypeptide as set forth herein. In one embodiment, the plant comprises a P184S mutation.

Certain CLC mutations do not affect biomass yield. The invention therefore comprises screening the mutant plants for increased biomass yield, selecting those mutants which show a yield increase of at least 1.5× in comparison to control plants not comprising said mutation(s), and cultivating those plants in which a desirable biomass yield is achieved.

Plants with increased biomass yield may or may not have altered nitrate production. In one embodiment, nitrate production is not affected compared to a control plant.

In one embodiment, there is provided a method for screening of a plant with increased biomass yield, comprising screening CLC chloride channel mutant for increased biomass yields and selecting those plants in which yield is increased. Suitably, yield is increased by at least 1.5×. Suitably, the mutation is selected from one or more of the CLC mutations described herein.

In another embodiment, CLC mutant plants may have increased biomass yield and decreased nitrate production compared to a control plant. The invention therefore comprises screening the mutant plants for increased biomass yield, selecting those mutants which show a yield increase of at least 1.5×, screening the selected mutants for modulated nitrate production compared to a control plant, and selecting those mutants which show a decrease in nitrate production compared to a control plant.

In one embodiment, SEQ ID NO. 5 includes one or more mutations at amino acid positions selected from the group consisting of 503, 471, 659, 566, 637, 597, 711, 135, 151, 690, 737, 135, 163, 480, 520, 514, 518, 476, 739, 517, 585 or 677 or a combination of two or more thereof. The type of mutation(s) at this position can be a deletion, an insertion, a substitution or a missense mutation or a combination thereof. The mutation(s) can be a heterozygous or homozygous mutation, suitably, a homozygous mutation. In one embodiment, the mutation(s) is a substitution mutation. In one embodiment, the substitution mutation(s) is selected from the group consisting of G503E, G471R, V6591, S566N, P637S, A597T, P711L, G135R, A151V, G690D, G737R, G135R, G163R, P480S, S520F, A514T, A518V, G476E, R739S, G517E, E585K or V677I or a combination of two or more thereof.

In one embodiment, SEQ ID NO. 6 includes one or more mutations at amino acid positions selected from the group consisting of 514, 537, 593, 749, 524, 408, 503, 547, 691, 478, 749, 713, 550, 586, 670, 678, 631, 657, 737, 525, 597, 674 or a combination of two or more thereof. The type of mutation(s) at this position can be a deletion, an insertion, a substitution or a missense mutation or a combination thereof. The mutation(s) can be a heterozygous or homozygous mutation, suitably, a homozygous mutation. In one embodiment, the mutation(s) is a substitution mutation. In one embodiment, the substitution mutation(s) is selected from the group consisting of A514T, L537F, R5931, A749T, G524D, S408F, G503R, P547S, G691D, A478V, A749V, T713I, M5501, P586S, R670K, R678K, D631N, L657F, G737R, S525L, A597T, E674K or a combination of two or more thereof.

In one embodiment, SEQ ID NO:7 includes one or more mutations at amino acid positions selected from the group consisting of 21, 58, 141, 175, 5, 34, 124, 40, 8, 35, 30, 177, 42, 88, 155, 158, 170, 174, 126 or 131 or a combination of two or more thereof. The type of mutation(s) at this position can be a deletion, an insertion, a substitution or a missense mutation or a combination thereof. The mutation(s) can be a heterozygous or homozygous mutation, suitably, a homozygous mutation. In one embodiment, the mutation(s) is a substitution mutation. In one embodiment, the substitution mutation(s) is selected from the group consisting of E21K, L58F, P141S, G175E, S5N, A34V, M1241, L40F, D8N, C35Y, A30V, A177V, G42D, G88D, G155R, D158N, A170V, A174V, A126V or G131R or a combination of two or more thereof.

The sequence shown in SEQ ID NO:12 corresponds to the sequence shown in SEQ ID NO:7 with an extra 88 amino acids at the 5′ end. SEQ ID NO:12 can include the same corresponding mutations as SEQ ID NO:7. SEQ ID NO:12 can include one or more mutations at amino acid positions selected from the group consisting of 109, 146, 229, 263, 93, 122, 212, 128, 96, 123, 118, 265, 130, 176, 243, 246, 258, 262, 214, or 219 or a combination of two or more thereof. The type of mutation(s) at this position can be a deletion, an insertion, a substitution or a missense mutation or a combination thereof. The mutation(s) can be a heterozygous or homozygous mutation, suitably, a homozygous mutation. In one embodiment, the mutation(s) is a substitution mutation. In one embodiment, the substitution mutation(s) is selected from the group consisting of E109K, L146F, P229S, G263E, S93N, A122V, M2121, L128F, D96N, C123Y, A118V, A265V, G130D, G176D, G243R, D246N, A258V, A262V, A214V or G219R or a combination of two or more thereof.

In one embodiment, SEQ ID NO:13 includes one or more mutations at amino acid positions selected from the group consisting of 184, 89, 166, 18, 76, 173, 143, 1, 4, 154, 89, 128, 137 or 181 or a combination of two or more thereof. The type of mutation(s) at this position can be a deletion, an insertion, a substitution or a missense mutation or a combination thereof. The mutation(s) can be a heterozygous or homozygous mutation, suitably, a homozygous mutation. In one embodiment, the mutation(s) is a substitution mutation. In one embodiment, the substitution mutation(s) is selected from the group consisting of P184S, G89D, K166N, G18R, G76R, G173R, P143L, M1I, S4N, V1541, G89D, A128V, S137F or G181S or a combination of two or more thereof. The sequence shown in SEQ ID NO:14 corresponds to the sequence shown in SEQ ID NO:13 with an extra 88 amino acids at the 5′ end. In one embodiment, SEQ ID NO:14 includes one or more mutations at amino acid positions selected from the group consisting of 272, 177, 254, 106, 164, 261, 231, 89, 92, 242, 177, 269 or 225 or a combination of two or more thereof. The type of mutation(s) at this position can be a deletion, an insertion, a substitution or a missense mutation or a combination thereof. The mutation(s) can be a heterozygous or homozygous mutation, suitably, a homozygous mutation. In one embodiment, the mutation(s) is a substitution mutation. In one embodiment, the substitution mutation(s) is selected from the group consisting of P272S, G177D, K254N, G106R, G164R, G261R, P231L, M891, S92N, V2421, G177D, A269V, S225F or G269S or a combination of two or more thereof.

Suitably, the mutation is a mutation at position G163 of SEQ ID NO:5. Suitably, the mutation is a homozygous mutation at position G163 of SEQ ID NO:5. Suitably, the mutation is a substitution mutation. Suitably, the substitution mutation is G163R. Suitably, the mutation is homozygous substitution mutation at G163R. When a polypeptide comprising this mutation is expressed in a mutant plant the nitrate level in the mutant plant is lower than the control plant during the early and mid-morning. Corresponding mutations can be made in SEQ ID NO:14, which corresponds to the sequence of SEQ ID NO:7 with additional amino acids at the 5′ end thereof.

Suitably, the mutation is a mutation at position G163 of SEQ ID NO:5. Suitably, the substitution mutation is G163R. Suitably, the mutation is homozygous substitution mutation at G163R. This mutation can decrease the level of nitrate in a mutant plant containing this mutation. The G163R homozygous mutant tobacco plant has a reduced level of nitrate in the early morning as compared to the control plant. The level of nitrate is reduced from about 11 mg/g in the control plant to about 6 mg/g in the mutant plant. The nitrate level continues to decrease in the mid-morning. The level of nitrate is reduced from about 7 mg/g in the control plant to about 4.5 mg/g in the mutant plant. By the late morning the nitrate level has increased in the mutant plant as compared to the mid-morning and reaches the nitrate level present in the early morning. For the control, the nitrate level in the control plant continues to decrease. By late morning, the level of nitrate increases to about 6 mg/g in the mutant plant and decreases to about 3 mg/g in the control plant. The level of nicotine is somewhat similar during the morning. The level of nicotine varies between about 13 mg/g and about 11 mg/g for the mutant plant and about 9 mg/g and 13 mg/g for the control plant. The nicotine result indicates that the metabolism of the mutant plant is normal. The biomass levels for the mutant and the control plant are also comparable.

Suitably, the mutation is a mutation at position P143 of SEQ ID NO:13. Suitably, the substitution mutation is P143L. Suitably, the mutation is homozygous substitution mutation at P143L. This mutation can increase the level of nitrate in a mutant plant containing this mutation. The P143L homozygous mutant tobacco plant has an increased level of nitrate in the early morning as compared to the control plant. The level of nitrate is increased from about 7 mg/g in the control plant to about 14 mg/g in the mutant plant. The nitrate level decreases in the mid-morning in the mutant plant and increases slightly in the control plant. The level of nitrate in the mutant plant is reduced to about 9 mg/g and the level of nitrate in the control plant increases to about 9 mg/g. By the late morning the nitrate level has continued to decrease in the mutant plant as compared to the mid-morning. For the control, the nitrate level in the control plant decreases. By late morning, the level of nitrate decreases to about 2 mg/g in the mutant plant and decreases to about 4 mg/g in the control plant. The level of nicotine is somewhat similar during the morning for each of the mutant and control plants. The level of nicotine varies between about 20 mg/g and about 24 mg/g for the mutant plant and about 15 mg/g and 17 mg/g for the control plant. The nicotine result indicates that the metabolism of the mutant plant is normal. The biomass levels for the mutant and the control plant are also comparable.

The diurnal regulation of nitrate metabolism is known and has been intensively investigated (see Stitt & Krapp Plant, Cell and Environment 22, 583-621 (1999)). In nitrogen replete plants, the level of the transcript for nitrate reductases is high at the end of the night, falls dramatically during the day, and recovers during the night. NIA activity increases three-fold in the first part of the light period, decreases during the second part of the light period and remains low during the night. The increase of NIA activity after illumination is due to an increase of NIA protein.

There is also disclosed a method for modulating the level of nitrate, total TSNA content or NNK in a tobacco plant, or a plant part thereof, said method comprising the steps of: (i) introducing into the genome of said plant one or more mutations within at least one allele of the one or more polynucleotide sequences described herein; and (ii) obtaining a mutant plant in which said mutation modulates the expression of said polynucleotide sequences or the activity of the polypeptide encoded thereby as compared to a control and the tobacco plant or a plant part thereof has a modulated level of nitrate and/or total TSNA content and/or NNK. In certain embodiments, the tobacco plant or plant part thereof is cured plant material.

Processes for preparing mutants are well known in the art and may include mutagenesis using exogenously added chemicals—such as mutagenic, teratogenic, or carcinogenic organic compounds, for example ethyl methanesulfonate (EMS), that produce random mutations in genetic material. By way of further example, the process may include one or more genetic engineering steps—such as one or more of the genetic engineering steps that are described herein or combinations thereof. By way of further example, the process may include one or more plant crossing steps. TILLING may also be used as described elsewherein herein.

A polypeptide may be prepared by culturing transformed or recombinant host cells under culture conditions suitable to express a polypeptide. The resulting expressed polypeptide may then be purified from such culture using known purification processes. The purification of the polypeptide may include an affinity column containing agents which will bind to the polypeptide; one or more column steps over such affinity resins; one or more steps involving hydrophobic interaction chromatography; or immunoaffinity chromatography. Alternatively, the polypeptide may also be expressed in a form that will facilitate purification. For example, it may be expressed as a fusion polypeptide, such as those of maltose binding polypeptide, glutathione-5-transferase or thioredoxin. Kits for expression and purification of fusion polypeptides are commercially available.

The polypeptide may be tagged with an epitope and subsequently purified by using a specific antibody directed to such epitope. One or more liquid chromatography steps—such as reverse-phase high performance liquid chromatography can be employed to further purify the polypeptide.

Some or all of the foregoing purification steps, in various combinations, can be employed to provide a substantially homogeneous recombinant polypeptide. The polypeptide thus purified may be substantially free of other polypeptides and is defined herein as a “substantially purified polypeptide”; such purified polypeptides include polypeptides, fragments, variants, and the like. Expression, isolation, and purification of the polypeptides and fragments can be accomplished by any suitable technique, including but not limited to the methods described herein.

It is also possible to utilise an affinity column such as a monoclonal antibody generated against polypeptides, to affinity-purify expressed polypeptides. These polypeptides can be removed from an affinity column using conventional techniques, for example, in a high salt elution buffer and then dialyzed into a lower salt buffer for use or by changing pH or other components depending on the affinity matrix utilized, or be competitively removed using the naturally occurring substrate of the affinity moiety.

A polypeptide may also be produced by known conventional chemical synthesis. Methods for constructing the polypeptides or fragments thereof by synthetic means are known to those skilled in the art. The synthetically-constructed polypeptide sequences, by virtue of sharing primary, secondary or tertiary structural or conformational characteristics with native polypeptides may possess biological properties in common therewith, including biological activity.

The term ‘non-naturally occurring’ as used herein describes an entity (for example, a polynucleotide, a genetic mutation, a polypeptide, a plant, a plant cell and plant material) that is not formed by nature or that does not exist in nature. Such non-naturally occurring entities or artificial entities may be made, synthesized, initiated, modified, intervened, or manipulated by methods described herein or that are known in the art. Such non-naturally occurring entities or artificial entities may be made, synthesized, initiated, modified, intervened, or manipulated by man. Thus, by way of example, a non-naturally occurring plant, a non-naturally occurring plant cell or non-naturally occurring plant material may be made using traditional plant breeding techniques—such as backcrossing—or by genetic manipulation technologies—such as antisense RNA, interfering RNA, meganuclease and the like. By way of further example, a non-naturally occurring plant, a non-naturally occurring plant cell or non-naturally occurring plant material may be made by introgression of or by transferring one or more genetic mutations (for example one or more polymorphisms) from a first plant or plant cell into a second plant or plant cell (which may itself be naturally occurring), such that the resulting plant, plant cell or plant material or the progeny thereof comprises a genetic constitution (for example, a genome, a chromosome or a segment thereof) that is not formed by nature or that does not exist in nature. The resulting plant, plant cell or plant material is thus artificial or non-naturally occurring. Accordingly, an artificial or non-naturally occurring plant or plant cell may be made by modifying a genetic sequence in a first naturally occurring plant or plant cell, even if the resulting genetic sequence occurs naturally in a second plant or plant cell that comprises a different genetic background from the first plant or plant cell. In certain embodiments, a mutation is not a naturally occurring mutation that exists naturally in a nucleotide sequence or a polypeptide—such as a gene or a protein.

Differences in genetic background can be detected by phenotypic differences or by molecular biology techniques known in the art—such as nucleic acid sequencing, presence or absence of genetic markers (for example, microsatellite RNA markers).

Antibodies that are immunoreactive with the polypeptides described herein are also provided. The polypeptides, fragments, variants, fusion polypeptides, and the like, as set forth herein, can be employed as “immunogens” in producing antibodies immunoreactive therewith. Such antibodies may specifically bind to the polypeptide via the antigen-binding sites of the antibody. Specifically binding antibodies are those that will specifically recognize and bind with a polypeptide, homologues, and variants, but not with other molecules. In one embodiment, the antibodies are specific for polypeptides having an amino acid sequence as set forth herein and do not cross-react with other polypeptides.

More specifically, the polypeptides, fragment, variants, fusion polypeptides, and the like contain antigenic determinants or epitopes that elicit the formation of antibodies. These antigenic determinants or epitopes can be either linear or conformational (discontinuous). Linear epitopes are composed of a single section of amino acids of the polypeptide, while conformational or discontinuous epitopes are composed of amino acids sections from different regions of the polypeptide chain that are brought into close proximity upon polypeptide folding. Epitopes can be identified by any of the methods known in the art. Additionally, epitopes from the polypeptides can be used as research reagents, in assays, and to purify specific binding antibodies from substances such as polyclonal sera or supernatants from cultured hybridomas. Such epitopes or variants thereof can be produced using techniques known in the art such as solid-phase synthesis, chemical or enzymatic cleavage of a polypeptide, or using recombinant DNA technology.

Both polyclonal and monoclonal antibodies to the polypeptides can be prepared by conventional techniques. Hybridoma cell lines that produce monoclonal antibodies specific for the polypeptides are also contemplated herein. Such hybridomas can be produced and identified by conventional techniques. For the production of antibodies, various host animals may be immunized by injection with a polypeptide, fragment, variant, or mutants thereof. Such host animals may include, but are not limited to, rabbits, mice, and rats, to name a few. Various adjutants may be used to increase the immunological response. Depending on the host species, such adjuvants include, but are not limited to, Freund's (complete and incomplete), mineral gels such as aluminium hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. The monoclonal antibodies can be recovered by conventional techniques. Such monoclonal antibodies may be of any immunoglobulin class including IgG, IgM, IgE, IgA, IgD, and any subclass thereof.

The antibodies can also be used in assays to detect the presence of the polypeptides or fragments, either in vitro or in vivo. The antibodies also can be employed in purifying polypeptides or fragments by immunoaffinity chromatography.

Compositions that can modulate the expression or the activity of one or more of the polynucleotides or polypeptides described herein (or any combination thereof as described herein) include, but are not limited to, sequence-specific polynucleotides that can interfere with the transcription of one or more endogenous gene(s); sequence-specific polynucleotides that can interfere with the translation of RNA transcripts (for example, double-stranded RNAs, siRNAs, ribozymes); sequence-specific polypeptides that can interfere with the stability of one or more proteins; sequence-specific polynucleotides that can interfere with the enzymatic activity of one or more proteins or the binding activity of one or more proteins with respect to substrates or regulatory proteins; antibodies that exhibit specificity for one or more proteins; small molecule compounds that can interfere with the stability of one or more proteins or the enzymatic activity of one or more proteins or the binding activity of one or more proteins; zinc finger proteins that bind one or more polynucleotides; and meganucleases that have activity towards one or more polynucleotides. Gene editing technologies, genetic editing technologies and genome editing technologies are well known in the art.

One method of gene editing involves the use of transcription activator-like effector nucleases (TALENs) which induce double-strand breaks which cells can respond to with repair mechanisms. Non-homologous end joining reconnects DNA from either side of a double-strand break where there is very little or no sequence overlap for annealing. This repair mechanism induces errors in the genome via insertion or deletion, or chromosomal rearrangement. Any such errors may render the gene products coded at that location non-functional.

Another method of gene editing involves the use of the bacterial CRISPR/Cas system. Bacteria and archaea exhibit chromosomal elements called clustered regularly interspaced short palindromic repeats (CRISPR) that are part of an adaptive immune system that protects against invading viral and plasmid DNA. In Type II CRISPR systems, CRISPR RNAs (crRNAs) function with trans-activating crRNA (tracrRNA) and CRISPR-associated (Cas) proteins to introduce double-stranded breaks in target DNA. Target cleavage by Cas9 requires base-pairing between the crRNA and tracrRNA as well as base pairing between the crRNA and the target DNA. Target recognition is facilitated by the presence of a short motif called a protospacer-adjacent motif (PAM) that conforms to the sequence NGG. This system can be harnessed for genome editing. Cas9 is normally programmed by a dual RNA consisting of the crRNA and tracrRNA. However, the core components of these RNAs can be combined into a single hybrid ‘guide RNA’ for Cas9 targeting.

The use of a noncoding RNA guide to target DNA for site-specific cleavage promises to be significantly more straightforward than existing technologies—such as TALENs. Using the CRISPR/Cas strategy, retargeting the nuclease complex only requires introduction of a new RNA sequence and there is no need to reengineer the specificity of protein transcription factors. Antisense technology is another well-known method that can be used to modulate the expression of a polypeptide. A polynucleotide of the gene to be repressed is cloned and operably linked to a regulatory region and a transcription termination sequence so that the antisense strand of RNA is transcribed. The recombinant construct is then transformed into plants and the antisense strand of RNA is produced. The polynucleotide need not be the entire sequence of the gene to be repressed, but typically will be substantially complementary to at least a portion of the sense strand of the gene to be repressed.

A polynucleotide may be transcribed into a ribozyme, or catalytic RNA, that affects expression of an mRNA. Ribozymes can be designed to specifically pair with virtually any target RNA and cleave the phosphodiester backbone at a specific location, thereby functionally inactivating the target RNA. Heterologous polynucleotides can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contains a 5′-UG-3′ nucleotide sequence. The construction and production of hammerhead ribozymes is known in the art. Hammerhead ribozyme sequences can be embedded in a stable RNA such as a transfer RNA (tRNA) to increase cleavage efficiency in vivo.

In one embodiment, the sequence-specific polynucleotide that can interfere with the translation of RNA transcript(s) is interfering RNA. RNA interference or RNA silencing is an evolutionarily conserved process by which specific mRNAs can be targeted for enzymatic degradation. A double-stranded RNA (double-stranded RNA) is introduced or produced by a cell (for example, double-stranded RNA virus, or interfering RNA polynucleotides) to initiate the interfering RNA pathway. The double-stranded RNA can be converted into multiple small interfering RNA duplexes of 21-23 bp length by RNases III, which are double-stranded RNA-specific endonucleases. The small interfering RNAs can be subsequently recognized by RNA-induced silencing complexes that promote the unwinding of small interfering RNA through an ATP-dependent process. The unwound antisense strand of the small interfering RNA guides the activated RNA-induced silencing complexes to the targeted mRNA comprising a sequence complementary to the small interfering RNA anti-sense strand. The targeted mRNA and the anti-sense strand can form an A-form helix, and the major groove of the A-form helix can be recognized by the activated RNA-induced silencing complexes. The target mRNA can be cleaved by activated RNA-induced silencing complexes at a single site defined by the binding site of the 5′-end of the small interfering RNA strand. The activated RNA-induced silencing complexes can be recycled to catalyze another cleavage event.

Interfering RNA expression vectors may comprise interfering RNA constructs encoding interfering RNA polynucleotides that exhibit RNA interference activity by reducing the expression level of mRNAs, pre-mRNAs, or related RNA variants. The expression vectors may comprise a promoter positioned upstream and operably-linked to an Interfering RNA construct, as further described herein. Interfering RNA expression vectors may comprise a suitable minimal core promoter, a Interfering RNA construct of interest, an upstream (5′) regulatory region, a downstream (3′) regulatory region, including transcription termination and polyadenylation signals, and other sequences known to persons skilled in the art, such as various selection markers.

The polynucleotides can be produced in various forms, including as double stranded structures (that is, a double-stranded RNA molecule comprising an antisense strand and a complementary sense strand), double-stranded hairpin-like structures, or single-stranded structures (that is, a ssRNA molecule comprising just an antisense strand). The structures may comprise a duplex, asymmetric duplex, hairpin or asymmetric hairpin secondary structure, having self-complementary sense and antisense strands. The double stranded interfering RNA can be enzymatically converted to double-stranded small interfering RNAs. One of the strands of the small interfering RNA duplex can anneal to a complementary sequence within the target mRNA and related RNA variants. The small interfering RNA/mRNA duplexes are recognized by RNA-induced silencing complexes that can cleave RNAs at multiple sites in a sequence-dependent manner, resulting in the degradation of the target mRNA and related RNA variants.

The double-stranded RNA molecules may include small interfering RNA molecules assembled from a single oligonucleotide in a stem-loop structure, wherein self-complementary sense and antisense regions of the small interfering RNA molecule are linked by means of a polynucleotide based or non-polynucleotide-based linker(s), as well as circular single-stranded RNA having two or more loop structures and a stem comprising self-complementary sense and antisense strands, wherein the circular RNA can be processed either in vivo or in vitro to generate an active small interfering RNA molecule capable of mediating interfering RNA.

The use of small hairpin RNA molecules is also contemplated. They comprise a specific antisense sequence in addition to the reverse complement (sense) sequence, typically separated by a spacer or loop sequence. Cleavage of the spacer or loop provides a single-stranded RNA molecule and its reverse complement, such that they may anneal to form a double-stranded RNA molecule (optionally with additional processing steps that may result in addition or removal of one, two, three or more nucleotides from the 3′ end or the 5′ end of either or both strands). The spacer can be of a sufficient length to permit the antisense and sense sequences to anneal and form a double-stranded structure (or stem) prior to cleavage of the spacer (and, optionally, subsequent processing steps that may result in addition or removal of one, two, three, four, or more nucleotides from the 3′ end or the 5′ end of either or both strands). The spacer sequence is typically an unrelated nucleotide sequence that is situated between two complementary nucleotide sequence regions which, when annealed into a double-stranded polynucleotide, comprise a small hairpin RNA. The spacer sequence generally comprises between about 3 and about 100 nucleotides.

Any RNA polynucleotide of interest can be produced by selecting a suitable sequence composition, loop size, and stem length for producing the hairpin duplex. A suitable range for designing stem lengths of a hairpin duplex, includes stem lengths of at least about 10, 11, 12, 13, 14, 15, 16, 17, 18, 19 or 20 nucleotides—such as about 14-30 nucleotides, about 30-50 nucleotides, about 50-100 nucleotides, about 100-150 nucleotides, about 150-200 nucleotides, about 200-300 nucleotides, about 300-400 nucleotides, about 400-500 nucleotides, about 500-600 nucleotides, and about 600-700 nucleotides. A suitable range for designing loop lengths of a hairpin duplex, includes loop lengths of about 4-25 nucleotides, about 25-50 nucleotides, or longer if the stem length of the hair duplex is substantial. In certain embodiments, a double-stranded RNA or ssRNA molecule is between about 15 and about 40 nucleotides in length. In another embodiment, the small interfering RNA molecule is a double-stranded RNA or ssRNA molecule between about 15 and about 35 nucleotides in length. In another embodiment, the small interfering RNA molecule is a double-stranded RNA or ssRNA molecule between about 17 and about 30 nucleotides in length.

In another embodiment, the small interfering RNA molecule is a double-stranded RNA or ssRNA molecule between about 19 and about 25 nucleotides in length. In another embodiment, the small interfering RNA molecule is a double-stranded RNA or ssRNA molecule between about 21 to about 23 nucleotides in length. In certain embodiments, hairpin structures with duplexed regions longer than 21 nucleotides may promote effective small interfering RNA-directed silencing, regardless of loop sequence and length. Exemplary sequences for RNA interference are set forth in SEQ ID NO: 8 or SEQ ID NO: 9.

The target mRNA sequence is typically between about 14 to about 50 nucleotides in length. The target mRNA can, therefore, be scanned for regions between about 14 and about 50 nucleotides in length that preferably meet one or more of the following criteria for a target sequence: an A+T/G+C ratio of between about 2:1 and about 1:2; an AA dinucleotide or a CA dinucleotide at the 5′ end of the target sequence; a sequence of at least 10 consecutive nucleotides unique to the target mRNA (that is, the sequence is not present in other mRNA sequences from the same plant); and no “runs” of more than three consecutive guanine (G) nucleotides or more than three consecutive cytosine (C) nucleotides. These criteria can be assessed using various techniques known in the art, for example, computer programs such as BLAST can be used to search publicly available databases to determine whether the selected target sequence is unique to the target mRNA. Alternatively, a target sequence can be selected (and a small interfering RNA sequence designed) using computer software available commercially (for example, OligoEngine, Target Finder and the small interfering RNA Design Tool which are commercially available).

In one embodiment, target mRNA sequences are selected that are between about 14 and about 30 nucleotides in length that meet one or more of the above criteria. In another embodiment, target sequences are selected that are between about 16 and about 30 nucleotides in length that meet one or more of the above criteria. In a further embodiment, target sequences are selected that are between about 19 and about 30 nucleotides in length that meet one or more of the above criteria. In another embodiment, target sequences are selected that are between about 19 and about 25 nucleotides in length that meet one or more of the above criteria.

In an exemplary embodiment, the small interfering RNA molecules comprise a specific antisense sequence that is complementary to at least 12, 13, 14, 15, 16, 17, 18, 19, 20, 21, 22, 23, 24, 25, 26, 27, 28, 29, 30, or more contiguous nucleotides of any one of the polynucleotide sequences described herein.

The specific antisense sequence comprised by the small interfering RNA molecule can be identical or substantially identical to the complement of the target sequence. In one embodiment, the specific antisense sequence comprised by the small interfering RNA molecule is at least about 75%, 80%, 85%, 90%, 95%, 96%, 97%, 98%, 99% or 100% identical to the complement of the target mRNA sequence. Methods of determining sequence identity are known in the art and can be determined, for example, by using the BLASTN program of the University of Wisconsin Computer Group (GCG) software or provided on the NCBI website.

The specific antisense sequence of the small interfering RNA molecules may exhibit variability by differing (for example, by nucleotide substitution, including transition or transversion) at one, two, three, four or more nucleotides from the sequence of the target mRNA. When such nucleotide substitutions are present in the antisense strand of a double-stranded RNA molecule, the complementary nucleotide in the sense strand with which the substitute nucleotide would typically form hydrogen bond base-pairing may or may not be correspondingly substituted. Double-stranded RNA molecules in which one or more nucleotide substitution occurs in the sense sequence, but not in the antisense strand, are also contemplated. When the antisense sequence of an small interfering RNA molecule comprises one or more mismatches between the nucleotide sequence of the small interfering RNA and the target nucleotide sequence, as described above, the mismatches may be found at the 3′ terminus, the 5′ terminus or in the central portion of the antisense sequence. In another embodiment, the small interfering RNA molecules comprise a specific antisense sequence that is capable of selectively hybridizing under stringent conditions to a portion of a naturally occurring target gene or target mRNA. As known to those of ordinary skill in the art, variations in stringency of hybridization conditions may be achieved by altering the time, temperature or concentration of the solutions used for the hybridization and wash steps. Suitable conditions can also depend in part on the particular nucleotide sequences used, for example the sequence of the target mRNA or gene.

One method for inducing double stranded RNA-silencing in plants is transformation with a gene construct producing hairpin RNA (see Smith et al. (2000) Nature, 407, 319-320). Such constructs comprise inverted regions of the target gene sequence, separated by an appropriate spacer. The insertion of a functional plant intron region as a spacer fragment additionally increases the efficiency of the gene silencing induction, due to generation of an intron spliced hairpin RNA (Wesley et al. (2001) Plant J., 27, 581-590). Suitably, the stem length is about 50 nucleotides to about 1 kilobases in length. Methods for producing intron spliced hairpin RNA are well described in the art (see for example, Bioscience, Biotechnology, and Biochemistry (2008) 72, 2, 615-617). Interfering RNA molecules having a duplex or double-stranded structure, for example double-stranded RNA or small hairpin RNA, can have blunt ends, or can have 3′ or 5′ overhangs. As used herein, “overhang” refers to the unpaired nucleotide or nucleotides that protrude from a duplex structure when a 3′-terminus of one RNA strand extends beyond the 5′-terminus of the other strand (3′ overhang), or vice versa (5′ overhang). The nucleotides comprising the overhang can be ribonucleotides, deoxyribonucleotides or modified versions thereof. In one embodiment, at least one strand of the interfering RNA molecule has a 3′ overhang from about 1 to about 6 nucleotides in length. In other embodiments, the 3′ overhang is from about 1 to about 5 nucleotides, from about 1 to about 3 nucleotides and from about 2 to about 4 nucleotides in length.

When the interfering RNA molecule comprises a 3′ overhang at one end of the molecule, the other end can be blunt-ended or have also an overhang (5′ or 3′). When the interfering RNA molecule comprises an overhang at both ends of the molecule, the length of the overhangs may be the same or different. In one embodiment, the interfering RNA molecule comprises 3′ overhangs of about 1 to about 3 nucleotides on both ends of the molecule. In a further embodiment, the interfering RNA molecule is a double-stranded RNA having a 3′ overhang of 2 nucleotides at both ends of the molecule. In yet another embodiment, the nucleotides comprising the overhang of the interfering RNA are TT dinucleotides or UU dinucleotides.

When determining the percentage identity of the interfering RNA molecule comprising one or more overhangs to the target mRNA sequence, the overhang(s) may or may not be taken into account. For example, the nucleotides from a 3′ overhang and up to 2 nucleotides from the 5′- or 3′-terminus of the double strand may be modified without significant loss of activity of the small interfering RNA molecule.

The interfering RNA molecules can comprise one or more 5′ or 3′-cap structures. The interfering RNA molecule can comprise a cap structure at the 3′-end of the sense strand, the antisense strand, or both the sense and antisense strands; or at the 5′-end of the sense strand, the antisense strand, or both the sense and antisense strands of the interfering RNA molecule. Alternatively, the interfering RNA molecule can comprise a cap structure at both the 3′-end and 5′-end of the interfering RNA molecule. The term “cap structure” refers to a chemical modification incorporated at either terminus of an oligonucleotide, which protects the molecule from exonuclease degradation, and may also facilitate delivery or localisation within a cell.

Another modification applicable to interfering RNA molecules is the chemical linkage to the interfering RNA molecule of one or more moieties or conjugates which enhance the activity, cellular distribution, cellular uptake, bioavailability or stability of the interfering RNA molecule. The polynucleotides may be synthesized or modified by methods well established in the art. Chemical modifications may include, but are not limited to 2′ modifications, introduction of non-natural bases, covalent attachment to a ligand, and replacement of phosphate linkages with thiophosphate linkages. In this embodiment, the integrity of the duplex structure is strengthened by at least one, and typically two, chemical linkages. Chemical linking may be achieved by any of a variety of well-known techniques, for example by introducing covalent, ionic or hydrogen bonds; hydrophobic interactions, van der Waals or stacking interactions; by means of metal-ion coordination, or through use of purine analogues.

The nucleotides at one or both of the two single strands may be modified to modulate the activation of cellular enzymes, such as, for example, without limitation, certain nucleases. Techniques for reducing or inhibiting the activation of cellular enzymes are known in the art including, but not limited to, 2′-amino modifications, 2′-fluoro modifications, 2′-alkyl modifications, uncharged backbone modifications, morpholino modifications, 2′-O-methyl modifications, and phosphoramidate. Thus, at least one 2′-hydroxyl group of the nucleotides on a double-stranded RNA is replaced by a chemical group. Also, at least one nucleotide may be modified to form a locked nucleotide. Such locked nucleotide contains a methylene or ethylene bridge that connects the 2′-oxygen of ribose with the 4′-carbon of ribose. Introduction of a locked nucleotide into an oligonucleotide improves the affinity for complementary sequences and increases the melting temperature by several degrees.

Ligands may be conjugated to an interfering RNA molecule, for example, to enhance its cellular absorption. In certain embodiments, a hydrophobic ligand is conjugated to the molecule to facilitate direct permeation of the cellular membrane. These approaches have been used to facilitate cell permeation of antisense oligonucleotides. In certain instances, conjugation of a cationic ligand to oligonucleotides often results in improved resistance to nucleases. Representative examples of cationic ligands include propylammonium and dimethylpropylammonium. Anti-sense oligonucleotides can retain their high binding affinity to mRNA when the cationic ligand is dispersed throughout the oligonucleotide.

The molecules and polynucleotides described herein may be prepared using well-known techniques of solid-phase synthesis. Any other means for such synthesis known in the art may additionally or alternatively be employed.

“Targeted Induced Local Lesions In Genomes” (TILLING) is another mutagenesis technology that can be used to generate and/or identify polynucleotides encoding polypeptides with modified expression and/or activity. TILLING also allows selection of plants carrying such mutants. TILLING combines high-density mutagenesis with high-throughput screening methods. Methods for TILLING are well known in the art (see McCallum et al., (2000) Nat Biotechnol 18: 455-457 and Stemple (2004) Nat Rev Genet 5(2): 145-50).

Various embodiments are directed to expression vectors comprising one or more of the polynucleotides or interfering RNA constructs that comprise one or more polynucleotides described herein.

Various embodiments are directed to expression vectors comprising one or more of the polynucleotides or one or more interfering RNA constructs described herein.

Various embodiments are directed to expression vectors comprising one or more polynucleotides or one or more interfering RNA constructs encoding one or more interfering RNA polynucleotides described herein that are capable of self-annealing to form a hairpin structure, in which the construct comprises (a) one or more of the polynucleotides described herein; (b) a second sequence encoding a spacer element that forms a loop of the hairpin structure; and (c) a third sequence comprising a reverse complementary sequence of the first sequence, positioned in the same orientation as the first sequence, wherein the second sequence is positioned between the first sequence and the third sequence, and the second sequence is operably-linked to the first sequence and to the third sequence.

The disclosed sequences can be utilised for constructing various polynucleotides that do not form hairpin structures. For example, a double-stranded RNA can be formed by (1) transcribing a first strand of the DNA by operably-linking to a first promoter, and (2) transcribing the reverse complementary sequence of the first strand of the DNA fragment by operably-linking to a second promoter. Each strand of the polynucleotide can be transcribed from the same expression vector, or from different expression vectors. The RNA duplex having RNA interference activity can be enzymatically converted to small interfering RNAs to modulate RNA levels.

Thus, various embodiments are directed to expression vectors comprising one or more polynucleotides or interfering RNA constructs described herein encoding interfering RNA polynucleotides capable of self-annealing, in which the construct comprises (a) one or more of the polynucleotides described herein; and (b) a second sequence comprising a complementary (for example, reverse complementary) sequence of the first sequence, positioned in the same orientation as the first sequence.

Various compositions and methods are provided for modulating the endogenous expression levels of one or more of the polypeptides described herein (or any combination thereof as described herein) by promoting co-suppression of gene expression. The phenomenon of co-suppression occurs as a result of introducing multiple copies of a transgene into a plant cell host. Integration of multiple copies of a transgene can result in modulated expression of the transgene and the targeted endogenous gene. The degree of co-suppression is dependent on the degree of sequence identity between the transgene and the targeted endogenous gene. The silencing of both the endogenous gene and the transgene can occur by extensive methylation of the silenced loci (that is, the endogenous promoter and endogenous gene of interest) that can preclude transcription. Alternatively, in some cases, co-suppression of the endogenous gene and the transgene can occur by post transcriptional gene silencing, in which transcripts can be produced but enhanced rates of degradation preclude accumulation of transcripts. The mechanism for co-suppression by post-transcriptional gene silencing is thought to resemble RNA interference, in that RNA seems to be both an important initiator and a target in these processes, and may be mediated at least in part by the same molecular machinery, possibly through RNA-guided degradation of mRNAs.

Co-suppression of nucleic acids can be achieved by integrating multiple copies of the nucleic acid or fragments thereof, as transgenes, into the genome of a plant of interest. The host plant can be transformed with an expression vector comprising a promoter operably-linked to the nucleic acid or fragments thereof. Various embodiments are directed to expression vectors for promoting co-suppression of endogenous genes comprising a promoter operably-linked to a polynucleotide.

Various embodiments are directed to methods for modulating the expression level of one or more of the polynucleotide(s) described herein (or any combination thereof as described herein) by integrating multiple copies of the polynucleotide(s) into a (tobacco) plant genome, comprising: transforming a plant cell host with an expression vector that comprises a promoter operably-linked to a polynucleotide.

Various compositions and methods are provided for modulating the endogenous gene expression level by modulating the translation of mRNA. A host (tobacco) plant cell can be transformed with an expression vector comprising: a promoter operably-linked to a polynucleotide, positioned in anti-sense orientation with respect to the promoter to enable the expression of RNA polynucleotides having a sequence complementary to a portion of mRNA.

Various expression vectors for modulating the translation of mRNA may comprise: a promoter operably-linked to a polynucleotide in which the sequence is positioned in anti-sense orientation with respect to the promoter. The lengths of anti-sense RNA polynucleotides can vary, and may be from about 15-20 nucleotides, about 20-30 nucleotides, about 30-50 nucleotides, about 50-75 nucleotides, about 75-100 nucleotides, about 100-150 nucleotides, about 150-200 nucleotides, and about 200-300 nucleotides.

Methods for obtaining mutant polynucleotides and polypeptides are also provided. Any plant of interest, including a plant cell or plant material can be genetically modified by various methods known to induce mutagenesis, including site-directed mutagenesis, oligonucleotide-directed mutagenesis, chemically-induced mutagenesis, irradiation-induced mutagenesis, mutagenesis utilizing modified bases, mutagenesis utilizing gapped duplex DNA, double-strand break mutagenesis, mutagenesis utilizing repair-deficient host strains, mutagenesis by total gene synthesis, DNA shuffling and other equivalent methods.

Alternatively, genes can be targeted for inactivation by introducing transposons (for example, IS elements) into the genomes of plants of interest. These mobile genetic elements can be introduced by sexual cross-fertilization and insertion mutants can be screened for loss in protein activity. The disrupted gene in a parent plant can be introduced into other plants by crossing the parent plant with plant not subjected to transposon-induced mutagenesis by, for example, sexual cross-fertilization. Any standard breeding techniques known to persons skilled in the art can be utilized. In one embodiment, one or more genes can be inactivated by the insertion of one or more transposons. Mutations can result in homozygous disruption of one or more genes, in heterozygous disruption of one or more genes, or a combination of both homozygous and heterozygous disruptions if more than one gene is disrupted. Suitable transposable elements include retrotransposons, retroposons, and SINE-like elements. Such methods are known to persons skilled in the art.

Alternatively, genes can be targeted for inactivation by introducing ribozymes derived from a number of small circular RNAs that are capable of self-cleavage and replication in plants. These RNAs can replicate either alone (viroid RNAs) or with a helper virus (satellite RNAs). Examples of suitable RNAs include those derived from avocado sunblotch viroid and satellite RNAs derived from tobacco ringspot virus, lucerne transient streak virus, velvet tobacco mottle virus, solanum nodiflorum mottle virus, and subterranean clover mottle virus. Various target RNA-specific ribozymes are known to persons skilled in the art.

In some embodiments, the expression of a polypeptide is modulated by non-transgenic means, such as creating a mutation in a gene. Methods that introduce a mutation randomly in a gene sequence can include chemical mutagenesis, EMS mutagenesis and radiation mutagenesis. Methods that introduce one or more targeted mutations into a cell include but are not limited to genome editing technology, particularly zinc finger nuclease-mediated mutagenesis, tilling (targeting induced local lesions in genomes), homologous recombination, oligonucleotide-directed mutagenesis, and meganuclease-mediated mutagenesis.

Some non-limiting examples of mutations are deletions, insertions and missense mutations of at least one nucleotide, single nucleotide polymorphisms and a simple sequence repeat. After mutation, screening can be performed to identify mutations that create premature stop codons or otherwise non-functional genes. After mutation, screening can be performed to identify mutations that create functional genes that are capable of being expressed at elevated levels. Screening of mutants can be carried out by sequencing, or by the use of one or more probes or primers specific to the gene or protein. Specific mutations in polynucleotides can also be created that can result in modulated gene expression, modulated stability of mRNA, or modulated stability of protein. Such plants are referred to herein as “non-naturally occurring” or “mutant” plants. Typically, the mutant or non-naturally occurring plants will include at least a portion of foreign or synthetic or man-made nucleic acid (for example, DNA or RNA) that was not present in the plant before it was manipulated. The foreign nucleic acid may be a single nucleotide, two or more nucleotides, two or more contiguous nucleotides or two or more non-contiguous nucleotides—such as at least 10, 20, 30, 40, 50, 100, 200, 300, 400, 500, 600, 700, 800, 900, 1000, 1100, 1200, 1300, 1400 or 1500 or more contiguous or non-contiguous nucleotides.

The mutant or non-naturally occurring plants can have any combination of one or more mutations which results in modulated protein levels. For example, the mutant or non-naturally occurring plants may have a single mutation in a single gene; multiple mutations in a single gene; a single mutation in two or more or three or more genes; or multiple mutations in two or more or three or more genes. By way of further example, the mutant or non-naturally occurring plants may have one or more mutations in a specific portion of the gene(s)—such as in a region of the gene that encodes an active site of the protein or a portion thereof. By way of further example, the mutant or non-naturally occurring plants may have one or more mutations in a region outside of one or more gene(s)—such as in a region upstream or downstream of the gene it regulates provided that they modulate the activity or expression of the gene(s). Upstream elements can include promoters, enhancers or transription factors. Some elements—such as enhancers—can be positioned upstream or downstream of the gene it regulates. The element(s) need not be located near to the gene that it regulates since some elements have been found located several hundred thousand base pairs upstream or downstream of the gene that it regulates. The mutant or non-naturally occurring plants may have one or more mutations located within the first 100 nucleotides of the gene(s), within the first 200 nucleotides of the gene(s), within the first 300 nucleotides of the gene(s), within the first 400 nucleotides of the gene(s), within the first 500 nucleotides of the gene(s), within the first 600 nucleotides of the gene(s), within the first 700 nucleotides of the gene(s), within the first 800 nucleotides of the gene(s), within the first 900 nucleotides of the gene(s), within the first 1000 nucleotides of the gene(s), within the first 1100 nucleotides of the gene(s), within the first 1200 nucleotides of the gene(s), within the first 1300 nucleotides of the gene(s), within the first 1400 nucleotides of the gene(s) or within the first 1500 nucleotides of the gene(s). The mutant or non-naturally occurring plants may have one or more mutations located within the first, second, third, fourth, fifth, sixth, seventh, eighth, ninth, tenth, eleventh, twelfth, thirteenth, fourteenth or fifteenth set of 100 nucleotides of the gene(s) or combinations thereof. Mutant or non-naturally occurring plants (for example, mutant, non-naturally occurring or transgenic plants and the like, as described herein) comprising the mutant polypeptide variants are disclosed.

In one embodiment, seeds from plants are mutagenised and then grown into first generation mutant plants. The first generation plants are then allowed to self-pollinate and seeds from the first generation plant are grown into second generation plants, which are then screened for mutations in their loci. Though the mutagenized plant material can be screened for mutations, an advantage of screening the second generation plants is that all somatic mutations correspond to germline mutations. One of skill in the art would understand that a variety of plant materials, including but not limited to, seeds, pollen, plant tissue or plant cells, may be mutagenised in order to create the mutant plants. However, the type of plant material mutagenised may affect when the plant nucleic acid is screened for mutations. For example, when pollen is subjected to mutagenesis prior to pollination of a non-mutagenized plant the seeds resulting from that pollination are grown into first generation plants. Every cell of the first generation plants will contain mutations created in the pollen; thus these first generation plants may then be screened for mutations instead of waiting until the second generation.

Mutagens that create primarily point mutations and short deletions, insertions, transversions, and or transitions, including chemical mutagens or radiation, may be used to create the mutations. Mutagens include, but are not limited to, ethyl methanesulfonate, methylmethane sulfonate, N-ethyl-N-nitrosurea, triethylmelamine, N-methyl-N-nitrosourea, procarbazine, chlorambucil, cyclophosphamide, diethyl sulfate, acrylamide monomer, melphalan, nitrogen mustard, vincristine, dimethylnitrosamine, N-methyl-N′-nitro-Nitrosoguanidine, nitrosoguanidine, 2-aminopurine, 7,12 dimethyl-benz(a)anthracene, ethylene oxide, hexamethylphosphoramide, bisulfan, diepoxyalkanes (diepoxyoctane, diepoxybutane, and the like), 2-methoxy-6-chloro-9[3-(ethyl-2-chloro-ethyl)aminopropylamino]acridine dihydrochloride and formaldehyde.

Spontaneous mutations in the locus that may not have been directly caused by the mutagen are also contemplated provided that they result in the desired phenotype. Suitable mutagenic agents can also include, for example, ionising radiation—such as X-rays, gamma rays, fast neutron irradiation and UV radiation. Any method of plant nucleic acid preparation known to those of skill in the art may be used to prepare the plant nucleic acid for mutation screening.

Prepared nucleic acid from individual plants, plant cells, or plant material can optionally be pooled in order to expedite screening for mutations in the population of plants originating from the mutagenized plant tissue, cells or material. One or more subsequent generations of plants, plant cells or plant material can be screened. The size of the optionally pooled group is dependent upon the sensitivity of the screening method used.

After the nucleic acid samples are optionally pooled, they can be subjected to polynucleotide-specific amplification techniques, such as Polymerase Chain Reaction. Any one or more primers or probes specific to the gene or the sequences immediately adjacent to the gene may be utilized to amplify the sequences within the optionally pooled nucleic acid sample. Suitably, the one or more primers or probes are designed to amplify the regions of the locus where useful mutations are most likely to arise. Most preferably, the primer is designed to detect mutations within regions of the polynucleotide. Additionally, it is preferable for the primer(s) and probe(s) to avoid known polymorphic sites in order to ease screening for point mutations. To facilitate detection of amplification products, the one or more primers or probes may be labelled using any conventional labelling method. Primer(s) or probe(s) can be designed based upon the sequences described herein using methods that are well understood in the art.

To facilitate detection of amplification products, the primer(s) or probe(s) may be labelled using any conventional labelling method. These can be designed based upon the sequences described herein using methods that are well understood in the art.

Polymorphisms may be identified by means known in the art and some have been described in the literature.

In a further aspect there is provided a method of preparing a mutant plant. The method involves providing at least one cell of a plant comprising a gene encoding a functional polynucleotide described herein (or any combination thereof as described herein). Next, the at least one cell of the plant is treated under conditions effective to modulate the activity of the polynucleotide(s) described herein. The at least one mutant plant cell is then propagated into a mutant plant, where the mutant plant has a modulated level of polypeptide(s) described (or any combination thereof as described herein) as compared to that of a control plant. In one embodiment of this method of making a mutant plant, the treating step involves subjecting the at least one cell to a chemical mutagenising agent as described above and under conditions effective to yield at least one mutant plant cell. In another embodiment of this method, the treating step involves subjecting the at least one cell to a radiation source under conditions effective to yield at least one mutant plant cell. The term “mutant plant” includes mutants plants in which the genotype is modified as compared to a control plant, suitably by means other than genetic engineering or genetic modification.

In certain embodiments, the mutant plant, mutant plant cell or mutant plant material may comprise one or more mutations that have occured naturally in another plant, plant cell or plant material and confer a desired trait. This mutation can be incorporated (for example, introgressed) into another plant, plant cell or plant material (for example, a plant, plant cell or plant material with a different genetic background to the plant from which the mutation was derived) to confer the trait thereto. Thus by way of example, a mutation that occurred naturally in a first plant may be introduced into a second plant—such as a second plant with a different genetic background to the first plant. The skilled person is therefore able to search for and identify a plant carrying naturally in its genome one or more mutant alleles of the genes described herein which confer a desired trait. The mutant allele(s) that occurs naturally can be transferred to the second plant by various methods including breeding, backcrossing and introgression to produce a lines, varieties or hybrids that have one or more mutations in the genes described herein. Plants showing a desired trait may be screened out of a pool of mutant plants. Suitably, the selection is carried out utilising the knowledge of the nucleotide sequences as described herein. Consequently, it is possible to screen for a genetic trait as compared to a control. Such a screening approach may involve the application of conventional nucleic acid amplification and/or hybridization techniques as discussed herein. Thus, a further aspect of the present invention relates to a method for identifying a mutant plant comprising the steps of: (a) providing a sample comprising nucleic acid from a plant; and (b) determining the nucleic acid sequence of the polynucleotide, wherein a difference in the sequence of the polynucleotide as compared to the polynucleotide sequence of a control plant is indicative that said plant is a mutant plant. In another aspect there is provided a method for identifying a mutant plant which accumulates reduced levels of at least NNK and/or nitrate as compared to a control plant comprising the steps of: (a) providing a sample from a plant to be screened; (b) determining if said sample comprises one or more mutations in one or more of the polynucleotides described herein; and (c) determining the (i) nitrate content; and/or (ii) at least the NNK content of said plant. Suitably at least the NNK and/or nitrate content is determined in green leaves. In another aspect there is provided a method for preparing a mutant plant which has reduced levels of at least NNK and/or nitrate as compared to a control plant comprising the steps of: (a) providing a sample from a first plant; (b) determining if said sample comprises one or more mutations in one or more the polynucleotides described herein that result in reduced levels of at least NNK and/or nitrate; and (c) transferring the one or more mutations into a second plant. Suitably the NNK and/or nitrate content is determined in green leaves. The mutation(s) can be transferred into the second plant using various methods that are known in the art—such as by genetic engineering, genetic manipulation, introgression, plant breeding, backcrossing and the like. In one embodiment, the first plant is a naturally occurring plant. In one embodiment, the second plant has a different genetic background to the first plant. In another aspect there is provided a method for preparing a mutant plant which has reduced levels of at least NNK and/or nitrate as compared to a control plant comprising the steps of: (a) providing a sample from a first plant; (b) determining if said sample comprises one or more mutations in one or more of the polynucleotides described herein that results in reduced levels of at least NNK and/or nitrate; and (c) introgressing the one or more mutations from the first plant into a second plant. Suitably the NNK and/or nitrate content is determined in green leaves. In one embodiment, the step of introgressing comprises plant breeding, optionally including backcrossing and the like. In one embodiment, the first plant is a naturally occurring plant. In one embodiment, the second plant has a different genetic background to the first plant. In one embodiment, the first plant is not a cultivar or an elite cultivar. In one embodiment, the second plant is a cultivar or an elite cultivar. A further aspect relates to a mutant plant (including a cultivar or elite cultivar mutant plant) obtained or obtainable by the methods described herein. In certain embodiments, the “mutant plants” may have one or more mutations localised only to a specific region of the plant—such as within the sequence of the one or more polynucleotide(s) described herein. According to this embodiment, the remaining genomic sequence of the mutant plant will be the same or substantially the same as the plant prior to the mutagenesis.

In certain embodiments, the mutant plants may have one or more mutations localised in more than one region of the plant—such as within the sequence of one or more of the polynucleotides described herein and in one or more further regions of the genome. According to this embodiment, the remaining genomic sequence of the mutant plant will not be the same or will not be substantially the same as the plant prior to the mutagenesis. In certain embodiments, the mutant plants may not have one or more mutations in one or more, two or more, three or more, four or more or five or more exons of the polynucleotide(s) described herein; or may not have one or more mutations in one or more, two or more, three or more, four or more or five or more introns of the polynucleotide(s) described herein; or may not have one or more mutations in a promoter of the polynucleotide(s) described herein; or may not have one or more mutations in the 3′ untranslated region of the polynucleotide(s) described herein; or may not have one or more mutations in the 5′ untranslated region of the polynucleotide(s) described herein; or may not have one or more mutations in the coding region of the polynucleotide(s) described herein; or may not have one or more mutations in the non-coding region of the polynucleotide(s) described herein; or any combination of two or more, three or more, four or more, five or more; or six or more thereof parts thereof.

In a further aspect there is provided a method of identifying a plant, a plant cell or plant material comprising a mutation in a gene encoding a polynucleotide described herein comprising: (a) subjecting a plant, a plant cell or plant material to mutagenesis; (b) obtaining a nucleic acid sample from said plant, plant cell or plant material or descendants thereof; and (c) determining the nucleic acid sequence of the gene encoding a polynucleotide described herein or a variant or a fragment thereof, wherein a difference in said sequence is indicative of one or more mutations therein.

Zinc finger proteins can be used to modulate the expression or the activity of one or more of the polynucleotides described herein. In various embodiments, a genomic DNA sequence comprising a part of or all of the coding sequence of the polynucleotide is modified by zinc finger nuclease-mediated mutagenesis. The genomic DNA sequence is searched for a unique site for zinc finger protein binding. Alternatively, the genomic DNA sequence is searched for two unique sites for zinc finger protein binding wherein both sites are on opposite strands and close together, for example, 1, 2, 3, 4, 5, 6 or more basepairs apart. Accordingly, zinc finger proteins that bind to polynucleotides are provided.

A zinc finger protein may be engineered to recognize a selected target site in a gene. A zinc finger protein can comprise any combination of motifs derived from natural zinc finger DNA-binding domains and non-natural zinc finger DNA-binding domains by truncation or expansion or a process of site-directed mutagenesis coupled to a selection method such as, but not limited to, phage display selection, bacterial two-hybrid selection or bacterial one-hybrid selection. The term “non-natural zinc finger DNA-binding domain” refers to a zinc finger DNA-binding domain that binds a three-base pair sequence within the target nucleic acid and that does not occur in the cell or organism comprising the nucleic acid which is to be modified. Methods for the design of zinc finger protein which binds specific nucleotide sequences which are unique to a target gene are known in the art.

A zinc finger nuclease may be constructed by making a fusion of a first polynucleotide coding for a zinc finger protein that binds to a polynucleotide, and a second polynucleotide coding for a non-specific endonuclease such as, but not limited to, those of a Type IIS endonuclease. A fusion protein between a zinc finger protein and the nuclease may comprise a spacer consisting of two base pairs or alternatively, the spacer can consist of three, four, five, six, seven or more base pairs. In various embodiments, a zinc finger nuclease introduces a double stranded break in a regulatory region, a coding region, or a non-coding region of a genomic DNA sequence of a polynucleotide and leads to a reduction of the level of expression of a polynucleotide, or a reduction in the activity of the protein encoded thereby. Cleavage by zinc finger nucleases frequently results in the deletion of DNA at the cleavage site following DNA repair by non-homologous end joining.

In other embodiments, a zinc finger protein may be selected to bind to a regulatory sequence of a polynucleotide. More specifically, the regulatory sequence may comprise a transcription initiation site, a start codon, a region of an exon, a boundary of an exon-intron, a terminator, or a stop codon. Accordingly, the invention provides a mutant, non-naturally occurring or transgenic plant or plant cells, produced by zinc finger nuclease-mediated mutagenesis in the vicinity of or within one or more polynucleotides described herein, and methods for making such a plant or plant cell by zinc finger nuclease-mediated mutagenesis. Methods for delivering zinc finger protein and zinc finger nuclease to a tobacco plant are similar to those described below for delivery of meganuclease.

In another aspect, methods for producing mutant, non-naturally occurring or transgenic or otherwise genetically-modified plants using meganucleases, such as I-Crel, are described. Naturally occurring meganucleases as well as recombinant meganucleases can be used to specifically cause a double-stranded break at a single site or at relatively few sites in the genomic DNA of a plant to allow for the disruption of one or more polynucleotides described herein. The meganuclease may be an engineered meganuclease with altered DNA-recognition properties. Meganuclease proteins can be delivered into plant cells by a variety of different mechanisms known in the art.

The inventions encompass the use of meganucleases to inactivate a polynucleotide(s) described herein (or any combination thereof as described herein) in a plant cell or plant. Particularly, the invention provides a method for inactivating a polynucleotide in a plant using a meganuclease comprising: a) providing a plant cell comprising a polynucleotide as described herein; (b) introducing a meganuclease or a construct encoding a meganuclease into said plant cell; and (c) allowing the meganuclease to substantially inactivate the polynucleotide(s)

Meganucleases can be used to cleave meganuclease recognition sites within the coding regions of a polynucleotide. Such cleavage frequently results in the deletion of DNA at the meganuclease recognition site following mutagenic DNA repair by non-homologous end joining. Such mutations in the gene coding sequence are typically sufficient to inactivate the gene. This method to modify a plant cell involves, first, the delivery of a meganuclease expression cassette to a plant cell using a suitable transformation method. For highest efficiency, it is desirable to link the meganuclease expression cassette to a selectable marker and select for successfully transformed cells in the presence of a selection agent. This approach will result in the integration of the meganuclease expression cassette into the genome, however, which may not be desirable if the plant is likely to require regulatory approval. In such cases, the meganuclease expression cassette (and linked selectable marker gene) may be segregated away in subsequent plant generations using conventional breeding techniques. Alternatively, plant cells may be initially be transformed with a meganuclease expression cassette lacking a selectable marker and may be grown on media lacking a selection agent. Under such conditions, a fraction of the treated cells will acquire the meganuclease expression cassette and will express the engineered meganuclease transiently without integrating the meganuclease expression cassette into the genome. Because it does not account for transformation efficiency, this latter transformation procedure requires that a greater number of treated cells be screened to obtain the desired genome modification. The above approach can also be applied to modify a plant cell when using a zinc finger protein or zinc finger nuclease.

Following delivery of the meganuclease expression cassette, plant cells are grown, initially, under conditions that are typical for the particular transformation procedure that was used. This may mean growing transformed cells on media at temperatures below 26° C., frequently in the dark. Such standard conditions can be used for a period of time, preferably 1-4 days, to allow the plant cell to recover from the transformation process. At any point following this initial recovery period, growth temperature may be raised to stimulate the activity of the engineered meganuclease to cleave and mutate the meganuclease recognition site.

For certain applications, it may be desirable to precisely remove the polynucleotide from the genome of the plant. Such applications are possible using a pair of engineered meganucleases, each of which cleaves a meganuclease recognition site on either side of the intended deletion. TAL Effector Nucleases (TALENs) that are able to recognize and bind to a gene and introduce a double-strand break into the genome can also be used. Thus, in another aspect, methods for producing mutant, non-naturally occurring or transgenic or otherwise genetically-modified plants as described herein using TAL Effector Nucleases are contemplated.

Plants suitable for use in genetic modification include, but are not limited to, monocotyledonous and dicotyledonous plants and plant cell systems, including species from one of the following families: Acanthaceae, Alliaceae, Alstroemeriaceae, Amaryllidaceae, Apocynaceae, Arecaceae, Asteraceae, Berberidaceae, Bixaceae, Brassicaceae, Bromeliaceae, Cannabaceae, Caryophyllaceae, Cephalotaxaceae, Chenopodiaceae, Colchicaceae, Cucurbitaceae, Dioscoreaceae, Ephedraceae, Erythroxylaceae, Euphorbiaceae, Fabaceae, Lamiaceae, Linaceae, Lycopodiaceae, Malvaceae, Melanthiaceae, Musaceae, Myrtaceae, Nyssaceae, Papaveraceae, Pinaceae, Plantaginaceae, Poaceae, Rosaceae, Rubiaceae, Salicaceae, Sapindaceae, Solanaceae, Taxaceae, Theaceae, or Vitaceae.

Suitable species may include members of the genera Abelmoschus, Abies, Acer, Agrostis, Allium, Alstroemeria, Ananas, Andrographis, Andropogon, Artemisia, Arundo, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Dianthus, Digitalis, Dioscorea, Elaeis, Ephedra, Erianthus, Erythroxylum, Eucalyptus, Festuca, Fragaria, Galanthus, Glycine, Gossypium, Helianthus, Hevea, Hordeum, Hyoscyamus, Jatropha, Lactuca, Linum, Lolium, Lupinus, Lycopersicon, Lycopodium, Manihot, Medicago, Mentha, Miscanthus, Musa, Nicotiana, Oryza, Panicum, Papaver, Parthenium, Pennisetum, Petunia, Phalaris, Phleum, Pinus, Poa, Poinsettia, Populus, Rauwolfia, Ricinus, Rosa, Saccharum, Salix, Sanguinaria, Scopolia, Secale, Solanum, Sorghum, Spartina, Spinacea, Tanacetum, Taxus, Theobroma, Triticosecale, Triticum, Uniola, Veratrum, Vinca, Vitis, and Zea.

Suitable species may include Panicum spp., Sorghum spp., Miscanthus spp., Saccharum spp., Erianthus spp., Populus spp., Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Medicago sativa (alfalfa), Arundo donax (giant reed), Secale cereale (rye), Salix spp. (willow), Eucalyptus spp. (eucalyptus), Triticosecale (tritic wheat times rye), bamboo, Helianthus annuus (sunflower), Carthamus tinctorius (safflower), Jatropha curcas (jatropha), Ricinus communis (castor), Elaeis guineensis (palm), Linum usitatissimum (flax), Brassica juncea, Beta vulgaris (sugarbeet), Manihot esculenta (cassaya), Lycopersicon esculentum (tomato), Lactuca sativa (lettuce), Musyclise alca (banana), Solanum tuberosum (potato), Brassica oleracea (broccoli, cauliflower, Brussels sprouts), Camellia sinensis (tea), Fragaria ananassa (strawberry), Theobroma cacao (cocoa), Coffe49ycliseca (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Rosa spp. (rose), Dianthus caryophyllus (carnation), Petunia spp. (petunia), Poinsettia pulcherrima (poinsettia), Lupinus albus (lupin), Uniola paniculata (oats), bentgrass (Agrostis spp.), Populus tremuloides (aspen), Pinus spp. (pine), Abies spp. (fir), Acer spp. (maple), Hordeum vulgare (barley), Poa pratensis (bluegrass), Lolium spp. (ryegrass) and Phleum pratense (timothy), Panicum virgatum (switchgrass), Sorghu49yclise49or (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), or Pennisetum glaucum (pearl millet).

Various embodiments are directed to mutant tobacco plants, non-naturally occurring tobacco plants or transgenic tobacco plants modified to modulate gene expression levels thereby producing plants—such as tobacco plant—in which the expression level of a polypeptide is modulated within plant tissues of interest as compared to a control plant. The disclosed compositions and methods can be applied to any species of the genus Nicotiana, including N. rustica and N. tabacum (for example, LA B21, LN KY171, TI 1406, Basma, Galpao, Perique, Beinhart 1000-1, and Petico). Other species include N. acaulis, N49yclise4949ta, N49yclise4949ta var. multiflora, N49yclise49na, N. alata, N. amplexicaulis, N. arentsii, N49yclise4949ta, N. benavidesii, N. benthamiana, N. bigelovii, N. bonariensis, N. cavicola, N. clevelandii, N. cordifolia, N. corymbosa, N. debneyi, N. excelsior, N. forgetiana, N. fragrans, N. glauca, N. glutinosa, N. goodspeedii, N. gossei, N. hybrid, N. ingulba, N. kawakamii, N. knightiana, N. langsdorffii, N. linearis, N. longiflora, N50yclise50ma, N. megalosiphon, N. miersii, N. noctiflora, N. nudicaulis, N. obtusifolia, N. occidentalis, N. occidentalis subsp. hesperis, N. otophora, N. paniculata, N. pauciflora, N. petunioides, N. plumbaginifolia, N. quadrivalvis, N. raimondii, N. repanda, N. rosulata, N. rosulata subsp. ingulba, N. rotundifolia, N. setchellii, N. simulans, N. solanifolia, N. spegazzinii, N. stocktonii, N. suaveolens, N. sylvestris, N. thyrsiflora, N. tomentosa, N. tomentosiformis, N. trigonophylla, N. umbratica, N50yclise50ta, N. velutina, N. wigandioides, and N. x sanderae.

The use of tobacco cultivars and elite tobacco cultivars is also contemplated herein. The transgenic, non-naturally occurring or mutant plant may therefore be a tobacco variety or elite tobacco cultivar that comprises one or more transgenes, or one or more genetic mutations or a combination thereof. The genetic mutation(s) (for example, one or more polymorphisms) can be mutations that do not exist naturally in the individual tobacco variety or tobacco cultivar (for example, elite tobacco cultivar) or can be genetic mutation(s) that do occur naturally provided that the mutation does not occur naturally in the individual tobacco variety or tobacco cultivar (for example, elite tobacco cultivar).

Particularly useful Nicotiana tabacum varieties include Burley type, dark type, flue-cured type, and Oriental type tobaccos. Non-limiting examples of varieties or cultivars are: BD 64, CC 101, CC 200, CC 27, CC 301, CC 400, CC 500, CC 600, CC 700, CC 800, CC 900, Coker 176, Coker 319, Coker 371 Gold, Coker 48, CD 263, DF911, DT 538 LC Galpao tobacco, GL 26H, GL 350, GL 600, GL 737, GL 939, GL 973, HB 04P, HB 04P LC, HB3307PLC, Hybrid 403LC, Hybrid 404LC, Hybrid 501 LC, K 149, K 326, K 346, K 358, K394, K 399, K 730, KDH 959, KT 200, KT204LC, KY10, KY14, KY 160, KY 17, KY 171, KY 907, KY907LC, KTY14xL8 LC, Little Crittenden, McNair 373, McNair 944, msKY 14xL8, Narrow Leaf Madole, Narrow Leaf Madole LC, NBH 98, N-126, N-777LC, N-7371LC, NC 100, NC 102, NC 2000, NC 291, NC 297, NC 299, NC 3, NC 4, NC 5, NC 6, NC7, NC 606, NC 71, NC 72, NC 810, NC BH 129, NC 2002, Neal Smith Madole, OXFORD 207, PD 7302 LC, PD 7309 LC, PD 7312 LC’ ‘Periq'e’ tobacco, PVH03, PVH09, PVH19, PVH50, PVH51, R 610, R 630, R 7-11, R 7-12, RG 17, RG 81, RG H51, RGH 4, RGH 51, RS 1410, Speight 168, Speight 172, Speight 179, Speight 210, Speight 220, Speight 225, Speight 227, Speight 234, Speight G-28, Speight G-70, Speight H-6, Speight H20, Speight NF3, TI 1406, TI 1269, TN 86, TN86LC, TN 90, TN 97, TN97LC, TN D94, TN D950, TR (Tom Rosson) Madole, VA 309, VA359, AA 37-1, B 13P, Xanthi (Mitchell-Mor), Bel-W3, 79-615, Samsun Holmes NN, KTRDC number 2 Hybrid 49, Burley 21, KY 8959, KY 9, MD 609, PG 01, PG 04, PO1, PO2, PO3, RG 11, RG 8, VA 509, AS44, Banket A1, Basma Drama B84/31, Basma I Zichna ZP4/B, Basma Xanthi BX 2A, Batek, Besuki Jember, C104, Coker 347, Criollo Misionero, Delcrest, Djebel 81, DVH 405, Galpão Comum, HB04P, Hicks Broadleaf, Kabakulak Elassona, Kutsage E1, LA BU 21, NC 2326, NC 297, PVH 2110, Red Russian, Samsun, Saplak, Simmaba, Talgar28, Wislica, Yayaldag, Prilep HC-72, Prilep P23, Prilep PB 156/1, Prilep P12-2/1, Yaka JK-48, Yaka JB 125/3, TI-1068, KDH-960, TI-1070, TW136, Basma, TKF 4028, L8, TKF 2002, GR141, Basma xanthi, GR149, GR153, Petit Havana. Low converter subvarieties of the above, even if not specifically identified herein, are also contemplated.

Embodiments are also directed to compositions and methods for producing mutant plants, non-naturally occurring plants, hybrid plants, or transgenic plants that have been modified to modulate the expression or activity of a polynucleotide(s) described herein (or any combination thereof as described herein). Advantageously, the mutant plants, non-naturally occurring plants, hybrid plants, or transgenic plants that are obtained may be similar or substantially the same in overall appearance to control plants. Various phenotypic characteristics such as degree of maturity, number of leaves per plant, stalk height, leaf insertion angle, leaf size (width and length), internode distance, and lamina-midrib ratio can be assessed by field observations.

One aspect relates to a seed of a mutant plant, a non-naturally occurring plant, a hybrid plant or a transgenic plant described herein. Preferably, the seed is a tobacco seed. A further aspect relates to pollen or an ovule of a mutant plant, a non-naturally occurring plant, a hybrid plant or a transgenic plant that is described herein. In addition, there is provided a mutant plant, a non-naturally occurring plant, a hybrid plant or a transgenic plant as described herein which further comprises a nucleic acid conferring male sterility.

Also provided is a tissue culture of regenerable cells of the mutant plant, non-naturally occurring plant, hybrid plant, or transgenic plant or a part thereof as described herein, which culture regenerates plants capable of expressing all the morphological and physiological characteristics of the parent. The regenerable cells include but are not limited to cells from leaves, pollen, embryos, cotyledons, hypocotyls, roots, root tips, anthers, flowers and a part thereof, ovules, shoots, stems, stalks, pith and capsules or callus or protoplasts derived therefrom.

One object is to provide mutant, transgenic or non-naturally occurring plants or parts thereof that exhibit modulated (eg. reduced) levels of TSNAs in the plant material, for example, in cured leaves. Suitably, mutant, transgenic or non-naturally occurring plants or parts thereof that exhibit modulated (eg. reduced) levels of at least NNK and/or nitrate as compared to a control plant. In certain embodiments, the level of at least NNN will be substantially the same. In certain embodiments, the level of at least NNN, NAB and NAT will be substantially the same. In certain embodiments, the level of at least NNN will be substantially the same and the level of NAB will be reduced as compared to a control plant. In certain embodiments, the level of at least NNN will be substantially the same and the level of NAT will be reduced as compared to a control plant. In certain embodiments, the level of at least NNN will be substantially the same and the level of NAT and NAB will be reduced as compared to a control plant. The nicotine content in the mutant, transgenic or non-naturally occurring plants or parts thereof can be substantially the same as the control or wild type plant or can be lower than the control or wild type plant. Suitably, the mutant, transgenic or non-naturally occurring plants or parts thereof have substantially the same visual appearance as the control plant.

The four principal TSNAs, those typically found to be present in the highest concentrations, are N-nitrosonicotine (NNN), 4-(methylnitrosamino)-1-(3-pyridyl)-1-butanone (NNK), N-nitrosoanabasine (NAB) and N-nitrosoanatabine (NAT). Minor compounds, those typically found at significantly lower levels than the principal TSNAs, include 4-(methylnitrosamino) 4-(3-pyridyl)butanal (NNA), 4-(methylnitrosamino)-1-(3-pyridyl)-1-butanol (NNAL), 4-(methylnitrosamino)4-(3-pyridyl)-1-butanol (iso-NNAL), and 4-(methylnitrosamino)-4-(3-pyridyl)-1-butyric acid (iso-NNAC). At least NNN and NNK have been reported to be carcinogenic when applied to animals in laboratory studies.

Accordingly, there is described herein mutant, transgenic or non-naturally occurring plants or parts thereof or plant cells that have modulated (eg. reduced) levels of at least NNK and/or nitrate as compared to control cells or control plants. In certain embodiments, the level of NNN will be substantially the same. The mutant, transgenic or non-naturally occurring plants or plant cells have been modified to modulate (eg. reduce) the synthesis or activity of one or more of the polypeptides described herein by modulating the expression of one or more of the corresponding polynucleotide sequences described herein. Suitably, the modulated levels of at least NNK and/or nitrate are observed in at least the green leaves, suitably cured leaves. In certain embodiments, the level of total TSNAs in the plant—such as the green leaves, suitably cured leaves or cured tobacco—may be modulated (eg. reduced). In certain embodiments, the level of nicotine in the plant—such as the green leaves, suitably cured leaves or cured tobacco—may be modulated (eg. reduced).

A further aspect, relates to a mutant, non-naturally occurring or transgenic plant or cell, wherein the expression of or the activity of one or more of the polypeptides described herein is modulated (eg. reduced) and a part of the plant (for example, the green leaves, suitably cured leaves or cured tobacco) have reduced levels of nitrate and/or at least NNK of at least 5% therein as compared to a control plant in which the expression or the activity said polypeptide(s) has not been modulated. In certain embodiments, the level of NNN will be substantially the same. In certain embodiments, the level of total TSNAs in the plant—such as the green leaves, suitably cured leaves or cured tobacco—may also be modulated (eg. reduced), for example, by at least about 5%. In certain embodiments, the level of nicotine in the plant—such as the green leaves, suitably cured leaves or cured tobacco—may also be modulated (eg. reduced), for example, by at least about 5%. In certain embodiments, the level of total TSNAs in the plant—such as in green leaves—may also be modulated (eg. reduced), for example, by at least about 5% and the level of nicotine in the plant—such as the green leaves, suitably cured leaves or cured tobacco—may also be modulated (eg. reduced), for example, by at least about 5%.

A still further aspect, relates to a cured plant material—such as cured leaf or cured tobacco—derived or derivable from a mutant, non-naturally occurring or transgenic plant or cell, wherein expression of one or more of the polynucleotides described herein or the activity of the protein encoded thereby is reduced and wherein the nitrate and/or NNK level is reduced by at least 5% as compared to a control plant. In certain embodiments, the level of NNN will be substantially the same.

A still further aspect, relates to mutant, non-naturally occurring or transgenic cured plant material—such as leaf or cured tobacco—which has nitrate and/or NNK levels that are reduced at least 5% as compared to a control plant. In certain embodiments, the level of NNN will be substantially the same. In certain embodiments, the level of total TSNAs in the cured plant material may also be reduced, for example, by at least about 5%. In certain embodiments, the level of nicotine in the cured plant material may also be reduced, for example, by at least about 5%. In certain embodiments, the level of total TSNAs in the cured plant material may also be reduced, for example, by at least about 5% and the level of nicotine in the cured plant material may also be reduced by at least about 5%.

In a still further aspect, there is provided a mutant, non-naturally occurring or transgenic plant or plant cell, wherein expression of one or more of the polypeptides described herein is reduced as compared to a control or a wild-type plant and wherein (i) the nitrate content is about 7 mg/g or less—such as about 6.9 mg/g or less, about 6.8 mg/g or less, about 6.7 mg/g or less, about 6.6 mg/g or less, about 6.5 mg/g or less, about 6.4 mg/g or less, about 6.3 mg/g or less, about 6.2 mg/g or less, about 6.1 mg/g or less, or about 6 mg/g or less; and (ii) the NNK content is about 110 ng/g or less—such as about 109 ng/g or less, about 108 ng/g or less, about 107 ng/g or less, about 106 ng/g or less, about 105 ng/g or less, about 104 ng/g or less, about 103 ng/g or less, about 102 ng/g or less, about 101 ng/g or less, or about 100 ng/g or less. In certain embodiments the level of nicotine is about 30 mg/g or less—such as about 29.9 mg/g or less, about 29.8 mg/g or less, about 29.7 mg/g or less, about 29.6 mg/g or less, about 29.5 mg/g or less, about 29.4 mg/g or less, about 29.3 mg/g or less, about 29.2 mg/g or less, about 29.1 mg/g or less, or about 29 mg/g or less. In certain embodiments, the total TSNA content is about 250 ng/g or less—such as about 240 ng/g or less, about 230 ng/g or less, about 220 ng/g or less, about 210 ng/g or less, about 200 ng/g or less, about 190 ng/g or less, about 180 ng/g or less, about 170 ng/g or less, about 160 ng/g or less, or about 150 ng/g or less.

In a still further aspect, there is provided a mutant, non-naturally occurring or transgenic leaf, wherein expression of one or more of the polypeptides described herein is reduced as compared to a control or a wild-type leaf and wherein (i) the nitrate content is about 7 mg/g or less—such as about 6.9 mg/g or less, about 6.8 mg/g or less, about 6.7 mg/g or less, about 6.6 mg/g or less, about 6.5 mg/g or less, about 6.4 mg/g or less, about 6.3 mg/g or less, about 6.2 mg/g or less, about 6.1 mg/g or less, or about 6 mg/g or less; and (ii) the NNK content is about 110 ng/g or less-such as about 109 ng/g or less, about 108 ng/g or less, about 107 ng/g or less, about 106 ng/g or less, about 105 ng/g or less, about 104 ng/g or less, about 103 ng/g or less, about 102 ng/g or less, about 101 ng/g or less, or about 100 ng/g or less. In certain embodiments the level of nicotine is about 30 mg/g or less—such as about 29.9 mg/g or less, about 29.8 mg/g or less, about 29.7 mg/g or less, about 29.6 mg/g or less, about 29.5 mg/g or less, about 29.4 mg/g or less, about 29.3 mg/g or less, about 29.2 mg/g or less, about 29.1 mg/g or less, or about 29 mg/g or less. In certain embodiments, the total TSNA content is about 250 ng/g or less—such as about 240 ng/g or less, about 230 ng/g or less, about 220 ng/g or less, about 210 ng/g or less, about 200 ng/g or less, about 190 ng/g or less, about 180 ng/g or less, about 170 ng/g or less, about 160 ng/g or less, or about 150 ng/g or less.

In a still further aspect, there is provided mutant, non-naturally occurring or transgenic cured plant material—such as cured leaf or cured tobacco—wherein expression of one or more of the polypeptides described herein is reduced as compared to control or a wild-type cured plant material and wherein: (i) the nitrate content is about 7 mg/g or less—such as about 6.9 mg/g or less, about 6.8 mg/g or less, about 6.7 mg/g or less, about 6.6 mg/g or less, about 6.5 mg/g or less, about 6.4 mg/g or less, about 6.3 mg/g or less, about 6.2 mg/g or less, about 6.1 mg/g or less, or about 6 mg/g or less; and (ii) the NNK content is about 110 ng/g or less—such as about 109 ng/g or less, about 108 ng/g or less, about 107 ng/g or less, about 106 ng/g or less, about 105 ng/g or less, about 104 ng/g or less, about 103 ng/g or less, about 102 ng/g or less, about 101 ng/g or less, or about 100 ng/g or less. In certain embodiments the level of nicotine is about 30 mg/g or less—such as about 29.9 mg/g or less, about 29.8 mg/g or less, about 29.7 mg/g or less, about 29.6 mg/g or less, about 29.5 mg/g or less, about 29.4 mg/g or less, about 29.3 mg/g or less, about 29.2 mg/g or less, about 29.1 mg/g or less, or about 29 mg/g or less. In certain embodiments, the total TSNA content is about 250 ng/g or less—such as about 240 ng/g or less, about 230 ng/g or less, about 220 ng/g or less, about 210 ng/g or less, about 200 ng/g or less, about 190 ng/g or less, about 180 ng/g or less, about 170 ng/g or less, about 160 ng/g or less, or about 150 ng/g or less.

Suitably the visual appearance of said plant or part thereof (for example, leaf) is substantially the same as the control plant. Suitably, the plant is a tobacco plant.

Embodiments are also directed to compositions and methods for producing mutant, non-naturally occurring or transgenic plants that have been modified to modulate the expression or activity of the one or more of the polynucleotides or polypeptides described herein which can result in plants or plant components (for example, leaves—such as green leaves or cured leaves—or tobacco) with modulated levels of nitrate and/or NNK and/or NNN and/or TSNAs and/or nicotine as compared to a control plant.

Advantageously, the mutant, non-naturally occurring or transgenic plants that are obtained according to the methods described herein are similar or substantially the same in visual appearance to the control plants. In one embodiment, the leaf weight of the mutant, non-naturally occurring or transgenic plant is substantially the same as the control plant. In one embodiment, the leaf number of the mutant, non-naturally occurring or transgenic plant is substantially the same as the control plant. In one embodiment, the leaf weight and the leaf number of the mutant, non-naturally occurring or transgenic plant is substantially the same as the control plant. In one embodiment, the stalk height of the mutant, non-naturally occurring or transgenic plants is substantially the same as the control plants at, for example, one, two or three or more months after field transplant or 10, 20, 30 or 36 or more days after topping. For example, the stalk height of the mutant, non-naturally occurring or transgenic plants is not less than the stalk height of the control plants. In another embodiment, the chlorophyll content of the mutant, non-naturally occurring or transgenic plants is substantially the same as the control plants. In another embodiment, the stalk height of the mutant, non-naturally occurring or transgenic plants is substantially the same as the control plants and the chlorophyll content of the mutant, non-naturally occurring or transgenic plants is substantially the same as the control plants. In other embodiments, the size or form or number or colouration of the leaves of the mutant, non-naturally occurring or transgenic plants is substantially the same as the control plants. Suitably, the plant is a tobacco plant.

In another aspect, there is provided a method for modulating (eg. reducing) the amount of nitrate and/or at least NNK in at least a part of a plant (for example, the leaves—such as cured leaves—or in tobacco), comprising the steps of: (i) modulating (eg. reducing) the expression or activity of an one or more of the polypeptides described herein (or any combination thereof as described herein), suitably, wherein the polypeptide(s) is encoded by the corresponding polynucleotide sequence described herein; (ii) measuring the nitrate and/or at least NNK content in at least a part (for example, the leaves—such as cured leaves—or tobacco) of the mutant, non-naturally occurring or transgenic plant obtained in step (i); and (iii) identifying a mutant, non-naturally occurring or transgenic plant in which the nitrate and/or at least NNK content therein has been modulated (eg. reduced) in comparison to a control plant. Suitably, the visual appearance of said mutant, non-naturally occurring or transgenic plant is substantially the same as the control plant. Suitably, the plant is a tobacco plant.

In another aspect, there is provided a method for modulating (eg. reducing) the amount of nitrate and/or at least NNK in at least a part of cured plant material—such as cured leaf—comprising the steps of: (i) modulating (eg. reducing) the expression or activity of an one or more of the polypeptides (or any combination thereof as described herein), suitably, wherein the polypeptide(s) is encoded by the corresponding polynucleotide sequence described herein; (ii) harvesting plant material—such as one or more of the leaves—and curing for a period of time; (iii) measuring the nitrate and/or at least NNK content in at least a part of the cured plant material obtained in step (ii); and (iv) identifying cured plant material in which the nitrate and/or at least NNK content therein has been modulated (eg. reduced) in comparison to a control plant.

The increase in expression as compared to the control plant may be from about 5% to about 100%, or an increase of at least 10%, at least 20%, at least 25%, at least 30%, at least 40%, at least 50%, at least 60%, at least 70%, at least 75%, at least 80%, at least 90%, at least 95%, at least 98%, or 100% or more—such as 200% or 300% or more, which includes an increase in transcriptional activity or protein expression or both.

The increase in the activity as compared to a control type plant may be from about 5% to about 100%, or an increase of at least 10%, at least 20%, at least 25%, at least 30%, at least 40%, at least 50%, at least 60%, at least 70%, at least 75%, at least 80%, at least 90%, at least 95%, at least 98%, or 100% or more—such as 200% or 300% or more.

The reduction in expression as compared to the control plant may be from about 5% to about 100%, or a reduction of at least 10%, at least 20%, at least 25%, at least 30%, at least 40%, at least 50%, at least 60%, at least 70%, at least 75%, at least 80%, at least 90%, at least 95%, at least 98%, or 100%, which includes a reduction in transcriptional activity or protein expression or both.

The reduction in activity as compared to a control type plant may be from about 5% to about 100%, or a reduction of at least 10%, at least 20%, at least 25%, at least 30%, at least 40%, at least 50%, at least 60%, at least 70%, at least 75%, at least 80%, at least 90%, at least 95%, at least 98%, or 100%.

Polynucleotides and recombinant constructs described herein can be used to modulate the expression of the enzymes described herein in a plant species of interest, suitably tobacco.

A number of polynucleotide based methods can be used to increase gene expression in plants. By way of example, a construct, vector or expression vector that is compatible with the plant to be transformed can be prepared which comprises the gene of interest together with an upstream promoter that is capable of overexpressing the gene in the plant. Exemplary promoters are described herein. Following transformation and when grown under suitable conditions, the promoter can drive expression in order to modulate (for example, reduce) the levels of this enzyme in the plant, or in a specific tissue thereof. In one exemplary embodiment, a vector carrying one or more polynucleotides described herein (or any combination thereof as described herein) is generated to overexpress the gene in a plant. The vector carries a suitable promoter—such as the cauliflower mosaic virus CaMV 35S promoter—upstream of the transgene driving its constitutive expression in all tissues of the plant. The vector also carries an antibiotic resistance gene in order to confer selection of the transformed calli and cell lines.

Various embodiments are therefore directed to methods for modulating (for example, reducing) the expression level of one or more polynucleotides described herein (or any combination thereof as described herein) by integrating multiple copies of the polynucleotide into a plant genome, comprising: transforming a plant cell host with an expression vector that comprises a promoter operably-linked to one or more polynucleotides described herein. The polypeptide encoded by a recombinant polynucleotide can be a native polypeptide, or can be heterologous to the cell.

A tobacco plant carrying a mutant allele of one or more polynucleotides described herein (or any combination thereof as described herein) can be used in a plant breeding program to create useful lines, varieties and hybrids. In particular, the mutant allele is introgressed into the commercially important varieties described above. Thus, methods for breeding plants are provided, that comprise crossing a mutant plant, a non-naturally occurring plant or a transgenic plant as described herein with a plant comprising a different genetic identity. The method may further comprise crossing the progeny plant with another plant, and optionally repeating the crossing until a progeny with the desirable genetic traits or genetic background is obtained. One purpose served by such breeding methods is to introduce a desirable genetic trait into other varieties, breeding lines, hybrids or cultivars, particularly those that are of commercial interest. Another purpose is to facilitate stacking of genetic modifications of different genes in a single plant variety, lines, hybrids or cultivars. Intraspecific as well as interspecific matings are contemplated. The progeny plants that arise from such crosses, also referred to as breeding lines, are examples of non-naturally occurring plants of the invention.

In one embodiment, a method is provided for producing a non-naturally occurring tobacco plant comprising: (a) crossing a mutant or transgenic tobacco plant with a second tobacco plant to yield progeny tobacco seed; (b) growing the progeny tobacco seed, under plant growth conditions, to yield the non-naturally occurring tobacco plant. The method may further comprises: (c) crossing the previous generation of non-naturally occurring tobacco plant with itself or another tobacco plant to yield progeny tobacco seed; (d) growing the progeny tobacco seed of step (c) under plant growth conditions, to yield additional non-naturally occurring tobacco plants; and (e) repeating the crossing and growing steps of (c) and (d) multiple times to generate further generations of non-naturally occurring tobacco plants. The method may optionally comprises prior to step (a), a step of providing a parent plant which comprises a genetic identity that is characterized and that is not identical to the mutant or transgenic plant. In some embodiments, depending on the breeding program, the crossing and growing steps are repeated from 0 to 2 times, from 0 to 3 times, from 0 to 4 times, 0 to 5 times, from 0 to 6 times, from 0 to 7 times, from 0 to 8 times, from 0 to 9 times or from 0 to 10 times, in order to generate generations of non-naturally occurring tobacco plants. Backcrossing is an example of such a method wherein a progeny is crossed with one of its parents or another plant genetically similar to its parent, in order to obtain a progeny plant in the next generation that has a genetic identity which is closer to that of one of the parents. Techniques for plant breeding, particularly tobacco plant breeding, are well known and can be used in the methods of the invention. The invention further provides non-naturally occurring tobacco plants produced by these methods. Certain embodiments exclude the step of selecting a plant.

In some embodiments of the methods described herein, lines resulting from breeding and screening for variant genes are evaluated in the field using standard field procedures. Control genotypes including the original unmutagenized parent are included and entries are arranged in the field in a randomized complete block design or other appropriate field design. For tobacco, standard agronomic practices are used, for example, the tobacco is harvested, weighed, and sampled for chemical and other common testing before and during curing. Statistical analyses of the data are performed to confirm the similarity of the selected lines to the parental line. Cytogenetic analyses of the selected plants are optionally performed to confirm the chromosome complement and chromosome pairing relationships.

DNA fingerprinting, single nucleotide polymorphism, microsatellite markers, or similar technologies may be used in a marker-assisted selection (MAS) breeding program to transfer or breed mutant alleles of a gene into other tobaccos, as described herein. For example, a breeder can create segregating populations from hybridizations of a genotype containing a mutant allele with an agronomically desirable genotype. Plants in the F2 or backcross generations can be screened using a marker developed from a genomic sequence or a fragment thereof, using one of the techniques listed herein. Plants identified as possessing the mutant allele can be backcrossed or self-pollinated to create a second population to be screened. Depending on the expected inheritance pattern or the MAS technology used, it may be necessary to self-pollinate the selected plants before each cycle of backcrossing to aid identification of the desired individual plants. Backcrossing or other breeding procedure can be repeated until the desired phenotype of the recurrent parent is recovered.

According to the disclosure, in a breeding program, successful crosses yield F1 plants that are fertile. Selected F1 plants can be crossed with one of the parents, and the first backcross generation plants are self-pollinated to produce a population that is again screened for variant gene expression (for example, the null version of the gene). The process of backcrossing, self-pollination, and screening is repeated, for example, at least 4 times until the final screening produces a plant that is fertile and reasonably similar to the recurrent parent. This plant, if desired, is self-pollinated and the progeny are subsequently screened again to confirm that the plant exhibits variant gene expression. In some embodiments, a plant population in the F2 generation is screened for variant gene expression, for example, a plant is identified that fails to express a polypeptide due to the absence of the gene according to standard methods, for example, by using a PCR method with primers based upon the nucleotide sequence information for the polynucleotide(s) described herein (or any combination thereof as described herein).

Hybrid tobacco varieties can be produced by preventing self-pollination of female parent plants (that is, seed parents) of a first variety, permitting pollen from male parent plants of a second variety to fertilize the female parent plants, and allowing F1 hybrid seeds to form on the female plants. Self-pollination of female plants can be prevented by emasculating the flowers at an early stage of flower development. Alternatively, pollen formation can be prevented on the female parent plants using a form of male sterility. For example, male sterility can be produced by cytoplasmic male sterility (CMS), or transgenic male sterility wherein a transgene inhibits microsporogenesis and/or pollen formation, or self-incompatibility. Female parent plants containing CMS are particularly useful. In embodiments in which the female parent plants are CMS, pollen is harvested from male fertile plants and applied manually to the stigmas of CMS female parent plants, and the resulting F1 seed is harvested.

Varieties and lines described herein can be used to form single-cross tobacco F1 hybrids. In such embodiments, the plants of the parent varieties can be grown as substantially homogeneous adjoining populations to facilitate natural cross-pollination from the male parent plants to the female parent plants. The F1 seed formed on the female parent plants is selectively harvested by conventional means. One also can grow the two parent plant varieties in bulk and harvest a blend of F1 hybrid seed formed on the female parent and seed formed upon the male parent as the result of self-pollination. Alternatively, three-way crosses can be carried out wherein a single-cross F1 hybrid is used as a female parent and is crossed with a different male parent. As another alternative, double-cross hybrids can be created wherein the F1 progeny of two different single-crosses are themselves crossed.

A population of mutant, non-naturally occurring or transgenic plants can be screened or selected for those members of the population that have a desired trait or phenotype. For example, a population of progeny of a single transformation event can be screened for those plants having a desired level of expression or activity of the polypeptide(s) encoded thereby. Physical and biochemical methods can be used to identify expression or activity levels. These include Southern analysis or PCR amplification for detection of a polynucleotide; Northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining and enzyme assays also can be used to detect the presence or expression or activity of polypeptides or polynucleotides.

Mutant, non-naturally occurring or transgenic plant cells and plants are described herein comprising one or more recombinant polynucleotides, one or more polynucleotide constructs, one or more double-stranded RNAs, one or more conjugates or one or more vectors/expression vectors.

Without limitation, the plants described herein may be modified for other purposes either before or after the expression or activity has been modulated according to the present invention. One or more of the following genetic modifications can be present in the mutant, non-naturally occurring or transgenic plants. In one embodiment, one or more genes that are involved in the conversion of nitrogenous metabolic intermediates is modified resulting in plants or parts of plants (such as leaves or tobacco) that when cured, produces lower levels of at least one tobacco-specific nitrosamine than control plants or parts thereof. Non-limiting examples of genes that can be modified include genes encoding a nicotine demethylase, such as CYP82E4, CYP82E5 and CYP82E10 which participate in the conversion of nicotine to nornicotine and are described in WO2006091194, WO2008070274, WO2009064771 and PCT/US2011/021088. In another embodiment, one or more genes that are involved in heavy metal uptake or heavy metal transport are modified resulting in plants or parts of plants (such as leaves) having a lower heavy metal content than control plants or parts thereof without the modification(s). Non-limiting examples include genes in the family of multidrug resistance associated proteins, the family of cation diffusion facilitators (CDF), the family of Zrt-, Irt-like proteins (ZIP), the family of cation exchangers (CAX), the family of copper transporters (COPT), the family of heavy-metal P-type ATPases (for example, HMAs, as described in WO2009074325), the family of homologs of natural resistance-associated macrophage proteins (NRAMP), and the family of ATP-binding cassette (ABC) transporters (for example, MRPs, as described in WO2012/028309, which participate in transport of heavy metals, such as cadmium. The term heavy metal as used herein includes transition metals. Examples of other modifications include herbicide tolerance, for example, glyphosate is an active ingredient of many broad spectrum herbicides. Glyphosate resistant transgenic plants have been developed by transferring the aroA gene (a glyphosate EPSP synthetase from Salmonella typhimurium and E. coli). Sulphonylurea resistant plants have been produced by transforming the mutant ALS (acetolactate synthetase) gene from Arabidopsis. OB protein of photosystem II from mutant Amaranthus hybridus has been transferred in to plants to produce atrazine resistant transgenic plants; and bromoxynil resistant transgenic plants have been produced by incorporating the bxn gene from the bacterium Klebsiella pneumoniae. Another exemplary modification results in plants that are resistant to insects. Bacillus thuringiensis (Bt) toxins can provide an effective way of delaying the emergence of Bt-resistant pests, as recently illustrated in broccoli where pyramided cry1Ac and cry1C Bt genes controlled diamondback moths resistant to either single protein and significantly delayed the evolution of resistant insects. Another exemplary modification results in plants that are resistant to diseases caused by pathogens (for example, viruses, bacteria, fungi). Plants expressing the Xa21 gene (resistance to bacterial blight) with plants expressing both a Bt fusion gene and a chitinase gene (resistance to yellow stem borer and tolerance to sheath) have been engineered. Another exemplary modification results in altered reproductive capability, such as male sterility. Another exemplary modification results in plants that are tolerant to abiotic stress (for example, drought, temperature, salinity), and tolerant transgenic plants have been produced by transferring acyl glycerol phosphate enzyme from Arabidopsis; genes coding mannitol dehydrogenase and sorbitol dehydrogenase which are involved in synthesis of mannitol and sorbitol improve drought resistance. Another exemplary modification results in plants that produce proteins which may have favourable immunogenic properties for use in humans. For example, plants capable of producing proteins which substantially lack alpha-1,3-linked fucose residues, beta-1,2-linked xylose residues, or both, in its N-glycan may be of use. Other exemplary modifications can result in plants with improved storage proteins and oils, plants with enhanced photosynthetic efficiency, plants with prolonged shelf life, plants with enhanced carbohydrate content, and plants resistant to fungi; plants encoding an enzyme involved in the biosynthesis of alkaloids. Transgenic plants in which the expression of S-adenosyl-L-methionine (SAM) and/or cystathionine gamma-synthase (CGS) has been modulated are also contemplated.

One or more such traits may be introgressed into the mutant, non-naturally occurring or transgenic tobacco plants from another tobacco cultivar or may be directly transformed into it. The introgression of the trait(s) into the mutant, non-naturally occurring or transgenic tobacco plants of the invention maybe achieved by any method of plant breeding known in the art, for example, pedigree breeding, backcrossing, doubled-haploid breeding, and the like (see, Wernsman, E. A, and Rufty, R. C. 1987. Chapter Seventeen. Tobacco. Pages 669-698 In: Cultivar Development. Crop Species. W. H. Fehr (ed.), MacMillan Publishing Co, Inc., New York, N.Y. 761 pp.). Molecular biology-based techniques described above, in particular RFLP and microsatelite markers, can be used in such backcrosses to identify the progenies having the highest degree of genetic identity with the recurrent parent. This permits one to accelerate the production of tobacco varieties having at least 90%, preferably at least 95%, more preferably at least 99% genetic identity with the recurrent parent, yet more preferably genetically identical to the recurrent parent, and further comprising the trait(s) introgressed from the donor parent. Such determination of genetic identity can be based on molecular markers known in the art.

The last backcross generation can be selfed to give pure breeding progeny for the nucleic acid(s) being transferred. The resulting plants generally have essentially all of the morphological and physiological characteristics of the mutant, non-naturally occurring or transgenic tobacco plants of the invention, in addition to the transferred trait(s) (for example, one or more single gene traits). The exact backcrossing protocol will depend on the trait being altered to determine an appropriate testing protocol. Although backcrossing methods are simplified when the trait being transferred is a dominant allele, a recessive allele may also be transferred. In this instance, it may be necessary to introduce a test of the progeny to determine if the desired trait has been successfully transferred.

Various embodiments provide mutant plants, non-naturally occurring plants or transgenic plants, as well as biomass in which the expression level of a polynucleotide (or any combination thereof as described herein) is modulated to modulate the nitrate and/or at least NNK content and/or biomass yield therein

Parts of such plants, particularly tobacco plants, and more particularly the leaf lamina and midrib of tobacco plants, can be incorporated into or used in making various consumable products including but not limited to aerosol forming materials, aerosol forming devices, smoking articles, smokable articles, smokeless products, and tobacco products. Examples of aerosol forming materials include but are not limited to tobacco compositions, tobaccos, tobacco extract, cut tobacco, cut filler, cured tobacco, expanded tobacco, homogenized tobacco, reconstituted tobacco, and pipe tobaccos. Smoking articles and smokable articles are types of aerosol forming devices. Examples of smoking articles or smokable articles include but are not limited to cigarettes, cigarillos, and cigars. Examples of smokeless products comprise chewing tobaccos, and snuffs. In certain aerosol forming devices, rather than combustion, a tobacco composition or another aerosol forming material is heated by one or more electrical heating elements to produce an aerosol. In another type of heated aerosol forming device, an aerosol is produced by the transfer of heat from a combustible fuel element or heat source to a physically separate aerosol forming material, which may be located within, around or downstream of the heat source. Smokeless tobacco products and various tobacco-containing aerosol forming materials may contain tobacco in any form, including as dried particles, shreds, granules, powders, or a slurry, deposited on, mixed in, surrounded by, or otherwise combined with other ingredients in any format, such as flakes, films, tabs, foams, or beads. As used herein, the term ‘smoke’ is used to describe a type of aerosol that is produced by smoking articles, such as cigarettes, or by combusting an aerosol forming material.

In one embodiment, there is also provided cured plant material from the mutant, transgenic and non-naturally occurring tobacco plants described herein. Processes of curing green tobacco leaves are known by those having skills in the art and include without limitation air-curing, fire-curing, flue-curing and sun-curing. The process of curing green tobacco leaves depends on the type of tobacco harvested. For example, Virginia flue (bright) tobacco is typically flue-cured, Burley and certain dark strains are usually air-cured, and pipe tobacco, chewing tobacco, and snuff are usually fire-cured.

In another embodiment, there is described tobacco products including tobacco-containing aerosol forming materials comprising plant material—such as leaves, preferably cured leaves—from the mutant tobacco plants, transgenic tobacco plants or non-naturally occurring tobacco plants described herein. The tobacco products described herein can be a blended tobacco product which may further comprise unmodified tobacco.

The amount of NNK in these smokable articles and smokeless products and aerosols thereof may be at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, and 100% lower—such as about 200% or 300% lower—when compared to consumable products derived from non-mutant, non-naturally occurring or non-transgenic counterparts.

The amount of NNN in these smokable articles and smokeless products and aerosols thereof may be at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, and 100% lower—such as about 200% or 300% lower—when compared to consumable products derived from non-mutant, non-naturally occurring or non-transgenic counterparts.

The amount of nitrate in these smokable articles and smokeless products and aerosols thereof may be at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, and 100% lower—such as about 200% or 300% lower—when compared to consumable products derived from non-mutant, non-naturally occurring or non-transgenic counterparts.

The amount of nicotine in these smokable articles and smokeless products and aerosols thereof may be at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, and 100% lower—such as about 200% or 300% lower—when compared to consumable products derived from non-mutant, non-naturally occurring or non-transgenic counterparts. The amount of nicotine in these smokable articles and smokeless products and aerosols thereof may be about the same as compared to consumable products derived from non-mutant, non-naturally occurring or non-transgenic counterparts.

The amount of total TSNAs in these smokable articles and smokeless products and aerosols thereof may be at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, and 100% lower—such as about 200% or 300% lower—when compared to consumable products derived from non-mutant, non-naturally occurring or non-transgenic counterparts.

The mutant, non-naturally occurring or transgenic plants may have other uses in, for example, agriculture. For example, mutant, non-naturally occurring or transgenic plants described herein can be used to make animal feed and human food products.

The invention also provides methods for producing seeds comprising cultivating the mutant plant, non-naturally occurring plant, or transgenic plant described herein, and collecting seeds from the cultivated plants. Seeds from plants described herein can be conditioned and bagged in packaging material by means known in the art to form an article of manufacture. Packaging material such as paper and cloth are well known in the art. A package of seed can have a label, for example, a tag or label secured to the packaging material, a label printed on the package that describes the nature of the seeds therein.

Compositions, methods and kits for genotyping plants for identification, selection, or breeding can comprise a means of detecting the presence of a polynucleotide (or any combination thereof as described herein) in a sample of polynucleotide. Accordingly, a composition is described comprising one of more primers for specifically amplifying at least a portion of one or more of the polynucleotides and optionally one or more probes and optionally one or more reagents for conducting the amplification or detection.

Accordingly, gene specific oligonucleotide primers or probes comprising about 10 or more contiguous polynucleotides corresponding to the polynucleotide(s) described herein are disclosed. Said primers or probes may comprise or consist of about 15, 20, 25, 30, 40, 45 or 50 more contiguous polynucleotides that hybridise (for example, specifically hybridise) to the polynucleotide(s) described herein. In some embodiments, the primers or probes may comprise or consist of about 10 to 50 contiguous nucleotides, about 10 to 40 contiguous nucleotides, about 10 to 30 contiguous nucleotides or about 15 to 30 contiguous nucleotides that may be used in sequence-dependent methods of gene identification (for example, Southern hybridization) or isolation (for example, in situ hybridization of bacterial colonies or bacteriophage plaques) or gene detection (for example, as one or more amplification primers in nucleic acid amplification or detection). The one or more specific primers or probes can be designed and used to amplify or detect a part or all of the polynucleotide(s). By way of specific example, two primers may be used in a polymerase chain reaction protocol to amplify a nucleic acid fragment encoding a nucleic acid—such as DNA or RNA. The polymerase chain reaction may also be performed using one primer that is derived from a nucleic acid sequence and a second primer that hybridises to the sequence upstream or downstream of the nucleic acid sequence—such as a promoter sequence, the 3′ end of the mRNA precursor or a sequence derived from a vector. Examples of thermal and isothermal techniques useful for in vitro amplification of polynucleotides are well known in the art. The sample may be or may be derived from a plant, a plant cell or plant material or a tobacco product made or derived from the plant, the plant cell or the plant material as described herein.

In a further aspect, there is also provided a method of detecting a polynucleotide(s) described herein (or any combination thereof as described herein) in a sample comprising the step of: (a) providing a sample comprising, or suspected of comprising, a polynucleotide; (b) contacting said sample with one of more primers or one or more probes for specifically detecting at least a portion of the polynucleotide(s); and (c) detecting the presence of an amplification product, wherein the presence of an amplification product is indicative of the presence of the polynucleotide(s) in the sample. In a further aspect, there is also provided the use of one of more primers or probes for specifically detecting at least a portion of the polynucleotide(s). Kits for detecting at least a portion of the polynucleotide(s) are also provided which comprise one of more primers or probes for specifically detecting at least a portion of the polynucleotide(s). The kit may comprise reagents for polynucleotide amplification—such as PCR—or reagents for probe hybridization-detection technology—such as Southern Blots, Northern Blots, in-situ hybridization, or microarray. The kit may comprise reagents for antibody binding-detection technology such as Western Blots, ELISAs, SELDI mass spectrometry or test strips. The kit may comprise reagents for DNA sequencing. The kit may comprise reagents and instructions for determining nitrate content and/or at least NNK content and/or NNN content and/or nictotine content and/or total TSNA content. Suitably, the kit comprises reagents and instructions for determining nitrate content and/or at least NNK content and/or nictotine content and/or NNN content and/or total TSNA content in plant material, cured plant material or cured leaves.

In some embodiments, a kit may comprise instructions for one or more of the methods described. The kits described may be useful for genetic identity determination, phylogenetic studies, genotyping, haplotyping, pedigree analysis or plant breeding particularly with co-dominant scoring. The present invention also provides a method of genotyping a plant, a plant cell or plant material comprising a polynucleotide as described herein. Genotyping provides a means of distinguishing homologs of a chromosome pair and can be used to differentiate segregants in a plant population. Molecular marker methods can be used for phylogenetic studies, characterizing genetic relationships among crop varieties, identifying crosses or somatic hybrids, localizing chromosomal segments affecting monogenic traits, map based cloning, and the study of quantitative inheritance. The specific method of genotyping may employ any number of molecular marker analytic techniques including amplification fragment length polymorphisms (AFLPs). AFLPs are the product of allelic differences between amplification fragments caused by nucleotide sequence variability. Thus, the present invention further provides a means to follow segregation of one or more genes or nucleic acids as well as chromosomal sequences genetically linked to these genes or nucleic acids using such techniques as AFLP analysis.

In one embodiment, there is also provided cured plant material from the mutant, transgenic and non-naturally occurring plants described herein. For example, processes of curing tobacco leaves are known by those having skills in the field and include without limitation air-curing, fire-curing, flue-curing and sun-curing. The process of curing green tobacco leaves depends on the type of tobacco harvested. For example, Virginia flue (bright) tobacco is typically flue-cured, Burley and certain dark strains are usually air-cured, and pipe tobacco, chewing tobacco, and snuff are usually fire-cured.

In another embodiment, there is described tobacco products including tobacco products comprising plant material—such as leaves, suitably cured plant material—such as cured leaves—from the mutant, transgenic and non-naturally occurring plants described herein or which are produced by the methods described herein. The tobacco products described herein may further comprise unmodified tobacco.

In another embodiment, there is described tobacco products comprising plant material, preferably leaves—such as cured leaves, from the mutant, transgenic and non-naturally occurring plants described herein. For example, the plant material may be added to the inside or outside of the tobacco product and so upon burning a desirable aroma is released. The tobacco product according to this embodiment may even be an unmodified tobacco or a modified tobacco. The tobacco product according to this embodiment may even be derived from a mutant, transgenic or non-naturally occurring plant which has modifications in one or more genes other than the genes disclosed herein.

The invention is further described in the Examples below, which are provided to describe the invention in further detail. These examples, which set forth a preferred mode presently contemplated for carrying out the invention, are intended to illustrate and not to limit the invention.

### EXAMPLES

### Example 1: Identification of NtCLCe-s Sequences

For the identification of NtCLCe-s, related transcripts are detected in N. tabacum leaves by RT-PCR analyses and the existence of potentially matching EST-contigs (NtCLCe-s: NCBI_43350-v4ctg-in). Data from an Affymetrix custom-made tobacco exon-array (sequence probes from NtPMlalg22230e1-st) is used to confirm that NtCLCe-s is equally expressed in roots, green and senescent leaves of N. tabacum. Furthermore, cold stress and strong cadmium stress is found not to affect NtCLCe-s expression levels, thereby suggesting that NtCLCe-s is constitutively expressed in tobacco root and leaf organs. Constitutive NtCLCe expression may be correlated with the maintenance of its essential cellular role in plastids which is presumably linked to the nitrogen assimilation pathway. According to WoLFPSORT software, NtCLCe-s is highly predicted to be a plastidial membrane protein. RNAseq studies confirms the presence of the transcript in its ancestor N. sylvestris.

### Example 2: Identification of NtCLCe-t Sequences

For the identification of NtCLCe-t, related transcripts are detected in N. tabacum leaves by RT-PCR analyses and the existence of corresponding EST-contigs. RNAseq studies confirm the presence of the transcript in the ancestor N. tomentosiformis, thereby suggesting that the expression of the NtCLCe-t copy is possibly lost in N. tabacum after entering the allotetraploid state, possibly due to gene disruption and/or rearrangement.

### Example 3: Expression of NtCLCe-s or NtCLCe-t in N. tabacum Leaves

Both CLC-Nt2-s and CLC-Nt2-t genes are expressed in N. tabacum leaves, as determined by the presence of both transcripts in N. tabacum leaves (custom made tobacco exon-array studies validated by RT-PCR) and corresponding EST-contigs (CLC-Nt2-s: MIRA_20760-v4ctg-in; CLC-Nt2-t: NCBI_56794-v4ctg-in). In addition RNAseq studies confirms the presence of the corresponding transcripts in the two ancestors N. sylvestris and N. tomentosiformis.

When looking more carefully at transcriptomic data from the tobacco exon-array with specific probes for CLC-Nt2-t and CLC-Nt2-s, NtPMlalg19904e2-st and NtPMlalg50210e2-st, respectively, it is seen that both copies are differentially expressed in N. tabacum. CLC-Nt2-s is poorly expressed in Burley root (TN90) and CLC-Nt2-t is sensitive to the circadian rhythm. Both genes are expressed in root and leaf of flue-cured tobacco and are insensitive to cadmium treatment.

### Example 4: Silencing of CLC-Nt2-t Expression in N. tabacum

A DNA fragment (SEQ ID NO: 8) identified in the coding sequence of CLC-Nt2 and flanking an intron (100% identity with CLC-Nt2-s and 97% identity with CLC-Nt2-t) in N. tabacum (Hicks broadleaf) is cloned in order to silence both CLC-Nt2 copies in tobacco using a RNAi approach. The corresponding DNA fragment is inserted into the Gateway vector pB7GWIWG2(II) via an entry vector, exactly as detailed by the manufacturer (Invitrogen). This vector contains a promoter for constitutive expression (the cauliflower mosaic virus CaMV 35S promoter) of the transgene in all tissues of the plant and the kan gene for kanamycin antibiotic resistance. The construct is then inserted in to the genome of the Burley tobacco Kentucky 14 (KY14) via Agrobacterium tumefasciens using a classical leaf disk procedure. From calli, individual lines are regenerated. The selection of transgenic lines is performed by PCR on isolated genomic DNA from plantlets. RNAi silencing T0 lines are monitored by RT-PCR using specific primers flanking the insert used for silencing and grown for seed production. T1 seeds are collected, re-grown on agar plates and monitored exactly as T0 plantlets. Positive plants are grown in pots and cultivated in the greenhouse. At harvest time (10 week old plants), one leaf at mid stalk position is sampled and subjected to nitrate determination using either a nitrate colorimetric assay kit (Cayman, US) or Skalar. All remaining leaves are cured plant by plant in a small experimental air-curing barn for two months using standard methods that are known in the art. After curing, leaves of each plant are assembled and subjected to TSNA analyses.

### Example 5: Silencing of NtCLCe Expression in N. tabacum

A DNA fragment (SEQ ID NO: 9) identified in the coding sequence of NtCLCe is cloned to silence both NtCLCe copies using a RNAi approach. The corresponding DNA fragment is then inserted into the Gateway vector pB7GWIWG2(II) via an entry vector, exactly as detailed by the manufacturer (Invitrogen). This vector contains a promoter for constitutive expression (the cauliflower mosaic virus CaMV 35S promoter) of the transgene in all tissues of the plant and the kan gene for kanamycin antibiotic resistance. The construct is then inserted in the genome of the Burley tobacco Kentucky 14 (KY14) via Agrobacterium tumefasciens using a classical leaf disk procedure. From calli, individual lines are regenerated. The selection on agar plates is performed by PCR on isolated genomic DNA from plantlets. RNAi silencing T0 lines is then monitored by RT-PCR using specific primers flanking the insert used for silencing and grown for seed production. T1 seeds are collected, re-grown on agar plates and monitored exactly as T0 plantlets. Positive plants are grown on pots and cultivated in the greenhouse. At harvest time (10 weeks old plants), one leaf at mid stalk position is sampled and subjected to nitrate determination using either a nitrate colorimetric assay kit (Cayman, US) or Skalar. The rest of the leaves are cured plant by plant in a small experimental air-curing barn for two months using standard methods that are known in the art. After curing, leaves of each plant are assembled and subjected to TSNA analyses.

### Example 6: TSNA Analysis in CLC-NT2-RNAi and NtCLCe-RNAi Plants

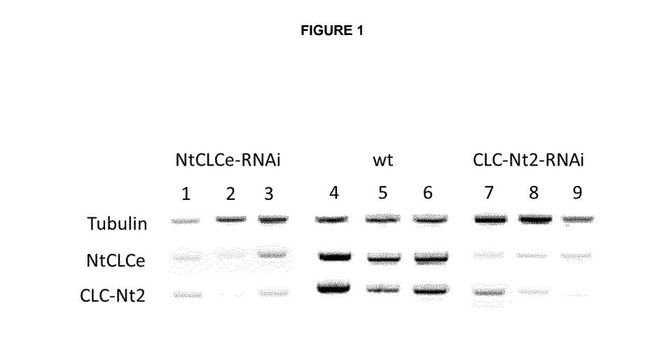

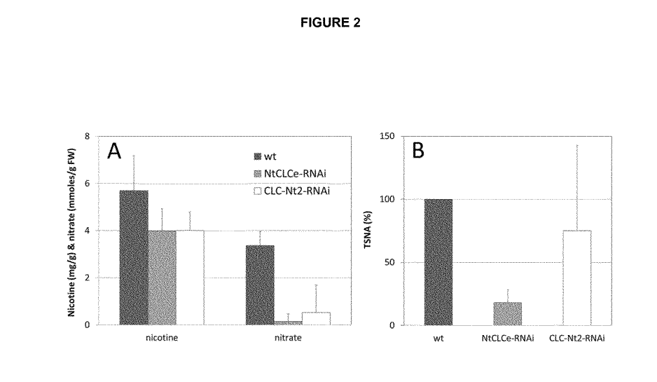

The selection of CLC-NT2-RNAi and NtCLCe-RNAi plants using PCR on genomic DNA to identify transgenic inserts followed by RT-PCR on cDNA (obtained from isolated total RNA) is performed. As shown in FIG. 1 (semi-quantitative RT-PCR analyses), CLC-Nt2 or NtCLCe genes are found to be fully or partially silenced in green leaves of CLC-Nt2-RNAi and NtCLCe-RNAi T1 plants compared to wild-type plants (three representative plants are shown). Interestingly, in both RNAi plants, NtCLCe and CLC-Nt2 genes are silenced independently of the construct used, thereby suggesting possible cross-talk regulation between these two genes in leaves. In a first experiment, T1 plantlets are grown in small pots (3 liter pots) after germination. At harvest time (10 weeks after transplanting), nitrate reduction is observed in both CLC-Nt2-RNAi and NtCLCe-RNAi green leaves (mid-stalk position), however the reduction of nitrate is significantly (P<0.01) more effective in NtCLCe-RNAi plants (˜95%) compared to CLC-Nt2 plants (about seq id no:5%, see FIG. 2A). Nicotine reduction is also seen in both transgenic plants when compared to wt plants (˜35%). This nicotine reduction suggests that NtCLCe and CLC-Nt2 affect nitrate redistribution in roots under certain growth conditions which influences nicotine synthesis. Total TSNA (NNN, NNK, NAT (N9-nitrosoanatabine) and NAB (N9-nitrosoanabasine) is determined in both CLC-RNAi plants after curing (see FIG. 2B). NNK, NNN, NAB and NAT are available commercially which can be of use as reference standards. Standard methods for the analysis of NNK, NNN, NAB and NAT are known in the art (see, for example, Nicotine & Tobacco Research (2006) 2:309-313). Ultra performance liquid chromatography-tandem mass spectrometry (UPLC-MS/MS) can be used. Methods for measuring nicotine are also known in the art (see, for example, International Journal of Cancer (2005); 116:16-19). The data indicate that the strong reduction of nitrate levels prevents the formation of TSNA in cured leaves, which may be because nitrate is the main source of nitrosating agent in leaves contributing to the formation of TSNA. The reduction in nitrate found in CLC-Nt2-RNAi plants does not result in such a strong TSNA effect when compared to NtCLCe-RNAi plants.

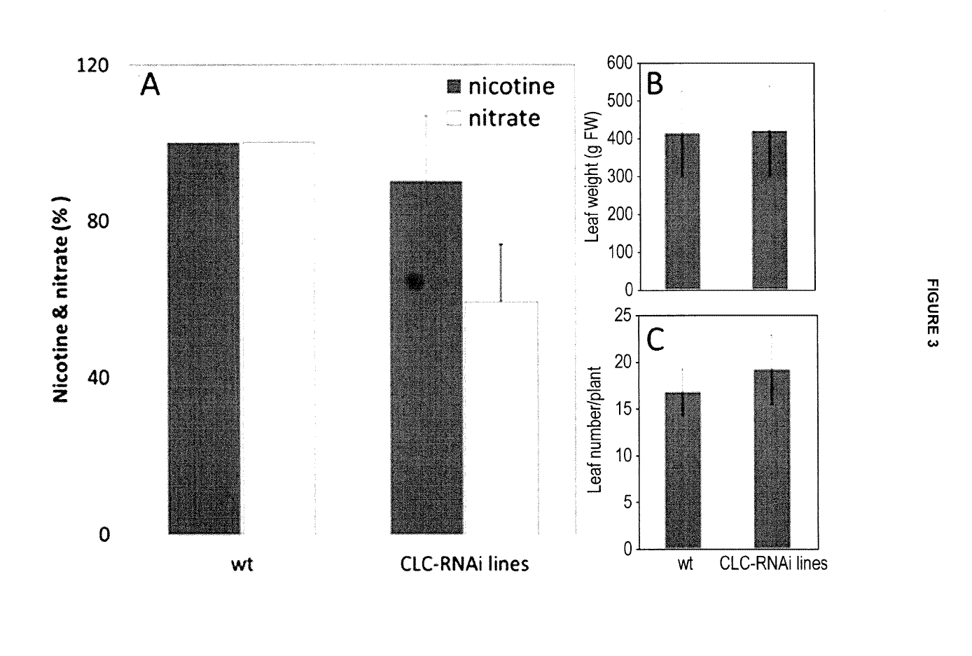

To prevent any stress conditions for root growth, the previous experiment is repeated using 10 liter pots. Under such conditions, wild-type tobacco plants accumulate about five times more nicotine when compared to the previous experiment. NtCLCe-RNAi and CLC-Nt2-RNAi plants showing reduced gene expression were selected exactly as described before. Since most of the transgenic plants from both constructs exhibited reduced expression for NtCLCe and CLC-Nt2 (see FIG. 1), the RNAi plants showing reduced expression for both CLCs were grouped together (CLC-RNAi plants) and subjected to nicotine and nitrate analyses (see FIG. 3A). The reduction of nicotine observed in the first experiments for CLC-RNAi plants was not found in this experiment, thereby confirming that confining root development by using small pots may trigger additional reduction of nicotine in both NtCLCe-RNAi and CLC-Nt2-RNAi plants compared to wild type plants (compare FIGS. 2A and 3A). However, nitrate was still significantly reduced (>40%) in both CLC-RNAi plants compared to wild type plants, thus confirming that reducing expression of NtCLCe and CLC-Nt2 leads to a nitrate content decrease in tobacco leaves. Under such growth conditions, transgenic plants did not show any phenotypic differences compared to wt plants, as can be seen by comparing total leaf weight and leaf numbers (see FIGS. 3B and 3C).

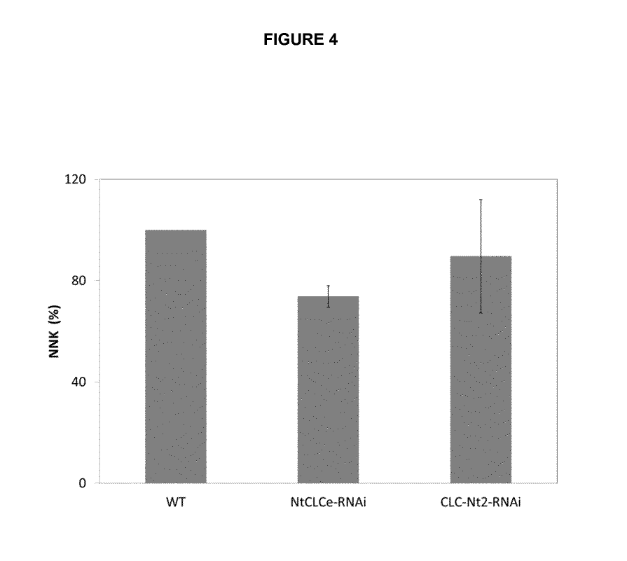

The analyses of TSNA in these plants showed that NNN was not reduced in air-cured leaves compared to wild type plants. However, 24 and 10% NNK reduction is seen in both NtCLCe-RNAi and CLC-Nt2-RNAi plants compared to wild type plants (see FIG. 4). The NNK reduction is more significant in NtCLCe-RNAi (P<0.01) than in CLC-Nt2-RNAi plants, thereby confirming the data obtained in the first experiment for total TSNA (see FIG. 2).

Although transgenic and wild type plants are not grown under a field environment and not cured in classical barns for air-curing tobacco, our data show that limiting the expression of NtCLCe (NtCLCe-s) and CLC-Nt2 (s and t copies) contributes to efficiently reduce nitrate in tobacco leaves. After curing, TSNA (NNK) is found to be reduced in the leaves, indicating that reducing the nitrate content in green leaves as a provider for nitrosating agents during curing will effectively contribute to reducing the formation of TSNA in the corresponding cured leaves. This reduction can correspond to an at least 20% reduction in NNK.

### Example 7: Ethyl-Methanesulfonate Mutagensis of CLC-Nt2-s, CLC-Nt2-t, NtCLCe-s or NtCLCe-t in N. tabacum

M0 seeds of Nicotiana tabacum AA37 are treated with ethyl-methanesulfonate (EMS) at different concentrations and exposure times, in order to generate a population of plants with random point mutations. A kill-curve is estimated at M1 generation for each treatment, together with lethality, fertility and rate of chimerism. M1 plants are self fertilized to generate M2 families of seeds, to allow recessive alleles to be recovered as homozygous and lethal alleles to be recovered as heterozygous. Genomic DNA from 8 M2 plants per each family of the EMS mutagenised population is extracted and screened for mutants, while M2 plant material and M3 seeds are collected and stored for future analyses. To identify and characterise the mutant variants, genomic DNA samples from M2 plants are pooled in groups and screened by sequencing of targeted gene fragments. Target gene fragments are amplified using the primers shown in Table 2. Mutations in the target genes are retrieved by sequencing the individual DNA fragments. The various mutants are shown in Table 1.

### Example 8: Analysis of Field Grown CLCNt2-s G163R Homozygous Mutant Tobacco Plant

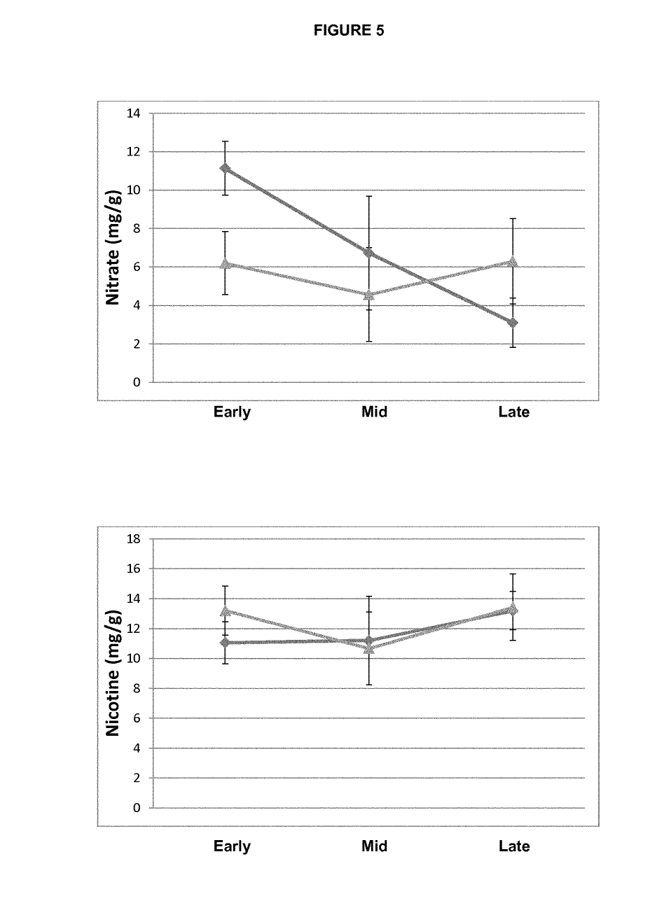

The time course of nitrate and nicotine levels in green leaves of field grown CLCNt2-s G163R mutant tobacco plants is shown in FIG. 5. Entire leaves are harvested at mid-stalk position from CLCNt2-s G163R homozygous mutant tobacco plants (triangle) and out-segregant wild type (diamond) tobacco plants grow in field under Burley regime. Samples are harvested at three different times during the morning (early, mid and late) and freeze-dried. Powdered lamina material is analyzed for nitrate and nicotine content. N=4 to 8 individual plants. Standard deviation is indicated in the figures.

The results of this experiment show that the CLCNt2-s G163R homozygous mutant tobacco plant has a reduced level of nitrate in the early morning as compared to the control plant. The level of nitrate is reduced from about 11 mg/g in the control plant to about 6 mg/g in the mutant plant. The nitrate level continues to decrease in the mid-morning. The level of nitrate is reduced from about 7 mg/g in the control plant to about 4.5 mg/g in the mutant plant. By the late morning the nitrate level has increased in the mutant plant as compared to the mid-morning and reaches the nitrate level present in the early morning. For the control, the nitrate level in the control plant continues to decrease. By late morning, the level of nitrate increases to about 6 mg/g in the mutant plant and decreases to about 3 mg/g in the control plant. The level of nicotine is somewhat similar during the morning. The level of nicotine varies between about 13 mg/g and about 11 mg/g for the mutant plant and about 9 mg/g and 13 mg/g for the control plant. The nicotine result indicates that the metabolism of the mutant plant is normal. The biomass levels for the mutant and the control plant are also comparable.

### Example 9: Analysis of Field Grown NtCLCe-t P143L Homozygous Mutant Tobacco Plant

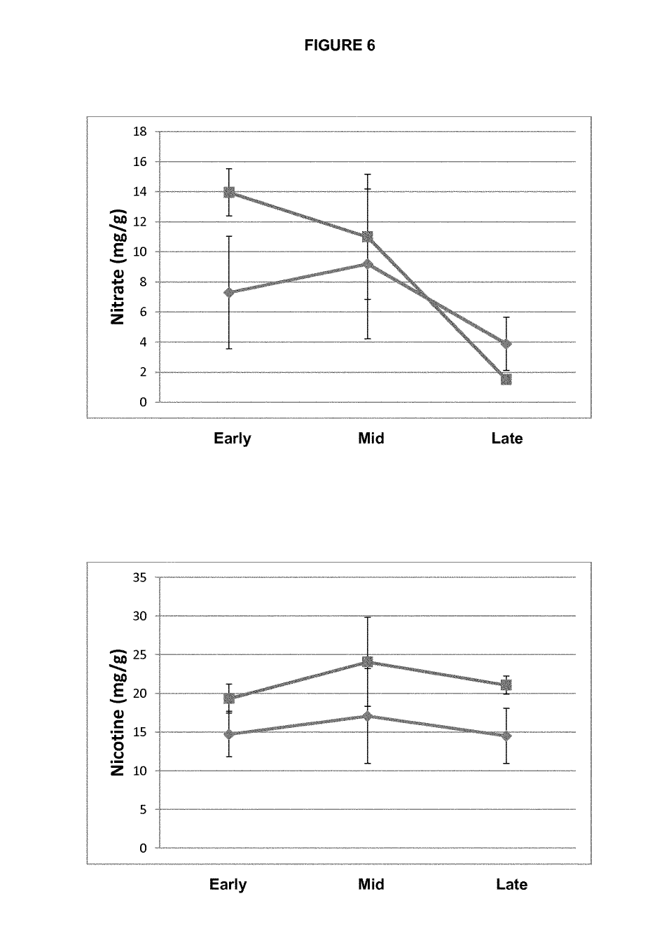

The time course of nitrate and nicotine levels in green leaves of field grown NtCLCe-t P143L mutant plants is shown in FIG. 6. Entire leaves are harvested at mid-stalk position from field grown NtCLCe-t P143L homozygous (square) and out-segregant wild type (diamond) plants growing under Burley regime. Samples are harvested at three different times during the morning (early, mid and late) and freeze-dried. Powdered lamina material is analyzed for nitrate and nicotine content. N=4 to 8 individual plants. Standard deviation is indicated in the Figure.

The results of this experiment show that the NtCLCe-t P143L homozygous mutant tobacco plant has an increased level of nitrate in the early morning as compared to the control plant. The level of nitrate is increased from about 7 mg/g in the control plant to about 14 mg/g in the mutant plant. The nitrate level decreases in the mid-morning in the mutant plant and increases slightly in the control plant. The level of nitrate in the mutant plant is reduced to about 9 mg/g and the level of nitrate in the control plant increases to about 9 mg/g. By the late morning the nitrate level has continued to decrease in the mutant plant as compared to the mid-morning. For the control, the nitrate level in the control plant decreases. By late morning, the level of nitrate decreases to about 2 mg/g in the mutant plant and decreases to about 4 mg/g in the control plant. The level of nicotine is somewhat similar during the morning for each of the mutant and control plants. The level of nicotine varies between about 20 mg/g and about 24 mg/g for the mutant plant and about 15 mg/g and 17 mg/g for the control plant. The nicotine result indicates that the metabolism of the mutant plant is normal. The biomass levels for the mutant and the control plant are also comparable.

### Example 10 Field Trial

Plants positive for different CLC variant mutations (including the variants selected for altered sensitivity to chlorine gas sterilization) were genotyped and tested in a field trial in La Sota (Payerne, Switzerland) under classical Burley fertilization regime, together with other mutant lines of different CLC genes or of other genes involved in nitrogen metabolism. At least three repetitions of 10 plant plots per genotype were randomly scattered along the available field, keeping out-segregant wild-type (in solid black), heterozygous (dotted) and homozygous (white) plots of each repetition adjacent in the field, for comparison reasons.

After topping, mature leaves were air-cured on the stalk. After curing, 15 leaves per plant (when possible) were detached from the stalk and collected in paper bags for further analysis. The number of plants per plot and the weight of cured leaf material was also recorded, for biomass detection.

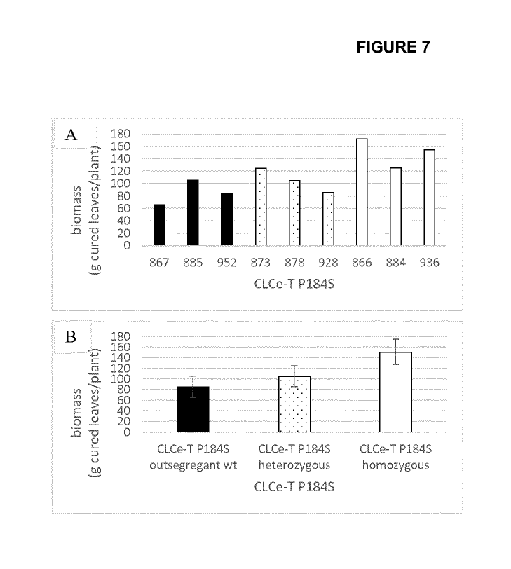

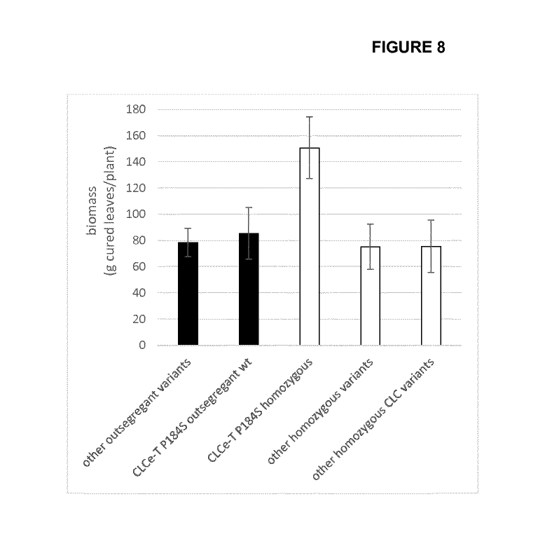

As shown in FIG. 7B, homozygous CLCe-T P184S mutant plants reached almost twice the biomass of both their heterozygous and wild-type out-segregant lines grown and cured under the same conditions. The improvement in biomass was statistically valid (confidence interval at 95%), consistent for all three homozygous plots (FIG. 7A) and exclusively observed for the plots corresponding to the NtCLCe-T P184S homozygous plants. As indicated in FIG. 8, in fact all other variant plots (out-segregant wild-type or homozygous lines of different mutations within CLC gene family or even in other analyzed genes) displayed the same average, and solely the NtCLCe-T P184S homozygous plants reached an average biomass of almost 2 fold the other field plots.

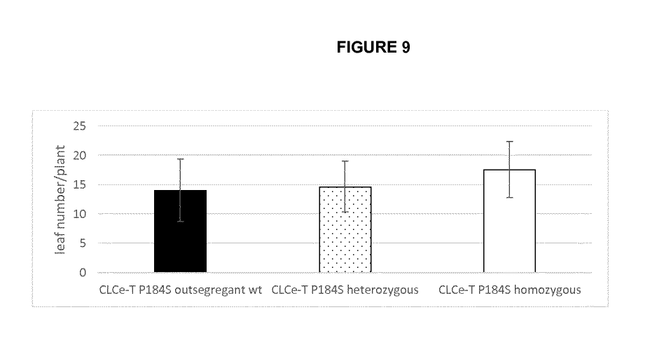

From an analysis of the number of leaves present on each plant at topping time, it appears that the biomass increase of the NtCLCe-T P184S homozygous plants is most likely due to an increase in single leaf biomass rather than leaf number, since as shown in FIG. 9 none of the genotypes of the NtCLCe-T P184S line differs significantly in leaf number per plant compared to the others.

Any publication cited or described herein provides relevant information disclosed prior to the filing date of the present application. Statements herein are not to be construed as an admission that the inventors are not entitled to antedate such disclosures. All publications mentioned in the above specification are herein incorporated by reference. Various modifications and variations of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in cellular, molecular and plant biology or related fields are intended to be within the scope of the following claims.

### Sequences

(DNA sequence of CLC-Nt2 from Nicotiana tabacum; sequence originating from
the ancestor N. sylvestris)
SEQ ID NO: 1
atggaggagccaactcgattagtagaagaagcaacgattaataacatggacggacaacagaatgaagaagaaagagatcc
agagagcaattcactgcatcagcctcttctcaagagaaacagaacactatcatccagtccatttgccttggttggagcta
aggtctcccacatcgaaagtttggattatgagtaagaacaactaataatcttatcatagatcaagtatagcttttcttta
cttgtgcattaaaagggccaacagaaattggatgtcctaattgtgtgtgtctgttttaggatcaacgagaatgatctctt
caagcatgactggagaaggagatctagagttcaagtattacagtatgtgttcttgaaatggacactggcatttttggtcg
gcctgcttacaggagttacagccaccctcatcaatcttgcaatcgaaaacatggctggttacaaacttcgagctgttgtg
aactatatcgaggatagaaggtaggtgatgttttccctatgatcaacaattcataaatgcttccagaagtcttactactg
attcttcaatacgataccactagctaatgactaagaacaagaccaaagatcacttatttgacttgaattatgttattgat
ttattcataattgagattgtaacaatggttacaggtaccttatgggatttgcatattttgcgggtgctaattttgtgctc
actttgatagctgcccttctctgcgtgtgctttgcacctactgctgcagggcctggaattcctgaaatcaaagcttatct
caacggtgtagatactcccaatatgtatggagcaaccacactttttgtcaaggtgcgtcacacacccaattttatcagtg
ctggcaattcagatagcaggcagattataacgccatcagtatagtattgagattctgtcgaaccagatgtataaatagat
agaatagcagcaaataacacatttttatcttagtcgtgatggcacctaatccgacccgctagataagccaaatacaatca
acacatatttatggaattcaatctcatttgggaagtgatctctatctttcagtaatcagataggaagtggtttaagaata
aaaagagaattttagaatcgaatgcactcatccagcgaggaagatccatcagtggtatctaatttactcttgaacttcca
gcagttcaatcctttggtaccgtcactgtaacttgtttttttcaatctttgtgactaacatggaagggaggaaaatcctg
actttcagtgattttcctcgcttacagtgaaagtcaggatatagcttcggtgagactcagcttatatgtcttaattgaat
atgctatttgttgactaacatggatttgccctatcatgaaaatgaaggaagcgccaaaaatacatatacttaaacagggg
cggacccaagtggtgagaagtgggttcaactgaacccgcttcgtcaaaaaaatactgtgtatatgtataaattatggcta
aagcaaggtaaattttgtatagaaataagcttatgttagttatggacttctcctgggtccgctactgtacttaaaagcac
atacgaagagatacacaaactaagggcaaaggttcataatttaaggcagttgtgtccagaagaacaaattttgcttgcat
gttgcagtgtgaatttaacaataaaagaattatgatcgcaaatttccacttgtaattgtactataagattctaaattttg
agagatttgacatgtttgctttccctttgactgaatcgtaaaagtgaaagtgaagttcatcagaagtagattatgatact
taccaacccctttttcccttaaacaatctttaatctgttcactcacagatcattggaagcattgcagcagtttctgctag
cttagaccttggaaaagaagggccattggttcacattggcgcttgctttgcttccttactaggtcaaggtggtccagata
attaccggctcaggtggcgttggctccgttacttcaacaacgatcgggacaggcgagatcttatcacatgtgggtcatca
tcaggtgtgtgtgctgctttccgttctccagtaggtggtgtcctatttgctttagaggaagtggcaacatggtggagaag
tgcactcctctggagaactttcttcagcacggcagttgtggtggtgatactgagggccttcattgaatactgcaaatctg
gcaactgtggactttttggaagaggagggcttatcatgtttgatgtgagtggtgtcagtgttagctaccatgttgtggac
atcatccctgttgtagtgattggaatcataggcggacttttgggaagcctctacaatcatgtcctccacaaaattctgag
gctctacaatctgatcaacgagtaagcacctactcttccacattcccaactggatcatcaaacattcagttggttctcta
tattttaaaggcaatgcatatccacacaaaaatgagcttacttggattagaatcatcttgagacattgatccaactgtct
tgcatctttttaagtttaaatcctaattcctatccaaacatggccttcttatcacatttaactgccaaaaaaaaagggaa
aactatagatgcaaaatcctgactttcaatctttgatccttttttatcttgcaggaagggaaaactacataaggttcttc
tcgctctgagtgtctcccttttcacctccatttgcatgtatggacttccttttttggccaaatgcaagccttgtgatcca
tcacttcccgggtcttgtcctggtactggagggacaggaaacttcaagcagttcaactgcccagacggctattacaatga
tcttgctactcttctccttacaaccaacgatgatgcagtccgaaacattttctccataaacactcccggtgaattccaag
ttatgtctcttattatctacttcgttctgtattgcatattgggactcatcacttttgggattgctgtgccatctggtctc
ttccttccaatcatcctcatgggttcagcttatggtcgcttgcttgccattgccatgggatcttatacaaaaattgatcc
agggctgtatgcggttctcggagcagcttcccttatggctggttcaatgagaatgactgtttctctttgcgtcatatttc
ttgagctaacaaacaatcttctccttctgccaataacaatgctggttcttctaattgccaaaagtgtaggagactgcttc
aacctaagtatttatgaaataatattggagctgaaaggtctacctttcctggatgccaacccggagccatggatgagaaa
tatcactgctggtgagcttgctgatgtaaagccaccagtagttacactctgtggagttgagaaggtgggacgtatcgtag
aggccttgaagaacaccacatataacggattccctgtcgtcgatgaaggagtagtgccaccggtgggtctgccagttggg
gcaactgaattgcacggtcttgtcctaagaactcaccttcttttggttctcaagaaaaagtggttccttcatgaaagacg
gaggacagaggagtgggaagtgagagagaaattcacctggattgatttagctgagaggggcggtaagatcgaagatgtgt
tagttacaaaggatgaaatggagatgtatgtcgatttgcatcccctgactaacacaaccccttatactgtggtagaaagc
ttgtcagtggctaaggcaatggtgcttttcaggcaggtggggctccgccacatgctcattgtacccaaataccaagcagc
aggggtgagattataagcaaatttcagttatttttcttatgcaaatatctccctcctatcatagtataaagatgcacaga
aatagtcatatggtaatataagcacttgtttagaataattataggtggcaaagttattttacattagaagtgataaaagc
attacttacatcacacttgtgctccttttgtaggtatctcctgtggtgggaatcttgaccaggcaagacttgagagccca
caacattttgagtgtcttccctcatctggagaagtcaaaaagcggtaaaaaggggaactga
(DNA sequence of CLC-Nt2 from Nicotiana tabacum; sequence originating from
the ancestor N. tomentosiformis)
SEQ ID NO: 2
atggaggagccaactcgattagtagaagaagcaacgattaataacatggacagacaacagaatgaagaagaaagagatcc
agagagcaattcactgcatcagcctctcctcaagagaaacagaacactatcatccagtccatttgccttggttggagcta
aggtctcccatattgaaagtttagactatgagtaagaacaactaataatcttatctttagatcaagtatagcttttcttt
ataaatgggccaacagaaattggatgtcctaattttgtgtatctgctttaggatcaacgagaatgatctcttcaagcatg
actggagaagaagatccagagttcaagtattacagtatgtattcttgaaatggacactggcatttttggtcgggcttctt
acaggagtgacagcctcccttatcaatcttgcaatcgaaaacattgctggctacaaacttagagctgttgtgaactatat
cgaggatagaaggttggtgatgttttccctatgatcagcaattcataaaggctactataattcttcaatatgattccact
agctaatgactaagaacaagatcaaagatcacttatttgacttgaattatgttattgatttgttcataattgagattgta
acaatggttacaggtaccttgtgggatttgcatattttgcgggtgctaattttgtgctcactttgatagctgcccttctc
tgcgtgtgttttgcgcctactgctgcagggcctggaattcctgaaatcaaagcttatctcaacggtgtagatactcccaa
catgtacggagcaaccacactttttgtcaaggtgcgtcacgcacccaattttatcagtgctggcaattcaggtagcaggc
agattataacgccatcagtatagtattgagatcctgttgacctagatgtataaatagaaagaatagcagcaaataacaca
tttttagcctacatatttatggaattcaatctcatttgggaagtgatatctatctttcagtaatcagataggaagttgtt
taagaataaaaagagaattttatcgaatgcactcatccagcaaggaagatccatcagtggtatctaatctactcttgaac
ttccagtagttcaatcctttggtactgtcactgtaacttgttttctcatccaccattaaaatacaatagcttccatgaga
ctcagcttatatgtctcaattgaatatgctatttggtgactaacatgaatttgccctatcatgaaaataaatggaagtga
caaaaatacatatacttaaaagcacatatgtagagacacgcagactaagggcaaaggttcacaattttaaggcagttgtg
tccagaagaacaaatgaagaattatgatcacaaatttccacttgtaattgtactataaaatttttaattttgagagattc
tgacatgtttgctttccctttgattgaatcgtaaaagtgaaagtgaagttcatcagaagtagattatgatacttaccaac
tcctttttccccctaaacaatctttaatctcttcacttacagatcattggaagcattgcagcagtttctgctagcttaga
ccttggaaaagaagggccgttggttcacattggcgcttgttttgcttccttactaggtcaaggtggtccagataattacc
ggctcaaatggcgctggctccgttacttcaacaacgatcgggacaggcgagatctcatcacatgtgggtcatcatcaggt
gtgtgtgctgctttccgttctccagtaggtggtgtcctatttgctttagaggaagtggcaacatggtggagaagtgcact
cctctggagaactttcttcagcacggcagttgtggtggtgatactgagggccttcatagaatactgcaaatctggctact
gtggactttttggaagaggagggcttatcatgtttgatgtgagtggtgtcagtgttagctaccatgttgtggacatcatc
cctgttgttgtgattggaatcataggcggacttttgggaagcctctacaattgtgtcctccacaaagttctgaggctcta
caatctcatcaacgagtaagcaccaactcttccacattcccaactggatcatcaaacattcagttggttctctatattta
aaaggcaatgcatatccacacaaaaatgagcttacttggattagaatcatcttgagacattgatccaactgccttgcatc
tttttaagtttgaatcccaattcctatccaaacatggtctttttatcacatttaactgccaaaaaaagttactctataga
tgtaaaatcctgactttcaaactttgatccttttttatcttgcaggaagggaaaactacataaggttcttctcgctctga
gcgtctcccttttcacctccatttgcatgtatggacttccttttttggccaaatgcaagccttgtgattcatcacttcaa
gggtcttgtcctggcactggaggtacaggaaacttcaagcagttcaactgccctgacggctattacaatgatctcgctac
tcttctccttacaaccaacgatgatgcagtccgaaacattttctccataaacactcccggtgaattccatgttacgtctc
ttattatctacttcgttctgtattgtatcttgggactcatcacttttgggattgctgtgccatctggtctcttccttcca
atcatcctcatgggttcagcttatggtcgcttgcttgccattgccatgggatcttatacaaaaattgatccagggctgta
tgccgttctgggagcagcttcccttatggctggttcaatgagaatgactgtttctctttgcgtcatatttcttgagctaa
caaacaatcttctccttctgccaataacaatgctggttcttctaattgccaaaagtgtaggagactgctttaacctaagt
atttatgaaataatattggaactgaaaggtctacctttcctggatgccaacccggagccatggatgagaaatatcactgc
tggtgagcttgctgatgtaaagccaccagtagttacactttgtggagttgagaaggtgggacgtatcgtcgaggtcttga
agaacaccacatataacggattccctgtcgtcgatgaaggagtggtgccaccggtgggtctgccagttggggcaactgaa
ttgcacggtcttgtcctaagaactcaccttcttttggttctcaagaaaaagtggttccttaatgaaagacgaaggacaga
ggagtgggaagtgagagagaaattcacctggattgatttagctgagaggggcggtaagatcgaagatgtggtagttacga
aggatgaaatggagatgtatgtcgatttgcatcccctgactaacacaaccccttatactgtggtagaaagcttgtcagtg
gctaaggcaatggtgcttttcaggcaggtggggctccgccacatgctcattgtacccaaataccaagcagcaggggtgag
attataagcaaatttcagttattattcttatgcaaatatctccctcctatcatagtattaagatgcacagaaatagtcat
atcgtggcaaagttattttacgttagtaagtgataaaagcattacttacatcacacttgtgctccttttgtaggtatctc
cggtggtgggaatcttgaccaggcaagacttgagagcccacaacattttgagtgtcttccctcatctggagaagtcaaaa
agcggtaaaaaggggaactga
(DNA sequence of NtCLCe from Nicotiana tabacum; sequence originating from
the ancestor N. sylvestris; one start codon)
SEQ ID NO: 3
atgaatcacggaagttgttgggtcgt
catccaaattgctggccttgggctcgacgaccatctcttcctccgggacgttcctctgac
ggaaacattgaaaaagaacaagatatgtgcgacagcagcaaagtcgatagtgatagtggc
atccagataggatctctgctcgaggaagttatcccacaaggcaataataccgctataatc
tcggcttgctttgttggcctcttcaccggtatcagtgtcgtgcttttcaacgctgcggta
cgtgcgctataggtctttcatttctcttttcatgtactattcctccttacttacttggcc
tcagtcaatcagccccctgcctactttaaattattgtacattttatcagaggagtgtcct
atacatcaaattcacataacttagtaaaatatgctgatattctgaattttaaacttacca
gcttagaacatccaggttagttcagaaacagataatctaaattggtctcatttataagtc
attttgttattcaagacatacaatttggctcttgataaaagattatgcagcgcccgatga
ttacctaatatttatcagcaacccatgtaatttaacaatattgtcaccatataaaagaga
actgaagagaatgttcaatttgtggtcatataacggatatctcccttggttaggttcatg
aaatacgtgatctttgttgggatggaattccatatcgagctgcctcagaggagcccattg
gagtacattggcaacgtgtaatcttagtaccagcttgtggcggtttggtagtcagctttt
tgaatgccttccgagccactctggaggtttcaactgaaggaagttggacatcatctgtta
aatctgtattggaaccagttttgaagacaatggccgcttgtgtcacattaggaactggga
attccttaggaccagaaggccctagtgttgaaattggcacatctgttgccaagggagttg
gagctctgcttgataaaggtggtcgtagaaagctgtcactcaaggctgctggatcagctg
ctggaatcgcttctggtttgttccccatattattcttggttctgaaccatacatggtaca
ttttccttataattacatgtagcctgttgtatgctttcctctttcccgggaagccttttt
gtaaatacaagtgtgtttgcactcaaaccaataaactgtaaaaaaggtgaactccttaag
caagcaaaagcattagaaatgtaaactagacatatttctcagattgagagtctgagagat
tagaacacgagtgtttccattagagagagaaaagagacttctagatatttctattatctc
tgtaagagtgaatccgttcctatacaaaaaataggccttcattaaatacaagcttgggct
gggtactactgggccaaagtaaaaaataaaaagaatcacccactatcaaatgggcctagt
ctaacaacccccttcaagctggagggtgacacaacccctagcttgcgaatatgaaaatga
tgagcaggcccaagtaacactttggtaagaacatcaaccacttgagaagcactggagttg
tgaaatagactgatcaggccattcccaagcttgccacaaacaaaatgacagtccagctta
atgtgtttagtgcgttcatggaaaacttggttttttgcaatgtggacttcctgattatca
caaaataaaggaacaggtaaagaaggagaaactccaatatcagacaataatttggtgagc
caagacacctctgcaacagccttactcatggacctatactcagcttcaattgatgatagt
gagacaacaggttgcttctttgatttccagctcaccaagctgccccccaagaaaaataca
aaaaccagtgacagacctgcggctgtctgggcaagaagcccaatcactgcacaataaagc
tgcaaagacaagtctggagagttattgcggaagattccaaagtcaaaagtgcccttgagg
tatcttagcaagtgcagggcagcctgcatgttaggaacacagggagactgcataaactga
ctcagatgctgaacaacaaaactaaggtcaggccttgtgcgtatcaaaaagtttagcttg
tgcattagactcctgtactcttcaggcctgggcaaaggagtgccaatcttagcttttaac
ttcacattcaattcaagggggcaagtgacagaagagcaattcgaggaatgaaaatcagcc
agcaaatcatgaatgaactttttctgatgaagaagaaccccagaatcagtgtataaaacc
tcaatgctaaggaagtaattaagagagcccatgtccttaatcttgaactggtcactgaga
aaggacttcaaagcagccaattcagctagatcacacctagtcaatatgatatcattcaca
tagacaaccaagatgaccaaggaatccctagaacccttggtaaaaatagagaaatcattc
aaggaacgagagaagccattagagcacaaggcttgagataatttagcatactattgtctt
gaagccagtcttaaaccataaagagacttctggagtttgcatactaaaggagcagaagaa
gagtgaggaacagttaggcccggtggcagcttcatgaatacctcctcatcaaggtcccca
tgtaagaagacattattcacatctagttgaaagaggggccagtgttgtttaacagctaca
acaataagagttttgacaatagacatattgaccacaggagaaaaagtttcattaaagtca
ataccctcaacttgagtgacctagctttatatctctcaatactttcattagccctatatt
taaccttgtatacccacttacaactagtaggtttcttgccaggaggcaattcaacaatgt
cccaagttctgttggcatccaaggcctcaaattcacatctcatggctgcctgccattcag
gaacagctgcaacctgagagtaagaataaggctcaggaacatgaagttgactaagagaag
gagcattagaaatagatctggagggaggaggagaagaagtggaggtgcagacataactct
tgagatagttggttggattgtgtggcacggaagatcttctcaaagcaggaggaggtacaa
gagagttagaataatgagaaggagaagagatggaagtgggaacagagaagattgagaagc
agtagaaggagaaagtgaaggagatgaaggagaggaagaagacggaaaggaacattcatc
aaaacaagcagaaaagggaaaggggaagacttgaggtactacatgagaggattgaaagaa
aggaaaaatggtgttcataaaaaatgacatcttttgatacaaaacaggtgttattctgaa
gattaaggcgcttgtagccctttttggcaaaagggtagccaatgaaaacacaaggaaggg
acctaggatgaaatttgttttgtgaggggtggtgacagttgagtaacagaggcacccaaa
agctctaaggtggtgataagtagggtggaagaatgaagcaattcatagggacttttgtga
ttaagaagaggaaaaggaaatctgttaattaaatatgtggcagttaaaaagcagtcaccc
caaaatttaagtggtagatgagactgaaacataagtgacctagcagtctctagtaaattt
ctgtgttctctttctacaataccattttattggggggtgtgaggacaggaggtttggtgt
actatccctttttctgaaaagaaaaggcaaccagaagaactagatcccagttccaaagca
ttatcactcctaacagtttgaactttagattggaattgggtttcaaccatagcaatgaaa
accttgagcaaatcaaaggcattgcggcacccattaaatgtgtccaagtagccctagagt
agtcatctacaatggttaaaaaatacctagaaccattataggtaggagtagaatagggtc
accaagtatttatgtgtattagctgaaaaggctgggtggagtgaatagaactatcaggga
aggacaacctggtctgcctcgctaaaggacaaaccggactagtgaatgaccgtttggaag
acagtttgcaattaagaccagaaatgcatttcattttatagaagggaatatggccaagtt
tgtaatgccaaacaacatcatctttattcacattatgcaaagcagtactagtatttacaa
ttggagtatcatcaggtacagaaataggagcagaaactgaattaagcaaacaagaaataa
ggaaattagaaagaggtaaaggagatgatgttggaggcctggcattctgaaatagtttgt
agagtccattgtccaatctaccaagaaccactggcttcctcactgaagggccctgtaggg
tacaagtagccttggtaaattgtacaatatcatcatcatgggaaagtaatttgtacacaa
agatgagattatattgaaaactaggaatatagagcacattataaagaatcaagtcaggga
acaaggctaaggaaccaatattagtgaccttaaccttatacccattaggaagggagacaa
ggtatggtacaggaagtgtttgaacattaaaaaaacaaatgtttaagggaggtcatgtgg
tcagatgcccagggtctattactcaaactacactatctatcatagtcagcataaatgcac
cataagacaacccttgtgaggtaataactcaccagcaaagttggtagaagcaagatagtt
ggttgaagaagtagatgatgctgatgaagacagttgagattgttgaagtaacattagctg
agaatattggttcttggtaagaccaggaactggataggactgttcaggagcagaggtacc
ttcaggaccagctgacattgcagaaccaccagaggtatccacctcagcatgggcaacaga
ccttctgggaggaagagatctatttgacttgaaatttggaggaaagccattgagcttata
gcacttatcaatgctatgtccgggtttcttacaatagtagacatgtgaagctcaaaagat
cccttagaggtagtaccggacctttgaggttcaaaatttattttaggagagggaggaggc
ctggatacaccaacactgaaagaagcagaatttgaggcatattgagttctagcaaaaatt
tgtctttgcttctcatcagatagcaaaatcccatatacattaccaatggaaggtaagggc
ttcatcatgatgatgttgcttcttgtttggacataagtatcattcagtcccataaagaac
tggtagaccttttgttccctgtcttcagcagatttacccccacaagtacacattcaaact
ctcccggcagacaaagatgcaatatcatcccatagtcgtttaattttgttgaaatatgat
gctatgtccatggacccttgggaaatatgagccagttccttctttagctcaaagatccta
gtacctctcttctaactcagtccaaatattcttagcaaactcagagtattcaacactctt
ggatatttccttgtacatagagttagtcaaccaagagaccacaaggtcattgcaacgtta
ccactgtctggctagaggagaaccttcaggaggtctgtgagaagtaccattaatgaaatc
tagcttgttacgaatagacaaggcaactaggacattacgtctccaattgccataacagct
tccatcaaaaggaccggaaactaaggaagttcccagcacgtctgatggatggacatataa
ggggcgacagggatgggtataatcatcttcatggaaaattaggcgtaagggagtagaaga
agtcgcatcagcactggtgttattatcatttgccatttttttcaacagattgtcaatcaa
ccaacacaatacagatacacatatatagattgtgagaaagcacgagagaaaaatctatat
tattgatattctatttaattataatacaatgagccctatttatacaatacatatcatact
cctattctatgtgggactaggactaattcatattatgtacataactatctaacactcccc
ctcaagccggtgcatacaaatcatatgtaccgaacttgttacatatgtaactaatacaag
gaccagtaaggaacttggtgaaaatatctgcaaactgatcatttgacttcacaaactttg
tagcaatatctcatgagagtatcttttctctgacgaaatgacaattaatctcaatgtgtt
tagttctctcatgaaacaccggatttgatgctatatgaatggcagcttggttatcacaca
tcagttccatcttgctgacctcaccaaatttcaactaattaagtaaatgtttgatccaaa
ctagctcacaagttgtcacagccattgctcgatattctgcttctgcactagaccgagcaa
ccacattttgtttcttgctcttccaagacacctaattacctcctactaaaacacaatatc
cagacgtagaacatctgtcaaaaggtgatcctgcctagccagcatttgagtacccaacaa
tttgctcatggcctcgatcttcaaacaataatctgttacctggagctgattttatatatc
gaagaatgcagacaactgcatcccaatgactatcacaaggagaatccaagaactgactta
ccacactcactggaaaggaaatatcaggtctaatcactgtgaggtaatttaatttaccaa
ccagccgcctatatctagcaggatcgctaagcggctccccctgtcctggtagaagtttag
aattccgatccataggagtgtcaataggtctacaacgtgtcattcctgtctcctcaagaa
tgtctaaggcatacttcctttgtgagataacaatacatgtgctagactaagcgacctcaa
tacctagaaaatactttaatctgcccagatccttagtctgaaagtgctgaaagagatgtt
gtttcaacttagtaataccatcttgatcattgccggtaataacaatattatcaacataaa
ccaccagataaatactaagatttgaagaagaatgccgataaaacacagagtgatcagctt
cactacgagtcatgccgaactcttgaataactgtgctgaacttaccaaaccaggctcgag
gagactgttttagaccatagagggaccgacgcaaccgacatacaaggccactagactccc
cctgagcaacaaaaccaggtggttgctccatataaacttcacctcaaggtcaccacgaag
aaaagcattcttaatgtccaactgatagagaggccaatggagaacaacaaccatggatag
aaaaaggcggactgatgctattttagccacaggagagaaagtatcactgtaatcaagccc
aaatatctgagtataccctttggcaacaagacgagccttaagtcgatcaacctggccatc
tggaccaactttgactgcatacacccaacgacaaccaacaataaatttacccgaaggaag
aggaacaaactcccaagtaccactcgtatgtaaagcagacatctcgtcaatcatagcctg
tcaccaccctagatgagacagtgcttcacctggatggaaatagaggacaaagatgataca
aatgcacaatagggtgatgacagacgatggtaacttaaaccgacataatggggattagca
tttagtgtagaccgttcacctttccggagtgcaatcaattgactaagaggagacaagtcc
gcagtattagcaggatcaggtgcaggacgtgaatcagctgggcctgatgctgggcgcgga
cgacgatgataagttaggagtggtagagctgtagaaggttgaactggactaggcagtgga
actgaagctatatgtggtggaactggagctataggtggtggagctggagctgtaggtgaa
gatgaatgggagatagtgactgaatctccaaaagatggaactggtagcacctcagatata
tctaagtgattacctggactggtgaagtatgattgggtttcaaagaaggtaacatcagca
gacataaggtaccacctgaggtcaggagaatagcatcgatatcccttttgtgttctcgag
taacccaaaaatacgcacttaagagcacgaggagctaatttatcttttcttggagtaagg
ttatgaacaaaacacgtgctcccaaaggcacggggtggaagagagaacaaaggtaagtgg
ggaaacaagacagagaatggaacttgattctggatagctgaagatggcatacgattaata
agatagcaagatgtaagaactgcatccccccaaaaacgcaacggaacgtgagattgtatg
agtaaggtacgagcagtttcaataagatgtctattctttctttcagctacccgattttgt
tgggatgtgtatggacaagatgttttatgaataatcccatgagagttcataaactgttga
aatgggaaagacaaatactctaaggcattatcactacgaaatatgcggatagaaacccca
aattgattttgaatttcagcgtggaaggtctggaaagtagaaaacaactcagatcgattt
tttatcaaaaatatccaagtgcacctgtaataatcatcaatgaaactgacaaagtagcgg
aatcccaaggtagaactgacctgactaggaccccaaacatctgaatggactaaagtaaaa
ggtgactgactctgctcgattatcaagacggcgagggaaatgggagcacgtatgcttacc
gagctgacatgactcacactctagagtggacaagtgagataaaccagataccattttttg
aagttttgacaaactgggatgtcccaaccgtttatgtaatagatctggtgaatcagtaac
aggacaagttgttgaagaaagacaagatgtaagtccatgtgattttgcaagaataaggta
gtaaaatccatttaattcacgcccggtaccaatgatccgccctgtactgcgttcctgtat
aaaaacaaggtcatcaagaaataaaacagagcatttaagtgatttggctaagcgactaac
ggctatgagattaaaaagactaacgagaacataaagaactgaatctaaaggtaaggaagg
aagtggacttacttggcttattccagttgccatggtttgagactcgttatccattgtgac
tgttgggagtgattgagaatatgaaatagtaatgaaaagagatttgttaccaaaaatatg
atcagatgcacctgaatcaatgacccaagactcagaggttgaagattgggagacacaagt
cacactactatctgtttgagcaacggaagctatccctgaagatgtttgtttacatgtttt
gaactgaaggaactcaatataatccggtagagaaaccatccaactcttcgtagtattgga
ttccattttgctacaaccaatttctcaaattcttgattacaacttgtgtggttaaccttg
gaatgccaaatcagaacaccccttttttttttttggaaaacattgttcactcgctggaaa
ataaaaaaggttgccggaatttgatgaaacttgaatagaccgactcggaataatgtccta
agaaggctgtccaaaaggagttttgtcagaaactgaccagaaggaggtccacgcaccggc
gcgtggacagatctcgccgaaaaaaaaaatcactttggttggcgcgtgatggcgcgtggg
tggggtttttccggtcgggttttgtggggtttgctcccccggagatggagaacactgtgg
tggtgttggtttatgcacaacactggtaaaaagtggttttgatgcgaacagctactcagg
tcaccaaaaaattgcacggtgacgactgatttcttcccggatgtcgttggaatgacgcac
aacgataattatctcaccaatgctctgataccatgtgagaaagtacgggagaaaaatcta
tattattgatattctatttaattataatacaatgagccctatttataagactaggattaa
ttcatattatgtacataactatctaacatagatcaaataggcatgcaattcacaataatg
gtgaataaaatgatacgaagttacccagctcttttcgcgatcgaaaaggagaaaatagcc
ttcaatcacaaacgagaaagaagaatctccggcttgacagtagacgacttcgaaacccta
gctcgagatgaaaaccacaaaatccccaaatcacattaccaaccaaacaatttgagatca
caaatgttgaatatgtgagaatccgactaagaaatcaacaaaaaatcaatagaaatggtt
gaagaataccgacttgaaccctaaatgagtcagacatcacctagaatgaaatacaccttc
gaaattgacgaaaacaggaccggttgaaagcggagaacgtgccatagaaggatctacgct
ctgataccatgtaaacttgacatacttctcagattgagagtctgagagattagaaaacga
gtgtttccattagaaagagagaaaagagacttctagatatttcgattatctgtgtaaaaa
tgaatccgttcctatacaaaaattaggccttcattaaatacaagattcggccgggtatta
ctggcccaaagtaaaatataaaaagaatcacccactatcaaatgggcctagtctaacaag
aaaaccaacaaatagtccccccccccccccccaaaagataccactgaaatgacaccgggt
gcccaaaaataaagcagcttacttcttgactttgagaggaactgcaatccttatcggttt
gagaggaactgcaatcagctataagtagcttattaatttccagtgcctgcattctgccaa
gtactatgatatatttctgaagctttgtttccccagttcctttttcagacgtttgctgtc
aataaagttgagccagccaacttggctcccacaagctactaattttgtccaagcttactc
tatgggagaagttaaatttcccaaattccttgagcggaaaatgaaaaatggactcaaagt
gtcatattatgcaactatctaaagaaaaatactcaattgaagtttagataagaaaagtga
atgtatattgatgtagtctccgttaggtgagaagcgtatcacttacccagcaacatatgg
acctaacattttactagtgaagttttcacattgtatcaaaagctcaacaaacggaaaggt
gactaatcctaaaatgttatttcacatatatgggcacacggtttgtcaaccttctcatac
gtgcattatttgttctctatctttctatttcatccgatataaccaatcgttattgtaaat
tctataatgcctgtggttacttttgtctttagtgacaaatgacatttaggataaccatgt
agttattgacttatttcacttgaggtctcttccaattatgtagtagtagagtgttgagat
atggatatgttaccttctaaaaaaaagagtgtagagatgcggatagtttgctagctggct
tttgtctcccttcaagttgaattagcaaaagcttgtctcataagttggatagctagacaa
gaaaaactccaaattactttatgtagagtattcttaagcttgagtcgcgagttggaaact
ggaattatgtaaaaaaacctggaattatttggttgagcctgctttttagttttgtcaata
tttccagtatctaacccaacatgtttagagtgattcccggagagcctcagtacaaggcat
ttgcagagtctttatgagagtccaggaaggggcacacattctgtagaggtatagtcttgt
ccttattttcagggttgaactagttctttagaagttacctaggcttcctaatttccaaat
ttctgccaggtccttttttggtgaagtacttgaagtttaataaatcaaattttaatttct
aacatatcctgagaaatttattcacaaattcaactggtgacttctgatgcagaaacataa
gcaactgcttatgggttcatatgttcctgcaattttattgttgacatggattggcttcat
atggttttgttcctgcaattttatcgctgacactaatcctttcatatggttttatgtgga
gtgttaaatagaggttaagagacaagaagaggctgaaaaaggtgggcagttcatttgtta
gtagactactctatttactaagagatatgatgtcccatacattactcgaattggctccga
atccagattccacttctttgccgagtttccttattgtacatagttcgactcgtcaaggga
aattcacttcctttgactgaataatgctagtttgagtagtaccttacattaaatggacca
tttagttctatctacttgatagaatagactggtcatcaactagttgcaaatacaatgaca
actttgccatgtttgcagagtcacctgatgaagaagtacctcaattagtagaacatttct
tgaatgttctacagtattctctatgcctacatgaccacatcacttttccttttgcgttgt
gagaacttgaacttggtgagcgggggttccccaggaatggcatcttgatggcagatgacc
attctgtccttgtcttagctaatgcttcttgcattgcctcactagatttattataccttt
aaaaaatgtttgccattgttctgccataatagaaggatgtacccagctggtgcttcaaaa
ctaatgaaatgctttacaattgtcgagtcctaaaggatgatttgtggaatcagatctcaa
acaattctttttgaggaagaaaaataccaaaggttttttctgtttgttggaagattaaaa
atcctttaaatggtaaagatttatgaacttaattcagcgtttttgtggccattgctggaa
aagagaaaaaacaatggcacttcttcgagtttgcttatccaaaaaaaagaagaagagaat
gtcacgtaatgcaatttcatcttaggaaactttgcaggagaaaagcaagagtgataaaac
agaactatttgttttttttaacaagttgttgtgacctatttcttgtcattcttatttgct
aataagctaatgtactatagttcctgtactatggtttgttttgacttaatacggggatgt
tcaatgagcattttcttgttttttctgctttcagcatctgctgccttacaggaattcatt
ttctggaaatttacttcttgttctgctaacattttcctgttatatcttgtcagtcatttt
ctctccatggttatactgtttgtgtcactttaaactctccttgttttctactttaaagga
tttaatgctgctgtcgggggctgtttctttgctgtggaatctgtgttatggccatcacct
gcagagtcctccttgtccttaacaaatacgacttcaatggttattctcagtgctgttata
gcttctgtagtctcagaaattggtcttggctctgaacctgcatttgcggtcccaggatat
gattttcgtacacctactggtaattttggacttctttctcgagtttgattcttaaataca
attgtacccgtcacttacagcaacaactacatttcaacagctagttggggttggctacac
agatcatcactatccatttcaattcatttagtcccatttctttcgaatattgagtacttt
gggattctataatatcaaggttctttatattttctactttgacgtacaaatctctaaata
gattaaagaagactcctagagacactggcctaatgcaaatgtaccaccatgaataaactt
taatctgaaatagctggtatcttatataaggacccttagctttaattgtgttctatattg
atcttttgggacaacttccttccaatattatgtcttacttatacagttatacttatcctt
aagccttactctttagagtggttatccctaattcaagcttttgttggcaccatagctagt
ttggttctaagtaaaaagttactctttagagtggtaactttttgtcaattttcttagtga
aaatataacctctgtgacaaatctaccaagtataaatccaatttggttctatgtcatcct
tgtagtttatccaagtcaatgctccatcactcttacaaaggttcatcgtatgactaatct
tttttggagaaaggtaacagtttgtattgataataagatcagcgccaggttggtcattag
tgctaatagctgtacgtacaactccaaaagagcaaaagacaagcacctgatgtaaggtaa
attacaagctgcctataaaatctatcaggtgtcctatctcactaaacatttcttgtttac
accaaaaaaataaaacaaggaaagacaatccatcttaatcttctgaatggagtttctttt
tccttcaaaacatctggagttccttccgttccatgcaatccaccatatacaagctgggat
gattttccatttgtctttatccatttcttctaccaattcccttccaattgattagaagtt
ccaatgtggttctagatatgacccaattaactcccaacagataaaagaagatgtgccacg
gatttgtagtgattctgcaatgtaggaacaagtgagcattactttctacttcctgtccac
aaagaaaacatcttgagcaaatctggaaacctcttctttgtaagttatcatgtgttaaac
atgcctttttcaccaccaaccagacaaaacatgatactttgggaggagttttaaccctcc
aaatgtgtttccaaggccacacctcagttgttgaaacattaggatgtagagtccagtatg
ctcttttactgaaaatgcaccttttctattcagcttttaaactactttatctatggtctg
tgatgtacccttgaaaggttcaagagtttggaggaagatagaaactctgtttatctccca
atcatccaaagatcttctaaagttccagctccatccttgtgagctccagactgacttacc
aatgcttggctttgaagacttagagagaataagtcaggaaaatatctttcaaccttcctt
gccctatccggtgatcttcccaaaaagatgtctgcaacccattgccaatattgatcttga
tattgctactgaaagatttcttttggtggcaggattactctcattaacaatgtacttgac
aatctccatacatactaatgtctctttaccctcttgccattaaggttgtaaagagacttg
tcaaattaagaaaaggtttcctatggaactgtttcaaggaaggaacctcctttcctttgg
tcaagtggagttaagtcatataatctaggaagtggaggcttgggtatgaaatagctgcaa
atacagaaaaggagcatcttatttaaatgatcacggaaatgtgcccaaaactttaaatat
ctgcacagcatatggttgtagcaaaatttgaatcttcctgtcaatggtgctcatgtccag
tgaatacccctgatggtgaaagtgtcctgaagggaagcaggaacttattggaagaattgg
catctaacactcagcttttcggtgggtcatagcccattgaaaattgagtgcccagattta
tatagttttgctctaaactgacgatgcagttgcacaacatacgacaaactaaggtgggac
atcatcttcttcggaaggaattttgaggattaagagatagagtggttgattcagttgcaa
atgaagcttcaagggttcaatatcatccaggagacaccggattctgatagataaaacaac
agaaagatgagcactactttgttaggcttgttacaagttgctatcgtctttcttatctcg
gtacacaatttagatttgggaacttagttggaaaagcagagtggttgtttttgtgaatag
catcagacaaagcttctgagctggtacgacagaaaactcaacagggagaatagaagactg
tggttcacaatttctgcatgcatcttgtaggttatttggtgggtaaattatttaatgttt
tgaagggaaggtagaacatgttcataggcttagattcaaatgtttgtatttttttggctc
tttggtgagagatgctgaacgtaaatgacataggcagctgactataatttctcagctcct
tgctttttaaattgacaggcactgatatgtacatgtgaacatccaacacttttgtggtgc
cgttccgatgaataaagaacattaatcacttactgatcaggagtaatagtttaggagttc
tagaatttttgtacataaaatgaaccaaaaagaagatcggaatgagaacatgtttctttt
tttgttttttctttttcgtgaaaacttcaataacacttctgatagaatagctaggtccat
ttgaattcctttggagacccttacacaaccaatgaatgacaagtatagcatttctaactc
cctcccacacgtataacccagattttagggtttagatgtggatctgatttgaccttattg
cctttttttgtttttgttctttttgaagtagagagtgaggaggctcaacaattaattcgg
ctcaacgggctaatgattggacttacatgctacgacaatgttaggagagagagagagaga
gagaagcccagagcagttacatgagttaagaaagagaagtccaaagcgatagaatatgaa
gagagaaagcggttgtgctaacaggctccctgaagtttggctctgagcatccaactcaaa
accttaaggcaatgagtagagtagcccaggaccatttaaattgctgttgaaaaccttaca
caaccaataagggaacaagtgtaacattctcttacaaccctaccgtcttataagtcagtg
ctctaatttagcataaaatcaaagtgaggcgatctacaatgaaatgaagtaaataactga
taaatacaaagaatgttaattctccaatatagcctgaatgttcccagaacaaaataaact
agtctcaggatttatcattaacatgatgttcctcttattttgagtgattaggaaggttaa
tcaaggtataaattctttctaatttgtatcgtctagaattatttatctaacaaattttca
gattaccggttcaaaagaggaatatattttgcatacaacgttaccataccttacaaaagg
gagatgaacatttttttattttattattgtcctttttttcaattagggattatgcagtct
tcctccacgtgatattactcttagaatcacgtttttgtcattgctattacttaatgtggt
aagtacaaatgtgttttgaactctttttggtatgtaatattgagttaatttttggtttcc
atttcagagctgccgctttatcttctgctgggcatcttttgtggcttagtttcagtggca
ttatcaagttgtacatcatttatgctgcaaatagtggaaaatattcaaacgaccagcggc
atgccaaaagcagcttttcctgtcctgggtggtcttctggttgggctggtagctttagca
tatcctgaaatcctttaccagggttttgagaatgttaatattttgctagaatctcgccca
ctagtgaaaggcctctccgctgatctgttgctccagcttgtagctgtcaaaatagtaaca
acttcattatgtcgagcctctggattggttggaggctactatgcaccatctctattcatc
ggtgctgctactggaactgcatatgggaaaattgttagctacattatctctcatgctgat
ccaatctttcatctttccatcttggaagttgcatccccacaagcatatggcctggtatga
atttgtcttttgttagaagtagcattacatatctggataagtgagttttttattattgaa
aagtaataacaggagagcaagagaatatagcacccaaatctacttctttcctctcttcta
ttcttctgaaattcaaggtcctttaactcctccacggcctgtctagttattgatcctgta
gacttaattcacataggtttaggacattcaagtttatccaaacttcgtgaaaaggtttct
aatttttttacattacagtatgagtcgtgtctacttgagaaacatatcactccatgtttc
tatagagtctgttttctcctcagtttattttgatatatggggtcctattaagacagttca
accttggattttcattatttttgttgtttcattgataattattcaagatgtacttggatt
ttcttaacaagagatagttctcagttgttttttgtgttcctaagtttttgtgctgcaata
caaaattagtttgatgtctctatttgcatttttcccaatgataatgccttagaatatttt
cttctcggtttcagtagcttatgatttctttagaaactctctatcagaaatctcaactga
gatagatgagaggaagaataagcatatcattgagacggctcgtacccttctcattcagtc
ccctgtcaagcttagtttcttgggcgatgcagtttcacgtcctttgattagattaattgg
atgcctcatctgctatccaaaatcagattcaactttcgatattgtttcctcgcttacctt
tatactctctttccctcgagtctttgggagcacatgttttgttcaataacatagctcctg
gaaagtgaccagcgcaaccgacaagcaaggccttcttaatatagaaggagggcatatgct
attctagccacgagggagaaagtaatattgtaatcaaacccaaatatctgagtataacct
ttggcaatggcgatcaatttgattatatggaccaactttgcctacatatacccaccgata
gatttacggggaggtagagaaataagctcccaagtaccactaatatgtaaagcagacatc
tctttgatcatagcctgtccttgtggacatagggatagaaattgaggactaagatgacac
aaaagcataatgctgtgatgataaacgatgataactcaaatcaatatgatggggatggga
attaagagtggattgaatatctttgcggaatgtgattggtagactaggaggagacaagtc
cgcaataggtaaaagatccagtacatggaatgaatcttctggacatgatgttggactgac
gtcaatgataagtcaagagtggtggagttgcagaacatggaactggagctgtaggtgaca
taatcgaagttgtagggggtggagctatagaggaaggtgaaggagagatagtgactgaat
ctccaaaatatgaaaccggtaatacctcaaaaaatgtctaagagatcatttggacctatg
aagtatggttgcgttttaaagaaggtaacatcagcagacataaggtaccgcggaaagtca
ggtgaataacattgatatccttgttgcgtcctcgagtaacttagaaatacatatttgaga
gcacggggagctaacttatcttttctggagtaaggttataaaaaaacacatgctcccata
gacacgaggtggaagagagaaaggtgagtggggaaacaagacagagtatgaaacttgatt
cttgatagttgaagatggcatacaattaataagacaataggatgtgagaactgtatcccc
acgtaaacacaacagaacatgagattgtacgagttgggtatgagcagtctcaatgagata
cctattcttcctttcagctatcccattttattgagatgtgtatggacaaaatatttgatg
tatgatcctatgagagttcatgaactgctgaaatggagaagacaaatactctggggcatt
atcactatgaaatgtgcggttagaaaccccaaattgattttggatttcagagtgaaaggt
ctgaaaaatagagaccaactcagattgatttttcatgagaaatatccaagtggacttgga
ataatcatcaatgaaactgacaaagtagcagaattccaaggtagaactaactcgacaagg
acctcaaacatctgaatggactaaagtgaaaggtgactctattcgattatcaagacaccg
aggaaaatgagagcgagtatgccttctgagcggatatgactgacgctctagagtggacaa
gtgagacaaaccaggtaccattttctgaagttctgataaattgggatgtcctaaccgttt
atgtaataaatctggtggatcagtaaaaggacaagctgtaaggggacaaaaataccaaat
atttccagaagatggcaaactacaacagaagaagcaactacattaacaggctcaggatat
gtgatgaaatgaggacaaagagttgatcaagaaggagattctggaattctaccagaactt
atatagtgaaaatgaaccgtggaggcccagtgcaaattttgaaggcatctcctcactaag
catagaagagaagaactagttggaagctccatttgaagaaatagaggtgcttgaagcttt
gaaatcatgtgcccctgataaagcaccaggtccagacggcttcaccatggctttctttca
gaaaaattgggatactcttaaaatggacatcatggccgcacttaatcactttcaccagag
ctgtcacatggttagggcttgcaatgccaccttcatcgccttaattccaaagaaaaaggg
tgctatggagctcagagactacagatctattgacaaactagtctcgggggaacaaaatgc
tttcatcaagaacaggcacatcactgatgcttccttgattgccagtgaagtgctggattg
gagaatgaaaagtggaaaaccaggcgtgttgtgcaaactggacattgaaaaggcttttga
tcaattaagatggtcttacctcatgagtatcttgaggcagatggctttggggagaaatgg
ataagatggataaactattgcatttcaactgtcaagaactctgttttggtgaatagtggc
ccgaccggttttttctcctgccaaaagggcctaaggcaggggatctcctctcccctttcc
tattcattttggcgatggaaggactcactaaaatgttggagaaggctaagcaactacaat
ggatacaaggctttcaggtgggaaggaatcctgccagctcagttacagtatcccatctac
tctttgcggatgatactcttattttttgtggtactgagagatcacaagcacgaaatctca
acctgacgctgatgatcttcgaggcactatcaggactccacaacaatatgataaagagca
tcatataccctgtgaatgcagtccccaacatacaggagctagcagacatcctatgctgca
aaacagatactttcccaacatatcttggacttcccttgggagctaaattcaaatcaaaag
aagtttggaatggagtcctagagaagtttgaaaagaggcttgcgacttggcgaatgcaat
acctctccatcggtggcaagttaactttaatcaatagtgtactggacagtcttcctacat
accacatgtctttgttcccaattccaatctcagtcctaaagcagatggacaaactcagaa
ggaagttcttacgggaaggatgcagcaaaacacacaaatttccactagtgaaatgactca
aggtaactcaaccaaaattcaaaggaggcttgagcatcagggatctacaagcacacaaca
aagctatgctcttaaaatggctctggagatatggacaggaggaatctaggctatggaagg
acatcatagttgctaaatatggagcacacaatcactggtgttccaagaaaacaaacactc
cttatggagttggtctgtggaagaacatcagcaaccactgggatgaattcttccaaaatg
taactttcaaagttgggaatggaactcgtattaagttttggaaggatagatggctcggaa
atacacctttgaaagacatgtttcccggtatgtatcagattgccttgaccaaagactcca
ctgttgctcaaaatagagacaatggcacttggtgcccattttcagaagaaatttgcagga
ttgggaggtcaacagcctactcacaatgttaagctccctagaaggtcataatatcgaaga
tcaacagcctgacaaacttatttggggaaattctgagagaggcaagtacacagtcaaaga
atgatacattcacctctgtgaccagaatccaataatagataactagccatggaaacacat
ctggagaactgaagtgcctaccaaggtgacttgcttcacatggttgactctaaatggggc
atgtctcactcaagacaacttaatcaagaggaatatcatactagttaatagatgctacat
gtgccaacaacagtcagaaagtgtaaaccacctattcctccactgctcagttgcaaaaga
catttggaacttcttctacactacctttggtctgaaatgggttatgccacaatcaacaaa
gcaagcttttgaaagttggtatttttggagagttgacaaatccatcaaaaaaatctggaa
aacggtgccggctgcatttttttggtgtatttggaaagaaaggaaccgaagatgttttga
tgacatattaactccactctactccctcaaggctgcgtgtttagttaacttatttagttt
tgtggattttattagctccctgatagtagcataggcttttgtaaatggagctaattatcc
tatctcttttgtactctttgcatcttcttgatgccttttaatgaatctaatttacttcat
aaaaaataaaaggacaagttgttgaaggaggaaaagatgtgagtccatgtgatttagcaa
ggataaggtactaaagtccatttgattcacgcccggtaccaatgatccatcccgcattgc
attcctgtattaaaacagagtcatcaagaaataaaatagagcaaataagtgattggccaa
acgactagtggatatgagattaaaaggactatcgggaacataaagaactgaattcaaagg
taaggaaggaagtggactagcttaacctattccagttgccatggtttgagaatagttggc
cattgtgactgttggaagtgattgagagtaagaaatagtagtgaaaagagatttgttacc
agaaatataatcagatgcaactgaatcaataacctaagagtcggaaaaagaaacacaagt
catgttattacctgtttgaacaatagaagttatctccgaagaggattatttacatgtttt
gtactgatggaactcaatataagccgataaagaaaccatccggatattcaaagtattgga
tcaacagcttataagccaaaagcatccgatacgagtgccattataatggatcaagagaga
tcaaacaacaaatcaccaaatatcataaacaaccaagaatctcgctggaatgtgaacaaa
gattgaaaaacaacaatgtagctcgccaaaaatgtgcaaagtgatcgaaaaatattgaat
cgtgagtggagagaaataggagcttcaatcgacccacacagtaccaaaaaatccaaaaac
ggttgtcggagctcaagaaagttgtcaaaaagtatattgtatgcttcgaaagtagccgaa
aaaggttggaagtgggatgtgtcaactccgaattatgatacgagcaccacagaagatcaa
tttgtgtcaaaactaccgaaaaaaatacttcacaccccgacgcgtggagtactcgctcgt
tggaacccttgctgccaacgtcgcatgtaggatcagttttcgaagaatcttattggggtt
tggtcgccggacgatgtcggatcttgtggtgccgttggaattcgcacaaccctgaaggaa
aagaaggttacacaaatcagatctgaaagtcaccgaaaagacacatggcgattgactttt
ttgtctcagatgtttctcaccgtcgctctgataccagttgttgggctcaactcgtttgaa
gatactcttaacatagtgtgatattgtcccttttggaatgtgagtcatcttagctcggta
agcatactcgctcttccaactagcccgaagatacttttaacagagtgtaatattatctgc
tttgagccaagctggcgcggttttcatcaaaagacctcatactattaaaagatccataca
ccttatatgtaggcttctaagttgctcggacacgggtgcgagtacccgacacaggtgcaa
atctagaggtcagatcctttaaaatgtaaattctaagatttggggatacgaatcctagta
cggatacgggtgcgaggatccgattaaaaataattcaaaaaaataagaaaataaaaaagt
ctctaaattatgtgaaattttgtggaataactacgtatagcttgtaaagtgtggatttat
tttttattctcaagttgtagataagtaaatgattgatttcctagataaggtatgttattt
tcttcaaatttaccctagtttggttcgaatttcgggaaattgtatcttgtctcgaatttt
tccttctgtcctgattaaactactcaaaatcgtctgaccagatccggtacggatcccata
cccacatccacactagtgtcgtgtggacaagggtgcggcacctaaacttccgtgtaggag
caatttaggtaggctcctaatcttttcagctattaatgtgggacttttacgcacctctat
caaattccccaataaactaagtttcacgtggtccatcatcgcaatccacgggtctcttcc
tctagttaagtcccacatggcccattaccatgatccacgggtcaattttcgtgattcatc
gtgtgccacccacatcgttagtatttatggtaactaaagtacgcaactagcttttgcttg
tgagcgtgtctccaagctcgtaaaggtaagaaaaccgagccgcatattccatcactctat
catcaccatactcgtcccgcgaaacttgtaagataaaggtggctggttggtcagttgaac
tacctcagagtgacttggtatagtatttcctttcttgtgaatatttaactcaattatgga
ctctctgtgtgatagtcattgagagccattttctatatagccggtgcacacaaatcatat
gtaccaagcttgttatatatgtaactaatacgaggaccagtgaaggactcggtgaaaata
tctgcaatctggtcattcgacatacaaggccaatagactccccagcaataaaatcagggg
gttgctgataaatagaattggccgaaatgttgccagaaaaatttgaaaatagtgagacta
agccgaattctacactacaaaataggttttaaaacacaaccagaaaacaaaaactttttt
ggaaattactgttcacatcgaaaaaataaaagttgtcagaatttgatgtaatttatatgg
ataggctcgtaatcactggacgagtaagttgtcctgaagaagttttgtcaaaaggtggcc
ggaatggctcacacatgccggaaaacttattgtagctcgccggaaccctagttctggcgg
tgcgtagaggcgtgtgactttctgccagactgattgactgtggtttgtcgcctgactttt
cctaacaagatggtagtattggttttcgcacaacaattaccgatgaggagataacgcaaa
tcaatcttgagtcgtcaatcggaaagacgcacggtggctgactttctatttagatgggac
tggaatttctggagtttaatcgcacaagcgttttggatctgatggtaatactggtatgca
cagtaccactgtagcagtgatgaaccctcaaaataagacaaagttgccagaaaattgcac
ggcgatgagatctttcttccggatgtcaccggaatgacgcacaacgataatttctcactg
aagctctgacaccatgtgagaatacacgggagaaaaatctatttttattaacaatgatac
aatgagccctatatataatacatattctactctactacatatgggaatagggcatatttt
actcctactacatatgagactaggactatttacacataactatctaacaagggctatatc
tcagatttatgagaatatctacccaacgacccagagagacgagcctaatcattttgcagt
ggcacagactataacaacaaaaaacctactcataatggttaaaccaactgattaagatgc
ttacaggactatcttgagaaatgtacatattatatagatgcttgagttgcgtcccaatcc
taaatagaagcttttattcgtaagcaagaagggaagcagctttacttgagccaatagctt
tcaaggtgcatgttgtcacaccaaggacatccagaatttgattttatagtgggaatatcg
tttaaagataaaaaagatagcgtgcagaagattgcatacattagagatgcaaaatacgga
atacccatactcccagataatgcagtatgccttttgcatgacctactggttgaatggaag
cacctggtgaatttactaggtgtgttagtgatttctgctgcttccttcccctttctaaac
tgcatactatctaaaatgttaggggggcagaagcccagtcaatctgactaggtgatgtta
gtggtttccgcttcttcctcccacttctaaatgcgtactttctcaaatttaggagcatag
aaacttaagcagctgcctacctgaggagttgcatgggaacataagagaatagactttacc
tgtcatattttccataccttagttaattacagtgttatcctgataatgatctgttttctg
gatctaggctgaatcgagattcaatcgcttttggttgaaaggatgctgctacagatcctt
agtttacatcattttggttcttattctataagtacttcccctatcaactacttccttctt
ttttcttaggttatttgcctctttaggttgtttggaaggaaaggaacagtagatgttttg
atggaatagcaactccaaaccacttccttaaggctaatatcctgattggccaagtttctc
caaagtccaaaacactttttttttccttcaaaaaagtacctttttttttcaaagttgagg
tgtttggccaagcttttggaaggaaaaaaagtgtttttgagtagaagcagatgctcttga
gaagcagaagaagtagcttcttcccggaagcacttttgagaaaaataaatttagaaacac
tttttaaaagcttggccaaacactaattgctgcttaaaagtattttcagatttattagac
aaacacaaactgcttctcaccaaaaatacttttttgaaaagtacttttcaaacaaagcac
ttttcaaaataagttttttagaagcttggctaaacaggctataaatgtcttttattttta
cagctggagtaccctaacacctgtaaattcccctatacatttttttcgactttggtagct
cattaaccctagtataggactctttgttttggagctagcaaactcttttgttttcctatt
tttgcatcttcttggtgccatttataatatctcttcaccaaaaaaaaaaagttcccaaac
tatgactaccttgagttggtcaaagcataaccaaagcatgggcacaccagtgtttgcgtg
aattttatggatgttccttacctttatccttctgtgcttatgtagcatctgtcttggtca
atcttttctgaagtctatattgtatttctgtgttgcaacatgagtttactgttaatctta
ctgtttgacctcaattttgggttctttttgattttggaagacatcgtttaacaggttggc
atggctgctactcttgctggtgtctgtcaggtgcctctcactgcggttttgcttctcttt
gaactgacacaggattatcggatagttctgcccctcttgggagctgtggggttgtcttct
tgggttacatctggacaaacaaggaaaagtgtagtgaaggatagagaaaaactaaaagat
gcaagagcccacatgatgcagcgacaaggaacttctttctccaacatttctagtttaact
tattcttcaggttcaccttcacagaaagagagtaacctctgcaaacttgagagttccctc
tgtctttatgaatctgatgatgaagaaaatgatttggcaaggacaattctagtttcacag
gcaatgagaacacgatatgtgacagttctaatgagcaccttgctaatggagaccatatcc
ctcatgctagctgagaagcaatcttgtgcaataatagttgatgaaaataattttctcatt
ggtctgctgacacttggtgatatccagaattacagcaagttgccaagaacagagggcaat
ttccaggaggtagcttcttggtacatttcaatattcttaactgatgaaaaaataagggaa
attgatctagcatgaaatgaagctaattataagttttacacagtagaactggtaaaacag
ggttggctggatatttctttgttgaatttttaggattatatatattgttttagttttgta
ggttgttttctgatgtgctttttgactcggcagaatcttaagatgaaatggaaggttgta
tcatcaaatgttaaataagggaatatgtgactttcaaagttaagcacggagtattttgga
gtcaatagttacttcctgaatcttttaggatggaggagacagtttctataggaataggaa
aaggggacctgatttcattatttgtgtgtatatacatttgttatctgaattcgcattact
ttctaacaaccaacaaaaggaaagtggacattcaatttgagccggagggagaaaatttaa
ctagaaaatgacctggccgtgaaataaaattattgatccgtcctttaactagttttcatg
gattgcctccttgcggatgatttttccaaccggtagaactactgttagtcgtccaaattc
tgaccccctactatgaataaaaatgtattagtaagtttagtgggtaatctccttgagaaa
taaaggaacaggagaaatattttattgatatatgctaagtgttttacaatagccctattt
atatacaatgtttacataaacctaaagccttctatataaatgtgggacactatacatgaa
ctaactctaacactatccctcaagctagtgcatataaattatatatatgcttgttacata
tataattaatttctctactttttggtatacttcttgtatacgggagttatctcccttttg
attaatacaatttaccttatcaaaaaaaaattaatacgaggaccagtgagggacttggtg
aaaatatctgcaagttgatcatttgacttctcaaactttgtaacaatatctcctgagaat
cttctctctcgtgaagtgacagtcaatctcagtgtgtttggtcctctcatggaacactgg
atttgatgcaatatgaaggacaacttgattatcacacacaagttccatctgactgattgc
tccaaattttaattatttgagcaattgtttgatccaaactagctcacatggtgcaagagt
catgactcgatattcggcttctgcgctagatcgagcaactacattctgtttcttgctttt
ccgagagacaaattacctcctattaaaacacaatatccagatacgtaacgtctatcagaa
ggtgaccctgcccaattagcatctgtgcgtccaacaatatgctcatggcatcgatcttcg
aatattagtcatttgtctggagctgattttatataacgaacaatgcgaacaactgcatcc
caatgactatcgcaaggaaattccataaactgacttacaacactcacaggaaataaaata
tcaggtctagtaattatgaggtaattcaattttccaaccaggcgcctatattttgcagga
ttgctaagaggctcccccctatcctggcagaagcttagcattcggattcataagagtatc
aatagttctgcagcccattattcatgtctcctcaagaatgtctaaagcatacttcctttg
cgaaataacaacctgaactagaccgagcgacctcaatacctacaaagtacttcaatctgc
taaggtcgttagtctggaagtgttgaaagtgatgttgtttcaaattagtaataccatcct
gatcattgcgagtaataacaatatcatcaacataaaccaccagataaatacagagattag
gagcagaatgccgataaaatacagagtgatcagcttcactattagtcatgccaaattccc
gaataattgtcctgaacttacgaaactaggctcgacgagattgttttaaaccatagagac
ttgcataagtgacatacaatacctctagactccccttgagcaacaaaaccaagtggttgc
tccatattaactttatcctcaagatcaccatggagaaaggcattctttatgtccaactga
taaagaggccaatgatgaacaatagccatggacaggaaaaggcgaacagatacgacttta
gccacgggagaaaagtgtcattattatcaagcccaaatagctgagtatatccttttgcaa
tcagacgagccttgagccaatcaacctggccatccaggtagactttgactgcataaaccc
aacgacaaccaacagtagacttacttgaaggaagagaacaaactcccatgtaccactcac
tcacatgtaaagcaaacatctcgtcaatcatagcctgtcgccatcctggatgagatagtg
cctcacctgtaaacttaggaatggaaacagtggacaaagatgatacaaaatcataatagg
gtgatgagatgcggtgataacttaaaccaacataatggggactaggattaagtttggatc
atacaccctttcgaagtgcaatcagtggactaggaggagccaagtccgcactagacgtgg
atgacaatgataagtcaagagtggtggcctcgtggttggagatgtaggatgagcaactgt
agactcctcagaagtcggtataggtaggagtacctgtgatgttgatgtggatttaagagg
aggaacaatagattcctcacaagtagatacaggtaagacctcagatatatcaagatgatt
agatgaagtaaagtaaggttgagactcaaaaaatgtgacatcgactgacataagatatct
acgaagatcaggtgagtagcagcgataccccttttgaacccgagaatagccaagaaagac
acacctgagaacacaaggagctattttatctttttcaggagctaagttatgaacaaatgt
actccttaaaacactaggaggaaagagtataaagatgacctagggaacaatactgagtgt
ggaaactgattctagatggaagatgaaggcatccgattaattaagtaacaggttgtaaga
actgcatcgtcccaaaaacgttgtggaacataggactgaatgagaagtgtgcgagcagtt
ttaatgagatacctattctttctctctactaccctataatgttgaggagtatacagacat
aggataatattttgagaagtcataaactattgaaactaagagaatacatattttaaggca
ttatcactacgaaaagcgaataaaaacaccaagcggagttttaatttcagcataaaaact
ctagaatattgaaaacaactcaaaacgatctttcatttggaaaatccaaatacatcttga
gtaatcattaatgaaactaacaaaatccaaatcttaaggttgtgactctactaagacccc
atatatcataatgaactaaagacaaaacagactctacacgactcttagcacgacgtgaaa
atgtagctcgaatatatttcccaagttgacacgaatcacaatctaatgtggacaaaccag
acaccatcttctgaagcttggataaactcggatgtcctaaacgtttgtgaattaggtcta
gaggatctgtagttggacatgttgtagagggattgagtgagttaagatagtcaaggtctt
gtgattcacgccatgtgccaatcgtctgtaccgtactgcggtcctgcatagtaaaagaat
catcaataaaatatatatcacaatggaattcacgagtcaaatgactaacagatgcgagat
taaaggacaaccggggacataaaaaatagaatctaaagtgacagaggacatgtgattagc
ttgtccaactccttttgcttttgtttagacttcatttgctaaagtatcattgggaagaga
ttgtgaataaacaattatttgacaaaagtgacatattaccactggggtatcaagttgctt
agtcatactaagaatgtttgggagagggtggtggaagtgagggtaaggaggacagtgtct
ctatccgagaaccagttcggattcatgcatgatcgttcaactgcggaagctatccgtctt
attaggaggctggtggaacagtacaaggataggaagaaggatttgcacatgatgtttacc
tagagtaagcgtatgacaaggtccctaaggaggttccttggagatgtcagaaggttaaag
gtgttccggtagcatatactagggtgatgaaggacatgtatgatggagctaagactcggg
ttaggacaatggaaagagactctaagcattgtttggttgttatggggttacagtaaggat
ctacgctcaaaccgttcttatttgccttggcgatggacgcattaacgtaccatattcagg
gagatgtgccatggtgtatgttattcgcggatgatatagttctgattgatgagacgcgag
gcggtgttaacgagaggttgggggtttggagacagacccttgaatttaaaggtttcaagt
tgagcaggactaagacagaatacttggaatgtaagttcagcgacgtgacggaggaagctg
acatggacgcgaggcttgattcataagtcatccccaagagaggaagtttcaagtatcttg
agtcagttatacagggagaagatggggagattgacaaggatgtcacgcaccgtattaagg
gcggggtggatgaaatggaggttagcattcggtatcttttgtcacaagaatgtgccacca
aaacttaaaggtaagttctatagagcggtggttagaccaaccatgttgtatggggcagag
tgttggccagtcaagaattctcatatctagaagatgaaagtagcagaaatgagaatgttg
agacggatatgcgggcatactacgttggaagattaagaatgaaaatatttgggtgaaggt
gggcgtggccccatggaagttgtgcccaccattaaagactgctatctgaaaactaattct
ttgggcccaaacattctggcccaaagtacctcgtgaataataatattgagctcatgtctg
acatgttggaagaggagttactagcaaacacttatacacctatgttggtaacacaattga
agaactacgaaaaacactcttctgcaaaggaaaatgagaagaagaagaagaagaagacga
agaagaaggatgatgcaatgatcattgaagaaaaaggagagcaggaggacccatctaaac
ttacaaagtctagaggaagaggaggacccagagtttgatgcttccctctgggtacaccaa
aacatcgtcaaacttaggcaaggagtttggggtaaacattcaggggtgtgagaaggaagc
tttggagcttttcgtaaaattacaactagaggcataaaaaaaaaaaaggcaatccaggca
tggaggtgacaaccttcgaaaagaaagggattcaaagaactgaaagggctggatttttgg
agtaacttcaagagtaatagaacaagaagtagggggttgcattattatcaaagatcaatg
aagattaacattgaagaagtgggaaatccaaaaagactccaccgagaaggatgatgcaat
gatcattgaagaaaaaggagagcatgagaaaaaacccgtagaaattgacagcactcacac
acaataagacgagataataaagtagtgagttggccaattgaagaagctttacctcttaac
ttacaaagtctagaggaagaggaggacccagagtttgatgcttccctctgggtacaccaa
aacatcgtcaaacttaggcaaggagtttggggtaaactttcaggggtgtgagaaggatgt
tttggagcttttcataaaattataacaagaggcatgggaaaaaaaaggaaatccaggcat
gcaggtgacaaaaccttccaaaagaaagggactggaagaactgaaagggctggatttttg
gcgtaacttcaagagtaataggacaagaagtacgggattgcattattatcaaagatcaat
gaagattaacattgtatcatggaatgtcagggggttaaatcgacatagaaaaagaatgtt
gattaggagtttaattcataggtggaaagcagatgttttctgtttccaagattcaaaatt
aaaaggggacattagggagtttataagagaactatgggcaaataggtggtttaaatatgc
acagttggaggctagtgggcctagagggggtattattgtcttatgggatagtaaaattgg
ggagggggagatcagcagcctgagctcctattctgttacttgtaaatttataggtaaaac
tcaggagtatacttggaatttatccactgtatacgctccaaatgatagggaggaaaggaa
agaagtatggtgggaattagcaggtgccaggggaatttttatggaccttgggtaatttct
ggggatttcaatactgtgaggtacccaccagagaaaaagaattacagcaaaatcactaga
gcaataaatgaattctcataatttattgaagatatggaactggtggatctacaacttgca
ggaggaagttacacttggaggacaggagatagacatgtgataacagctagactggatagg
ttcttggtttttatggattggaatgagagcatcagaaacaccaagcaatcagttctccat
tgaattacctctgaccattcccctgtgatgcttcaatgtggtaaccggtaccctgtcaaa
tcctattacaagtttgagaattggtggctggaaacagagggcttcaaagaaaggattaaa
gtctggtggagctcttttgcttgtgaaggaagacgtgactttattctggctttcaaactt
aaagcatcgaaggaaaaaattgaagaaatggagtaaatctattcaaggaaacttggagat
gcagaaattgagtattcttagtcaacttgcagaactagaagagacacatgatcaaaggag
ccttactgaagaagaaatacacactaaatatgcagtctatggagtttggggagattgcaa
aacatgaggaggtggcttggagacaaagatctagggctctttggttgaaagaagggacaa
aaacatcaattttttcctcaaaattgcaagtgcacataggaaatacaataacatagacca
actgttacttgaaggaaaatttgtggcgaatccaacatacataacaaataatattggtac
attttatcaaaaactatatataaagattgctagaggacaatcttatgttgcaaagtcttt
tcgaagcttaggaaatttgggatagtgtcaggcatgtgaaagggataaagcacctggacc
tgagaactgggaggtgataaacacggatatgatagctgcagttctttgttcatggaatgt
ttgaggaaagctttaatgttacctttgtggtattgattcctaagaagatggaagctaagg
aatagaaggactttaggcctattatgataggcaatgtgtacaagatcttgatagaaagac
ttaagaaattggtgaacaagttggtgaagggtcaacggatgacttttattaaaggtagac
agataatggatgttgttctaattgccaaatgaatgtgtagatgcaagaacaaaggcgaga
aacctacaatactatgcaaactagatattgagaaggcatatgaccatctaaattggaact
ttctattggaatcgctgatgaggatgggctttggtgtaagatgggtcagctggatcaaat
tctgcatcagcacaatgaaattctcaattttgataaatgtttcaccagtaggtttcttcc
cttctcagagggatttgagacagggtgatccactatctccttttattattcattagtgct
atgggaggcttaaatgatatgttaaagactactcaagataacaactgcatacggggtttt
aaggtgaagtccagggcagacagtactattgagatttttcatcttcgatatgcagatgac
gcacttatgttctgtgaggttgacaatgaacaattgaaagtgctgaaggtgatcttcatt
ctgtttgaagccacatctgtattacaaattaactggaatgaaagctttatctatctagtt
aatgaggtaactaagatccactttttggttggaatcctagaaggtaaaattggggaattg
cctacagttatttggggatgccatgggggccaagagcaattttaaggggatttggactag
ggtcgtagagatatgtgaaaaaattttaacaaactggaagagttagtatttatccttaag
ggacaaactaatactaatcaattctatacttgatgattttcctacttacatgatgttcct
cttctcaatccatgtgaatgttgtgaagagaatatatacccttagaaggaacttcctatg
gggaggaaactatgacaaggaaagatctatttggtcaaatggaagtctctcacagtcagc
aagaagtaagagtgttttggaatcaagaattggagaattcagaaccaaagtttgatgatg
aagtggctatggagatttactacagaagaacattgtttgtggaaagaggtgatcatggag
aagtatggcatagaagataaacggataacaaagtctgtaaatagatcttatggagttagt
cgatggaaatccatcagggacctatagcttcagctcttgaataagtccaaattctgaata
ggaaatggattgaaaatatctttttggaaggataattggctaaccaaggaactttgaaac
aactctttcttgacatttacattccaaatcaacagcataaagcaataatagtagaattat
gggctaatcaaggttggaatctcacatacagaagactatcaaaagacccggagattggca
ggtcaacagagttcaaaggcactttggaacaatttaaagaggtctatacttctatagact
atttgacttggcaagggaagtttattgttaattcagcctataaggaattcaacttctcag
ctaactggattggttgttggccatagaagttgatttggaaagttaaaattccttatagag
ttgcttgtttctcttggcttttggctaaagaggcagttctgacgcatgataatctaacca
agagagattaccatttatgttcaagatgttatttatgtgaagagcaggcagagacaacca
atccacttttttttgcattgtaagttcactgcagttatggaggattttcattagtttaaa
gggtatcatgtgggctatgcgtagaagtatacctgaagttctagcatactggaaaaaaga
aagaaatctttccaattataaaaagagatggaggattatcctagcttgcatctggtggac
catttgggaagaaagaaatcaaagatgcttcaaagataaatcagtcatattcagataatt
aaaatgaagtggctagtcttgttttatttttggtgttaagtgttagatagttatgtatta
tgtataagttgtctagtcccacattggaacgggagtaatatgtactatgtagagtatagc
tataaataggacttcttgtactttattgtagagaatatattaataatatatttttcccgt
gttgtctcacatggtatcagagaaaccgtgagatatcagtcgttgtgaaaaataccagcg
gcttcgggaagaaaaaaatcaatcaactgctaggtatattagtcttcggcgaccgatcca
ttaaatttctctggcaaagaaccactcatgggccctcacgcgcccaccgaaagaaatatt
tccggcgaggttccaatttcatgcgcccgcgcgtgaggcagtttccggtcaaattttgac
aaaggtcctttttgacagtttgttcaccctgtaattcccagtctatccatcatttttttt
atttcgatcacttcgcaatttctcgggcagctacagtgatttttccggcagaagcggtgt
ttcctttgcctgcttcagcgagatacagttgattatttctattatttgtttctagacctc
tctccaatccaacgatgtctttggaatttgatgtatttggttctgaaaacacgagttcta
gaaagtcaagcttcatgattactttagagccattaatggggagttcaaactatttagctt
gggtttcctctgttgaattgtggtgtaaaggtcaaggtgttcgagatcacttaatcaaaa
aggctagtgagggctgtgaaaaggtcaatttaagcagtttatgacgtctgtataccactc
agcagaataggatagcaaagaaagaatatgcacatcattgagactgctcgcacacttctc
attgagtctcacgttctgctacattttctgagcgatgcagttctaacggcttgttatttg
attaatcggatgcctttatcttccatccagaatcagattctgcagttagtattgttttct
cagtcacccttatacttttttcgtcctcgtgcttttgggagcatgtgtttgttcataact
tagctcccgaaaaaaataagttagctcctcgtgctctcaagtgtgtcttccttggatatt
cccgagtttaaaagtgatattgttgctactcacctgatcgtaggtaccttatgtcagttg
atgttgcattttttgagtctagaccttactttacctcttctgaccaccttgatatatata
tgaggtcttacctataccgactcttgaggggtttactatagctcctcctctacatactga
gccacagaaatcttactcatacctaccattggggaatctagtgttgctcctcctagatcc
ccagctacaggaacacttttaacttatcgtcgtcgtccgcgcccagcatcatgtccagct
gattcacgttctgcacctgctcctactgcggactagtctcatcctaatctaccaattgca
cttcggaaaggtatatagtccacacttaatcctaatccatattatgtcggtttgagttat
catcgtgtcatcacctcattatgcttttataacttctttgtccactgtttcaattcataa
gtttacaggtgaagcactgtcacatccaggatggcaacatgctatgattgacgagatgtc
tgctttacatacgagtagtacttgtgaacttgttcctcttccttcaggcaaatctactgt
tggttatcgttgggtttatgccgtcaaagttggtccagatgaccagattgccaaagggta
tagtcaaatatttggggcttggttacagtgatattttctctcccgtggctaaaataccat
cagttcatctctttatatccatggttgttgttcgtcattggcatctctatcagtttgaca
ttaagaatgtttttcttcacagtgagattgaggatgaagtttatatgaattaaccaccta
attttgttgcttagggggagtctagtggctttgtatgttggttgcctcagacgctctatg
gtctaaagtaatctcctcgagccttgtttagtaagttgagcacagttattcgggaatttg
gccaactcgtagtgaagcttatcactttgtgctttattggcattttacttcaaatctctg
tatttatttggtggtttatgttgacgatattgttattaccggcaatgaacaggatggtat
tactgagttgaagcaacatctctttcagcacttttagactaaggatctgagtagattgaa
gtattttttaggtattgtgattgctcagtctagcttaggttttgttatttcacattggaa
gtagaaaaacttcaatcatttttctttatttgaaaggaagaaaaaaaaggtaatatctag
acctaaatattaatctgaagacaagtgaggcttgctcagttggtaaaagcacctccacct
acgatcgttaggtcctgggttcgagtcaccatggaggggaagtgtggaaacactatagat
cctcctaatttgggagggggaaaaaaatattaatctgaattgacatgaatctcaatgaca
atgaccaacgatttcctgcaattcttttcagtatggaatgaataaaaaatcaagctacaa
gtctctattaaacgaaatgcactaacagggatcactctcaagaaaggaagtggttttggt
tgttgttattccaggttggataaatcactttctttataaatatcataaaagacaagggct
ttcttgcttcagcacatgtgggaaatgccggggggcttggctggtaccaagctcgagcgg
tctttctatctttttggattgcatgcccaaggcaatgctttttgtagattgggatggatt
gatcttcgcagaagtatgctttagacattcttgaggagacaggaatgacggattgtagac
ccattgacacacctatggatccaaatgccacacttctaccaggatagggggagcctctta
gtgatcctgcaagatataggcggctggttggcaagttgaattacctcacagtaactagac
cttatatatcctttcctgtgagtgttgtaagtcagtttatggactctccttgtgatagtc
attgggatgtggttttccgaattcttcgatataaaatcagctccaagcaaagaactgttg
ttcgaggatcgaggcccatgagcagatgttgattgggcacgatcaccttctaatagacat
tctatatctggatattgtatgttaataggagttaatttggtgtcttggaagatcaagacg
taaaatgtagttgatcggtctagtgcggaagcaaataatcgagcaattgttatggtaaca
cgtgagctagtttggatcaaacaactgctcaaagaattgaaatttggagaaattgatgga
accagtgtgtaataatcaagcagctcttcatattgcgtcaaatccggtgttccatgacag
aattaaacacattgagattgactctcactttgccggagaaaagatactctcaggagatac
cgttacaaagattgtgaagtcgaatgatcagcttagagatatttttaccaagtcccttgc
tggtcctcgtattagttatatttgtagcaaactcggtatatatgatttatatgcaccaac
ttaagggagagtgtgagatagttatgtacaacaaaatacccggtataatcccacaagtgg
ggtatggagggtagtgtatacgtagagcttacccttaccctgtgaaggtagagaagctgt
ttccaaataccctcggctccagtacaaatgaaaaggagcagtagcaacaagcagtaacaa
caatgatatagtaaaataactgaagaaagaaataacatgtagacatataactccactaac
aaacatgcaaggttaatactattgccacgagaatggcaaaggaatgttagatagttatgt
attatatgtatattaatagtctagtctcacgttggaataggagtaatatgtactatgtag
agtatagctataactaggacttcttgtaatatattgcatagagatatcaataatatattt
ttcctgtgctttctcacgtaaaggaatgtaatgtacttagaagatcatgaatctatcttt
gatgttttagacacctcgtgagaacacaaaggtttaggaactttattgtgttctttgtaa
ttatgggtgactgccaatatgttaccttttcataaaaatgattatttggccattggatta
gtttcaacagcctctctgcccctccgggtaggggtaaggtctgcgtacatattaccctct
ccagaccccacttgtgggattatactgggttgttgttgttgttgttgtggattagtttca
acaattttgatagttcttttatttgaatcaaactactcattcacatggattttgtatcgt
atcattgagttaaaaaaattggttttgctaatttatcctcatgtataacaactacctatt
tttcaatatattggattcaggagcttgtagtagctggagtttgctcttcaaagggcaata
agtgccgggtatcatgcacagtgactccaaatacagatctcctttctgctctaactctta
tggagaaacatgatctaagtcagctacctgttatactaggggacgtggaggatgaaggca
tccatcctgtgggcattttggacagagaatgcatcaatgtagcttgcaggtttttgacat
tcaacttttacttcaaagatataatgctttctggaaccattgatgataaaatatgcaaga
aacttgtgcagaagtcgcactttactatcgattaccagataaagttacttatcaagaagt
caaatatattgaacatatttctctaaaacactttgactggactgtaagcagaaacttact
aaagtaggtcgtaagaaatggtttgatagggaaatcaccatctacacttaaaagagttgt
gtgaatttgaattcttaaagcatgtgaaagttataaaaacttgttattatctaagcatct
gaagcattttggccatccaaaggatcaaaaataggaaataatttcatttgtacaatgaac
tccctgcacaaattctcacactaggtgtattctctattcatcactagcactacatgtgtc
actacgaatcatatacaataaatctttgtaacataaaagacgacacataatatggaagta
agccgagtatacaagggaagtttcatcattacggtgagctttttataagataatcaagtt
ttactggaaaagggcaaaaactctcccgtatagaagtataccaaaaagtagaatacctta
caaaaatatgattttctatgaacaacaccctatcttctatacttgtagggatctcatcgg
ggcaccaaaaagagataaagggataagaggcttttcctcaaatgtacaaaatccttctct
attccttcaaaagctctcctatttctctctctgcacactgtccacataagttcaatggag
caacatccacgccctgtgtcttcttttccgtcttctataggtccagctgaacatggcttc
tttgactgagtgtggcatcaacgttgaagaccaaaccatcccagtacttccaaccacaaa
cgagacactatatgacaatttagaagaagatgattcacatcttctcccgaacatttacac
ataaaacaccagctgatacatgtaatcttcctcttcctcaaattatcagccgtcaggatc
acccgtctcgtagctaactaggtgaagaagcacacctttctcgaaaacctcaggatccat
acagagagatatggaaaagctgattcctccatgcccagaagcttctcataataagactta
acaaagaaacaccactacttccccccccccccaaaaaaaaaaaatctccatacatcgact
ttcatgtgtaattcttgttcgtgaaacgacccaatcaacctttggcacaaatctcccagt
cttgcgagttcctcctaaacttcaaatcacaatgaacttctccaccttgtagcctccgtg
tcccttggactggcaactcctttggcatgaaactttgtacatattaggagatgtgatact
caaagtgttgttcctgcaccaattgtacccccaaaaaacttaccatgctcccatcaccta
acattgaatgatacgttccaaaatcttcgcactccttcaagaaacttttccgtaggcccc
acccataagggagtgtgattttttttgctctccatcccctctccaagaatccattcccta
aaccactgcaggacactttaacaatcactatgtcactttttctactagttctacattgag
tgatatcttgatgtcattgaaatgcctctggaaaatcttcttctcatctaaaagaacact
tgtttgccttttgaatccccctctaacattttctatgtttcattcatctttggtggaaca
gagcattagcaactagagaacagctttgctag
(DNA sequence of NtCLCe from Nicotiana tabacum; sequence originating from
the ancestor N. tomentosiformis; one start codon)
SEQ ID NO: 4
atgaatcacgaaagttgttgggtcgt
catccaaattgctggccttgggctcgacgaccatctcttcctccgggacgttcctgtgac
ggaaacattgaaaaagaacaagatatgtgcgacagcagcaaagacgatagtgatagtgat
agtggtatccagataggatctctgctcgaggaagttatcccacaaggcaataataccgct
ataatctcggcttgctttgttggcctcttcaccggtatcagtgtcgtgcttttcaacgct
gcggtaagtgcgctataggtctttcatttctcttttcatctactattctcccttacttac
ttggcctcagtcaatcagccccctgcctactttaaattattgtacaatttatcagaggag
tatcctatacatcaaattcacataacttagtaaaatatgctgacattctgaattttaacc
ttaccagcttagaacatccaggctagttcagaaacagataatctaaattggcctcattta
taagtcattttgttaatcaagacatacaatttggctcttgataaaagattatgcagcgcc
cgatgataacctaatatttatcagcaacccatatgtcactttcttttgtttaaatgctct
cccatgtaatttaacaatattgtcaccatacaaaagagaactgaagtgaatgttccattt
gtggtcatataacggatatctcccttggttaggttcatgaaatacgtgatctttgttggg
atggaattccatatcgagctgcctcagaggagcccattggagtacattggcaacgtgtaa
tcttagtaccagcttgtggcggtttggtagtcagctttttgaatgccttccgagccactc
tggaggtttcaactgaagaaagttggacatcatctgttaaatctgtgttggggccagttt
tgaagacaatggccgcttgtgtcacattaggaactgggaattccttaggaccagaaggcc
ctagtgttgaaattggtacatctgttgccaagggagttggagctctgcttgataaaggtg
gtcgtagaaagctgtcactcaaggctgctggatcagctgctggaatcgcttctggtttgt
tccccatattattcttggttctgaaccatacatggtacattttccttataattacatgta
gcctgttgtatgctttcctctttcctgggaagcctttctgtaaatgcaaatgtgtttgca
ctcaaaccaataaactgtaaaaacagtgaaccccttgagcaagcaaaagcactagaaaac
caacaaatagatcccccccccaagataccagtgaaatgacaccgggtgacccaaaaataa
agcagcttacatcttgactttgagaggaactgcaatcagctataagtaggttattaattt
ccagtgcctgcattctgcccaagtactatgatatatttctgaagctttgtttccccagtt
cctttttcagacgtttgctgtcaataaagttgagccagccaacttggttcccacaagcta
ctaattttgtccaagcttactctatgggagaagttaaatttcccaaattccttgagcaga
aaatgaaaaatgaactcaaagtgtcatattaggcaactatctaaagaaaaatacttaatt
gaagtttagataagaaaagtgaatatatattgatgtagtctccgttaggtgagaagcgca
tcacttacccagcaacatatggacctaaaatttactagtgaacttttcacattgtatcaa
aagctcaacaaacagaaagatgactagtcctaaaatgttatttcacatcaaccttatcat
acgtgcattatttgttctctatatttctatttcatccgatataaccaatcgtcattgtaa
attctataatgcctgtggttacttttgtctttagtgacaaatgacatttaggctaaccat
gtagttattgactgatttcgcttgacgtctcttccaattatgtagtagtagagtgttgag
atatggatatgttaccttctaaaaaaaaagagtgttgagatgcggatggtttgctagctg
gcttttgtctcccttcaagttgaattagcaaaagcaatgtctcataagttggatagctag
acaagaaaaactccaaattactttatgtagagtattcttaagcttgagtcgcgagttgga
aattggaattatgtaaaaaaacctggaattatttggttgagcctgctttttatttttgtc
aatatttccagtatctaacccaacatgtttagagcaattcccagagagcctcaatacgag
gcatttgcagagtctttatgagagtccaggaaggggcacacactgtagaggtatagtgtt
gtccttatttttttttttttgataaggtaagattttattaaaaggtaccaagatggtgca
aaattacaaacatccaaactaatacaacaaagcaactacattcctcctagctcctctaga
aaattcatatattgttccatatttttcattacatgtcttttacaccagaaatacaagttt
aataagcatctgtttttaatcctggatacatgctgcctttccccttcaaagcaaatcctg
tttctttccaaccatattgtccagaacacacatagaggaattgttcttcatactatctgt
tgactctttgccactttttgttgttgccatgtctccaacaaactttacactggcaggcat
tgcccacttgacatcatatatatttaggaagagctaccaacactgctttgccactttgaa
atggatgattagatggttgactgtttctgcctcttcttcacacatgtaacaccggttaca
tagagcaaaacctctcttctgcaagttctcctgagttagaaaagcttcctttgctccaat
ccaaccaaaacgggctactttaataagtgcttttgacttccatattgctttccatggcca
atttgactgataaagcccttgtagtttttgtaacaagctataacaactgctgactgtgaa
aataccatcattacttgctgcccagattaatgagtctctcctgttttcctccaatctaac
attattcaataactgcatcaattgggaaaattcatcaacttcccagtcattgaggcccct
cttgaagattagctgccagccggtgcttgaatagaagtctaacactcttccatttttgtt
aatagagcagctatatagaccaggaaactttgatctaagacttccattttccaaccacat
atcagaccaaaacagggtattattaccatttccaagtttcagtttcacaaactgactata
tttattccaaagattactaattgtgctccaaactcccccttttgaagaagattgaattga
acgaggagcccacatgtccttcataccatacttggcatctatcacctttttccataatct
attcccatcataattatatctccatagccatttaaataaaagacttttgttatgcatctt
tagattcctcactcctaatccccctctttctttttttttcatcacctcttgccatttgac
caagtgaaatttcttgttatcattattaccttcccacaaaaatttattcctcatagtatt
caattttttctccactgatgttggcattttaacgagagatattagataagtaggtatacc
atccatcacactattgaccagtgtaagcctaccaccaagagataaatattgtcttttcca
tgacaccagtttactgctacatctatccaagaccccctgccacatctttgcatcattctt
ttttgctccaagtggtaggcccagataggtggatggtagctgctccactttacaacccaa
aacatctgccagatcatcaatacaatgctcggcattaatactaaacacattactctttgc
caagttcactttcaatcccgagacagcttcaaaagctagtagtactcctatgaggtgtaa
gagttgctctttttcagcttcacataatatcaatgtatcatcagcatagagtatgtgtga
gaaatacagttcttccccctctctttttctaattttcaatcctctaatccaccctaactt
ttctgcttttaaaagcattctgctaaagatttccatcaccaacaaaaataaataggggga
tattggatccccctgtcttaaccccctctgagaattaaagtatctatgtggactcccatt
aattaaaactgagaagctaattgaggatatgcagaattttatccacccaatccatctttc
cccaaaattcgtatgtttcatcagatttaacagacatgaccaatttacatgatcataagc
cttttccacgtcaagtttgcaggccacccctttaatcttcctcttgaatagatattcaag
acactcattagctaccatagcagcatcaataaattgccttcctcttacaaaggcattctg
attatctaatatcaattttcctatcaccatctttaatctttcagctatcgactttgcaat
tattttatagacactgcccaacaagctgataggtctaaaatctttcacttccgctgcccc
ctttttcttaggaataagagcaatgaaaattgagtttaggctcttagtcttgtccttatt
ttcagggttgaactagttctttagaagtttcctaggcttcctaatttccaaagttctgcc
aggtccttttctagtgaagtacttgaagtttaataaatcaaattttaatttctaacatat
cccgagaaattcattcacaaattcaactggtgacttctgatgcagaaacataagcaactg
cttatgggttcatatgttcctgcaattttattgttgacatggattggcttcatatggttt
tgttcctgcaattttatcgctgacactaatcctttcatatggttttatgtggggtggtaa
atagaggttaagagacaagaagaggctggaaaaggtgggcagttcatttgttagtagact
actctatttactaagagatatgatgtcccatacattactcgaattggctccaaatacaga
ttccacttctttgtcgagtttccttattgtacagagttcgactcgtcaagggaaattcac
ttcctttgactgaataatgctagtttgagtagtaccttaaattaaatggaccatttaatt
ctatctacttgatagaatagactggtcatcaactagttgcaaatataatgacaactccgc
catgtttgcagagtcacctgatgaagaagtacctcaattagtagaccatttcttgaatgt
tctacagtattctctatgcctacatgaccacatcacttttccttttgcgttgtgagaact
tgaacttggtgagcgggggttccccaggaatggcatcttggtggcagatgaccattctgt
ccttatcttagctaatgcttcttggattgcctcactagatttattatacctttaataaat
gtttgccattgttctgccataatagagggatgtacctagctggtgcttcacatcacatag
tccaaaactaatgaaatgctttacaattgtcgagtactaaaggatgatttgtggaatcag
atctcaaacaatttattttgaggaagaaaaataccaaaggttttttctgtttgttggaag
attaaaaatcctttaaaaggtaaagatttatgaacttaattcagcatttttgtggccatt
gctgaaaaagagaaaacaatggcacttattcgagtttgcttatccaaaaaaaaagaagaa
gagaatgtcacgtaatgcaatttcatcttaggaaactttgcaggagaaaagcaagagtga
taaaacagaactatttgtttttttgataagttgttgtgacctatttctttgtcattctta
tttgctaataagctaatgtaccctgtactatggttgttttgacttaatccggggatgttc
agtgagcattttcttgttttttctgctgtcagcatctgctgccttacaggaattcatttt
ctggaaatttacttcttgttctgctaacattttcctgttatatcttgtcagtcattttct
ctccatggttatactgtttgtgtcactttgaaactctccttgttttctactttaaaggat
ttaatgctgctgtcgggggctgtttctttgctgtggaatctgtgttatggccatcacctg
cagagtcctccttgtacttgacaaatacgacttcaatggttattctcagtgctgttatag
cttctgtagtctcagaaattggtcttggctctgaacctgcatttgcagttccaggatatg
atttccgtacacctactggtaattttggacttctttctcgagtttgattcttaaatacaa
ttgtacccgtcacttacagcaacaacaactacatttcaacagctagttggggttggctac
acagatcatcactatccatttcaatttctttagtcccatttctttcgaatattcagtact
ttgggattctctattatcagaggttctctttattttctactttgacgtacaaatctctaa
atagattaaagaagactcctagagacactggcctaatgcaaatgtaccaccatgaataaa
ccttaatctgaaatagctggtatcgtatataagaacctttagctttaattgtgttctata
ttgatcttttgggacaacttccgtccaataatattatgtcttacttatacagttatactt
atccttaaactttactctttagagtggttatccgtagttcaagcttttgttggcaccata
gctagtttggttcttagtaaaaagttactctttagagtggtaactttttgtcaattttct
tagtgaaaatataacctctgtgacaaatctaccaagtataaatccaatatggttctgtgt
catacttgtagtttatccaagtctatgctccatcactcttacaaaggctcatcgtatgac
taattttttttgagaaaggtaacagtttgtattgataataagatcagcgccaggttagtc
attagtgctaatagctgtatgtacaactccaaaagagcaaaagacaagcacctggtgtaa
cgtaaattacaagctgcctataaaatctatcaggtctcctacctcactaaacatttcttg
tttacaccaaaaaaataaaacaaggaaagacaatccatcttaatcttctgaatggagttt
cttttgccttcaaacatctcgagttcctttcgttccatgcaatccaccatatacaagctg
ggatgcttttccatttgtctttatccattttttctaccaattcccttccaattgactaga
agttccaatgtggttctagatatgacccaattaactcccaacatataaaagaacatgttc
cacggatttgtagtgattctgcaatgtaggaacaagtgagcattactttctacttcctgt
ccacaaagaaaacatcttgagcaaatctggaaacctcttctttgtaagttatcatgtgtt
aaacatgcttttttaccactaaccagacaaaacatgatactttgggaggagttttaaccc
tccaaatgtgtttccaaggccacacctcagtcattgaaacattatgatttagagtccagt
atgcatcttttactgaaaatgcacctttgctattcagcttccaaactattttatctatgg
tcttgttagtttacagctatgtatatagtgtagtcttgtcccacattggaataggagtag
tatgtccttgtatagtatagctataaataaggacctcttgtattgtattgaacatccaat
atcaataacatattttctcccgtgctttctcacatggtatcagagcaattgtgagagatt
tatcgctgcgcataaattccagcgactccgggaagagaaatcagtcaccggaagtctttt
tccgacgactctttcaaggttgtttgcgtttgctttataaatccaacactaccacaagag
taatcactgtccggcgaccaaaccccagtaaaaatctccggcagcagcctcctcacgcca
ccagaagctcacgcgccggcgcgtacgaccacttccgtccattttttgaaaaacttcctt
cagaacagttgggtcgcctggtaattcctatcctacccctactgttttcatttcattccg
accactttgagttttttccggctgctacagtactattccggcagctatagtactattccg
acaactacagtaagattccggctgctacagtatttcattattctgtttttgtgtttcctt
actctgtttcagtggattacaattgattctttctcttatttggtaataatttgcaacaat
gtctatgggatttgatgtttttgggtctagaaacatgagttctggaagctctagtgttat
tattacctcagaaccttaaatgggaggttcaaactacttagcttgggcttcatctgtcga
gttgtggtgtagaggccaaggtgttcaagatcatctaatcaaaccgtctagcgaaggaga
tgaaaaggcaataacactttggacaaaaatcgatgctcagttatgtagcatcttgtggcg
atctattgattccaagttgatgcccttgtttcgtccattcctgacatgttatttggtttg
ggcaaaggcacacaccttatacactaatgacatatctcgcttctatgatgtgatatcgcg
gatgacaaactgaaagaagcaagaattagatatgtctacttacttgggtcaagtacaagc
aatcatgggggaatttgagaagttgatgccagtttctgctagtgttgaaaaacaacaaga
gcagcgacaaaagatgtttctcgctcttaccctcgctgaacttcctaatgatcttgattc
agtacgcgaccatattttagctagtccgactgtcccgacagttgatgaattattctctcg
attactccgccttgctgtagcaccaagtcacccagtgatctcatcacagatacttgattc
ctctgttcttgcatcccagacaatggatgttcgggcatctcaaactatggagcatagacg
aggaggaggtcgttttggaagatctagacccaagtgttcttattgtcacaaacttggaca
cactcgtgaaatgtgttattccttacatggtcgtccacccaaaaatgcttacattgctca
gaccgagactccaggtaaccagggattttctttatctaaagaagaatataatgaactcct
tcagtatcgaacaagtaagcagacatctccacaagtagcctcagttgcttagactgatac
ttcttttactggtaatttttttgcttgtgtttcccagtctagcactcttggcccatgggt
catggactcaggcgcttctgatcacatctctggtaatatatcacttttgttaaatattgt
atattcatagtctcttcccattgttactttagccaatggatgtcaaattacggcaaaagg
agttggacaagctaatcccttgtcttctatcaccctagattctgttctttatgtccctgg
ctgtctttttcgtcttgcatctgttagtcgtttgactcgtgccctccattgtggtatata
ttttattgacgattcttttattatgcaggactgcagtacgggacagacaattggtggagg
acgtgaatcagaaggcctttactaccttaactcacccagtccttccacaacatgtctggt
tacagatcctccagatctaatccacagacgtttaggacatccgagtttatccaaacttca
gaagatggtgcctagtttatctagtttgtctacattagattgtgagtcgtgtcagcttgg
gaaacatacccgagcctccttttcgcgtagtgttgagagtcttgcatagtctgccttctc
cttagttcattctgatatatggggtcctagtagagtaagttcaaccttgggatttcgtta
ttttgttagtttcattgatgattattcaagatgtacttggcttttcttaatgaaagaccg
ttctgagttattttctatattccagagtttctgtgctgaaatgaaaaaccaatttggtgt
ttctattcgcatttttcgcagtgataatgccttagaatatttatcttttcaatttcagca
gtttatgacttctcaaggaattattcatcagacatcttgtccttatacccctcaacaaaa
tggggttgctgagagaaagaataggcaccttattgagattgctcgcacacttctaattga
atctcgtgttccgttgcgtttttggggcgatgcagtgctcacaacttgttatttgattaa
tcggatgccttcatctcccatcaaggatcagattccacattcagtattgtttccccagtc
acccttatactctcttccaccccgtatttttggaagcacgtgttttgttcataacttagc
ccctgggaaagataagttagctcttcgtgctctcaagtgtgtcttccttggttattctcg
tgttcagaagggatatcgttattattctccagatcttcgtaggtaccttatgtcagctga
cgtcacattttttgagtctaaacctttctttacttttgctgaccaccatgatatatctga
ggtcttacctataccgacctttgaggagtttactatagctcctcctccaccttcgaccac
agaggtttcatccataccagccgttgaggagtctagtgttgttcctcgtagttccccagc
cacaggaacaccactcttgacttatcatcatcgttcgcgccctacatcgggcccaactgg
ttctcgtcctgcacctgacccttctcctgctgcggaccctgctcctagtacactgattgc
acttcggaaaggtatacgaaccatacttaaccctaatcctcattatgtcggtttgagtta
tcatcgtctgtcatttccccattatgcttttatatcttctttgaactcggtttccatccc
taagtctacaggtgaaacgttgtctcacccaggatggcgacaggctatgagtgacgagat
gtctgctttacatacaagtggtacttgggagcttgttcctcttccctcaggtaaatctac
tgttggttgtcgttgggtttatgcagtcaaagttggtcccgatggccagattgatcgact
taaggcccgtcttgttgccaaaggatatactcagatatttgggctcgattacagtgatac
cttctctcccgtggctaaagtggcttcagtccgtctttttctatccatggctgcggttcg
tcattggcccctctatcagctgaacactaagaatgccttttttcacggtgatcttgagga
tgaggtttatatagagcaaccacctggttttgttgctcaggagggggtctcgtggccttg
tatgtcgcttgcgtcggtcactttatggtctaaagcagtctcctagagcctggtttggta
agttcagcacggttatccaggagtttggcatgactcgtagtgaagctgatcactctgtgt
tttatcggcaccctgttgacattccgatggatccgaattctaaacttatgccaggacagg
gggagccgcttagcgatcctgcaagctataggcggctggttggaaaattaaattatctca
cagtgactagacccgatatttcttatcctgtaagtgttgtgagtcgatttatgaattctc
cctgtgatagtcattgggttgcagttgtccgcattattcggtatataaaatcggctccag
gcaaagggttactgtttgaggatcaaggtcatgagcagatcgttggatactcagatgctg
attgggcaggatcaccttctgatagacgttctacgtctggatgttgtgttttagtaggag
gcaatttggtgtcttggaagagcaagaaacagaatgtagttgctcggtctagtgcagaag
cagaatatcgagcaatggctatggcaacatatgagctagtctcgaccaaacaattgctca
aggagttgaaatttggtgaaatcaatcggatggaacttgtgtgcgataatcaagctgccc
ttcatattgcatcaaatccggtgttccatgagagaactaaacacattgagattgattgtc
acttcgtcagagaaaagatactttcaggagagattgctacaaagtttgtgaggtcgaatg
atcaacttgcagatattttcaccaagtctctcactggtcctcgtattggttatatatgta
acaagctcggtacatatgatttgtatgcaccggcttgagggggagtgttagtttacagct
atgtatatagtgtagtcttgtctcacattggaataggagtagtatgtccttgtatagtat
agctataaataagacagtactaacgtcccttttgccgggggttctgcatctttaaataga
tgcacgtggttccatagcagaccgtgttgatcacagatcgtgctgcatcctcttcccagc
ggactcggtgagcccctcttgtattgtattgaacatccaatatcaataacatattttctc
tcgtgctttctcacaggtctgtgatgtacccttgaaaggttcaagagtttggaggaagat
agaaactctgtttatctcccaatcatccaaagatcttctaaagttccagttccatccttg
tgagctccagactgacttaccaatgcttggctttgaagacttagagagaataagtcagga
aaaatctttcaaccttccttgccctatccggtgatcttcccaaaaagatgtcttcaaccc
attgccaacattgatcctgatattgctactgaaagatttcttttggtggcaggattactc
tcattaacaatgtacttgacaatctccatacatacgaatgtctctttaccctcttgccat
taaggttgtaaagagacttgtcaaattaagaagaggtttcctatggaactgtttcaagga
aggaacctcctttcctttggtcaagtggagttaagtcatataatctaggaagtggagact
tgggtataaaatagctgcaactacagaaaaggagcatcttatttaaatgatcacgcaaat
gtgcccaaaactttaaatatctgcggagcatatggttgtagcaaaatttgaatcttccgg
tcaatgttgctcatgtccagtgaatacccctgatggtgaaagtgtcctgaagggaagcag
gaacttattggaggaattggcatttaacactcagcatttcgttaggtcatagcccgctga
aaattgagtgcccagatttatatagttttgctctaaactgacgatgcagttgcacaacat
acgacaaactaaggtgggacatcttcttcggaaggaattttgaggattaagagatagagt
ggttgattcagttgcaaatgaagcttcaagggttcaatatcatccaggagacaccggatt
ctgatagataaaacaacagaaagatgaacactactttgttaggcttgttacaagttgcta
tcgtctttcttatctcggcacacaatttagatttgggaacttatttggaaaatagagtgg
ttgtttttgtgaatagcatcagacaaagcttctgagctggtacgacagaaaactcaacag
ggagaataaaagactgtggttcacgatttctgcatgcatcttgtaggttatttggtgggt
aaaatatttaatgttttgaagggaaggtagaacatgttcataggcttagattcaaatgtt
tgtatttttttggctctttggtgagagatgctgaatgtaaatgacataggcagctgacta
taatttctcagctccttgctttttaaattggcaggcactgatatgtacatgtgaacatcc
aacacttttgtggtgccgttccgatgaataaagcacattaatcacttactgatcaggagt
aatagtttaggagttctagaatttttgtacataaaatgaaccaaaaagaatatcggaatg
agaacatgtttctttttttgtttcttctttttcgtacaaatttcaataacacttctgata
gaatagctaggtccatttgaattcctttggagacccttacacaaccaatgaatggcaagt
atagcattttctaacaccctcccacatgtataatccagtttttagggtttagatgtggat
ttgatttgaccttattgcctttttttgtttttgttctttttgaagtagagagtgaggagg
ctcacaacgacgggctacgtagagcgagattaattcggctcaacgggctaatgattggac
ttacatgctacaacaatgttaggagaaagagagagagagagagagaagcccagagcagtt
ccacgagttaagaaagagaagtccaaagcgattgaatatgaagagagaaagcggttgtgc
taacaggctccctcaagtttggctctgagcatccaactcaaaaccttaaggcaatgagta
gagtagcccaggaccatttaaactcctgttgaaaaccttacacaaccaataagggaacaa
gtgtaacattctcttacaaccctaccgtcttataagtcagggctctaatttagcataaaa
tcaaagtgaggcgatctactatgaaatgaagaaaataactgataaatataaagaatgtta
attctcccatatagcctgaatgttcccagaacaaaataaattagtctcatgatttatcat
taacatgatgttcctcttattttgagtgattaggaaggttaatcaaggagtaaattcttt
ctaatttgtatcgtctagaattatttgtctaacaaattttcagattaccggtgatcaaaa
gaggaaaatattttgcatacaacgttaccataccttacaaaagggcgatgaacatttttt
tattttattattgtcctttttttcaattaggggttatgcagtcttcctccacgtgatatt
actcttagaatcacgtttttgtcattgctattacttactgtggtaagtacaaatgtgttt
tgaactctttttggtatgtattattgagttaatttttcgtttccatttcagagctgccgc
tttatcttctgctgggcatcttttgtggcttagtttcagtggcattatcaagttgtacat
catttatgctgcaaatagtggaaaatattcaaatgaccagcggcatgccaaaagcagctt
ttcctgtcctgggcggtcttctggttgggctggtagctttagcatatcctgaaatccttt
accagggttttgagaatgttaatattctgctagaatctcgcccactagtgaaaggcctct
ccgctgatctgttgctccagcttgtagctgtcaaaatagtaacaacttcattatgccgag
cctctggattggttggaggctactatgcgccatctctattcatcggtgctgctactggaa
ctgcatatgggaaaattgttagctacattatctctcatgctgatccaatctttcatcttt
ccatcttggaagttgcatccccacaagcttatggcctggtatgaatttgtcttttgttag
aagtagcattacatatctggataagtgagttttttattattgaaaagtaataacaggaga
acaagagaatatatcacccaaatctacttctttcctctcttctattcttctgaaattcaa
ggtcctttaactcctccacagtctgtctagttattgatcctgtagacttaattcacatag
gtttaggacattcgagtttatccaaacttcatgaaaaggtttctaatttttttacattac
attatgagtcgtgtctacttgagaaacatatcactccatgtttctatagtctgttttctc
cttagtttattctgatatgtggggtcctattaagtcagttcaaccttgtattttcattat
ttttgcagtatcattgataattattcaagatgtacttggattttctttacaagagatagt
tctcagttgttttttgtgttcctaagtttttatgctgcaatacaaaattggtttgatgtc
tctatttgcatttttcccaatgataatgccttagaatattttcttttccgtttcagtagc
ttattatttctttaggaactctttatcagaaatctcaactgagatagatgagaggaagaa
taagcatatcattggtctcattcagtcccctgtcaagcttagtttcttgagcgatgcggt
ttcacgtccttttattagattaattggatgcctcatctgctatccaaaatcagttaactt
tcgatattgtttcctcgcttacctttatactctctttccctcgagtctttgggagcacat
gttttgttcaataacatagctcctggaaagtgaccagcgcaaccgacaaacaaggccttc
ttaatgtagaaggtggacatatgctattctagccacgggaaagaaagtaatattgtaatc
aaacccaaatatctgagtataacctttggcaatggcgatcaatttgattatatggaccaa
ctttgcctgcatatacccaccgacaaccaataatagatttaccgggaggtagagaaacaa
gctcccaaataccactaatatgtaaagcagatatatctctgatcatagcttgtccttgtg
gacatagggatagaaattaaggacaaagatgacacaaaagcataatgcggtgatgataaa
cgatgataactcaaatcaatataatggggatggggattgagagtggatcgaatatctttg
cggaatgcgattggtagactaggaggagagaagtctgtggacatgatgttggactgagat
caataataagtcaagaatggtggagctacagaacatggaactggagctgtaggtgacata
atcggagctgtaggaggtggagctatagaggaaggtgaaggagagatagcgactgaatct
ccaaaagatgaaaccggtaatacctcaaaaaatgtctaagagatcatttggacctatgaa
gtatgattgcgtttttaaaaaggtaacatcataaggtcaggtgaataacattgatatccc
cgttgcatcctcgagtaacttagaaatatacatttgagagcacggagagctaacttatct
tttctggagcaaggttgtaaacaaaacacgtgctcccaaagacacgaggtggaagagaga
aaggtgagtggggaaacaagacagaggatgaaacttgactcttgatagttgaagatgaca
tacaattaataagacaataggatgtgagatccaatgacagttctcatgaactgctgaaat
ggagaagacaaatactctggggcgttatcactacgaaatgtgcagttagaaaccccaaat
tgattttggatttcagtgtggaaggtctaaaaaatagagaacaactcagattgatttttc
atcaagaatatccaagtggacttggaataatcatcaatgaaactgacaaagtagcggaat
tccaaggtagaactaacccgacaaggaccccaaacatctgaatggactaaagtgaaaggt
aactctacccgattatcaggatgtcgagggaaatgagagtgagtatgccttctgagcgga
tatgactcacgctctagagtggacaagtgagacaaacgaggtactattttctaaagttct
gataaattgggatgtcctaactgtatatgtaataaatctggtggatcagtaaaaggacaa
gctgtagggggaaaaaaataccaaatatttccagaagatggcaaactacaacagaagatg
caactgcattaacatgctcaggataggtgatgaaatcattgaggacaaagagttgatcaa
gaaggagattctggaattttaccagaacttatatagtgaaaatgaaccctggaggcgcag
tgcaaatttcgaagacatctcctcactaagcatagaagagaagaactggttggaagctcc
atttgtagaaatagaggtgcttgaagctttgaaatcatgtgccccttataaagcaccagg
tccagaaggcttcactatggatttctttcagaaaaattgggatactcttaaaacagacat
catggctgcacttaatcattttcaccagagctgtcacatggttagggcttgcaatgccac
cttcattgccctaattccaaagaaaaatggtgctatggagctcagagactacagacctat
tagcttgacaggtattgtatacaaattggtttcaaagattttagcagagaggctcaagaa
ggtaattgacaaactagtctcgggggaacaaaatgctttcatcaagaacaggcagatcac
tgatgcttccttgattgccaatgaagtgctggattggagaatgaaaagtggagaaccagg
cgtgttgtgcaaactggacattaaaaaggcttttgatcaattaagctggtcttacctcat
gagtatcttgaggcagatgggctttggggagaaatggagaagatggataaactattgcat
ttcaactgtcaagtactctgttttggtgaatagggacccaatcggttttttctcccccca
aaagggcctaaggcagggggatcccctctcccccttcctattcattctggcgatggaagg
actcactaaaatgttggagaaggctaagcaactgcaatggatacaaggctttcaggtggg
aaggaatcctgccagctcagttacagtatctcatctactctttgcggatgatactcttat
tttctgtggtactgagagatcacaagcacgaaatctcaacctgacactgatgatcttcga
ggcactatcaggactccacatcaatatgataaagagcatcatataccctgtgaatgcagt
ccccaacatacaagagctagcagacatcctatgccgcaaaacagacactttcccaaccac
atatcttggacttcccttgggagctaaattcaaatcaaaagaagtttggaatggagtcct
agagaagtttgaaaagaggcttgcgacttggcaaatgcaatacctccccatgggtggcag
gttaactttaatcaatagtgtactggacagtcttcccacataccacatatctttgttccc
aattccaatctcagtcctaaagcagatggacaaactcagaaggaagttcttatgggaagg
atgcagcaaaacacacaaatttccactagtgaaatggctgaaggtaactcaaccaaaatt
caaaggagtcttgggaatcagggatgctatgctcttaaaatggctctggagatatggaca
ggaggaatctaggctatggaaggacatcatatttgctaaatatggagcacacaaccactg
gtgttccaagaaaacaaactctccttatggagttggtctgtggaagaacatcagcaacca
ctgggatgaattcttccaaaatgtaactttcaaagttgggaatgtaactcgtataagttt
tggaaggatagatggcttggaaatacacctttgaaagacatgtttcccagtatgtatcag
attgccgtgaccaaagactccactgttgctcataatagaaacaatgacacttggtaccca
cttttcagaagaaatttgcaggattgggaggtcaacaacctactcacaatgttaagctcc
ctagaatgtcataacattgaagatcaacaacctgacaaacttatttgggaaaattctaag
agaggcaagtacacagtcaaagaatgatacattcacctctgtgaccagaatccaatatat
aactggccatggaaacatatctggagaactaaagtgcctaccaagatgacttgcttcaca
tgattgtctctaaatggggcctgtctcactcaagacaacttaatcaagaggaacatcata
taagttaatagatgctacatgtgccaacaacagtcagaaagtgtaaagcacttattcctt
cactgctcagttgcaaaagaaatttggaacttcttctacactacctttggtctaaaatgg
gttatgccacaatcaactaagcaagcttttgaaagttggtatttttggagagttgataaa
tccattagaaaaatctggaaaatggtgtcggccgcaagtttttggtgtatttggaaagaa
aggaactgaagatgttttgatggcatatcaactccactcaaggctgcgtgtttagttaac
ttattttgctggaactatctcacccctgttaatagtgctgatacttctgtggatttcatt
agccccctgatagtagcataggcttttgtaaatggagctaattatcctttctcttttgta
ctctttgcatcttcttgatgccttttaatgaatctaatttacttcatcaaaaagaaaatg
acaagttgttgaaggaggaaaagatgtgagtccatgtgatttagcaaggataaggtacta
aagtccatttgattcacgtccggtaccaatgatccgtctcgtgctgcattcctgtattaa
aacagagtcatcaagaaataaaatagagcaaataagtgattggccaagcgactagtggat
atgagattaaaaggactatggggaacataaaaaactgaattcaaaggtaaggaaggaagt
ggactagcttaacctattctagttgccatggtttgagaatcgttggccattgtgactatt
ggaagtgattgagagtaagaaatagtagtgaaaggagatttgttacccgaaatataatta
gatgcacctgaatcaatgacccaaaagtcggaagaagaggaaacacaagtcacgctatta
cctgtttgaacaatagagattagtttggatcaaatagttgtatagagaactgaaatttgg
agaaatcaatcatatagaacttgtatgtgattattgttgccctttatattgcgtcaaatc
ctaaaacacattgagattaactgccacttatcacagaaaagatattctctagagacattg
ttacaatttcatgaagtcaagtaattagcttgaacatatcttcagcaagtccctcgtcag
tcctcatattagttacatttgtaacaatgtcggtacataagacttataagcaccagtttg
aggaggagtggtagagagttgatgtacatagttaaagtagatatacttacacttagtgtt
atgtaaagagtggatataaaaagggatcagcataagacaattgtcttcgcgcgtcttaac
atttttttcctgtctttatttctctcatggtatcagataacctatctctatcttggttta
cccaatggttggcccccatattgtattagccatgctccagttgactaggcttggacgggc
agaggtgttaaattatcccatattggttgaaagaatgagctattgtctccttatatggtc
ttagacaattctccaactcatgagatattttgttttggctgagttagccctaaggtttat
tttttgtcatattctttaaccttatggcaatgcttgtacacggaaaaaccggagtgcaag
acttaaattaggagaaggaaactattgaaggtgaggaacttaaagggttgtgagaataca
cgggagaaaaaaatcttaatactatctagtggccttgtatatcaaatgatcagcttgcaa
atattttcaccaagtccctcactggtcctcgtattagttacatatgtaacaagttcggta
tatatgatttgtatgcaccggcttgaggttatgcatattctattcctcctactatatatg
tgactaggaaatattttactcctactgcatatgggactaggactatttacacataactat
ctaacattcccctcaagccagtgcacacaagtcatatgtaccgagcttgttacatatgta
actaatacgaggaccagtgagggatttagtaaaaatatctgcaagctggtcattcgacat
acaaggccactagactccccccgagcaacaaaaccaggtggttgctgataaacagaaact
ggccgaaaagttgccggaaaaatttgaaaatagtgagactaagccgaattctacactaca
aaataggttctaaaacaccaccagaaaacaaaaacttttctagaaattactcttcacacc
ggaaaaaataaaagttgtcagaatttgatgtaatttatatagataggttcggaatcactg
gaggagtaagttgtcccgaagaagttttgtcaaaaagtggccggaatggctcacatgcgc
cggaaaacttactgtagctcgcaggaaccctagttctggcggtgcgtggaggcgcgtgac
ttaagattaagatgcttacaggactatcttgagaaatatacatattatatagacgcttga
gttgcttcccaatcctaaatagaagcttttattcgtaggcaagaagggaagcagctttac
ttgagccaatagctttcaaggtgcacgttgtcacaccaaggacatccagaatttgatttt
atagggggtgtgagaaagcacgggagaaaatatgttattgatatttggataataaataca
atacaagaggtccctatttatagctatacactacaaggagatattactcctcttccaatg
tgggacaagaatacactatacatatctgtaaactaacactccccctcaagtcggtgcata
cacatcatatgtaccgatcttgttacacatgtagctaatacgagaaccaataagagactt
agtgaaaatatctgctagttgatcattcgactttacaaactttgtaacaatatctcctga
aagtattttttctctgacaaagtgacagtcgatctcaatgtgtttagtcctctcatggaa
caccggatttgacacaatatgaagagtagcttggttatcacacattagttccatcttgct
gatttctccgaattttaactccttgagcaactgcttgacccaaaataactcacacgtcgt
catagccatggcccgatattcggcttcggcgctagatcgagcaactacattctgtttctt
gctcttccacgagaccaaattacctcctactagaacacaatatccagacatagaacgtct
atcaaaaggtgatcttgcccaatcagcatctgtgtacccaacaatctgctcgtggccttg
atcctcgaatagtaatcctttgcccggagctgactttatataccgaagaatgcgaacaac
tgcatcccagtgactatcacagggagaatccataaactgacttacaacactcaccggaaa
agaaatgtcaggtctagtcactgtgaggtaattcaatttgccaaccaacctcctatatct
cgtagggtctctaagaggctccccctgtccaggcagaagcttagcattcagatccatagg
agagtcaataggtctgcaacccatcattccagtctcctcaagaatgtctaagacatactt
ccgctgtgaaataacaatacctgagctagactgagcgacctcaatacctaaaaaatactt
caatctgcccagatccttagtctggaagtgctgaaagagatgttgcttcagattagtaat
accatcctgatcattgccagtaataacaatatcatcaacataaatcactagataaataca
cagattaggagcagaatgccgataaaacacagagtgatcagcctcactacgagtcatacc
gaactcctgaataattgtgctgaacttaccaaaccaagctcgaggggactgtttcaaacc
atatagtgacctgcgcaatctgcacacacaaccattaaactcccctaagcaacaaaacca
ggtggttgctccatataaacttcttcctcaagatcactgtggagaaaagcattcttaatg
tctaactgataaagaggccaatgacgtacaacagccatggacaaaaagagacgaacagat
gctactttagccacgggagagaacatatcactataatcaagcccaaaaatctgagtatat
ccttttgcaacaagacgagccttaaaccgatcaacctggccatccggaccgactttgact
gcataaacccaacgacaaccaacagtagacttacctgcaggaagaggaacaagctcccaa
gtgcaactcgcatgtaaagcagacatctcgtcaatcatagcatgtcgccatcctggatga
gatagtgcctcacctgtagacttagggatagaaacagtggacaaagaagatataaaagca
taatgaggtgacgacagacgatgataacttaaaccgacatagtggggattaggattaagt
gtggatcatacacctttgcggagtgcaattggttgactaagaggagacaagtccgcagta
ggtgcagaatctgatgcggggcgtgaatcacctgggcctgatgctggatatggacgacga
tgataagtcaagagtggtggagctgccgaaggttgaactggattatgtggaggaactgga
gctataggtggtggagctacaactggagctgtaggtggtggaactagagtaactgaatct
ccaaaagatgaaactggtagtacctcagaaatatctaagtgatgacctgaacctgtgaag
tatgattgggtttcaaagaaggtaacatcagcagacataaggtactgctggaggttagga
gagtagcatcgataccccttttgtgttctcgagaaacctagaaatacgcacttaagagca
cgaggagctaacttatccgttcctggaataaggttatgcacaaaacaagtgcttccaaag
atacgaggtggaagagagaacaaaggtaagtggtaaaacatgacagagaatggaacttgg
ttctggatagctgatgatgtcatacgattaataagatagcaagatgtaagaactgtatcc
cccaaaaacgcaacggagcatgagattgtatgagtagggtacgagcagtttcaataaaat
gtctattctttctttcagctaccccattttgttgagatgtgtacagacaagatgtttgat
gaataatcccatgagatttcataaactgctgaaatggggaagacaaatactctcgggcat
tatcactacgaaatgtgcgaatagaaaccccaaattgattttgaatttcagcgtggaagg
tctggaaaatagaaaacagctcagatcgattttttatcaaaaatatccaagtgcacctgg
aataatcatcaatgaaactgacaaaatagcagaatcccaaggtggaactgacccgactag
gaccccaaacatctgaatggactaaagtaaaaggtgactctgctcgattatcaagacgcc
taaggaaatgggagcgagtatgcttaccgagctgacatgactcacactctagagctgaca
agtgagataaaccagataccattttctgaagttttgacaaactgggatgtcccaaccgtt
tatgtaataaatctggtgaatcagtaacaggacatattgtagatggaagacaagatgcga
gtccatgtatttagcaaggataaggtaataaagtccgtttgattcacgcccggtaccaat
gatccgccccgtactgcgttcttgtataaaaacatggtcatcaagaaataaaataacgca
tttaagtgatttggctaagcgactaacaactatgagattaaaaggactattgcgaacata
aaggactgaatctaaaggtaaggaagaaagtgggcttgcttgacctattgcagttgccat
ggtttgagacccattggctattgtgacttttggaaaagattgagaatacgaaatagtagt
gaaaagagatttgttaccagaaatatgatctgatgcacctgaatcaatgacccaagactc
agaggatgaagattgggaaaaacaagtcacgctattacctgtttgaacaacagaagctat
ctcagaagatgtctgcttacatgctttgtactaaaggaactcaatataatctgctaaaga
aaccatccgactattcaaagcatcggttcccatgtcgctacaatttgtagtagtagggtt
aacttgaaatagtggaaataagtaactccggtgagaaaactgaagaaatagcttgaaaac
actgtttacaacagtaaaaacagaacactgttctgcgccggaatctactgtagctgacgg
aaaaactcaaagtagtcggaatgaaacgaaaaacagtaggggtaggatcggaattaccag
gcgacccaactattctgaaggaagtttttcaaaaaatggccggaagtggtcgtacgtgtc
ggcgcgtgagctcacgcgcgtgagcttctggtggcgcgtggaggcgcgtgaggaggctgc
tgccggagattttcactggggtttggtcgccggacagtgactactcttgtggtagtgttg
gattttgcacaacactgacggagataaagcagacgcaaacagccttgaaaaagtcgccgg
aaaagacttccggtgactgatttctcttcctggaatcgctggaatttatgcacagcgata
aatctctcacaattgctctgataccatgtgagaaagcatgggagaaaatatgttattgat
atttggataataaatacaatacaagaggtccctatttatagctatacactacaaggagat
attacttctcttccaatgtgggacaaaaatacactatacatatctgtaaactaacaaggg
gaatatcgtttaaagataaaaaagatagcgtgcagaagattgcatacattagagatgcaa
aatacagaatacccatactcccagataatgcagtatgccttttgcatgacccactggttg
aatggaagcacctggtcaatttactaggtgtgttagtgatttttgctgcttccttcccct
ttctaaactacatactatctaaaatgttagggggacagaagcccagtcaatctgactagg
tgatgttagtggtttccgcttctttctcccacttctaaatgcgtactttctcaaatttag
gagcatagaaacttaagcagctgcctacctgaggaggtgcatgggaacataagagaatag
actttacctgtcatattttccataccttagttaattacagtgttatcctgataatgatct
gttttctgtatctaggctgaatcgagattcaatcgcttttggctgaaaggatgctgctac
agatccttagtttacatcattgtggttcttattctataagtacttcccctatcaactact
tccttcttttttcttaggttatttgcctcttaggttgtttgcaaggaaaggaacaataga
tgttttgatggaatagcaactccaaaccacttccttaaggctaatatactgtttggccaa
gcttcttcaaagtccaaagcccttttttgtcttcaaaaaagtatctttttttcccaaagt
tgaggtgtttggccaaacttttggaaggaaaaaaaagtgcttttgagtaaagcagaagct
cttgagaagtagaaaaagtagttttttcccggaagcatttttttgaaaagcacttttgag
aaaaataaacttagaaacactttttaaaagtttggccaaacactaattgctgcttaaaag
tgtttttcagatttattagccaaacacaaactgcttctcaccaaaagtacttttttgaaa
aatacttttttgaaaagtgattttcaaacaaagcacttttcaaaataagtttattttaga
agcttgtcaaccggctataaatgtcttttatttttacagctagagtaccctaacacctgt
aaattcccctagacatttttttcgactttgttagctcattaaccctagtataggactctt
tgttttggagctagcaaactcttttgttttcctatttttgcatcttcttggtgccattta
taatatctcttacttcaccaaaaaaaataagttcccaaaatatgactaccttgagttggc
caaagcataaccaaagcttgggcacaccagtgtttgcgtgaattttatggatgttcctta
cctttatccttctgtgcttatgtagcatctgtcttggttaatcttttctgaagtctatag
tgtatttctgtgttgcaacatgagtttactgtcaatcttactgtttgacctcaattttgg
gttctttttgattttgaaagacatcgtttaacaggttggcatggctgctactcttgctgg
tgtctgtcaggtgcctctcactgctgttttgcttctctttgaactgacacagaattatcg
gatagttctgcccctcttgggagctgtggggttgtcttcttgggttacatctggacaaac
aaggaaaagtgtagtgaaggatagagaaagactaaaagatgcaagagcccacatgatgca
gcgacaaggaacttctttctccaacatttctagtttaacttattcttcaggtgtgaaacc
ttcacagaaagagagtaacctatgcaaacttgagagttccctctgtctttatgaatctga
tgatgaagaaaatgatttggcaaggacaattctagtttcacaggcaatgagaacacgata
tgtgacagttctaatgagcaccttgctaacggagaccatatccctcatgctagctgagaa
gcaatcttgtgcaataatagttgatgaaaataattttctcattggtctgctgacacttag
tgatatccagaattacagcaagttgccaagagcagagggcaatttccaggaggtagcttc
ttggtacatttcaatattcttaactgatgaaaaaataagggaaattgatctagcatgaaa
ttaagctaattataagttttacactgtagaactggtaaaacagggttggctggatatttc
tttgttgaatttttaggattatatgtattgttttagttttgtaggttgttttctgatgtg
ctttttgacttggcagaatcttaagatgaaatggaaggtgtttaaccaaaaaatagaatt
ttcagtcaaagcctatatttagaagaaaacgggttattgataaccaagttttactttact
tccccaacaatctatttggtaaatagcaaaagtaatgcgtatgtgagaaagcacgggaga
aaatatattattgatattagatattcaatataatacaagaggtcctacacatcatatagc
tatagtctacaaactacatattactctcattccaatgtgggactacacataactaacact
ccccctcaagccggtgcatacatatcatatgtaccgagcttgttacacatgtaactaata
cgagaaccagtaagagacttagtgaaaatatctgctagttgatcatttgactttacaaac
tttgtaaaaatatctcctgaaagtattttttctctgacaaagtaacagtcgatctcaatg
tgtttagtcctctcatggaatagcggatttgacgcaatatgaagagcagcttggttatca
cacaccagttccatcttgctgatttctccaaactttaactccttgagcaactgcttgacc
caaactaactctcacgttgccatagccattgcccgatattcgacgtcggcgccagatcga
gcaactacattctgtttcttgctcttccacgagaccaaattacctcctactagaacacaa
tatccaggcgtagaacgtctatcaaaaggtgatcctgcccaatcagcatttgtgtaccca
acaatttgctcgtggcctcgatcctcgagtagtaatcctttgcttggagatgactttata
taccgaagaatgcgaacaactgcatcccagtgactatcacagggagaatccataaactga
cttacaacactcaccggaaaagaaatgtcaggtctagtcactgtgaggtaattcaatttg
ccaaccaacctcctatatctcgtagggtctctaagaggctccccgtgtctaggcagaagc
ttagcattcggatccataagagagtcaataggtctgtaacccatcattccagtctcctca
aaaatgtctaaggcataattccgctgtgaaataacaatacctgagctagactgaggcact
gagcaacctcaatacctagaaaatacttcaatctgcccagatccttagtctggaagtgct
gaaagagatgttgcttcagattagtaatatcatcctgatcattgccagtaataacaatat
catcaacataaaccactagataaatacacagattaggagtaaagtgccgataaaacacag
agagatcagcctcactacgagtcatggcgaactcctgaataattatgctgaacttaccaa
accaagctcgaggggactgtttcaaaccatataatgacctgcacaatctacacacacaac
cattaaactccccctgagcaacaaaaccaggtggttactccatataaacttcttcctcaa
gatcaccgtggagaaaagcattcttaatgtctaactgataaagaggccaatgacgtacaa
cagccatggacaaaaagagacgaacaaatgctattttagccacgggagagaaagtatcac
tataatcaagcccaaaaatctgagtatatccttttgcaacaagacgagccttaagccgat
caacctggccatccgggccgactttgaccgcataaacctaatgacaaccaacattagact
tacctgcaggaagaggaacaagctcccaagtgccactcgcatgtaaagcagacatctcgt
caatcatagcatgtcgccatcctggatgagatagtgcctcacctgtagacttagggatag
aaacagtggacaaagaagatataaaagcataatgaggtgatgacacacgatgatgactta
aaccgacatagtggggattaggattacgtgtggatcgtacgcctttgcggagtgcaattg
gttgactaagaggagacaagatcgtagtaggtgcagaatctgatgcagggcgtgaatcac
ttgggcatgatgttggatgtggacgacgatgataagtcaagagtggtggagctgcagaag
gttgaactggattatgtggaggaactggaggtggagctacaactggagctgtaggtggtg
gaactggagctataagtggtggagctacaactggagctggagatgtagaggaagatgaat
gagagatagtgactgaatctccaaaaaataaaattggtagtacctcagaaatatctaagt
gatgacatgaacctgtgaagtatgattgagtttcaaagaaggtaacatcagcggacataa
ggtaccgctgaaggtcaagagagtagcatcgataccccttttgtgttctcgagtaaccta
gaaatacgcacttaagagcacgaggagctaacttatctgttcctggagtaaggttatgga
caaaacaagtgattccaaagatacagggtggaagagagaacaaaggtaagtggggaaaca
tgacaaagaatggaacttggttttggataactgaagatggcatacgattaataagatagc
aagatataagaactgcatccccccaaaaacgaaacggagcatgagattgtatgagtaggg
tacgagcaatttcaataagatgtctattttttctttcagctaccccattttgttgagatg
tgtacagacaagatgtttgatgaataatcccatgagatttcataaactgctgaaatgggg
aagacaaatactctcgggcattatcactaggaaatgtgcgaatagaaaccccaaattgat
tttgaatttttagcgtggaaggtctggaaaaatagaaaacaactcagatcgattttttat
caaaaatatccaagtgcaccttgaataatcatcaattattcaataaaactgacaaagtag
cagaatcccaaggtggaactgacccgactaggaccccaaacatttgagaatggactaaag
taaaaggtgactctgcttgattatcaagacgccgagggaaatggaagcgagtatgcttat
cgaactgacatgactcacactctagagctgacaagtgagataaaccagataccattttat
gaagttttgacaaattgggatgtcccgaccgtttatgtaataaatttggtgtattagtaa
caggacaagttgttgaaggaagacaagatgtgagtccgtgtgatttagcaaggataaggt
aataaagtccgtttgattcacgtccggtaccaataattcgtcccgtactgcgttcctgta
taaaaacatggtcatcaagaaataaaacaacgcatttaagtgatttggctaagcgactaa
tagttatgagattaaaaggactattgggaacataaatgactgaatataaaggtaaggaag
gaagtgagcttgcttgacttattgttgttgccattgtttgagacctattggccattgtga
ctcttgaaagagattgaaaatacgaaatagtagtgaaaagagatttgttaccagaaatat
gatctgatgcacctgaatcaatgacccaaaactcagatgatgaagattgggagaaacaag
tcacgctattacctgtttaaacaacagaagctatcacagaagatgtctgcttacatgctt
tgtaccgaaggaactcaatataatctgctaaagaaaccatccgactattcaaagtatcgg
ttcccatgtcgctacaatttgtagtaataggatggatagactcggaaaattgtaaagtta
tcggaatttgtcgtaaccaggatcgagcaagctgtcttgaagaaatggtttcaaaaaatg
tccggaaaggtcacttttacgccggaaaaatataaaaatggtcgaaatttgatttgaatt
agatgggtaggctcggaattgtgaggagagcagactgtcctgaagaagcttaatgaaaaa
atggccggaaagtggccggaaccctcgccgtaaaagttgttaccggcgcgtgaaggcgcg
tggcattttttctgccagataaattttcaggggttggtcgtcggagggtgatcccttgtg
gtggtgttggtttttgcacaataccgacaggccttaggtcacccgaaaatttgcacgatg
actaagttctttcttcccggttaacgctggaatgacgcacatcgatcttttctcactaat
gctatgataccatgtgagaaagcacgggagaaaatatattattgatattagatactcaat
ataatacaagaggtcatatttatagctatagtctacaaagtacatattactctcattcaa
atgtgggactacacataactaacaacgtaaattaacaaagagaaataaggaatgtaacaa
cagtcaatccctaaaatcaaggtagaaaactttgataaagcagagaattatagaatgtat
ttcagtagtacttggaacttgtccttacaaataaaattctttatccttatataggggcgt
acaatcataacatttttcgcacttaattcgaattcattatgagcattaattgtattgatt
gcccgttatcatagataaccataactgacgtatttgtaactataaatgccttataacggc
tctgattccccttccttatttacttctggtttgtgtatctttccttctttttagccttta
ttcattcagttctcgcctcttctttgacaactgtcaagcccgatcctctgttctgtactg
tctcgtgggtgtttcccccgtaccttccttatattcttaattctgttaattgagagtgtc
acttgtcactatgccattgttccacgcgtcatgtttcatccacgtgtaatatcttttttc
caccaatacagataatcccccactttctgaatattctcaactgaatattcgggtaagttt
ttatggcgggaattctttgccgtcgtttttcgagtatcatcgtgtcatcttcagaaccga
tgtgacgtacgtcacgtctatttaatgcctatgccaggtggcttctatcgattggctctg
cagttttttagcgctttttagggtttttcagcggctgcgtcagtcacgaagtgacggttc
cattatgacgcttcataatgactaactttaatgatggtcgtgtcttcttattaatacttc
attcctttttgatctcttggagtcttccttcttcagtatccaccacattacttctttgta
tttctgcatcttctctttgatattcctttggacaatcatgtcttcttctacaccagaccc
ccgtaaggttgtgattgttgacgaacttgatctttctactgctcctactagaagtaggag
aggtggtagacttcgtagtcttggttcactatctaatcgtggttcttcttcccagggtag
tgctgctaagccatcttcttctagacctagggctcctttaacccctagatcttcttctag
gaatagagatttaaatgatccagtgcgcgaacctacagttgcagagattgttcctcaaga
attttcttttgtaactgaccgtgaaaccataaggaatcaaatttcttctatagcctccct
caataccgctaacctttatccaagtttaatcagtaatggtcttctctcccgggttcgaag
agaatattactgaaaccagatttcccaattttagtccctggtgccaaccagagaattact
ccataccatgttggtttttcctttgtttacacctacccttttactttagggttcaaacca
cctattgaaccagtaatcattgaattctgtcgttatttcaacgtgtgtcttggccagatt
gaccacatagtatggagggctgttcatgccttcgttatttatcagatttggtttccatgc
ctttcacttttcagcacttgcttcatctctactcccctaaattgtttcgtgaagtagttt
ttactctcgtggctagaagtaagagagtgttggttagccttgaagacgattgggaccgtg
gctggtacgctcgttttgttgctgctcccactagtgcattagtgggtgaagaaaatatgc
ctttcccggagaaatggaactttgcacgtaagctttcttctcctctttttttttgtctta
aaaaaactccatgtaatcatatacccacttcttcagcaactatggaagttttttatgctt
gggtagaaaagatgttaactgctgcgcctatggagaaaagatcctggaaatacttttctc
aaagatttggttggaaagtgaagacgcacggtactttttaccttcattgtttttcctttt
ctcttccttgtttgttcaatgatttctcatccttccctttttttttactagggtttccga
ttcgtggtattagtcccgcgtctgttccatcaactaggctttccgtgattcttgttcagg
aaagaattttaagtgcttcttcttcaaaaaggaaaactgacggagcccgtggctctgatg
acgaagaagaaacagaggagggttctttggtgcgaaggtcacgcgtcaggagacgcgtgg
tttctgatgatgaaactactccttctcatgaccctctatctagttcaatcccttttagac
tcacggatgagctagagagtacccctttagtgatttcttatgatgatgctgttgatcccc
ctccaagttctgttgatagattgtttgctcatggcttcgagggtgatgaagttttgggcc
tgtttctgaagaattgccccttgcttcccttccagtttcagttttcattaacccttccgt
gtccttacctgatgatactcctgttgttattctcgtggctgcttctactccgtcatctat
tcccgtgactgcttctcatgcagaggccaaaccttctagcagcagaagggcaatgaaaag
agttgttgttgaggttcctgaaggtgagaacttattaagaaaatccggtcaagccgacgt
gtagttgaaacctatgctcggccccgtagagaagaagaagttagaaagccatagctcact
cactttaatgaatgatatcgttcattcttccttgaaagtacaagcttaattatatttcct
ttcttttctctttcttattcataactcttcctccttttttgcagatcaacttgattggca
cagagcttatgaaaagagtttctcaggcggaccggcaagttatagatttgcgcaccgagg
ctgataactggaaggaacaattcgaaggtcttcaattggaaaaagaggttccggcggaag
agaagaatgctttggaacaacagatgagagtgattgcctctgaattagcagttgaaaaag
cttcctcgagccaggttggaaaggataagtatatacttgaatcctcctttgctgaacaac
tttccaaggcaactgaagaaataaggagtttgaaggaactccttaatcaaaaagaggttt
atgcgagagaattggttcaaacacttactcaagttcaggaagatctccgtgcctctactt
ataagattcagttcttggaaagttctctcgcttctttgaagacagcttacgatgcctctg
aagcagaaaaagaagagctgagagctgagatttaccagtgggagaaggattatgagattc
tcgaggataatctatcgttggatgtaagttgggctttcttaaacactcgtctcgagactc
tagttgaagccaaccatgagggttttgaccttaatgctgagattgctaaggctaaagaag
caattgataaaactcagcaacgtcaaatcttttcctcacctgaagacgaaggtcccgaag
gtgatggagattga
(Protein sequence of CLC-Nt2 from Nicotiana tabacum, translated
from SEQ ID NO: 1)
SEQ ID NO: 5
MEEPTRLVEEATINNMDGQQNEEERDPESNSLHQPLLKRNRTLSSSPFALVGAKVSHIES
LDYEINENDLFKHDWRRRSRVQVLQYVFLKWTLAFLVGLLTGVTATLINLAIENMAGYKL
RAVVNYIEDRRYLMGFAYFAGANFVLTLIAALLCVCFAPTAAGPGIPEIKAYLNGVDTPN
MYGATTLFVKIIGSIAAVSASLDLGKEGPLVHIGACFASLLGQGGPDNYRLRWRWLRYFN
NDRDRRDLITCGSSSGVCAAFRSPVGGVLFALEEVATWWRSALLWRTFFSTAVVVVILRA
FIEYCKSGNCGLFGRGGLIMFDVSGVSVSYHVVDIIPVVVIGIIGGLLGSLYNHVLHKIL
RLYNLINEKGKLHKVLLALSVSLFTSICMYGLPFLAKCKPCDPSLPGSCPGTGGTGNFKQ
FNCPDGYYNDLATLLLTTNDDAVRNIFSINTPGEFQVMSLIIYFVLYCILGLITFGIAVP
SGLFLPIILMGSAYGRLLAIAMGSYTKIDPGLYAVLGAASLMAGSMRMTVSLCVIFLELT
NNLLLLPITMLVLLIAKSVGDCFNLSIYEIILELKGLPFLDANPEPWMRNITAGELADVK
PPVVTLCGVEKVGRIVEALKNTTYNGFPVVDEGVVPPVGLPVGATELHGLVLRTHLLLVL
KKKWFLHERRRTEEWEVREKFTWIDLAERGGKIEDVLVTKDEMEMYVDLHPLTNTTPYTV
VESLSVAKAMVLFRQVGLRHMLIVPKYQAAGVSPVVGILTRQDLRAHNILSVFPHLEKSK
SGKKGN
(Protein sequence of CLC-Nt2 from Nicotiana tabacum, translated
from SEQ ID NO: 2)
SEQ ID NO: 6
MEEPTRLVEEATINNMDRQQNEEERDPESNSLHQPLLKRNRTLSSSPFALVGAKVSHIES
LDYEINENDLFKHDWRRRSRVQVLQYVFLKWTLAFLVGLLTGVTASLINLAIENIAGYKL
RAVVNYIEDRRYLVGFAYFAGANFVLTLIAALLCVCFAPTAAGPGIPEIKAYLNGVDTPN
MYGATTLFVKIIGSIAAVSASLDLGKEGPLVHIGACFASLLGQGGPDNYRLKWRWLRYFN
NDRDRRDLITCGSSSGVCAAFRSPVGGVLFALEEVATWWRSALLWRTFFSTAVVVVILRA
FIEYCKSGYCGLFGRGGLIMFDVSGVSVSYHVVDIIPVVVIGIIGGLLGSLYNCVLHKVL
RLYNLINEKGKLHKVLLALSVSLFTSICMYGLPFLAKCKPCDSSLQGSCPGTGGTGNFKQ
FNCPDGYYNDLATLLLTTNDDAVRNIFSINTPGEFHVTSLIIYFVLYCILGLITFGIAVP
SGLFLPIILMGSAYGRLLAIAMGSYTKIDPGLYAVLGAASLMAGSMRMTVSLCVIFLELT
NNLLLLPITMLVLLIAKSVGDCFNLSIYEIILELKGLPFLDANPEPWMRNITAGELADVK
PPVVTLCGVEKVGRIVEVLKNTTYNGFPVVDEGVVPPVGLPVGATELHGLVLRTHLLLVL
KKKWFLNERRRTEEWEVREKFTWIDLAERGGKIEDVVVTKDEMEMYVDLHPLTNTTPYTV
VESLSVAKAMVLFRQVGLRHMLIVPKYQAAGVSPVVGILTRQDLRAHNILSVFPHLEKSK
SGKKGN
(Protein sequence of NtCLCe from Nicotiana tabacum; sequence originating from
the ancestor N. sylvestris; one start codon, translated from SEQ ID NO: 3)
SEQ ID NO: 7
MCDSSKVDSDSGIQIGSLLEEVIPQGNNTAIISACFVGLFTGISVVLFNA
AVHEIRDLCWDGIPYRAASEEPIGVHWQRVILVPACGGLVVSFLNAFRAT
LEVSTEGSWTSSVKSVLEPVLKTMAACVTLGTGNSLGPEGPSVEIGTSVA
KGVGALLDKGGRRKLSLKAAGSAAGIASGFNAAVGGCFFAVESVLWPSPA
ESSLSLTNTTSMVILSAVIASVVSEIGLGSEPAFAVPGYDFRTPTELPLY
LLLGIFCGLVSVALSSCTSFMLQIVENIQTTSGMPKAAFPVLGGLLVGLV
ALAYPEILYQGFENVNILLESRPLVKGLSADLLLQLVAVKIVTTSLCRAS
GLVGGYYAPSLFIGAATGTAYGKIVSYIISHADPIFHLSILEVASPQAYG
LVGMAATLAGVCQVPLTAVLLLFELTQDYRIVLPLLGAVGLSSWVTSGQT
RKSVVKDREKLKDARAHMMQRQGTSFSNISSLTYSSGSPSQKESNLCKLE
SSLCLYESDDEENDLARTILVSQAMRTRYVTVLMSTLLMETISLMLAEKQ
SCAIIVDENNFLIGLLTLGDIQNYSKLPRTEGNFQEELVVAGVCSSKGNK
CRVSCTVTPNTDLLSALTLMEKHDLSQLPVILGDVEDEGIHPVGILDREC
INVACRALATREQLC
(RNAi sequence used to silence CLC-Nt2)
SEQ ID NO: 8
gtcatcatcaggtgtgtgtgctgctttccgttctccagtaggtggtgtcctatttgctttagaggaagtggcaacatggt
ggagaagtgcactcctctggagaactttcttcagcacggcagttgtggtggtgatactgagggccttcattgaatactgc
aaatctggcaactgtggactttttggaagaggagggcttatcatgtttgatgtgagtggtgtcagtgttagctaccatgt
tgtggacatcatccctgttgtagtgattggaatcataggcggacttttgggaagcctctacaatcatgtcctccacaaaa
ttctgaggctctacaatctgatcaacgagaagggaaaactacataaggttcttctcgctctgagtgtctcccttttcacc
tccatttg
(RNAi sequence used to silence CLCe)
SEQ ID NO: 9
gaaatcctttaccagggttttgagaatgttaatattctgctagaatctcgcccactagtgaaaggcctctccgctgatct
gttgctccagcttgtagctgtcaaaatagtaacaacttcattatgccgagcctctggattggttggaggctactatgcgc
catctctattcatcggtgctgctactggaactgcatatgggaaaattgttagctacattatctctcatgctgatccaatc
tttcatctttccatcttggaagttgcatccccacaagcttat
(DNA sequence of NtCLCe from Nicotiana tabacum; sequence originating from
the ancestor N. sylvestris; two start codons)
SEQ ID NO: 10
atgattagcggccaaaacactgtgctgcacaatcctcctaattcgctcttcaattcctta
tctcctcgccatatctgtatatctttctgtaacgacaaagctttaaaaaagtcagtcacg
cactccgcccctcggtttgctcgtctgttaaacaatgaatcacggaagttgttgggtcgt
catccaaattgctggccttgggctcgacgaccatctcttcctccgggacgttcctctgac
ggaaacattgaaaaagaacaagatatgtgcgacagcagcaaagtcgatagtgatagtggc
atccagataggatctctgctcgaggaagttatcccacaaggcaataataccgctataatc
tcggcttgctttgttggcctcttcaccggtatcagtgtcgtgcttttcaacgctgcggta
cgtgcgctataggtctttcatttctcttttcatgtactattcctccttacttacttggcc
tcagtcaatcagccccctgcctactttaaattattgtacattttatcagaggagtgtcct
atacatcaaattcacataacttagtaaaatatgctgatattctgaattttaaacttacca
gcttagaacatccaggttagttcagaaacagataatctaaattggtctcatttataagtc
attttgttattcaagacatacaatttggctcttgataaaagattatgcagcgcccgatga
ttacctaatatttatcagcaacccatgtaatttaacaatattgtcaccatataaaagaga
actgaagagaatgttcaatttgtggtcatataacggatatctcccttggttaggttcatg
aaatacgtgatctttgttgggatggaattccatatcgagctgcctcagaggagcccattg
gagtacattggcaacgtgtaatcttagtaccagcttgtggcggtttggtagtcagctttt
tgaatgccttccgagccactctggaggtttcaactgaaggaagttggacatcatctgtta
aatctgtattggaaccagttttgaagacaatggccgcttgtgtcacattaggaactggga
attccttaggaccagaaggccctagtgttgaaattggcacatctgttgccaagggagttg
gagctctgcttgataaaggtggtcgtagaaagctgtcactcaaggctgctggatcagctg
ctggaatcgcttctggtttgttccccatattattcttggttctgaaccatacatggtaca
ttttccttataattacatgtagcctgttgtatgctttcctctttcccgggaagccttttt
gtaaatacaagtgtgtttgcactcaaaccaataaactgtaaaaaaggtgaactccttaag
caagcaaaagcattagaaatgtaaactagacatatttctcagattgagagtctgagagat
tagaacacgagtgtttccattagagagagaaaagagacttctagatatttctattatctc
tgtaagagtgaatccgttcctatacaaaaaataggccttcattaaatacaagcttgggct
gggtactactgggccaaagtaaaaaataaaaagaatcacccactatcaaatgggcctagt
ctaacaacccccttcaagctggagggtgacacaacccctagcttgcgaatatgaaaatga
tgagcaggcccaagtaacactttggtaagaacatcaaccacttgagaagcactggagttg
tgaaatagactgatcaggccattcccaagcttgccacaaacaaaatgacagtccagctta
atgtgtttagtgcgttcatggaaaacttggttttttgcaatgtggacttcctgattatca
caaaataaaggaacaggtaaagaaggagaaactccaatatcagacaataatttggtgagc
caagacacctctgcaacagccttactcatggacctatactcagcttcaattgatgatagt
gagacaacaggttgcttctttgatttccagctcaccaagctgccccccaagaaaaataca
aaaaccagtgacagacctgcggctgtctgggcaagaagcccaatcactgcacaataaagc
tgcaaagacaagtctggagagttattgcggaagattccaaagtcaaaagtgcccttgagg
tatcttagcaagtgcagggcagcctgcatgttaggaacacagggagactgcataaactga
ctcagatgctgaacaacaaaactaaggtcaggccttgtgcgtatcaaaaagtttagcttg
tgcattagactcctgtactcttcaggcctgggcaaaggagtgccaatcttagcttttaac
ttcacattcaattcaagggggcaagtgacagaagagcaattcgaggaatgaaaatcagcc
agcaaatcatgaatgaactttttctgatgaagaagaaccccagaatcagtgtataaaacc
tcaatgctaaggaagtaattaagagagcccatgtccttaatcttgaactggtcactgaga
aaggacttcaaagcagccaattcagctagatcacacctagtcaatatgatatcattcaca
tagacaaccaagatgaccaaggaatccctagaacccttggtaaaaatagagaaatcattc
aaggaacgagagaagccattagagcacaaggcttgagataatttagcatactattgtctt
gaagccagtcttaaaccataaagagacttctggagtttgcatactaaaggagcagaagaa
gagtgaggaacagttaggcccggtggcagcttcatgaatacctcctcatcaaggtcccca
tgtaagaagacattattcacatctagttgaaagaggggccagtgttgtttaacagctaca
acaataagagttttgacaatagacatattgaccacaggagaaaaagtttcattaaagtca
ataccctcaacttgagtgacctagctttatatctctcaatactttcattagccctatatt
taaccttgtatacccacttacaactagtaggtttcttgccaggaggcaattcaacaatgt
cccaagttctgttggcatccaaggcctcaaattcacatctcatggctgcctgccattcag
gaacagctgcaacctgagagtaagaataaggctcaggaacatgaagttgactaagagaag
gagcattagaaatagatctggagggaggaggagaagaagtggaggtgcagacataactct
tgagatagttggttggattgtgtggcacggaagatcttctcaaagcaggaggaggtacaa
gagagttagaataatgagaaggagaagagatggaagtgggaacagagaagattgagaagc
agtagaaggagaaagtgaaggagatgaaggagaggaagaagacggaaaggaacattcatc
aaaacaagcagaaaagggaaaggggaagacttgaggtactacatgagaggattgaaagaa
aggaaaaatggtgttcataaaaaatgacatcttttgatacaaaacaggtgttattctgaa
gattaaggcgcttgtagccctttttggcaaaagggtagccaatgaaaacacaaggaaggg
acctaggatgaaatttgttttgtgaggggtggtgacagttgagtaacagaggcacccaaa
agctctaaggtggtgataagtagggtggaagaatgaagcaattcatagggacttttgtga
ttaagaagaggaaaaggaaatctgttaattaaatatgtggcagttaaaaagcagtcaccc
caaaatttaagtggtagatgagactgaaacataagtgacctagcagtctctagtaaattt
ctgtgttctctttctacaataccattttattggggggtgtgaggacaggaggtttggtgt
actatccctttttctgaaaagaaaaggcaaccagaagaactagatcccagttccaaagca
ttatcactcctaacagtttgaactttagattggaattgggtttcaaccatagcaatgaaa
accttgagcaaatcaaaggcattgcggcacccattaaatgtgtccaagtagccctagagt
agtcatctacaatggttaaaaaatacctagaaccattataggtaggagtagaatagggtc
accaagtatttatgtgtattagctgaaaaggctgggtggagtgaatagaactatcaggga
aggacaacctggtctgcctcgctaaaggacaaaccggactagtgaatgaccgtttggaag
acagtttgcaattaagaccagaaatgcatttcattttatagaagggaatatggccaagtt
tgtaatgccaaacaacatcatctttattcacattatgcaaagcagtactagtatttacaa
ttggagtatcatcaggtacagaaataggagcagaaactgaattaagcaaacaagaaataa
ggaaattagaaagaggtaaaggagatgatgttggaggcctggcattctgaaatagtttgt
agagtccattgtccaatctaccaagaaccactggcttcctcactgaagggccctgtaggg
tacaagtagccttggtaaattgtacaatatcatcatcatgggaaagtaatttgtacacaa
agatgagattatattgaaaactaggaatatagagcacattataaagaatcaagtcaggga
acaaggctaaggaaccaatattagtgaccttaaccttatacccattaggaagggagacaa
ggtatggtacaggaagtgtttgaacattaaaaaaacaaatgtttaagggaggtcatgtgg
tcagatgcccagggtctattactcaaactacactatctatcatagtcagcataaatgcac
cataagacaacccttgtgaggtaataactcaccagcaaagttggtagaagcaagatagtt
ggttgaagaagtagatgatgctgatgaagacagttgagattgttgaagtaacattagctg
agaatattggttcttggtaagaccaggaactggataggactgttcaggagcagaggtacc
ttcaggaccagctgacattgcagaaccaccagaggtatccacctcagcatgggcaacaga
ccttctgggaggaagagatctatttgacttgaaatttggaggaaagccattgagcttata
gcacttatcaatgctatgtccgggtttcttacaatagtagacatgtgaagctcaaaagat
cccttagaggtagtaccggacctttgaggttcaaaatttattttaggagagggaggaggc
ctggatacaccaacactgaaagaagcagaatttgaggcatattgagttctagcaaaaatt
tgtctttgcttctcatcagatagcaaaatcccatatacattaccaatggaaggtaagggc
ttcatcatgatgatgttgcttcttgtttggacataagtatcattcagtcccataaagaac
tggtagaccttttgttccctgtcttcagcagatttacccccacaagtacacattcaaact
ctcccggcagacaaagatgcaatatcatcccatagtcgtttaattttgttgaaatatgat
gctatgtccatggacccttgggaaatatgagccagttccttctttagctcaaagatccta
gtacctctcttctaactcagtccaaatattcttagcaaactcagagtattcaacactctt
ggatatttccttgtacatagagttagtcaaccaagagaccacaaggtcattgcaacgtta
ccactgtctggctagaggagaaccttcaggaggtctgtgagaagtaccattaatgaaatc
tagcttgttacgaatagacaaggcaactaggacattacgtctccaattgccataacagct
tccatcaaaaggaccggaaactaaggaagttcccagcacgtctgatggatggacatataa
ggggcgacagggatgggtataatcatcttcatggaaaattaggcgtaagggagtagaaga
agtcgcatcagcactggtgttattatcatttgccatttttttcaacagattgtcaatcaa
ccaacacaatacagatacacatatatagattgtgagaaagcacgagagaaaaatctatat
tattgatattctatttaattataatacaatgagccctatttatacaatacatatcatact
cctattctatgtgggactaggactaattcatattatgtacataactatctaacactcccc
ctcaagccggtgcatacaaatcatatgtaccgaacttgttacatatgtaactaatacaag
gaccagtaaggaacttggtgaaaatatctgcaaactgatcatttgacttcacaaactttg
tagcaatatctcatgagagtatcttttctctgacgaaatgacaattaatctcaatgtgtt
tagttctctcatgaaacaccggatttgatgctatatgaatggcagcttggttatcacaca
tcagttccatcttgctgacctcaccaaatttcaactaattaagtaaatgtttgatccaaa
ctagctcacaagttgtcacagccattgctcgatattctgcttctgcactagaccgagcaa
ccacattttgtttcttgctcttccaagacacctaattacctcctactaaaacacaatatc
cagacgtagaacatctgtcaaaaggtgatcctgcctagccagcatttgagtacccaacaa
tttgctcatggcctcgatcttcaaacaataatctgttacctggagctgattttatatatc
gaagaatgcagacaactgcatcccaatgactatcacaaggagaatccaagaactgactta
ccacactcactggaaaggaaatatcaggtctaatcactgtgaggtaatttaatttaccaa
ccagccgcctatatctagcaggatcgctaagcggctccccctgtcctggtagaagtttag
aattccgatccataggagtgtcaataggtctacaacgtgtcattcctgtctcctcaagaa
tgtctaaggcatacttcctttgtgagataacaatacatgtgctagactaagcgacctcaa
tacctagaaaatactttaatctgcccagatccttagtctgaaagtgctgaaagagatgtt
gtttcaacttagtaataccatcttgatcattgccggtaataacaatattatcaacataaa
ccaccagataaatactaagatttgaagaagaatgccgataaaacacagagtgatcagctt
cactacgagtcatgccgaactcttgaataactgtgctgaacttaccaaaccaggctcgag
gagactgttttagaccatagagggaccgacgcaaccgacatacaaggccactagactccc
cctgagcaacaaaaccaggtggttgctccatataaacttcacctcaaggtcaccacgaag
aaaagcattcttaatgtccaactgatagagaggccaatggagaacaacaaccatggatag
aaaaaggcggactgatgctattttagccacaggagagaaagtatcactgtaatcaagccc
aaatatctgagtataccctttggcaacaagacgagccttaagtcgatcaacctggccatc
tggaccaactttgactgcatacacccaacgacaaccaacaataaatttacccgaaggaag
aggaacaaactcccaagtaccactcgtatgtaaagcagacatctcgtcaatcatagcctg
tcaccaccctagatgagacagtgcttcacctggatggaaatagaggacaaagatgataca
aatgcacaatagggtgatgacagacgatggtaacttaaaccgacataatggggattagca
tttagtgtagaccgttcacctttccggagtgcaatcaattgactaagaggagacaagtcc
gcagtattagcaggatcaggtgcaggacgtgaatcagctgggcctgatgctgggcgcgga
cgacgatgataagttaggagtggtagagctgtagaaggttgaactggactaggcagtgga
actgaagctatatgtggtggaactggagctataggtggtggagctggagctgtaggtgaa
gatgaatgggagatagtgactgaatctccaaaagatggaactggtagcacctcagatata
tctaagtgattacctggactggtgaagtatgattgggtttcaaagaaggtaacatcagca
gacataaggtaccacctgaggtcaggagaatagcatcgatatcccttttgtgttctcgag
taacccaaaaatacgcacttaagagcacgaggagctaatttatcttttcttggagtaagg
ttatgaacaaaacacgtgctcccaaaggcacggggtggaagagagaacaaaggtaagtgg
ggaaacaagacagagaatggaacttgattctggatagctgaagatggcatacgattaata
agatagcaagatgtaagaactgcatccccccaaaaacgcaacggaacgtgagattgtatg
agtaaggtacgagcagtttcaataagatgtctattctttctttcagctacccgattttgt
tgggatgtgtatggacaagatgttttatgaataatcccatgagagttcataaactgttga
aatgggaaagacaaatactctaaggcattatcactacgaaatatgcggatagaaacccca
aattgattttgaatttcagcgtggaaggtctggaaagtagaaaacaactcagatcgattt
tttatcaaaaatatccaagtgcacctgtaataatcatcaatgaaactgacaaagtagcgg
aatcccaaggtagaactgacctgactaggaccccaaacatctgaatggactaaagtaaaa
ggtgactgactctgctcgattatcaagacggcgagggaaatgggagcacgtatgcttacc
gagctgacatgactcacactctagagtggacaagtgagataaaccagataccattttttg
aagttttgacaaactgggatgtcccaaccgtttatgtaatagatctggtgaatcagtaac
aggacaagttgttgaagaaagacaagatgtaagtccatgtgattttgcaagaataaggta
gtaaaatccatttaattcacgcccggtaccaatgatccgccctgtactgcgttcctgtat
aaaaacaaggtcatcaagaaataaaacagagcatttaagtgatttggctaagcgactaac
ggctatgagattaaaaagactaacgagaacataaagaactgaatctaaaggtaaggaagg
aagtggacttacttggcttattccagttgccatggtttgagactcgttatccattgtgac
tgttgggagtgattgagaatatgaaatagtaatgaaaagagatttgttaccaaaaatatg
atcagatgcacctgaatcaatgacccaagactcagaggttgaagattgggagacacaagt
cacactactatctgtttgagcaacggaagctatccctgaagatgtttgtttacatgtttt
gaactgaaggaactcaatataatccggtagagaaaccatccaactcttcgtagtattgga
ttccattttgctacaaccaatttctcaaattcttgattacaacttgtgtggttaaccttg
gaatgccaaatcagaacaccccttttttttttttggaaaacattgttcactcgctggaaa
ataaaaaaggttgccggaatttgatgaaacttgaatagaccgactcggaataatgtccta
agaaggctgtccaaaaggagttttgtcagaaactgaccagaaggaggtccacgcaccggc
gcgtggacagatctcgccgaaaaaaaaaatcactttggttggcgcgtgatggcgcgtggg
tggggtttttccggtcgggttttgtggggtttgctcccccggagatggagaacactgtgg
tggtgttggtttatgcacaacactggtaaaaagtggttttgatgcgaacagctactcagg
tcaccaaaaaattgcacggtgacgactgatttcttcccggatgtcgttggaatgacgcac
aacgataattatctcaccaatgctctgataccatgtgagaaagtacgggagaaaaatcta
tattattgatattctatttaattataatacaatgagccctatttataagactaggattaa
ttcatattatgtacataactatctaacatagatcaaataggcatgcaattcacaataatg
gtgaataaaatgatacgaagttacccagctcttttcgcgatcgaaaaggagaaaatagcc
ttcaatcacaaacgagaaagaagaatctccggcttgacagtagacgacttcgaaacccta
gctcgagatgaaaaccacaaaatccccaaatcacattaccaaccaaacaatttgagatca
caaatgttgaatatgtgagaatccgactaagaaatcaacaaaaaatcaatagaaatggtt
gaagaataccgacttgaaccctaaatgagtcagacatcacctagaatgaaatacaccttc
gaaattgacgaaaacaggaccggttgaaagcggagaacgtgccatagaaggatctacgct
ctgataccatgtaaacttgacatacttctcagattgagagtctgagagattagaaaacga
gtgtttccattagaaagagagaaaagagacttctagatatttcgattatctgtgtaaaaa
tgaatccgttcctatacaaaaattaggccttcattaaatacaagattcggccgggtatta
ctggcccaaagtaaaatataaaaagaatcacccactatcaaatgggcctagtctaacaag
aaaaccaacaaatagtccccccccccccccccaaaagataccactgaaatgacaccgggt
gcccaaaaataaagcagcttacttcttgactttgagaggaactgcaatccttatcggttt
gagaggaactgcaatcagctataagtagcttattaatttccagtgcctgcattctgccaa
gtactatgatatatttctgaagctttgtttccccagttcctttttcagacgtttgctgtc
aataaagttgagccagccaacttggctcccacaagctactaattttgtccaagcttactc
tatgggagaagttaaatttcccaaattccttgagcggaaaatgaaaaatggactcaaagt
gtcatattatgcaactatctaaagaaaaatactcaattgaagtttagataagaaaagtga
atgtatattgatgtagtctccgttaggtgagaagcgtatcacttacccagcaacatatgg
acctaacattttactagtgaagttttcacattgtatcaaaagctcaacaaacggaaaggt
gactaatcctaaaatgttatttcacatatatgggcacacggtttgtcaaccttctcatac
gtgcattatttgttctctatctttctatttcatccgatataaccaatcgttattgtaaat
tctataatgcctgtggttacttttgtctttagtgacaaatgacatttaggataaccatgt
agttattgacttatttcacttgaggtctcttccaattatgtagtagtagagtgttgagat
atggatatgttaccttctaaaaaaaagagtgtagagatgcggatagtttgctagctggct
tttgtctcccttcaagttgaattagcaaaagcttgtctcataagttggatagctagacaa
gaaaaactccaaattactttatgtagagtattcttaagcttgagtcgcgagttggaaact
ggaattatgtaaaaaaacctggaattatttggttgagcctgctttttagttttgtcaata
tttccagtatctaacccaacatgtttagagtgattcccggagagcctcagtacaaggcat
ttgcagagtctttatgagagtccaggaaggggcacacattctgtagaggtatagtcttgt
ccttattttcagggttgaactagttctttagaagttacctaggcttcctaatttccaaat
ttctgccaggtccttttttggtgaagtacttgaagtttaataaatcaaattttaatttct
aacatatcctgagaaatttattcacaaattcaactggtgacttctgatgcagaaacataa
gcaactgcttatgggttcatatgttcctgcaattttattgttgacatggattggcttcat
atggttttgttcctgcaattttatcgctgacactaatcctttcatatggttttatgtgga
gtgttaaatagaggttaagagacaagaagaggctgaaaaaggtgggcagttcatttgtta
gtagactactctatttactaagagatatgatgtcccatacattactcgaattggctccga
atccagattccacttctttgccgagtttccttattgtacatagttcgactcgtcaaggga
aattcacttcctttgactgaataatgctagtttgagtagtaccttacattaaatggacca
tttagttctatctacttgatagaatagactggtcatcaactagttgcaaatacaatgaca
actttgccatgtttgcagagtcacctgatgaagaagtacctcaattagtagaacatttct
tgaatgttctacagtattctctatgcctacatgaccacatcacttttccttttgcgttgt
gagaacttgaacttggtgagcgggggttccccaggaatggcatcttgatggcagatgacc
attctgtccttgtcttagctaatgcttcttgcattgcctcactagatttattataccttt
aaaaaatgtttgccattgttctgccataatagaaggatgtacccagctggtgcttcaaaa
ctaatgaaatgctttacaattgtcgagtcctaaaggatgatttgtggaatcagatctcaa
acaattctttttgaggaagaaaaataccaaaggttttttctgtttgttggaagattaaaa
atcctttaaatggtaaagatttatgaacttaattcagcgtttttgtggccattgctggaa
aagagaaaaaacaatggcacttcttcgagtttgcttatccaaaaaaaagaagaagagaat
gtcacgtaatgcaatttcatcttaggaaactttgcaggagaaaagcaagagtgataaaac
agaactatttgttttttttaacaagttgttgtgacctatttcttgtcattcttatttgct
aataagctaatgtactatagttcctgtactatggtttgttttgacttaatacggggatgt
tcaatgagcattttcttgttttttctgctttcagcatctgctgccttacaggaattcatt
ttctggaaatttacttcttgttctgctaacattttcctgttatatcttgtcagtcatttt
ctctccatggttatactgtttgtgtcactttaaactctccttgttttctactttaaagga
tttaatgctgctgtcgggggctgtttctttgctgtggaatctgtgttatggccatcacct
gcagagtcctccttgtccttaacaaatacgacttcaatggttattctcagtgctgttata
gcttctgtagtctcagaaattggtcttggctctgaacctgcatttgcggtcccaggatat
gattttcgtacacctactggtaattttggacttctttctcgagtttgattcttaaataca
attgtacccgtcacttacagcaacaactacatttcaacagctagttggggttggctacac
agatcatcactatccatttcaattcatttagtcccatttctttcgaatattgagtacttt
gggattctataatatcaaggttctttatattttctactttgacgtacaaatctctaaata
gattaaagaagactcctagagacactggcctaatgcaaatgtaccaccatgaataaactt
taatctgaaatagctggtatcttatataaggacccttagctttaattgtgttctatattg
atcttttgggacaacttccttccaatattatgtcttacttatacagttatacttatcctt
aagccttactctttagagtggttatccctaattcaagcttttgttggcaccatagctagt
ttggttctaagtaaaaagttactctttagagtggtaactttttgtcaattttcttagtga
aaatataacctctgtgacaaatctaccaagtataaatccaatttggttctatgtcatcct
tgtagtttatccaagtcaatgctccatcactcttacaaaggttcatcgtatgactaatct
tttttggagaaaggtaacagtttgtattgataataagatcagcgccaggttggtcattag
tgctaatagctgtacgtacaactccaaaagagcaaaagacaagcacctgatgtaaggtaa
attacaagctgcctataaaatctatcaggtgtcctatctcactaaacatttcttgtttac
accaaaaaaataaaacaaggaaagacaatccatcttaatcttctgaatggagtttctttt
tccttcaaaacatctggagttccttccgttccatgcaatccaccatatacaagctgggat
gattttccatttgtctttatccatttcttctaccaattcccttccaattgattagaagtt
ccaatgtggttctagatatgacccaattaactcccaacagataaaagaagatgtgccacg
gatttgtagtgattctgcaatgtaggaacaagtgagcattactttctacttcctgtccac
aaagaaaacatcttgagcaaatctggaaacctcttctttgtaagttatcatgtgttaaac
atgcctttttcaccaccaaccagacaaaacatgatactttgggaggagttttaaccctcc
aaatgtgtttccaaggccacacctcagttgttgaaacattaggatgtagagtccagtatg
ctcttttactgaaaatgcaccttttctattcagcttttaaactactttatctatggtctg
tgatgtacccttgaaaggttcaagagtttggaggaagatagaaactctgtttatctccca
atcatccaaagatcttctaaagttccagctccatccttgtgagctccagactgacttacc
aatgcttggctttgaagacttagagagaataagtcaggaaaatatctttcaaccttcctt
gccctatccggtgatcttcccaaaaagatgtctgcaacccattgccaatattgatcttga
tattgctactgaaagatttcttttggtggcaggattactctcattaacaatgtacttgac
aatctccatacatactaatgtctctttaccctcttgccattaaggttgtaaagagacttg
tcaaattaagaaaaggtttcctatggaactgtttcaaggaaggaacctcctttcctttgg
tcaagtggagttaagtcatataatctaggaagtggaggcttgggtatgaaatagctgcaa
atacagaaaaggagcatcttatttaaatgatcacggaaatgtgcccaaaactttaaatat
ctgcacagcatatggttgtagcaaaatttgaatcttcctgtcaatggtgctcatgtccag
tgaatacccctgatggtgaaagtgtcctgaagggaagcaggaacttattggaagaattgg
catctaacactcagcttttcggtgggtcatagcccattgaaaattgagtgcccagattta
tatagttttgctctaaactgacgatgcagttgcacaacatacgacaaactaaggtgggac
atcatcttcttcggaaggaattttgaggattaagagatagagtggttgattcagttgcaa
atgaagcttcaagggttcaatatcatccaggagacaccggattctgatagataaaacaac
agaaagatgagcactactttgttaggcttgttacaagttgctatcgtctttcttatctcg
gtacacaatttagatttgggaacttagttggaaaagcagagtggttgtttttgtgaatag
catcagacaaagcttctgagctggtacgacagaaaactcaacagggagaatagaagactg
tggttcacaatttctgcatgcatcttgtaggttatttggtgggtaaattatttaatgttt
tgaagggaaggtagaacatgttcataggcttagattcaaatgtttgtatttttttggctc
tttggtgagagatgctgaacgtaaatgacataggcagctgactataatttctcagctcct
tgctttttaaattgacaggcactgatatgtacatgtgaacatccaacacttttgtggtgc
cgttccgatgaataaagaacattaatcacttactgatcaggagtaatagtttaggagttc
tagaatttttgtacataaaatgaaccaaaaagaagatcggaatgagaacatgtttctttt
tttgttttttctttttcgtgaaaacttcaataacacttctgatagaatagctaggtccat
ttgaattcctttggagacccttacacaaccaatgaatgacaagtatagcatttctaactc
cctcccacacgtataacccagattttagggtttagatgtggatctgatttgaccttattg
cctttttttgtttttgttctttttgaagtagagagtgaggaggctcaacaattaattcgg
ctcaacgggctaatgattggacttacatgctacgacaatgttaggagagagagagagaga
gagaagcccagagcagttacatgagttaagaaagagaagtccaaagcgatagaatatgaa
gagagaaagcggttgtgctaacaggctccctgaagtttggctctgagcatccaactcaaa
accttaaggcaatgagtagagtagcccaggaccatttaaattgctgttgaaaaccttaca
caaccaataagggaacaagtgtaacattctcttacaaccctaccgtcttataagtcagtg
ctctaatttagcataaaatcaaagtgaggcgatctacaatgaaatgaagtaaataactga
taaatacaaagaatgttaattctccaatatagcctgaatgttcccagaacaaaataaact
agtctcaggatttatcattaacatgatgttcctcttattttgagtgattaggaaggttaa
tcaaggtataaattctttctaatttgtatcgtctagaattatttatctaacaaattttca
gattaccggttcaaaagaggaatatattttgcatacaacgttaccataccttacaaaagg
gagatgaacatttttttattttattattgtcctttttttcaattagggattatgcagtct
tcctccacgtgatattactcttagaatcacgtttttgtcattgctattacttaatgtggt
aagtacaaatgtgttttgaactctttttggtatgtaatattgagttaatttttggtttcc
atttcagagctgccgctttatcttctgctgggcatcttttgtggcttagtttcagtggca
ttatcaagttgtacatcatttatgctgcaaatagtggaaaatattcaaacgaccagcggc
atgccaaaagcagcttttcctgtcctgggtggtcttctggttgggctggtagctttagca
tatcctgaaatcctttaccagggttttgagaatgttaatattttgctagaatctcgccca
ctagtgaaaggcctctccgctgatctgttgctccagcttgtagctgtcaaaatagtaaca
acttcattatgtcgagcctctggattggttggaggctactatgcaccatctctattcatc
ggtgctgctactggaactgcatatgggaaaattgttagctacattatctctcatgctgat
ccaatctttcatctttccatcttggaagttgcatccccacaagcatatggcctggtatga
atttgtcttttgttagaagtagcattacatatctggataagtgagttttttattattgaa
aagtaataacaggagagcaagagaatatagcacccaaatctacttctttcctctcttcta
ttcttctgaaattcaaggtcctttaactcctccacggcctgtctagttattgatcctgta
gacttaattcacataggtttaggacattcaagtttatccaaacttcgtgaaaaggtttct
aatttttttacattacagtatgagtcgtgtctacttgagaaacatatcactccatgtttc
tatagagtctgttttctcctcagtttattttgatatatggggtcctattaagacagttca
accttggattttcattatttttgttgtttcattgataattattcaagatgtacttggatt
ttcttaacaagagatagttctcagttgttttttgtgttcctaagtttttgtgctgcaata
caaaattagtttgatgtctctatttgcatttttcccaatgataatgccttagaatatttt
cttctcggtttcagtagcttatgatttctttagaaactctctatcagaaatctcaactga
gatagatgagaggaagaataagcatatcattgagacggctcgtacccttctcattcagtc
ccctgtcaagcttagtttcttgggcgatgcagtttcacgtcctttgattagattaattgg
atgcctcatctgctatccaaaatcagattcaactttcgatattgtttcctcgcttacctt
tatactctctttccctcgagtctttgggagcacatgttttgttcaataacatagctcctg
gaaagtgaccagcgcaaccgacaagcaaggccttcttaatatagaaggagggcatatgct
attctagccacgagggagaaagtaatattgtaatcaaacccaaatatctgagtataacct
ttggcaatggcgatcaatttgattatatggaccaactttgcctacatatacccaccgata
gatttacggggaggtagagaaataagctcccaagtaccactaatatgtaaagcagacatc
tctttgatcatagcctgtccttgtggacatagggatagaaattgaggactaagatgacac
aaaagcataatgctgtgatgataaacgatgataactcaaatcaatatgatggggatggga
attaagagtggattgaatatctttgcggaatgtgattggtagactaggaggagacaagtc
cgcaataggtaaaagatccagtacatggaatgaatcttctggacatgatgttggactgac
gtcaatgataagtcaagagtggtggagttgcagaacatggaactggagctgtaggtgaca
taatcgaagttgtagggggtggagctatagaggaaggtgaaggagagatagtgactgaat
ctccaaaatatgaaaccggtaatacctcaaaaaatgtctaagagatcatttggacctatg
aagtatggttgcgttttaaagaaggtaacatcagcagacataaggtaccgcggaaagtca
ggtgaataacattgatatccttgttgcgtcctcgagtaacttagaaatacatatttgaga
gcacggggagctaacttatcttttctggagtaaggttataaaaaaacacatgctcccata
gacacgaggtggaagagagaaaggtgagtggggaaacaagacagagtatgaaacttgatt
cttgatagttgaagatggcatacaattaataagacaataggatgtgagaactgtatcccc
acgtaaacacaacagaacatgagattgtacgagttgggtatgagcagtctcaatgagata
cctattcttcctttcagctatcccattttattgagatgtgtatggacaaaatatttgatg
tatgatcctatgagagttcatgaactgctgaaatggagaagacaaatactctggggcatt
atcactatgaaatgtgcggttagaaaccccaaattgattttggatttcagagtgaaaggt
ctgaaaaatagagaccaactcagattgatttttcatgagaaatatccaagtggacttgga
ataatcatcaatgaaactgacaaagtagcagaattccaaggtagaactaactcgacaagg
acctcaaacatctgaatggactaaagtgaaaggtgactctattcgattatcaagacaccg
aggaaaatgagagcgagtatgccttctgagcggatatgactgacgctctagagtggacaa
gtgagacaaaccaggtaccattttctgaagttctgataaattgggatgtcctaaccgttt
atgtaataaatctggtggatcagtaaaaggacaagctgtaaggggacaaaaataccaaat
atttccagaagatggcaaactacaacagaagaagcaactacattaacaggctcaggatat
gtgatgaaatgaggacaaagagttgatcaagaaggagattctggaattctaccagaactt
atatagtgaaaatgaaccgtggaggcccagtgcaaattttgaaggcatctcctcactaag
catagaagagaagaactagttggaagctccatttgaagaaatagaggtgcttgaagcttt
gaaatcatgtgcccctgataaagcaccaggtccagacggcttcaccatggctttctttca
gaaaaattgggatactcttaaaatggacatcatggccgcacttaatcactttcaccagag
ctgtcacatggttagggcttgcaatgccaccttcatcgccttaattccaaagaaaaaggg
tgctatggagctcagagactacagatctattgacaaactagtctcgggggaacaaaatgc
tttcatcaagaacaggcacatcactgatgcttccttgattgccagtgaagtgctggattg
gagaatgaaaagtggaaaaccaggcgtgttgtgcaaactggacattgaaaaggcttttga
tcaattaagatggtcttacctcatgagtatcttgaggcagatggctttggggagaaatgg
ataagatggataaactattgcatttcaactgtcaagaactctgttttggtgaatagtggc
ccgaccggttttttctcctgccaaaagggcctaaggcaggggatctcctctcccctttcc
tattcattttggcgatggaaggactcactaaaatgttggagaaggctaagcaactacaat
ggatacaaggctttcaggtgggaaggaatcctgccagctcagttacagtatcccatctac
tctttgcggatgatactcttattttttgtggtactgagagatcacaagcacgaaatctca
acctgacgctgatgatcttcgaggcactatcaggactccacaacaatatgataaagagca
tcatataccctgtgaatgcagtccccaacatacaggagctagcagacatcctatgctgca
aaacagatactttcccaacatatcttggacttcccttgggagctaaattcaaatcaaaag
aagtttggaatggagtcctagagaagtttgaaaagaggcttgcgacttggcgaatgcaat
acctctccatcggtggcaagttaactttaatcaatagtgtactggacagtcttcctacat
accacatgtctttgttcccaattccaatctcagtcctaaagcagatggacaaactcagaa
ggaagttcttacgggaaggatgcagcaaaacacacaaatttccactagtgaaatgactca
aggtaactcaaccaaaattcaaaggaggcttgagcatcagggatctacaagcacacaaca
aagctatgctcttaaaatggctctggagatatggacaggaggaatctaggctatggaagg
acatcatagttgctaaatatggagcacacaatcactggtgttccaagaaaacaaacactc
cttatggagttggtctgtggaagaacatcagcaaccactgggatgaattcttccaaaatg
taactttcaaagttgggaatggaactcgtattaagttttggaaggatagatggctcggaa
atacacctttgaaagacatgtttcccggtatgtatcagattgccttgaccaaagactcca
ctgttgctcaaaatagagacaatggcacttggtgcccattttcagaagaaatttgcagga
ttgggaggtcaacagcctactcacaatgttaagctccctagaaggtcataatatcgaaga
tcaacagcctgacaaacttatttggggaaattctgagagaggcaagtacacagtcaaaga
atgatacattcacctctgtgaccagaatccaataatagataactagccatggaaacacat
ctggagaactgaagtgcctaccaaggtgacttgcttcacatggttgactctaaatggggc
atgtctcactcaagacaacttaatcaagaggaatatcatactagttaatagatgctacat
gtgccaacaacagtcagaaagtgtaaaccacctattcctccactgctcagttgcaaaaga
catttggaacttcttctacactacctttggtctgaaatgggttatgccacaatcaacaaa
gcaagcttttgaaagttggtatttttggagagttgacaaatccatcaaaaaaatctggaa
aacggtgccggctgcatttttttggtgtatttggaaagaaaggaaccgaagatgttttga
tgacatattaactccactctactccctcaaggctgcgtgtttagttaacttatttagttt
tgtggattttattagctccctgatagtagcataggcttttgtaaatggagctaattatcc
tatctcttttgtactctttgcatcttcttgatgccttttaatgaatctaatttacttcat
aaaaaataaaaggacaagttgttgaaggaggaaaagatgtgagtccatgtgatttagcaa
ggataaggtactaaagtccatttgattcacgcccggtaccaatgatccatcccgcattgc
attcctgtattaaaacagagtcatcaagaaataaaatagagcaaataagtgattggccaa
acgactagtggatatgagattaaaaggactatcgggaacataaagaactgaattcaaagg
taaggaaggaagtggactagcttaacctattccagttgccatggtttgagaatagttggc
cattgtgactgttggaagtgattgagagtaagaaatagtagtgaaaagagatttgttacc
agaaatataatcagatgcaactgaatcaataacctaagagtcggaaaaagaaacacaagt
catgttattacctgtttgaacaatagaagttatctccgaagaggattatttacatgtttt
gtactgatggaactcaatataagccgataaagaaaccatccggatattcaaagtattgga
tcaacagcttataagccaaaagcatccgatacgagtgccattataatggatcaagagaga
tcaaacaacaaatcaccaaatatcataaacaaccaagaatctcgctggaatgtgaacaaa
gattgaaaaacaacaatgtagctcgccaaaaatgtgcaaagtgatcgaaaaatattgaat
cgtgagtggagagaaataggagcttcaatcgacccacacagtaccaaaaaatccaaaaac
ggttgtcggagctcaagaaagttgtcaaaaagtatattgtatgcttcgaaagtagccgaa
aaaggttggaagtgggatgtgtcaactccgaattatgatacgagcaccacagaagatcaa
tttgtgtcaaaactaccgaaaaaaatacttcacaccccgacgcgtggagtactcgctcgt
tggaacccttgctgccaacgtcgcatgtaggatcagttttcgaagaatcttattggggtt
tggtcgccggacgatgtcggatcttgtggtgccgttggaattcgcacaaccctgaaggaa
aagaaggttacacaaatcagatctgaaagtcaccgaaaagacacatggcgattgactttt
ttgtctcagatgtttctcaccgtcgctctgataccagttgttgggctcaactcgtttgaa
gatactcttaacatagtgtgatattgtcccttttggaatgtgagtcatcttagctcggta
agcatactcgctcttccaactagcccgaagatacttttaacagagtgtaatattatctgc
tttgagccaagctggcgcggttttcatcaaaagacctcatactattaaaagatccataca
ccttatatgtaggcttctaagttgctcggacacgggtgcgagtacccgacacaggtgcaa
atctagaggtcagatcctttaaaatgtaaattctaagatttggggatacgaatcctagta
cggatacgggtgcgaggatccgattaaaaataattcaaaaaaataagaaaataaaaaagt
ctctaaattatgtgaaattttgtggaataactacgtatagcttgtaaagtgtggatttat
tttttattctcaagttgtagataagtaaatgattgatttcctagataaggtatgttattt
tcttcaaatttaccctagtttggttcgaatttcgggaaattgtatcttgtctcgaatttt
tccttctgtcctgattaaactactcaaaatcgtctgaccagatccggtacggatcccata
cccacatccacactagtgtcgtgtggacaagggtgcggcacctaaacttccgtgtaggag
caatttaggtaggctcctaatcttttcagctattaatgtgggacttttacgcacctctat
caaattccccaataaactaagtttcacgtggtccatcatcgcaatccacgggtctcttcc
tctagttaagtcccacatggcccattaccatgatccacgggtcaattttcgtgattcatc
gtgtgccacccacatcgttagtatttatggtaactaaagtacgcaactagcttttgcttg
tgagcgtgtctccaagctcgtaaaggtaagaaaaccgagccgcatattccatcactctat
catcaccatactcgtcccgcgaaacttgtaagataaaggtggctggttggtcagttgaac
tacctcagagtgacttggtatagtatttcctttcttgtgaatatttaactcaattatgga
ctctctgtgtgatagtcattgagagccattttctatatagccggtgcacacaaatcatat
gtaccaagcttgttatatatgtaactaatacgaggaccagtgaaggactcggtgaaaata
tctgcaatctggtcattcgacatacaaggccaatagactccccagcaataaaatcagggg
gttgctgataaatagaattggccgaaatgttgccagaaaaatttgaaaatagtgagacta
agccgaattctacactacaaaataggttttaaaacacaaccagaaaacaaaaactttttt
ggaaattactgttcacatcgaaaaaataaaagttgtcagaatttgatgtaatttatatgg
ataggctcgtaatcactggacgagtaagttgtcctgaagaagttttgtcaaaaggtggcc
ggaatggctcacacatgccggaaaacttattgtagctcgccggaaccctagttctggcgg
tgcgtagaggcgtgtgactttctgccagactgattgactgtggtttgtcgcctgactttt
cctaacaagatggtagtattggttttcgcacaacaattaccgatgaggagataacgcaaa
tcaatcttgagtcgtcaatcggaaagacgcacggtggctgactttctatttagatgggac
tggaatttctggagtttaatcgcacaagcgttttggatctgatggtaatactggtatgca
cagtaccactgtagcagtgatgaaccctcaaaataagacaaagttgccagaaaattgcac
ggcgatgagatctttcttccggatgtcaccggaatgacgcacaacgataatttctcactg
aagctctgacaccatgtgagaatacacgggagaaaaatctatttttattaacaatgatac
aatgagccctatatataatacatattctactctactacatatgggaatagggcatatttt
actcctactacatatgagactaggactatttacacataactatctaacaagggctatatc
tcagatttatgagaatatctacccaacgacccagagagacgagcctaatcattttgcagt
ggcacagactataacaacaaaaaacctactcataatggttaaaccaactgattaagatgc
ttacaggactatcttgagaaatgtacatattatatagatgcttgagttgcgtcccaatcc
taaatagaagcttttattcgtaagcaagaagggaagcagctttacttgagccaatagctt
tcaaggtgcatgttgtcacaccaaggacatccagaatttgattttatagtgggaatatcg
tttaaagataaaaaagatagcgtgcagaagattgcatacattagagatgcaaaatacgga
atacccatactcccagataatgcagtatgccttttgcatgacctactggttgaatggaag
cacctggtgaatttactaggtgtgttagtgatttctgctgcttccttcccctttctaaac
tgcatactatctaaaatgttaggggggcagaagcccagtcaatctgactaggtgatgtta
gtggtttccgcttcttcctcccacttctaaatgcgtactttctcaaatttaggagcatag
aaacttaagcagctgcctacctgaggagttgcatgggaacataagagaatagactttacc
tgtcatattttccataccttagttaattacagtgttatcctgataatgatctgttttctg
gatctaggctgaatcgagattcaatcgcttttggttgaaaggatgctgctacagatcctt
agtttacatcattttggttcttattctataagtacttcccctatcaactacttccttctt
ttttcttaggttatttgcctctttaggttgtttggaaggaaaggaacagtagatgttttg
atggaatagcaactccaaaccacttccttaaggctaatatcctgattggccaagtttctc
caaagtccaaaacactttttttttccttcaaaaaagtacctttttttttcaaagttgagg
tgtttggccaagcttttggaaggaaaaaaagtgtttttgagtagaagcagatgctcttga
gaagcagaagaagtagcttcttcccggaagcacttttgagaaaaataaatttagaaacac
tttttaaaagcttggccaaacactaattgctgcttaaaagtattttcagatttattagac
aaacacaaactgcttctcaccaaaaatacttttttgaaaagtacttttcaaacaaagcac
ttttcaaaataagttttttagaagcttggctaaacaggctataaatgtcttttattttta
cagctggagtaccctaacacctgtaaattcccctatacatttttttcgactttggtagct
cattaaccctagtataggactctttgttttggagctagcaaactcttttgttttcctatt
tttgcatcttcttggtgccatttataatatctcttcaccaaaaaaaaaaagttcccaaac
tatgactaccttgagttggtcaaagcataaccaaagcatgggcacaccagtgtttgcgtg
aattttatggatgttccttacctttatccttctgtgcttatgtagcatctgtcttggtca
atcttttctgaagtctatattgtatttctgtgttgcaacatgagtttactgttaatctta
ctgtttgacctcaattttgggttctttttgattttggaagacatcgtttaacaggttggc
atggctgctactcttgctggtgtctgtcaggtgcctctcactgcggttttgcttctcttt
gaactgacacaggattatcggatagttctgcccctcttgggagctgtggggttgtcttct
tgggttacatctggacaaacaaggaaaagtgtagtgaaggatagagaaaaactaaaagat
gcaagagcccacatgatgcagcgacaaggaacttctttctccaacatttctagtttaact
tattcttcaggttcaccttcacagaaagagagtaacctctgcaaacttgagagttccctc
tgtctttatgaatctgatgatgaagaaaatgatttggcaaggacaattctagtttcacag
gcaatgagaacacgatatgtgacagttctaatgagcaccttgctaatggagaccatatcc
ctcatgctagctgagaagcaatcttgtgcaataatagttgatgaaaataattttctcatt
ggtctgctgacacttggtgatatccagaattacagcaagttgccaagaacagagggcaat
ttccaggaggtagcttcttggtacatttcaatattcttaactgatgaaaaaataagggaa
attgatctagcatgaaatgaagctaattataagttttacacagtagaactggtaaaacag
ggttggctggatatttctttgttgaatttttaggattatatatattgttttagttttgta
ggttgttttctgatgtgctttttgactcggcagaatcttaagatgaaatggaaggttgta
tcatcaaatgttaaataagggaatatgtgactttcaaagttaagcacggagtattttgga
gtcaatagttacttcctgaatcttttaggatggaggagacagtttctataggaataggaa
aaggggacctgatttcattatttgtgtgtatatacatttgttatctgaattcgcattact
ttctaacaaccaacaaaaggaaagtggacattcaatttgagccggagggagaaaatttaa
ctagaaaatgacctggccgtgaaataaaattattgatccgtcctttaactagttttcatg
gattgcctccttgcggatgatttttccaaccggtagaactactgttagtcgtccaaattc
tgaccccctactatgaataaaaatgtattagtaagtttagtgggtaatctccttgagaaa
taaaggaacaggagaaatattttattgatatatgctaagtgttttacaatagccctattt
atatacaatgtttacataaacctaaagccttctatataaatgtgggacactatacatgaa
ctaactctaacactatccctcaagctagtgcatataaattatatatatgcttgttacata
tataattaatttctctactttttggtatacttcttgtatacgggagttatctcccttttg
attaatacaatttaccttatcaaaaaaaaattaatacgaggaccagtgagggacttggtg
aaaatatctgcaagttgatcatttgacttctcaaactttgtaacaatatctcctgagaat
cttctctctcgtgaagtgacagtcaatctcagtgtgtttggtcctctcatggaacactgg
atttgatgcaatatgaaggacaacttgattatcacacacaagttccatctgactgattgc
tccaaattttaattatttgagcaattgtttgatccaaactagctcacatggtgcaagagt
catgactcgatattcggcttctgcgctagatcgagcaactacattctgtttcttgctttt
ccgagagacaaattacctcctattaaaacacaatatccagatacgtaacgtctatcagaa
ggtgaccctgcccaattagcatctgtgcgtccaacaatatgctcatggcatcgatcttcg
aatattagtcatttgtctggagctgattttatataacgaacaatgcgaacaactgcatcc
caatgactatcgcaaggaaattccataaactgacttacaacactcacaggaaataaaata
tcaggtctagtaattatgaggtaattcaattttccaaccaggcgcctatattttgcagga
ttgctaagaggctcccccctatcctggcagaagcttagcattcggattcataagagtatc
aatagttctgcagcccattattcatgtctcctcaagaatgtctaaagcatacttcctttg
cgaaataacaacctgaactagaccgagcgacctcaatacctacaaagtacttcaatctgc
taaggtcgttagtctggaagtgttgaaagtgatgttgtttcaaattagtaataccatcct
gatcattgcgagtaataacaatatcatcaacataaaccaccagataaatacagagattag
gagcagaatgccgataaaatacagagtgatcagcttcactattagtcatgccaaattccc
gaataattgtcctgaacttacgaaactaggctcgacgagattgttttaaaccatagagac
ttgcataagtgacatacaatacctctagactccccttgagcaacaaaaccaagtggttgc
tccatattaactttatcctcaagatcaccatggagaaaggcattctttatgtccaactga
taaagaggccaatgatgaacaatagccatggacaggaaaaggcgaacagatacgacttta
gccacgggagaaaagtgtcattattatcaagcccaaatagctgagtatatccttttgcaa
tcagacgagccttgagccaatcaacctggccatccaggtagactttgactgcataaaccc
aacgacaaccaacagtagacttacttgaaggaagagaacaaactcccatgtaccactcac
tcacatgtaaagcaaacatctcgtcaatcatagcctgtcgccatcctggatgagatagtg
cctcacctgtaaacttaggaatggaaacagtggacaaagatgatacaaaatcataatagg
gtgatgagatgcggtgataacttaaaccaacataatggggactaggattaagtttggatc
atacaccctttcgaagtgcaatcagtggactaggaggagccaagtccgcactagacgtgg
atgacaatgataagtcaagagtggtggcctcgtggttggagatgtaggatgagcaactgt
agactcctcagaagtcggtataggtaggagtacctgtgatgttgatgtggatttaagagg
aggaacaatagattcctcacaagtagatacaggtaagacctcagatatatcaagatgatt
agatgaagtaaagtaaggttgagactcaaaaaatgtgacatcgactgacataagatatct
acgaagatcaggtgagtagcagcgataccccttttgaacccgagaatagccaagaaagac
acacctgagaacacaaggagctattttatctttttcaggagctaagttatgaacaaatgt
actccttaaaacactaggaggaaagagtataaagatgacctagggaacaatactgagtgt
ggaaactgattctagatggaagatgaaggcatccgattaattaagtaacaggttgtaaga
actgcatcgtcccaaaaacgttgtggaacataggactgaatgagaagtgtgcgagcagtt
ttaatgagatacctattctttctctctactaccctataatgttgaggagtatacagacat
aggataatattttgagaagtcataaactattgaaactaagagaatacatattttaaggca
ttatcactacgaaaagcgaataaaaacaccaagcggagttttaatttcagcataaaaact
ctagaatattgaaaacaactcaaaacgatctttcatttggaaaatccaaatacatcttga
gtaatcattaatgaaactaacaaaatccaaatcttaaggttgtgactctactaagacccc
atatatcataatgaactaaagacaaaacagactctacacgactcttagcacgacgtgaaa
atgtagctcgaatatatttcccaagttgacacgaatcacaatctaatgtggacaaaccag
acaccatcttctgaagcttggataaactcggatgtcctaaacgtttgtgaattaggtcta
gaggatctgtagttggacatgttgtagagggattgagtgagttaagatagtcaaggtctt
gtgattcacgccatgtgccaatcgtctgtaccgtactgcggtcctgcatagtaaaagaat
catcaataaaatatatatcacaatggaattcacgagtcaaatgactaacagatgcgagat
taaaggacaaccggggacataaaaaatagaatctaaagtgacagaggacatgtgattagc
ttgtccaactccttttgcttttgtttagacttcatttgctaaagtatcattgggaagaga
ttgtgaataaacaattatttgacaaaagtgacatattaccactggggtatcaagttgctt
agtcatactaagaatgtttgggagagggtggtggaagtgagggtaaggaggacagtgtct
ctatccgagaaccagttcggattcatgcatgatcgttcaactgcggaagctatccgtctt
attaggaggctggtggaacagtacaaggataggaagaaggatttgcacatgatgtttacc
tagagtaagcgtatgacaaggtccctaaggaggttccttggagatgtcagaaggttaaag
gtgttccggtagcatatactagggtgatgaaggacatgtatgatggagctaagactcggg
ttaggacaatggaaagagactctaagcattgtttggttgttatggggttacagtaaggat
ctacgctcaaaccgttcttatttgccttggcgatggacgcattaacgtaccatattcagg
gagatgtgccatggtgtatgttattcgcggatgatatagttctgattgatgagacgcgag
gcggtgttaacgagaggttgggggtttggagacagacccttgaatttaaaggtttcaagt
tgagcaggactaagacagaatacttggaatgtaagttcagcgacgtgacggaggaagctg
acatggacgcgaggcttgattcataagtcatccccaagagaggaagtttcaagtatcttg
agtcagttatacagggagaagatggggagattgacaaggatgtcacgcaccgtattaagg
gcggggtggatgaaatggaggttagcattcggtatcttttgtcacaagaatgtgccacca
aaacttaaaggtaagttctatagagcggtggttagaccaaccatgttgtatggggcagag
tgttggccagtcaagaattctcatatctagaagatgaaagtagcagaaatgagaatgttg
agacggatatgcgggcatactacgttggaagattaagaatgaaaatatttgggtgaaggt
gggcgtggccccatggaagttgtgcccaccattaaagactgctatctgaaaactaattct
ttgggcccaaacattctggcccaaagtacctcgtgaataataatattgagctcatgtctg
acatgttggaagaggagttactagcaaacacttatacacctatgttggtaacacaattga
agaactacgaaaaacactcttctgcaaaggaaaatgagaagaagaagaagaagaagacga
agaagaaggatgatgcaatgatcattgaagaaaaaggagagcaggaggacccatctaaac
ttacaaagtctagaggaagaggaggacccagagtttgatgcttccctctgggtacaccaa
aacatcgtcaaacttaggcaaggagtttggggtaaacattcaggggtgtgagaaggaagc
tttggagcttttcgtaaaattacaactagaggcataaaaaaaaaaaaggcaatccaggca
tggaggtgacaaccttcgaaaagaaagggattcaaagaactgaaagggctggatttttgg
agtaacttcaagagtaatagaacaagaagtagggggttgcattattatcaaagatcaatg
aagattaacattgaagaagtgggaaatccaaaaagactccaccgagaaggatgatgcaat
gatcattgaagaaaaaggagagcatgagaaaaaacccgtagaaattgacagcactcacac
acaataagacgagataataaagtagtgagttggccaattgaagaagctttacctcttaac
ttacaaagtctagaggaagaggaggacccagagtttgatgcttccctctgggtacaccaa
aacatcgtcaaacttaggcaaggagtttggggtaaactttcaggggtgtgagaaggatgt
tttggagcttttcataaaattataacaagaggcatgggaaaaaaaaggaaatccaggcat
gcaggtgacaaaaccttccaaaagaaagggactggaagaactgaaagggctggatttttg
gcgtaacttcaagagtaataggacaagaagtacgggattgcattattatcaaagatcaat
gaagattaacattgtatcatggaatgtcagggggttaaatcgacatagaaaaagaatgtt
gattaggagtttaattcataggtggaaagcagatgttttctgtttccaagattcaaaatt
aaaaggggacattagggagtttataagagaactatgggcaaataggtggtttaaatatgc
acagttggaggctagtgggcctagagggggtattattgtcttatgggatagtaaaattgg
ggagggggagatcagcagcctgagctcctattctgttacttgtaaatttataggtaaaac
tcaggagtatacttggaatttatccactgtatacgctccaaatgatagggaggaaaggaa
agaagtatggtgggaattagcaggtgccaggggaatttttatggaccttgggtaatttct
ggggatttcaatactgtgaggtacccaccagagaaaaagaattacagcaaaatcactaga
gcaataaatgaattctcataatttattgaagatatggaactggtggatctacaacttgca
ggaggaagttacacttggaggacaggagatagacatgtgataacagctagactggatagg
ttcttggtttttatggattggaatgagagcatcagaaacaccaagcaatcagttctccat
tgaattacctctgaccattcccctgtgatgcttcaatgtggtaaccggtaccctgtcaaa
tcctattacaagtttgagaattggtggctggaaacagagggcttcaaagaaaggattaaa
gtctggtggagctcttttgcttgtgaaggaagacgtgactttattctggctttcaaactt
aaagcatcgaaggaaaaaattgaagaaatggagtaaatctattcaaggaaacttggagat
gcagaaattgagtattcttagtcaacttgcagaactagaagagacacatgatcaaaggag
ccttactgaagaagaaatacacactaaatatgcagtctatggagtttggggagattgcaa
aacatgaggaggtggcttggagacaaagatctagggctctttggttgaaagaagggacaa
aaacatcaattttttcctcaaaattgcaagtgcacataggaaatacaataacatagacca
actgttacttgaaggaaaatttgtggcgaatccaacatacataacaaataatattggtac
attttatcaaaaactatatataaagattgctagaggacaatcttatgttgcaaagtcttt
tcgaagcttaggaaatttgggatagtgtcaggcatgtgaaagggataaagcacctggacc
tgagaactgggaggtgataaacacggatatgatagctgcagttctttgttcatggaatgt
ttgaggaaagctttaatgttacctttgtggtattgattcctaagaagatggaagctaagg
aatagaaggactttaggcctattatgataggcaatgtgtacaagatcttgatagaaagac
ttaagaaattggtgaacaagttggtgaagggtcaacggatgacttttattaaaggtagac
agataatggatgttgttctaattgccaaatgaatgtgtagatgcaagaacaaaggcgaga
aacctacaatactatgcaaactagatattgagaaggcatatgaccatctaaattggaact
ttctattggaatcgctgatgaggatgggctttggtgtaagatgggtcagctggatcaaat
tctgcatcagcacaatgaaattctcaattttgataaatgtttcaccagtaggtttcttcc
cttctcagagggatttgagacagggtgatccactatctccttttattattcattagtgct
atgggaggcttaaatgatatgttaaagactactcaagataacaactgcatacggggtttt
aaggtgaagtccagggcagacagtactattgagatttttcatcttcgatatgcagatgac
gcacttatgttctgtgaggttgacaatgaacaattgaaagtgctgaaggtgatcttcatt
ctgtttgaagccacatctgtattacaaattaactggaatgaaagctttatctatctagtt
aatgaggtaactaagatccactttttggttggaatcctagaaggtaaaattggggaattg
cctacagttatttggggatgccatgggggccaagagcaattttaaggggatttggactag
ggtcgtagagatatgtgaaaaaattttaacaaactggaagagttagtatttatccttaag
ggacaaactaatactaatcaattctatacttgatgattttcctacttacatgatgttcct
cttctcaatccatgtgaatgttgtgaagagaatatatacccttagaaggaacttcctatg
gggaggaaactatgacaaggaaagatctatttggtcaaatggaagtctctcacagtcagc
aagaagtaagagtgttttggaatcaagaattggagaattcagaaccaaagtttgatgatg
aagtggctatggagatttactacagaagaacattgtttgtggaaagaggtgatcatggag
aagtatggcatagaagataaacggataacaaagtctgtaaatagatcttatggagttagt
cgatggaaatccatcagggacctatagcttcagctcttgaataagtccaaattctgaata
ggaaatggattgaaaatatctttttggaaggataattggctaaccaaggaactttgaaac
aactctttcttgacatttacattccaaatcaacagcataaagcaataatagtagaattat
gggctaatcaaggttggaatctcacatacagaagactatcaaaagacccggagattggca
ggtcaacagagttcaaaggcactttggaacaatttaaagaggtctatacttctatagact
atttgacttggcaagggaagtttattgttaattcagcctataaggaattcaacttctcag
ctaactggattggttgttggccatagaagttgatttggaaagttaaaattccttatagag
ttgcttgtttctcttggcttttggctaaagaggcagttctgacgcatgataatctaacca
agagagattaccatttatgttcaagatgttatttatgtgaagagcaggcagagacaacca
atccacttttttttgcattgtaagttcactgcagttatggaggattttcattagtttaaa
gggtatcatgtgggctatgcgtagaagtatacctgaagttctagcatactggaaaaaaga
aagaaatctttccaattataaaaagagatggaggattatcctagcttgcatctggtggac
catttgggaagaaagaaatcaaagatgcttcaaagataaatcagtcatattcagataatt
aaaatgaagtggctagtcttgttttatttttggtgttaagtgttagatagttatgtatta
tgtataagttgtctagtcccacattggaacgggagtaatatgtactatgtagagtatagc
tataaataggacttcttgtactttattgtagagaatatattaataatatatttttcccgt
gttgtctcacatggtatcagagaaaccgtgagatatcagtcgttgtgaaaaataccagcg
gcttcgggaagaaaaaaatcaatcaactgctaggtatattagtcttcggcgaccgatcca
ttaaatttctctggcaaagaaccactcatgggccctcacgcgcccaccgaaagaaatatt
tccggcgaggttccaatttcatgcgcccgcgcgtgaggcagtttccggtcaaattttgac
aaaggtcctttttgacagtttgttcaccctgtaattcccagtctatccatcatttttttt
atttcgatcacttcgcaatttctcgggcagctacagtgatttttccggcagaagcggtgt
ttcctttgcctgcttcagcgagatacagttgattatttctattatttgtttctagacctc
tctccaatccaacgatgtctttggaatttgatgtatttggttctgaaaacacgagttcta
gaaagtcaagcttcatgattactttagagccattaatggggagttcaaactatttagctt
gggtttcctctgttgaattgtggtgtaaaggtcaaggtgttcgagatcacttaatcaaaa
aggctagtgagggctgtgaaaaggtcaatttaagcagtttatgacgtctgtataccactc
agcagaataggatagcaaagaaagaatatgcacatcattgagactgctcgcacacttctc
attgagtctcacgttctgctacattttctgagcgatgcagttctaacggcttgttatttg
attaatcggatgcctttatcttccatccagaatcagattctgcagttagtattgttttct
cagtcacccttatacttttttcgtcctcgtgcttttgggagcatgtgtttgttcataact
tagctcccgaaaaaaataagttagctcctcgtgctctcaagtgtgtcttccttggatatt
cccgagtttaaaagtgatattgttgctactcacctgatcgtaggtaccttatgtcagttg
atgttgcattttttgagtctagaccttactttacctcttctgaccaccttgatatatata
tgaggtcttacctataccgactcttgaggggtttactatagctcctcctctacatactga
gccacagaaatcttactcatacctaccattggggaatctagtgttgctcctcctagatcc
ccagctacaggaacacttttaacttatcgtcgtcgtccgcgcccagcatcatgtccagct
gattcacgttctgcacctgctcctactgcggactagtctcatcctaatctaccaattgca
cttcggaaaggtatatagtccacacttaatcctaatccatattatgtcggtttgagttat
catcgtgtcatcacctcattatgcttttataacttctttgtccactgtttcaattcataa
gtttacaggtgaagcactgtcacatccaggatggcaacatgctatgattgacgagatgtc
tgctttacatacgagtagtacttgtgaacttgttcctcttccttcaggcaaatctactgt
tggttatcgttgggtttatgccgtcaaagttggtccagatgaccagattgccaaagggta
tagtcaaatatttggggcttggttacagtgatattttctctcccgtggctaaaataccat
cagttcatctctttatatccatggttgttgttcgtcattggcatctctatcagtttgaca
ttaagaatgtttttcttcacagtgagattgaggatgaagtttatatgaattaaccaccta
attttgttgcttagggggagtctagtggctttgtatgttggttgcctcagacgctctatg
gtctaaagtaatctcctcgagccttgtttagtaagttgagcacagttattcgggaatttg
gccaactcgtagtgaagcttatcactttgtgctttattggcattttacttcaaatctctg
tatttatttggtggtttatgttgacgatattgttattaccggcaatgaacaggatggtat
tactgagttgaagcaacatctctttcagcacttttagactaaggatctgagtagattgaa
gtattttttaggtattgtgattgctcagtctagcttaggttttgttatttcacattggaa
gtagaaaaacttcaatcatttttctttatttgaaaggaagaaaaaaaaggtaatatctag
acctaaatattaatctgaagacaagtgaggcttgctcagttggtaaaagcacctccacct
acgatcgttaggtcctgggttcgagtcaccatggaggggaagtgtggaaacactatagat
cctcctaatttgggagggggaaaaaaatattaatctgaattgacatgaatctcaatgaca
atgaccaacgatttcctgcaattcttttcagtatggaatgaataaaaaatcaagctacaa
gtctctattaaacgaaatgcactaacagggatcactctcaagaaaggaagtggttttggt
tgttgttattccaggttggataaatcactttctttataaatatcataaaagacaagggct
ttcttgcttcagcacatgtgggaaatgccggggggcttggctggtaccaagctcgagcgg
tctttctatctttttggattgcatgcccaaggcaatgctttttgtagattgggatggatt
gatcttcgcagaagtatgctttagacattcttgaggagacaggaatgacggattgtagac
ccattgacacacctatggatccaaatgccacacttctaccaggatagggggagcctctta
gtgatcctgcaagatataggcggctggttggcaagttgaattacctcacagtaactagac
cttatatatcctttcctgtgagtgttgtaagtcagtttatggactctccttgtgatagtc
attgggatgtggttttccgaattcttcgatataaaatcagctccaagcaaagaactgttg
ttcgaggatcgaggcccatgagcagatgttgattgggcacgatcaccttctaatagacat
tctatatctggatattgtatgttaataggagttaatttggtgtcttggaagatcaagacg
taaaatgtagttgatcggtctagtgcggaagcaaataatcgagcaattgttatggtaaca
cgtgagctagtttggatcaaacaactgctcaaagaattgaaatttggagaaattgatgga
accagtgtgtaataatcaagcagctcttcatattgcgtcaaatccggtgttccatgacag
aattaaacacattgagattgactctcactttgccggagaaaagatactctcaggagatac
cgttacaaagattgtgaagtcgaatgatcagcttagagatatttttaccaagtcccttgc
tggtcctcgtattagttatatttgtagcaaactcggtatatatgatttatatgcaccaac
ttaagggagagtgtgagatagttatgtacaacaaaatacccggtataatcccacaagtgg
ggtatggagggtagtgtatacgtagagcttacccttaccctgtgaaggtagagaagctgt
ttccaaataccctcggctccagtacaaatgaaaaggagcagtagcaacaagcagtaacaa
caatgatatagtaaaataactgaagaaagaaataacatgtagacatataactccactaac
aaacatgcaaggttaatactattgccacgagaatggcaaaggaatgttagatagttatgt
attatatgtatattaatagtctagtctcacgttggaataggagtaatatgtactatgtag
agtatagctataactaggacttcttgtaatatattgcatagagatatcaataatatattt
ttcctgtgctttctcacgtaaaggaatgtaatgtacttagaagatcatgaatctatcttt
gatgttttagacacctcgtgagaacacaaaggtttaggaactttattgtgttctttgtaa
ttatgggtgactgccaatatgttaccttttcataaaaatgattatttggccattggatta
gtttcaacagcctctctgcccctccgggtaggggtaaggtctgcgtacatattaccctct
ccagaccccacttgtgggattatactgggttgttgttgttgttgttgtggattagtttca
acaattttgatagttcttttatttgaatcaaactactcattcacatggattttgtatcgt
atcattgagttaaaaaaattggttttgctaatttatcctcatgtataacaactacctatt
tttcaatatattggattcaggagcttgtagtagctggagtttgctcttcaaagggcaata
agtgccgggtatcatgcacagtgactccaaatacagatctcctttctgctctaactctta
tggagaaacatgatctaagtcagctacctgttatactaggggacgtggaggatgaaggca
tccatcctgtgggcattttggacagagaatgcatcaatgtagcttgcaggtttttgacat
tcaacttttacttcaaagatataatgctttctggaaccattgatgataaaatatgcaaga
aacttgtgcagaagtcgcactttactatcgattaccagataaagttacttatcaagaagt
caaatatattgaacatatttctctaaaacactttgactggactgtaagcagaaacttact
aaagtaggtcgtaagaaatggtttgatagggaaatcaccatctacacttaaaagagttgt
gtgaatttgaattcttaaagcatgtgaaagttataaaaacttgttattatctaagcatct
gaagcattttggccatccaaaggatcaaaaataggaaataatttcatttgtacaatgaac
tccctgcacaaattctcacactaggtgtattctctattcatcactagcactacatgtgtc
actacgaatcatatacaataaatctttgtaacataaaagacgacacataatatggaagta
agccgagtatacaagggaagtttcatcattacggtgagctttttataagataatcaagtt
ttactggaaaagggcaaaaactctcccgtatagaagtataccaaaaagtagaatacctta
caaaaatatgattttctatgaacaacaccctatcttctatacttgtagggatctcatcgg
ggcaccaaaaagagataaagggataagaggcttttcctcaaatgtacaaaatccttctct
attccttcaaaagctctcctatttctctctctgcacactgtccacataagttcaatggag
caacatccacgccctgtgtcttcttttccgtcttctataggtccagctgaacatggcttc
tttgactgagtgtggcatcaacgttgaagaccaaaccatcccagtacttccaaccacaaa
cgagacactatatgacaatttagaagaagatgattcacatcttctcccgaacatttacac
ataaaacaccagctgatacatgtaatcttcctcttcctcaaattatcagccgtcaggatc
acccgtctcgtagctaactaggtgaagaagcacacctttctcgaaaacctcaggatccat
acagagagatatggaaaagctgattcctccatgcccagaagcttctcataataagactta
acaaagaaacaccactacttccccccccccccaaaaaaaaaaaatctccatacatcgact
ttcatgtgtaattcttgttcgtgaaacgacccaatcaacctttggcacaaatctcccagt
cttgcgagttcctcctaaacttcaaatcacaatgaacttctccaccttgtagcctccgtg
tcccttggactggcaactcctttggcatgaaactttgtacatattaggagatgtgatact
caaagtgttgttcctgcaccaattgtacccccaaaaaacttaccatgctcccatcaccta
acattgaatgatacgttccaaaatcttcgcactccttcaagaaacttttccgtaggcccc
acccataagggagtgtgattttttttgctctccatcccctctccaagaatccattcccta
aaccactgcaggacactttaacaatcactatgtcactttttctactagttctacattgag
tgatatcttgatgtcattgaaatgcctctggaaaatcttcttctcatctaaaagaacact
tgtttgccttttgaatccccctctaacattttctatgtttcattcatctttggtggaaca
gagcattagcaactagagaacagctttgctag
(DNA sequence of NtCLCe from Nicotiana tabacum; sequence originating from
the ancestor N. tomentosiformis; two start codons)
SEQ ID NO: 11
atgattagcggccaaaacaccgtgctgcaccatcctcctaattcgctcttcaattcctta
tctcctcgccatatctgtgtatctttctgtaacgacaaagctttaaaaaagtcagtcacg
cactccgcccctcggtttgctcgtctgttaaacaatgaatcacgaaagttgttgggtcgt
catccaaattgctggccttgggctcgacgaccatctcttcctccgggacgttcctgtgac
ggaaacattgaaaaagaacaagatatgtgcgacagcagcaaagacgatagtgatagtgat
agtggtatccagataggatctctgctcgaggaagttatcccacaaggcaataataccgct
ataatctcggcttgctttgttggcctcttcaccggtatcagtgtcgtgcttttcaacgct
gcggtaagtgcgctataggtctttcatttctcttttcatctactattctcccttacttac
ttggcctcagtcaatcagccccctgcctactttaaattattgtacaatttatcagaggag
tatcctatacatcaaattcacataacttagtaaaatatgctgacattctgaattttaacc
ttaccagcttagaacatccaggctagttcagaaacagataatctaaattggcctcattta
taagtcattttgttaatcaagacatacaatttggctcttgataaaagattatgcagcgcc
cgatgataacctaatatttatcagcaacccatatgtcactttcttttgtttaaatgctct
cccatgtaatttaacaatattgtcaccatacaaaagagaactgaagtgaatgttccattt
gtggtcatataacggatatctcccttggttaggttcatgaaatacgtgatctttgttggg
atggaattccatatcgagctgcctcagaggagcccattggagtacattggcaacgtgtaa
tcttagtaccagcttgtggcggtttggtagtcagctttttgaatgccttccgagccactc
tggaggtttcaactgaagaaagttggacatcatctgttaaatctgtgttggggccagttt
tgaagacaatggccgcttgtgtcacattaggaactgggaattccttaggaccagaaggcc
ctagtgttgaaattggtacatctgttgccaagggagttggagctctgcttgataaaggtg
gtcgtagaaagctgtcactcaaggctgctggatcagctgctggaatcgcttctggtttgt
tccccatattattcttggttctgaaccatacatggtacattttccttataattacatgta
gcctgttgtatgctttcctctttcctgggaagcctttctgtaaatgcaaatgtgtttgca
ctcaaaccaataaactgtaaaaacagtgaaccccttgagcaagcaaaagcactagaaaac
caacaaatagatcccccccccaagataccagtgaaatgacaccgggtgacccaaaaataa
agcagcttacatcttgactttgagaggaactgcaatcagctataagtaggttattaattt
ccagtgcctgcattctgcccaagtactatgatatatttctgaagctttgtttccccagtt
cctttttcagacgtttgctgtcaataaagttgagccagccaacttggttcccacaagcta
ctaattttgtccaagcttactctatgggagaagttaaatttcccaaattccttgagcaga
aaatgaaaaatgaactcaaagtgtcatattaggcaactatctaaagaaaaatacttaatt
gaagtttagataagaaaagtgaatatatattgatgtagtctccgttaggtgagaagcgca
tcacttacccagcaacatatggacctaaaatttactagtgaacttttcacattgtatcaa
aagctcaacaaacagaaagatgactagtcctaaaatgttatttcacatcaaccttatcat
acgtgcattatttgttctctatatttctatttcatccgatataaccaatcgtcattgtaa
attctataatgcctgtggttacttttgtctttagtgacaaatgacatttaggctaaccat
gtagttattgactgatttcgcttgacgtctcttccaattatgtagtagtagagtgttgag
atatggatatgttaccttctaaaaaaaaagagtgttgagatgcggatggtttgctagctg
gcttttgtctcccttcaagttgaattagcaaaagcaatgtctcataagttggatagctag
acaagaaaaactccaaattactttatgtagagtattcttaagcttgagtcgcgagttgga
aattggaattatgtaaaaaaacctggaattatttggttgagcctgctttttatttttgtc
aatatttccagtatctaacccaacatgtttagagcaattcccagagagcctcaatacgag
gcatttgcagagtctttatgagagtccaggaaggggcacacactgtagaggtatagtgtt
gtccttatttttttttttttgataaggtaagattttattaaaaggtaccaagatggtgca
aaattacaaacatccaaactaatacaacaaagcaactacattcctcctagctcctctaga
aaattcatatattgttccatatttttcattacatgtcttttacaccagaaatacaagttt
aataagcatctgtttttaatcctggatacatgctgcctttccccttcaaagcaaatcctg
tttctttccaaccatattgtccagaacacacatagaggaattgttcttcatactatctgt
tgactctttgccactttttgttgttgccatgtctccaacaaactttacactggcaggcat
tgcccacttgacatcatatatatttaggaagagctaccaacactgctttgccactttgaa
atggatgattagatggttgactgtttctgcctcttcttcacacatgtaacaccggttaca
tagagcaaaacctctcttctgcaagttctcctgagttagaaaagcttcctttgctccaat
ccaaccaaaacgggctactttaataagtgcttttgacttccatattgctttccatggcca
atttgactgataaagcccttgtagtttttgtaacaagctataacaactgctgactgtgaa
aataccatcattacttgctgcccagattaatgagtctctcctgttttcctccaatctaac
attattcaataactgcatcaattgggaaaattcatcaacttcccagtcattgaggcccct
cttgaagattagctgccagccggtgcttgaatagaagtctaacactcttccatttttgtt
aatagagcagctatatagaccaggaaactttgatctaagacttccattttccaaccacat
atcagaccaaaacagggtattattaccatttccaagtttcagtttcacaaactgactata
tttattccaaagattactaattgtgctccaaactcccccttttgaagaagattgaattga
acgaggagcccacatgtccttcataccatacttggcatctatcacctttttccataatct
attcccatcataattatatctccatagccatttaaataaaagacttttgttatgcatctt
tagattcctcactcctaatccccctctttctttttttttcatcacctcttgccatttgac
caagtgaaatttcttgttatcattattaccttcccacaaaaatttattcctcatagtatt
caattttttctccactgatgttggcattttaacgagagatattagataagtaggtatacc
atccatcacactattgaccagtgtaagcctaccaccaagagataaatattgtcttttcca
tgacaccagtttactgctacatctatccaagaccccctgccacatctttgcatcattctt
ttttgctccaagtggtaggcccagataggtggatggtagctgctccactttacaacccaa
aacatctgccagatcatcaatacaatgctcggcattaatactaaacacattactctttgc
caagttcactttcaatcccgagacagcttcaaaagctagtagtactcctatgaggtgtaa
gagttgctctttttcagcttcacataatatcaatgtatcatcagcatagagtatgtgtga
gaaatacagttcttccccctctctttttctaattttcaatcctctaatccaccctaactt
ttctgcttttaaaagcattctgctaaagatttccatcaccaacaaaaataaataggggga
tattggatccccctgtcttaaccccctctgagaattaaagtatctatgtggactcccatt
aattaaaactgagaagctaattgaggatatgcagaattttatccacccaatccatctttc
cccaaaattcgtatgtttcatcagatttaacagacatgaccaatttacatgatcataagc
cttttccacgtcaagtttgcaggccacccctttaatcttcctcttgaatagatattcaag
acactcattagctaccatagcagcatcaataaattgccttcctcttacaaaggcattctg
attatctaatatcaattttcctatcaccatctttaatctttcagctatcgactttgcaat
tattttatagacactgcccaacaagctgataggtctaaaatctttcacttccgctgcccc
ctttttcttaggaataagagcaatgaaaattgagtttaggctcttagtcttgtccttatt
ttcagggttgaactagttctttagaagtttcctaggcttcctaatttccaaagttctgcc
aggtccttttctagtgaagtacttgaagtttaataaatcaaattttaatttctaacatat
cccgagaaattcattcacaaattcaactggtgacttctgatgcagaaacataagcaactg
cttatgggttcatatgttcctgcaattttattgttgacatggattggcttcatatggttt
tgttcctgcaattttatcgctgacactaatcctttcatatggttttatgtggggtggtaa
atagaggttaagagacaagaagaggctggaaaaggtgggcagttcatttgttagtagact
actctatttactaagagatatgatgtcccatacattactcgaattggctccaaatacaga
ttccacttctttgtcgagtttccttattgtacagagttcgactcgtcaagggaaattcac
ttcctttgactgaataatgctagtttgagtagtaccttaaattaaatggaccatttaatt
ctatctacttgatagaatagactggtcatcaactagttgcaaatataatgacaactccgc
catgtttgcagagtcacctgatgaagaagtacctcaattagtagaccatttcttgaatgt
tctacagtattctctatgcctacatgaccacatcacttttccttttgcgttgtgagaact
tgaacttggtgagcgggggttccccaggaatggcatcttggtggcagatgaccattctgt
ccttatcttagctaatgcttcttggattgcctcactagatttattatacctttaataaat
gtttgccattgttctgccataatagagggatgtacctagctggtgcttcacatcacatag
tccaaaactaatgaaatgctttacaattgtcgagtactaaaggatgatttgtggaatcag
atctcaaacaatttattttgaggaagaaaaataccaaaggttttttctgtttgttggaag
attaaaaatcctttaaaaggtaaagatttatgaacttaattcagcatttttgtggccatt
gctgaaaaagagaaaacaatggcacttattcgagtttgcttatccaaaaaaaaagaagaa
gagaatgtcacgtaatgcaatttcatcttaggaaactttgcaggagaaaagcaagagtga
taaaacagaactatttgtttttttgataagttgttgtgacctatttctttgtcattctta
tttgctaataagctaatgtaccctgtactatggttgttttgacttaatccggggatgttc
agtgagcattttcttgttttttctgctgtcagcatctgctgccttacaggaattcatttt
ctggaaatttacttcttgttctgctaacattttcctgttatatcttgtcagtcattttct
ctccatggttatactgtttgtgtcactttgaaactctccttgttttctactttaaaggat
ttaatgctgctgtcgggggctgtttctttgctgtggaatctgtgttatggccatcacctg
cagagtcctccttgtacttgacaaatacgacttcaatggttattctcagtgctgttatag
cttctgtagtctcagaaattggtcttggctctgaacctgcatttgcagttccaggatatg
atttccgtacacctactggtaattttggacttctttctcgagtttgattcttaaatacaa
ttgtacccgtcacttacagcaacaacaactacatttcaacagctagttggggttggctac
acagatcatcactatccatttcaatttctttagtcccatttctttcgaatattcagtact
ttgggattctctattatcagaggttctctttattttctactttgacgtacaaatctctaa
atagattaaagaagactcctagagacactggcctaatgcaaatgtaccaccatgaataaa
ccttaatctgaaatagctggtatcgtatataagaacctttagctttaattgtgttctata
ttgatcttttgggacaacttccgtccaataatattatgtcttacttatacagttatactt
atccttaaactttactctttagagtggttatccgtagttcaagcttttgttggcaccata
gctagtttggttcttagtaaaaagttactctttagagtggtaactttttgtcaattttct
tagtgaaaatataacctctgtgacaaatctaccaagtataaatccaatatggttctgtgt
catacttgtagtttatccaagtctatgctccatcactcttacaaaggctcatcgtatgac
taattttttttgagaaaggtaacagtttgtattgataataagatcagcgccaggttagtc
attagtgctaatagctgtatgtacaactccaaaagagcaaaagacaagcacctggtgtaa
cgtaaattacaagctgcctataaaatctatcaggtctcctacctcactaaacatttcttg
tttacaccaaaaaaataaaacaaggaaagacaatccatcttaatcttctgaatggagttt
cttttgccttcaaacatctcgagttcctttcgttccatgcaatccaccatatacaagctg
ggatgcttttccatttgtctttatccattttttctaccaattcccttccaattgactaga
agttccaatgtggttctagatatgacccaattaactcccaacatataaaagaacatgttc
cacggatttgtagtgattctgcaatgtaggaacaagtgagcattactttctacttcctgt
ccacaaagaaaacatcttgagcaaatctggaaacctcttctttgtaagttatcatgtgtt
aaacatgcttttttaccactaaccagacaaaacatgatactttgggaggagttttaaccc
tccaaatgtgtttccaaggccacacctcagtcattgaaacattatgatttagagtccagt
atgcatcttttactgaaaatgcacctttgctattcagcttccaaactattttatctatgg
tcttgttagtttacagctatgtatatagtgtagtcttgtcccacattggaataggagtag
tatgtccttgtatagtatagctataaataaggacctcttgtattgtattgaacatccaat
atcaataacatattttctcccgtgctttctcacatggtatcagagcaattgtgagagatt
tatcgctgcgcataaattccagcgactccgggaagagaaatcagtcaccggaagtctttt
tccgacgactctttcaaggttgtttgcgtttgctttataaatccaacactaccacaagag
taatcactgtccggcgaccaaaccccagtaaaaatctccggcagcagcctcctcacgcca
ccagaagctcacgcgccggcgcgtacgaccacttccgtccattttttgaaaaacttcctt
cagaacagttgggtcgcctggtaattcctatcctacccctactgttttcatttcattccg
accactttgagttttttccggctgctacagtactattccggcagctatagtactattccg
acaactacagtaagattccggctgctacagtatttcattattctgtttttgtgtttcctt
actctgtttcagtggattacaattgattctttctcttatttggtaataatttgcaacaat
gtctatgggatttgatgtttttgggtctagaaacatgagttctggaagctctagtgttat
tattacctcagaaccttaaatgggaggttcaaactacttagcttgggcttcatctgtcga
gttgtggtgtagaggccaaggtgttcaagatcatctaatcaaaccgtctagcgaaggaga
tgaaaaggcaataacactttggacaaaaatcgatgctcagttatgtagcatcttgtggcg
atctattgattccaagttgatgcccttgtttcgtccattcctgacatgttatttggtttg
ggcaaaggcacacaccttatacactaatgacatatctcgcttctatgatgtgatatcgcg
gatgacaaactgaaagaagcaagaattagatatgtctacttacttgggtcaagtacaagc
aatcatgggggaatttgagaagttgatgccagtttctgctagtgttgaaaaacaacaaga
gcagcgacaaaagatgtttctcgctcttaccctcgctgaacttcctaatgatcttgattc
agtacgcgaccatattttagctagtccgactgtcccgacagttgatgaattattctctcg
attactccgccttgctgtagcaccaagtcacccagtgatctcatcacagatacttgattc
ctctgttcttgcatcccagacaatggatgttcgggcatctcaaactatggagcatagacg
aggaggaggtcgttttggaagatctagacccaagtgttcttattgtcacaaacttggaca
cactcgtgaaatgtgttattccttacatggtcgtccacccaaaaatgcttacattgctca
gaccgagactccaggtaaccagggattttctttatctaaagaagaatataatgaactcct
tcagtatcgaacaagtaagcagacatctccacaagtagcctcagttgcttagactgatac
ttcttttactggtaatttttttgcttgtgtttcccagtctagcactcttggcccatgggt
catggactcaggcgcttctgatcacatctctggtaatatatcacttttgttaaatattgt
atattcatagtctcttcccattgttactttagccaatggatgtcaaattacggcaaaagg
agttggacaagctaatcccttgtcttctatcaccctagattctgttctttatgtccctgg
ctgtctttttcgtcttgcatctgttagtcgtttgactcgtgccctccattgtggtatata
ttttattgacgattcttttattatgcaggactgcagtacgggacagacaattggtggagg
acgtgaatcagaaggcctttactaccttaactcacccagtccttccacaacatgtctggt
tacagatcctccagatctaatccacagacgtttaggacatccgagtttatccaaacttca
gaagatggtgcctagtttatctagtttgtctacattagattgtgagtcgtgtcagcttgg
gaaacatacccgagcctccttttcgcgtagtgttgagagtcttgcatagtctgccttctc
cttagttcattctgatatatggggtcctagtagagtaagttcaaccttgggatttcgtta
ttttgttagtttcattgatgattattcaagatgtacttggcttttcttaatgaaagaccg
ttctgagttattttctatattccagagtttctgtgctgaaatgaaaaaccaatttggtgt
ttctattcgcatttttcgcagtgataatgccttagaatatttatcttttcaatttcagca
gtttatgacttctcaaggaattattcatcagacatcttgtccttatacccctcaacaaaa
tggggttgctgagagaaagaataggcaccttattgagattgctcgcacacttctaattga
atctcgtgttccgttgcgtttttggggcgatgcagtgctcacaacttgttatttgattaa
tcggatgccttcatctcccatcaaggatcagattccacattcagtattgtttccccagtc
acccttatactctcttccaccccgtatttttggaagcacgtgttttgttcataacttagc
ccctgggaaagataagttagctcttcgtgctctcaagtgtgtcttccttggttattctcg
tgttcagaagggatatcgttattattctccagatcttcgtaggtaccttatgtcagctga
cgtcacattttttgagtctaaacctttctttacttttgctgaccaccatgatatatctga
ggtcttacctataccgacctttgaggagtttactatagctcctcctccaccttcgaccac
agaggtttcatccataccagccgttgaggagtctagtgttgttcctcgtagttccccagc
cacaggaacaccactcttgacttatcatcatcgttcgcgccctacatcgggcccaactgg
ttctcgtcctgcacctgacccttctcctgctgcggaccctgctcctagtacactgattgc
acttcggaaaggtatacgaaccatacttaaccctaatcctcattatgtcggtttgagtta
tcatcgtctgtcatttccccattatgcttttatatcttctttgaactcggtttccatccc
taagtctacaggtgaaacgttgtctcacccaggatggcgacaggctatgagtgacgagat
gtctgctttacatacaagtggtacttgggagcttgttcctcttccctcaggtaaatctac
tgttggttgtcgttgggtttatgcagtcaaagttggtcccgatggccagattgatcgact
taaggcccgtcttgttgccaaaggatatactcagatatttgggctcgattacagtgatac
cttctctcccgtggctaaagtggcttcagtccgtctttttctatccatggctgcggttcg
tcattggcccctctatcagctgaacactaagaatgccttttttcacggtgatcttgagga
tgaggtttatatagagcaaccacctggttttgttgctcaggagggggtctcgtggccttg
tatgtcgcttgcgtcggtcactttatggtctaaagcagtctcctagagcctggtttggta
agttcagcacggttatccaggagtttggcatgactcgtagtgaagctgatcactctgtgt
tttatcggcaccctgttgacattccgatggatccgaattctaaacttatgccaggacagg
gggagccgcttagcgatcctgcaagctataggcggctggttggaaaattaaattatctca
cagtgactagacccgatatttcttatcctgtaagtgttgtgagtcgatttatgaattctc
cctgtgatagtcattgggttgcagttgtccgcattattcggtatataaaatcggctccag
gcaaagggttactgtttgaggatcaaggtcatgagcagatcgttggatactcagatgctg
attgggcaggatcaccttctgatagacgttctacgtctggatgttgtgttttagtaggag
gcaatttggtgtcttggaagagcaagaaacagaatgtagttgctcggtctagtgcagaag
cagaatatcgagcaatggctatggcaacatatgagctagtctcgaccaaacaattgctca
aggagttgaaatttggtgaaatcaatcggatggaacttgtgtgcgataatcaagctgccc
ttcatattgcatcaaatccggtgttccatgagagaactaaacacattgagattgattgtc
acttcgtcagagaaaagatactttcaggagagattgctacaaagtttgtgaggtcgaatg
atcaacttgcagatattttcaccaagtctctcactggtcctcgtattggttatatatgta
acaagctcggtacatatgatttgtatgcaccggcttgagggggagtgttagtttacagct
atgtatatagtgtagtcttgtctcacattggaataggagtagtatgtccttgtatagtat
agctataaataagacagtactaacgtcccttttgccgggggttctgcatctttaaataga
tgcacgtggttccatagcagaccgtgttgatcacagatcgtgctgcatcctcttcccagc
ggactcggtgagcccctcttgtattgtattgaacatccaatatcaataacatattttctc
tcgtgctttctcacaggtctgtgatgtacccttgaaaggttcaagagtttggaggaagat
agaaactctgtttatctcccaatcatccaaagatcttctaaagttccagttccatccttg
tgagctccagactgacttaccaatgcttggctttgaagacttagagagaataagtcagga
aaaatctttcaaccttccttgccctatccggtgatcttcccaaaaagatgtcttcaaccc
attgccaacattgatcctgatattgctactgaaagatttcttttggtggcaggattactc
tcattaacaatgtacttgacaatctccatacatacgaatgtctctttaccctcttgccat
taaggttgtaaagagacttgtcaaattaagaagaggtttcctatggaactgtttcaagga
aggaacctcctttcctttggtcaagtggagttaagtcatataatctaggaagtggagact
tgggtataaaatagctgcaactacagaaaaggagcatcttatttaaatgatcacgcaaat
gtgcccaaaactttaaatatctgcggagcatatggttgtagcaaaatttgaatcttccgg
tcaatgttgctcatgtccagtgaatacccctgatggtgaaagtgtcctgaagggaagcag
gaacttattggaggaattggcatttaacactcagcatttcgttaggtcatagcccgctga
aaattgagtgcccagatttatatagttttgctctaaactgacgatgcagttgcacaacat
acgacaaactaaggtgggacatcttcttcggaaggaattttgaggattaagagatagagt
ggttgattcagttgcaaatgaagcttcaagggttcaatatcatccaggagacaccggatt
ctgatagataaaacaacagaaagatgaacactactttgttaggcttgttacaagttgcta
tcgtctttcttatctcggcacacaatttagatttgggaacttatttggaaaatagagtgg
ttgtttttgtgaatagcatcagacaaagcttctgagctggtacgacagaaaactcaacag
ggagaataaaagactgtggttcacgatttctgcatgcatcttgtaggttatttggtgggt
aaaatatttaatgttttgaagggaaggtagaacatgttcataggcttagattcaaatgtt
tgtatttttttggctctttggtgagagatgctgaatgtaaatgacataggcagctgacta
taatttctcagctccttgctttttaaattggcaggcactgatatgtacatgtgaacatcc
aacacttttgtggtgccgttccgatgaataaagcacattaatcacttactgatcaggagt
aatagtttaggagttctagaatttttgtacataaaatgaaccaaaaagaatatcggaatg
agaacatgtttctttttttgtttcttctttttcgtacaaatttcaataacacttctgata
gaatagctaggtccatttgaattcctttggagacccttacacaaccaatgaatggcaagt
atagcattttctaacaccctcccacatgtataatccagtttttagggtttagatgtggat
ttgatttgaccttattgcctttttttgtttttgttctttttgaagtagagagtgaggagg
ctcacaacgacgggctacgtagagcgagattaattcggctcaacgggctaatgattggac
ttacatgctacaacaatgttaggagaaagagagagagagagagagaagcccagagcagtt
ccacgagttaagaaagagaagtccaaagcgattgaatatgaagagagaaagcggttgtgc
taacaggctccctcaagtttggctctgagcatccaactcaaaaccttaaggcaatgagta
gagtagcccaggaccatttaaactcctgttgaaaaccttacacaaccaataagggaacaa
gtgtaacattctcttacaaccctaccgtcttataagtcagggctctaatttagcataaaa
tcaaagtgaggcgatctactatgaaatgaagaaaataactgataaatataaagaatgtta
attctcccatatagcctgaatgttcccagaacaaaataaattagtctcatgatttatcat
taacatgatgttcctcttattttgagtgattaggaaggttaatcaaggagtaaattcttt
ctaatttgtatcgtctagaattatttgtctaacaaattttcagattaccggtgatcaaaa
gaggaaaatattttgcatacaacgttaccataccttacaaaagggcgatgaacatttttt
tattttattattgtcctttttttcaattaggggttatgcagtcttcctccacgtgatatt
actcttagaatcacgtttttgtcattgctattacttactgtggtaagtacaaatgtgttt
tgaactctttttggtatgtattattgagttaatttttcgtttccatttcagagctgccgc
tttatcttctgctgggcatcttttgtggcttagtttcagtggcattatcaagttgtacat
catttatgctgcaaatagtggaaaatattcaaatgaccagcggcatgccaaaagcagctt
ttcctgtcctgggcggtcttctggttgggctggtagctttagcatatcctgaaatccttt
accagggttttgagaatgttaatattctgctagaatctcgcccactagtgaaaggcctct
ccgctgatctgttgctccagcttgtagctgtcaaaatagtaacaacttcattatgccgag
cctctggattggttggaggctactatgcgccatctctattcatcggtgctgctactggaa
ctgcatatgggaaaattgttagctacattatctctcatgctgatccaatctttcatcttt
ccatcttggaagttgcatccccacaagcttatggcctggtatgaatttgtcttttgttag
aagtagcattacatatctggataagtgagttttttattattgaaaagtaataacaggaga
acaagagaatatatcacccaaatctacttctttcctctcttctattcttctgaaattcaa
ggtcctttaactcctccacagtctgtctagttattgatcctgtagacttaattcacatag
gtttaggacattcgagtttatccaaacttcatgaaaaggtttctaatttttttacattac
attatgagtcgtgtctacttgagaaacatatcactccatgtttctatagtctgttttctc
cttagtttattctgatatgtggggtcctattaagtcagttcaaccttgtattttcattat
ttttgcagtatcattgataattattcaagatgtacttggattttctttacaagagatagt
tctcagttgttttttgtgttcctaagtttttatgctgcaatacaaaattggtttgatgtc
tctatttgcatttttcccaatgataatgccttagaatattttcttttccgtttcagtagc
ttattatttctttaggaactctttatcagaaatctcaactgagatagatgagaggaagaa
taagcatatcattggtctcattcagtcccctgtcaagcttagtttcttgagcgatgcggt
ttcacgtccttttattagattaattggatgcctcatctgctatccaaaatcagttaactt
tcgatattgtttcctcgcttacctttatactctctttccctcgagtctttgggagcacat
gttttgttcaataacatagctcctggaaagtgaccagcgcaaccgacaaacaaggccttc
ttaatgtagaaggtggacatatgctattctagccacgggaaagaaagtaatattgtaatc
aaacccaaatatctgagtataacctttggcaatggcgatcaatttgattatatggaccaa
ctttgcctgcatatacccaccgacaaccaataatagatttaccgggaggtagagaaacaa
gctcccaaataccactaatatgtaaagcagatatatctctgatcatagcttgtccttgtg
gacatagggatagaaattaaggacaaagatgacacaaaagcataatgcggtgatgataaa
cgatgataactcaaatcaatataatggggatggggattgagagtggatcgaatatctttg
cggaatgcgattggtagactaggaggagagaagtctgtggacatgatgttggactgagat
caataataagtcaagaatggtggagctacagaacatggaactggagctgtaggtgacata
atcggagctgtaggaggtggagctatagaggaaggtgaaggagagatagcgactgaatct
ccaaaagatgaaaccggtaatacctcaaaaaatgtctaagagatcatttggacctatgaa
gtatgattgcgtttttaaaaaggtaacatcataaggtcaggtgaataacattgatatccc
cgttgcatcctcgagtaacttagaaatatacatttgagagcacggagagctaacttatct
tttctggagcaaggttgtaaacaaaacacgtgctcccaaagacacgaggtggaagagaga
aaggtgagtggggaaacaagacagaggatgaaacttgactcttgatagttgaagatgaca
tacaattaataagacaataggatgtgagatccaatgacagttctcatgaactgctgaaat
ggagaagacaaatactctggggcgttatcactacgaaatgtgcagttagaaaccccaaat
tgattttggatttcagtgtggaaggtctaaaaaatagagaacaactcagattgatttttc
atcaagaatatccaagtggacttggaataatcatcaatgaaactgacaaagtagcggaat
tccaaggtagaactaacccgacaaggaccccaaacatctgaatggactaaagtgaaaggt
aactctacccgattatcaggatgtcgagggaaatgagagtgagtatgccttctgagcgga
tatgactcacgctctagagtggacaagtgagacaaacgaggtactattttctaaagttct
gataaattgggatgtcctaactgtatatgtaataaatctggtggatcagtaaaaggacaa
gctgtagggggaaaaaaataccaaatatttccagaagatggcaaactacaacagaagatg
caactgcattaacatgctcaggataggtgatgaaatcattgaggacaaagagttgatcaa
gaaggagattctggaattttaccagaacttatatagtgaaaatgaaccctggaggcgcag
tgcaaatttcgaagacatctcctcactaagcatagaagagaagaactggttggaagctcc
atttgtagaaatagaggtgcttgaagctttgaaatcatgtgccccttataaagcaccagg
tccagaaggcttcactatggatttctttcagaaaaattgggatactcttaaaacagacat
catggctgcacttaatcattttcaccagagctgtcacatggttagggcttgcaatgccac
cttcattgccctaattccaaagaaaaatggtgctatggagctcagagactacagacctat
tagcttgacaggtattgtatacaaattggtttcaaagattttagcagagaggctcaagaa
ggtaattgacaaactagtctcgggggaacaaaatgctttcatcaagaacaggcagatcac
tgatgcttccttgattgccaatgaagtgctggattggagaatgaaaagtggagaaccagg
cgtgttgtgcaaactggacattaaaaaggcttttgatcaattaagctggtcttacctcat
gagtatcttgaggcagatgggctttggggagaaatggagaagatggataaactattgcat
ttcaactgtcaagtactctgttttggtgaatagggacccaatcggttttttctcccccca
aaagggcctaaggcagggggatcccctctcccccttcctattcattctggcgatggaagg
actcactaaaatgttggagaaggctaagcaactgcaatggatacaaggctttcaggtggg
aaggaatcctgccagctcagttacagtatctcatctactctttgcggatgatactcttat
tttctgtggtactgagagatcacaagcacgaaatctcaacctgacactgatgatcttcga
ggcactatcaggactccacatcaatatgataaagagcatcatataccctgtgaatgcagt
ccccaacatacaagagctagcagacatcctatgccgcaaaacagacactttcccaaccac
atatcttggacttcccttgggagctaaattcaaatcaaaagaagtttggaatggagtcct
agagaagtttgaaaagaggcttgcgacttggcaaatgcaatacctccccatgggtggcag
gttaactttaatcaatagtgtactggacagtcttcccacataccacatatctttgttccc
aattccaatctcagtcctaaagcagatggacaaactcagaaggaagttcttatgggaagg
atgcagcaaaacacacaaatttccactagtgaaatggctgaaggtaactcaaccaaaatt
caaaggagtcttgggaatcagggatgctatgctcttaaaatggctctggagatatggaca
ggaggaatctaggctatggaaggacatcatatttgctaaatatggagcacacaaccactg
gtgttccaagaaaacaaactctccttatggagttggtctgtggaagaacatcagcaacca
ctgggatgaattcttccaaaatgtaactttcaaagttgggaatgtaactcgtataagttt
tggaaggatagatggcttggaaatacacctttgaaagacatgtttcccagtatgtatcag
attgccgtgaccaaagactccactgttgctcataatagaaacaatgacacttggtaccca
cttttcagaagaaatttgcaggattgggaggtcaacaacctactcacaatgttaagctcc
ctagaatgtcataacattgaagatcaacaacctgacaaacttatttgggaaaattctaag
agaggcaagtacacagtcaaagaatgatacattcacctctgtgaccagaatccaatatat
aactggccatggaaacatatctggagaactaaagtgcctaccaagatgacttgcttcaca
tgattgtctctaaatggggcctgtctcactcaagacaacttaatcaagaggaacatcata
taagttaatagatgctacatgtgccaacaacagtcagaaagtgtaaagcacttattcctt
cactgctcagttgcaaaagaaatttggaacttcttctacactacctttggtctaaaatgg
gttatgccacaatcaactaagcaagcttttgaaagttggtatttttggagagttgataaa
tccattagaaaaatctggaaaatggtgtcggccgcaagtttttggtgtatttggaaagaa
aggaactgaagatgttttgatggcatatcaactccactcaaggctgcgtgtttagttaac
ttattttgctggaactatctcacccctgttaatagtgctgatacttctgtggatttcatt
agccccctgatagtagcataggcttttgtaaatggagctaattatcctttctcttttgta
ctctttgcatcttcttgatgccttttaatgaatctaatttacttcatcaaaaagaaaatg
acaagttgttgaaggaggaaaagatgtgagtccatgtgatttagcaaggataaggtacta
aagtccatttgattcacgtccggtaccaatgatccgtctcgtgctgcattcctgtattaa
aacagagtcatcaagaaataaaatagagcaaataagtgattggccaagcgactagtggat
atgagattaaaaggactatggggaacataaaaaactgaattcaaaggtaaggaaggaagt
ggactagcttaacctattctagttgccatggtttgagaatcgttggccattgtgactatt
ggaagtgattgagagtaagaaatagtagtgaaaggagatttgttacccgaaatataatta
gatgcacctgaatcaatgacccaaaagtcggaagaagaggaaacacaagtcacgctatta
cctgtttgaacaatagagattagtttggatcaaatagttgtatagagaactgaaatttgg
agaaatcaatcatatagaacttgtatgtgattattgttgccctttatattgcgtcaaatc
ctaaaacacattgagattaactgccacttatcacagaaaagatattctctagagacattg
ttacaatttcatgaagtcaagtaattagcttgaacatatcttcagcaagtccctcgtcag
tcctcatattagttacatttgtaacaatgtcggtacataagacttataagcaccagtttg
aggaggagtggtagagagttgatgtacatagttaaagtagatatacttacacttagtgtt
atgtaaagagtggatataaaaagggatcagcataagacaattgtcttcgcgcgtcttaac
atttttttcctgtctttatttctctcatggtatcagataacctatctctatcttggttta
cccaatggttggcccccatattgtattagccatgctccagttgactaggcttggacgggc
agaggtgttaaattatcccatattggttgaaagaatgagctattgtctccttatatggtc
ttagacaattctccaactcatgagatattttgttttggctgagttagccctaaggtttat
tttttgtcatattctttaaccttatggcaatgcttgtacacggaaaaaccggagtgcaag
acttaaattaggagaaggaaactattgaaggtgaggaacttaaagggttgtgagaataca
cgggagaaaaaaatcttaatactatctagtggccttgtatatcaaatgatcagcttgcaa
atattttcaccaagtccctcactggtcctcgtattagttacatatgtaacaagttcggta
tatatgatttgtatgcaccggcttgaggttatgcatattctattcctcctactatatatg
tgactaggaaatattttactcctactgcatatgggactaggactatttacacataactat
ctaacattcccctcaagccagtgcacacaagtcatatgtaccgagcttgttacatatgta
actaatacgaggaccagtgagggatttagtaaaaatatctgcaagctggtcattcgacat
acaaggccactagactccccccgagcaacaaaaccaggtggttgctgataaacagaaact
ggccgaaaagttgccggaaaaatttgaaaatagtgagactaagccgaattctacactaca
aaataggttctaaaacaccaccagaaaacaaaaacttttctagaaattactcttcacacc
ggaaaaaataaaagttgtcagaatttgatgtaatttatatagataggttcggaatcactg
gaggagtaagttgtcccgaagaagttttgtcaaaaagtggccggaatggctcacatgcgc
cggaaaacttactgtagctcgcaggaaccctagttctggcggtgcgtggaggcgcgtgac
ttaagattaagatgcttacaggactatcttgagaaatatacatattatatagacgcttga
gttgcttcccaatcctaaatagaagcttttattcgtaggcaagaagggaagcagctttac
ttgagccaatagctttcaaggtgcacgttgtcacaccaaggacatccagaatttgatttt
atagggggtgtgagaaagcacgggagaaaatatgttattgatatttggataataaataca
atacaagaggtccctatttatagctatacactacaaggagatattactcctcttccaatg
tgggacaagaatacactatacatatctgtaaactaacactccccctcaagtcggtgcata
cacatcatatgtaccgatcttgttacacatgtagctaatacgagaaccaataagagactt
agtgaaaatatctgctagttgatcattcgactttacaaactttgtaacaatatctcctga
aagtattttttctctgacaaagtgacagtcgatctcaatgtgtttagtcctctcatggaa
caccggatttgacacaatatgaagagtagcttggttatcacacattagttccatcttgct
gatttctccgaattttaactccttgagcaactgcttgacccaaaataactcacacgtcgt
catagccatggcccgatattcggcttcggcgctagatcgagcaactacattctgtttctt
gctcttccacgagaccaaattacctcctactagaacacaatatccagacatagaacgtct
atcaaaaggtgatcttgcccaatcagcatctgtgtacccaacaatctgctcgtggccttg
atcctcgaatagtaatcctttgcccggagctgactttatataccgaagaatgcgaacaac
tgcatcccagtgactatcacagggagaatccataaactgacttacaacactcaccggaaa
agaaatgtcaggtctagtcactgtgaggtaattcaatttgccaaccaacctcctatatct
cgtagggtctctaagaggctccccctgtccaggcagaagcttagcattcagatccatagg
agagtcaataggtctgcaacccatcattccagtctcctcaagaatgtctaagacatactt
ccgctgtgaaataacaatacctgagctagactgagcgacctcaatacctaaaaaatactt
caatctgcccagatccttagtctggaagtgctgaaagagatgttgcttcagattagtaat
accatcctgatcattgccagtaataacaatatcatcaacataaatcactagataaataca
cagattaggagcagaatgccgataaaacacagagtgatcagcctcactacgagtcatacc
gaactcctgaataattgtgctgaacttaccaaaccaagctcgaggggactgtttcaaacc
atatagtgacctgcgcaatctgcacacacaaccattaaactcccctaagcaacaaaacca
ggtggttgctccatataaacttcttcctcaagatcactgtggagaaaagcattcttaatg
tctaactgataaagaggccaatgacgtacaacagccatggacaaaaagagacgaacagat
gctactttagccacgggagagaacatatcactataatcaagcccaaaaatctgagtatat
ccttttgcaacaagacgagccttaaaccgatcaacctggccatccggaccgactttgact
gcataaacccaacgacaaccaacagtagacttacctgcaggaagaggaacaagctcccaa
gtgcaactcgcatgtaaagcagacatctcgtcaatcatagcatgtcgccatcctggatga
gatagtgcctcacctgtagacttagggatagaaacagtggacaaagaagatataaaagca
taatgaggtgacgacagacgatgataacttaaaccgacatagtggggattaggattaagt
gtggatcatacacctttgcggagtgcaattggttgactaagaggagacaagtccgcagta
ggtgcagaatctgatgcggggcgtgaatcacctgggcctgatgctggatatggacgacga
tgataagtcaagagtggtggagctgccgaaggttgaactggattatgtggaggaactgga
gctataggtggtggagctacaactggagctgtaggtggtggaactagagtaactgaatct
ccaaaagatgaaactggtagtacctcagaaatatctaagtgatgacctgaacctgtgaag
tatgattgggtttcaaagaaggtaacatcagcagacataaggtactgctggaggttagga
gagtagcatcgataccccttttgtgttctcgagaaacctagaaatacgcacttaagagca
cgaggagctaacttatccgttcctggaataaggttatgcacaaaacaagtgcttccaaag
atacgaggtggaagagagaacaaaggtaagtggtaaaacatgacagagaatggaacttgg
ttctggatagctgatgatgtcatacgattaataagatagcaagatgtaagaactgtatcc
cccaaaaacgcaacggagcatgagattgtatgagtagggtacgagcagtttcaataaaat
gtctattctttctttcagctaccccattttgttgagatgtgtacagacaagatgtttgat
gaataatcccatgagatttcataaactgctgaaatggggaagacaaatactctcgggcat
tatcactacgaaatgtgcgaatagaaaccccaaattgattttgaatttcagcgtggaagg
tctggaaaatagaaaacagctcagatcgattttttatcaaaaatatccaagtgcacctgg
aataatcatcaatgaaactgacaaaatagcagaatcccaaggtggaactgacccgactag
gaccccaaacatctgaatggactaaagtaaaaggtgactctgctcgattatcaagacgcc
taaggaaatgggagcgagtatgcttaccgagctgacatgactcacactctagagctgaca
agtgagataaaccagataccattttctgaagttttgacaaactgggatgtcccaaccgtt
tatgtaataaatctggtgaatcagtaacaggacatattgtagatggaagacaagatgcga
gtccatgtatttagcaaggataaggtaataaagtccgtttgattcacgcccggtaccaat
gatccgccccgtactgcgttcttgtataaaaacatggtcatcaagaaataaaataacgca
tttaagtgatttggctaagcgactaacaactatgagattaaaaggactattgcgaacata
aaggactgaatctaaaggtaaggaagaaagtgggcttgcttgacctattgcagttgccat
ggtttgagacccattggctattgtgacttttggaaaagattgagaatacgaaatagtagt
gaaaagagatttgttaccagaaatatgatctgatgcacctgaatcaatgacccaagactc
agaggatgaagattgggaaaaacaagtcacgctattacctgtttgaacaacagaagctat
ctcagaagatgtctgcttacatgctttgtactaaaggaactcaatataatctgctaaaga
aaccatccgactattcaaagcatcggttcccatgtcgctacaatttgtagtagtagggtt
aacttgaaatagtggaaataagtaactccggtgagaaaactgaagaaatagcttgaaaac
actgtttacaacagtaaaaacagaacactgttctgcgccggaatctactgtagctgacgg
aaaaactcaaagtagtcggaatgaaacgaaaaacagtaggggtaggatcggaattaccag
gcgacccaactattctgaaggaagtttttcaaaaaatggccggaagtggtcgtacgtgtc
ggcgcgtgagctcacgcgcgtgagcttctggtggcgcgtggaggcgcgtgaggaggctgc
tgccggagattttcactggggtttggtcgccggacagtgactactcttgtggtagtgttg
gattttgcacaacactgacggagataaagcagacgcaaacagccttgaaaaagtcgccgg
aaaagacttccggtgactgatttctcttcctggaatcgctggaatttatgcacagcgata
aatctctcacaattgctctgataccatgtgagaaagcatgggagaaaatatgttattgat
atttggataataaatacaatacaagaggtccctatttatagctatacactacaaggagat
attacttctcttccaatgtgggacaaaaatacactatacatatctgtaaactaacaaggg
gaatatcgtttaaagataaaaaagatagcgtgcagaagattgcatacattagagatgcaa
aatacagaatacccatactcccagataatgcagtatgccttttgcatgacccactggttg
aatggaagcacctggtcaatttactaggtgtgttagtgatttttgctgcttccttcccct
ttctaaactacatactatctaaaatgttagggggacagaagcccagtcaatctgactagg
tgatgttagtggtttccgcttctttctcccacttctaaatgcgtactttctcaaatttag
gagcatagaaacttaagcagctgcctacctgaggaggtgcatgggaacataagagaatag
actttacctgtcatattttccataccttagttaattacagtgttatcctgataatgatct
gttttctgtatctaggctgaatcgagattcaatcgcttttggctgaaaggatgctgctac
agatccttagtttacatcattgtggttcttattctataagtacttcccctatcaactact
tccttcttttttcttaggttatttgcctcttaggttgtttgcaaggaaaggaacaataga
tgttttgatggaatagcaactccaaaccacttccttaaggctaatatactgtttggccaa
gcttcttcaaagtccaaagcccttttttgtcttcaaaaaagtatctttttttcccaaagt
tgaggtgtttggccaaacttttggaaggaaaaaaaagtgcttttgagtaaagcagaagct
cttgagaagtagaaaaagtagttttttcccggaagcatttttttgaaaagcacttttgag
aaaaataaacttagaaacactttttaaaagtttggccaaacactaattgctgcttaaaag
tgtttttcagatttattagccaaacacaaactgcttctcaccaaaagtacttttttgaaa
aatacttttttgaaaagtgattttcaaacaaagcacttttcaaaataagtttattttaga
agcttgtcaaccggctataaatgtcttttatttttacagctagagtaccctaacacctgt
aaattcccctagacatttttttcgactttgttagctcattaaccctagtataggactctt
tgttttggagctagcaaactcttttgttttcctatttttgcatcttcttggtgccattta
taatatctcttacttcaccaaaaaaaataagttcccaaaatatgactaccttgagttggc
caaagcataaccaaagcttgggcacaccagtgtttgcgtgaattttatggatgttcctta
cctttatccttctgtgcttatgtagcatctgtcttggttaatcttttctgaagtctatag
tgtatttctgtgttgcaacatgagtttactgtcaatcttactgtttgacctcaattttgg
gttctttttgattttgaaagacatcgtttaacaggttggcatggctgctactcttgctgg
tgtctgtcaggtgcctctcactgctgttttgcttctctttgaactgacacagaattatcg
gatagttctgcccctcttgggagctgtggggttgtcttcttgggttacatctggacaaac
aaggaaaagtgtagtgaaggatagagaaagactaaaagatgcaagagcccacatgatgca
gcgacaaggaacttctttctccaacatttctagtttaacttattcttcaggtgtgaaacc
ttcacagaaagagagtaacctatgcaaacttgagagttccctctgtctttatgaatctga
tgatgaagaaaatgatttggcaaggacaattctagtttcacaggcaatgagaacacgata
tgtgacagttctaatgagcaccttgctaacggagaccatatccctcatgctagctgagaa
gcaatcttgtgcaataatagttgatgaaaataattttctcattggtctgctgacacttag
tgatatccagaattacagcaagttgccaagagcagagggcaatttccaggaggtagcttc
ttggtacatttcaatattcttaactgatgaaaaaataagggaaattgatctagcatgaaa
ttaagctaattataagttttacactgtagaactggtaaaacagggttggctggatatttc
tttgttgaatttttaggattatatgtattgttttagttttgtaggttgttttctgatgtg
ctttttgacttggcagaatcttaagatgaaatggaaggtgtttaaccaaaaaatagaatt
ttcagtcaaagcctatatttagaagaaaacgggttattgataaccaagttttactttact
tccccaacaatctatttggtaaatagcaaaagtaatgcgtatgtgagaaagcacgggaga
aaatatattattgatattagatattcaatataatacaagaggtcctacacatcatatagc
tatagtctacaaactacatattactctcattccaatgtgggactacacataactaacact
ccccctcaagccggtgcatacatatcatatgtaccgagcttgttacacatgtaactaata
cgagaaccagtaagagacttagtgaaaatatctgctagttgatcatttgactttacaaac
tttgtaaaaatatctcctgaaagtattttttctctgacaaagtaacagtcgatctcaatg
tgtttagtcctctcatggaatagcggatttgacgcaatatgaagagcagcttggttatca
cacaccagttccatcttgctgatttctccaaactttaactccttgagcaactgcttgacc
caaactaactctcacgttgccatagccattgcccgatattcgacgtcggcgccagatcga
gcaactacattctgtttcttgctcttccacgagaccaaattacctcctactagaacacaa
tatccaggcgtagaacgtctatcaaaaggtgatcctgcccaatcagcatttgtgtaccca
acaatttgctcgtggcctcgatcctcgagtagtaatcctttgcttggagatgactttata
taccgaagaatgcgaacaactgcatcccagtgactatcacagggagaatccataaactga
cttacaacactcaccggaaaagaaatgtcaggtctagtcactgtgaggtaattcaatttg
ccaaccaacctcctatatctcgtagggtctctaagaggctccccgtgtctaggcagaagc
ttagcattcggatccataagagagtcaataggtctgtaacccatcattccagtctcctca
aaaatgtctaaggcataattccgctgtgaaataacaatacctgagctagactgaggcact
gagcaacctcaatacctagaaaatacttcaatctgcccagatccttagtctggaagtgct
gaaagagatgttgcttcagattagtaatatcatcctgatcattgccagtaataacaatat
catcaacataaaccactagataaatacacagattaggagtaaagtgccgataaaacacag
agagatcagcctcactacgagtcatggcgaactcctgaataattatgctgaacttaccaa
accaagctcgaggggactgtttcaaaccatataatgacctgcacaatctacacacacaac
cattaaactccccctgagcaacaaaaccaggtggttactccatataaacttcttcctcaa
gatcaccgtggagaaaagcattcttaatgtctaactgataaagaggccaatgacgtacaa
cagccatggacaaaaagagacgaacaaatgctattttagccacgggagagaaagtatcac
tataatcaagcccaaaaatctgagtatatccttttgcaacaagacgagccttaagccgat
caacctggccatccgggccgactttgaccgcataaacctaatgacaaccaacattagact
tacctgcaggaagaggaacaagctcccaagtgccactcgcatgtaaagcagacatctcgt
caatcatagcatgtcgccatcctggatgagatagtgcctcacctgtagacttagggatag
aaacagtggacaaagaagatataaaagcataatgaggtgatgacacacgatgatgactta
aaccgacatagtggggattaggattacgtgtggatcgtacgcctttgcggagtgcaattg
gttgactaagaggagacaagatcgtagtaggtgcagaatctgatgcagggcgtgaatcac
ttgggcatgatgttggatgtggacgacgatgataagtcaagagtggtggagctgcagaag
gttgaactggattatgtggaggaactggaggtggagctacaactggagctgtaggtggtg
gaactggagctataagtggtggagctacaactggagctggagatgtagaggaagatgaat
gagagatagtgactgaatctccaaaaaataaaattggtagtacctcagaaatatctaagt
gatgacatgaacctgtgaagtatgattgagtttcaaagaaggtaacatcagcggacataa
ggtaccgctgaaggtcaagagagtagcatcgataccccttttgtgttctcgagtaaccta
gaaatacgcacttaagagcacgaggagctaacttatctgttcctggagtaaggttatgga
caaaacaagtgattccaaagatacagggtggaagagagaacaaaggtaagtggggaaaca
tgacaaagaatggaacttggttttggataactgaagatggcatacgattaataagatagc
aagatataagaactgcatccccccaaaaacgaaacggagcatgagattgtatgagtaggg
tacgagcaatttcaataagatgtctattttttctttcagctaccccattttgttgagatg
tgtacagacaagatgtttgatgaataatcccatgagatttcataaactgctgaaatgggg
aagacaaatactctcgggcattatcactaggaaatgtgcgaatagaaaccccaaattgat
tttgaatttttagcgtggaaggtctggaaaaatagaaaacaactcagatcgattttttat
caaaaatatccaagtgcaccttgaataatcatcaattattcaataaaactgacaaagtag
cagaatcccaaggtggaactgacccgactaggaccccaaacatttgagaatggactaaag
taaaaggtgactctgcttgattatcaagacgccgagggaaatggaagcgagtatgcttat
cgaactgacatgactcacactctagagctgacaagtgagataaaccagataccattttat
gaagttttgacaaattgggatgtcccgaccgtttatgtaataaatttggtgtattagtaa
caggacaagttgttgaaggaagacaagatgtgagtccgtgtgatttagcaaggataaggt
aataaagtccgtttgattcacgtccggtaccaataattcgtcccgtactgcgttcctgta
taaaaacatggtcatcaagaaataaaacaacgcatttaagtgatttggctaagcgactaa
tagttatgagattaaaaggactattgggaacataaatgactgaatataaaggtaaggaag
gaagtgagcttgcttgacttattgttgttgccattgtttgagacctattggccattgtga
ctcttgaaagagattgaaaatacgaaatagtagtgaaaagagatttgttaccagaaatat
gatctgatgcacctgaatcaatgacccaaaactcagatgatgaagattgggagaaacaag
tcacgctattacctgtttaaacaacagaagctatcacagaagatgtctgcttacatgctt
tgtaccgaaggaactcaatataatctgctaaagaaaccatccgactattcaaagtatcgg
ttcccatgtcgctacaatttgtagtaataggatggatagactcggaaaattgtaaagtta
tcggaatttgtcgtaaccaggatcgagcaagctgtcttgaagaaatggtttcaaaaaatg
tccggaaaggtcacttttacgccggaaaaatataaaaatggtcgaaatttgatttgaatt
agatgggtaggctcggaattgtgaggagagcagactgtcctgaagaagcttaatgaaaaa
atggccggaaagtggccggaaccctcgccgtaaaagttgttaccggcgcgtgaaggcgcg
tggcattttttctgccagataaattttcaggggttggtcgtcggagggtgatcccttgtg
gtggtgttggtttttgcacaataccgacaggccttaggtcacccgaaaatttgcacgatg
actaagttctttcttcccggttaacgctggaatgacgcacatcgatcttttctcactaat
gctatgataccatgtgagaaagcacgggagaaaatatattattgatattagatactcaat
ataatacaagaggtcatatttatagctatagtctacaaagtacatattactctcattcaa
atgtgggactacacataactaacaacgtaaattaacaaagagaaataaggaatgtaacaa
cagtcaatccctaaaatcaaggtagaaaactttgataaagcagagaattatagaatgtat
ttcagtagtacttggaacttgtccttacaaataaaattctttatccttatataggggcgt
acaatcataacatttttcgcacttaattcgaattcattatgagcattaattgtattgatt
gcccgttatcatagataaccataactgacgtatttgtaactataaatgccttataacggc
tctgattccccttccttatttacttctggtttgtgtatctttccttctttttagccttta
ttcattcagttctcgcctcttctttgacaactgtcaagcccgatcctctgttctgtactg
tctcgtgggtgtttcccccgtaccttccttatattcttaattctgttaattgagagtgtc
acttgtcactatgccattgttccacgcgtcatgtttcatccacgtgtaatatcttttttc
caccaatacagataatcccccactttctgaatattctcaactgaatattcgggtaagttt
ttatggcgggaattctttgccgtcgtttttcgagtatcatcgtgtcatcttcagaaccga
tgtgacgtacgtcacgtctatttaatgcctatgccaggtggcttctatcgattggctctg
cagttttttagcgctttttagggtttttcagcggctgcgtcagtcacgaagtgacggttc
cattatgacgcttcataatgactaactttaatgatggtcgtgtcttcttattaatacttc
attcctttttgatctcttggagtcttccttcttcagtatccaccacattacttctttgta
tttctgcatcttctctttgatattcctttggacaatcatgtcttcttctacaccagaccc
ccgtaaggttgtgattgttgacgaacttgatctttctactgctcctactagaagtaggag
aggtggtagacttcgtagtcttggttcactatctaatcgtggttcttcttcccagggtag
tgctgctaagccatcttcttctagacctagggctcctttaacccctagatcttcttctag
gaatagagatttaaatgatccagtgcgcgaacctacagttgcagagattgttcctcaaga
attttcttttgtaactgaccgtgaaaccataaggaatcaaatttcttctatagcctccct
caataccgctaacctttatccaagtttaatcagtaatggtcttctctcccgggttcgaag
agaatattactgaaaccagatttcccaattttagtccctggtgccaaccagagaattact
ccataccatgttggtttttcctttgtttacacctacccttttactttagggttcaaacca
cctattgaaccagtaatcattgaattctgtcgttatttcaacgtgtgtcttggccagatt
gaccacatagtatggagggctgttcatgccttcgttatttatcagatttggtttccatgc
ctttcacttttcagcacttgcttcatctctactcccctaaattgtttcgtgaagtagttt
ttactctcgtggctagaagtaagagagtgttggttagccttgaagacgattgggaccgtg
gctggtacgctcgttttgttgctgctcccactagtgcattagtgggtgaagaaaatatgc
ctttcccggagaaatggaactttgcacgtaagctttcttctcctctttttttttgtctta
aaaaaactccatgtaatcatatacccacttcttcagcaactatggaagttttttatgctt
gggtagaaaagatgttaactgctgcgcctatggagaaaagatcctggaaatacttttctc
aaagatttggttggaaagtgaagacgcacggtactttttaccttcattgtttttcctttt
ctcttccttgtttgttcaatgatttctcatccttccctttttttttactagggtttccga
ttcgtggtattagtcccgcgtctgttccatcaactaggctttccgtgattcttgttcagg
aaagaattttaagtgcttcttcttcaaaaaggaaaactgacggagcccgtggctctgatg
acgaagaagaaacagaggagggttctttggtgcgaaggtcacgcgtcaggagacgcgtgg
tttctgatgatgaaactactccttctcatgaccctctatctagttcaatcccttttagac
tcacggatgagctagagagtacccctttagtgatttcttatgatgatgctgttgatcccc
ctccaagttctgttgatagattgtttgctcatggcttcgagggtgatgaagttttgggcc
tgtttctgaagaattgccccttgcttcccttccagtttcagttttcattaacccttccgt
gtccttacctgatgatactcctgttgttattctcgtggctgcttctactccgtcatctat
tcccgtgactgcttctcatgcagaggccaaaccttctagcagcagaagggcaatgaaaag
agttgttgttgaggttcctgaaggtgagaacttattaagaaaatccggtcaagccgacgt
gtagttgaaacctatgctcggccccgtagagaagaagaagttagaaagccatagctcact
cactttaatgaatgatatcgttcattcttccttgaaagtacaagcttaattatatttcct
ttcttttctctttcttattcataactcttcctccttttttgcagatcaacttgattggca
cagagcttatgaaaagagtttctcaggcggaccggcaagttatagatttgcgcaccgagg
ctgataactggaaggaacaattcgaaggtcttcaattggaaaaagaggttccggcggaag
agaagaatgctttggaacaacagatgagagtgattgcctctgaattagcagttgaaaaag
cttcctcgagccaggttggaaaggataagtatatacttgaatcctcctttgctgaacaac
tttccaaggcaactgaagaaataaggagtttgaaggaactccttaatcaaaaagaggttt
atgcgagagaattggttcaaacacttactcaagttcaggaagatctccgtgcctctactt
ataagattcagttcttggaaagttctctcgcttctttgaagacagcttacgatgcctctg
aagcagaaaaagaagagctgagagctgagatttaccagtgggagaaggattatgagattc
tcgaggataatctatcgttggatgtaagttgggctttcttaaacactcgtctcgagactc
tagttgaagccaaccatgagggttttgaccttaatgctgagattgctaaggctaaagaag
caattgataaaactcagcaacgtcaaatcttttcctcacctgaagacgaaggtcccgaag
gtgatggagattga
(Protein sequence of NtCLCe from Nicotiana tabacum; sequence
originating from the ancestor N. sylvestris; two start codons,
translated from SEQ ID NO: 10)
SEQ ID NO: 12
MISGQNTVLHNPPNSLFNSLSPRHICISFCNDKALKKSVTHSAPRFARLL
NNESRKLLGRHPNCWPWARRPSLPPGRSSDGNIEKEQDMCDSSKVDSDSG
IQIGSLLEEVIPQGNNTAIISACFVGLFTGISVVLFNAAVHEIRDLCWDG
IPYRAASEEPIGVHWQRVILVPACGGLVVSFLNAFRATLEVSTEGSWTSS
VKSVLEPVLKTMAACVTLGTGNSLGPEGPSVEIGTSVAKGVGALLDKGGR
RKLSLKAAGSAAGIASGFNAAVGGCFFAVESVLWPSPAESSLSLTNTTSM
VILSAVIASVVSEIGLGSEPAFAVPGYDFRTPTELPLYLLLGIFCGLVSV
ALSSCTSFMLQIVENIQTTSGMPKAAFPVLGGLLVGLVALAYPEILYQGF
ENVNILLESRPLVKGLSADLLLQLVAVKIVTTSLCRASGLVGGYYAPSLF
IGAATGTAYGKIVSYIISHADPIFHLSILEVASPQAYGLVGMAATLAGVC
QVPLTAVLLLFELTQDYRIVLPLLGAVGLSSWVTSGQTRKSVVKDREKLK
DARAHMMQRQGTSFSNISSLTYSSGSPSQKESNLCKLESSLCLYESDDEE
NDLARTILVSQAMRTRYVTVLMSTLLMETISLMLAEKQSCAIIVDENNFL
IGLLTLGDIQNYSKLPRTEGNFQEELVVAGVCSSKGNKCRVSCTVTPNTD
LLSALTLMEKHDLSQLPVILGDVEDEGIHPVGILDRECINVACRALATRE
QLC
(Protein sequence of NtCLCe from Nicotiana tabacum; sequence
originating from the ancestor N. tomentosiformis; one start codon,
translated from SEQ ID NO: 4)
SEQ ID NO: 13
MCDSSKDDSDSDSGIQIGSLLEEVIPQGNNTAIISACFVGLFTGISVVLF
NAAVHEIRDLCWDGIPYRAASEEPIGVHWQRVILVPACGGLVVSFLNAFR
ATLEVSTEESWTSSVKSVLGPVLKTMAACVTLGTGNSLGPEGPSVEIGTS
VAKGVGALLDKGGRRKLSLKAAGSAAGIASGFNAAVGGCFFAVESVLWPS
PAESSLYLTNTTSMVILSAVIASVVSEIGLGSEPAFAVPGYDFRTPTELP
LYLLLGIFCGLVSVALSSCTSFMLQIVENIQMTSGMPKAAFPVLGGLLVG
LVALAYPEILYQGFENVNILLESRPLVKGLSADLLLQLVAVKIVTTSLCR
ASGLVGGYYAPSLEIGAATGTAYGKIVSYIISHADPIFHLSILEVASPQA
YGLVGMAATLAGVCQVPLTAVLLLFELTQNYRIVLPLLGAVGLSSWVTSG
QTRKSVVKDRERLKDARAHMMQRQGTSFSNISSLTYSSGVKPSQKESNLC
KLESSLCLYESDDEENDLARTILVSQAMRTRYVTVLMSTLLTETISLMLA
EKQSCAIIVDENNFLIGLLTLSDIQNYSKLPRAEGNFQEINLIGTELMKR
VSQADRQVIDLRTEADNWKEQFEGLQLEKEVPAEEKNALEQQMRVIASEL
AVEKASSSQVGKDKYILESSFAEQLSKATEEIRSLKELLNQKEVYARELV
QTLTQVQEDLRASTYKIQFLESSLASLKTAYDASEAEKEELRAEIYQWEK
DYEILEDNLSLDVSWAFLNTRLETLVEANHEGFDLNAEIAKAKEAIDKTQ
QRQIFSSPEDEGPEGDGD
(Protein sequence of NtCLCe from Nicotiana tabacum; sequence
originating from the ancestor N. tomentosiformis; two start codons,
translated from SEQ ID NO: 11)
SEQ ID NO: 14
MISGQNTVLHHPPNSLFNSLSPRHICVSFCNDKALKKSVTHSAPRFARLL
NNESRKLLGRHPNCWPWARRPSLPPGRSCDGNIEKEQDMCDSSKDDSDSD
SGIQIGSLLEEVIPQGNNTAIISACFVGLFTGISVVLFNAAVHEIRDLCW
DGIPYRAASEEPIGVHWQRVILVPACGGLVVSFLNAFRATLEVSTEESWT
SSVKSVLGPVLKTMAACVTLGTGNSLGPEGPSVEIGTSVAKGVGALLDKG
GRRKLSLKAAGSAAGIASGFNAAVGGCFFAVESVLWPSPAESSLYLTNTT
SMVILSAVIASVVSEIGLGSEPAFAVPGYDFRTPTELPLYLLLGIFCGLV
SVALSSCTSFMLQIVENIQMTSGMPKAAFPVLGGLLVGLVALAYPEILYQ
GFENVNILLESRPLVKGLSADLLLQLVAVKIVTTSLCRASGLVGGYYAPS
LEIGAATGTAYGKIVSYIISHADPIFHLSILEVASPQAYGLVGMAATLAG
VCQVPLTAVLLLFELTQNYRIVLPLLGAVGLSSWVTSGQTRKSVVKDRER
LKDARAHMMQRQGTSFSNISSLTYSSGVKPSQKESNLCKLESSLCLYESD
DEENDLARTILVSQAMRTRYVTVLMSTLLTETISLMLAEKQSCAIIVDEN
NFLIGLLTLSDIQNYSKLPRAEGNFQEINLIGTELMKRVSQADRQVIDLR
TEADNWKEQFEGLQLEKEVPAEEKNALEQQMRVIASELAVEKASSSQVGK
DKYILESSFAEQLSKATEEIRSLKELLNQKEVYARELVQTLTQVQEDLRA
STYKIQFLESSLASLKTAYDASEAEKEELRAEIYQWEKDYEILEDNLSLD
VSWAFLNTRLETLVEANHEGFDLNAEIAKAKEAIDKTQQRQIFSSPEDEG
PEGDGD
(Protein sequence of NtCLCe from Nicotiana tabacum; sequence
originating from the ancestor N. tomentosiformis; one start codon,
translated from SEQ ID NO: 4) including a P184S mutation
SEQ ID NO: 15
MCDSSKDDSDSDSGIQIGSLLEEVIPQGNNTAIISACFVGLFTGISVVLF
NAAVHEIRDLCWDGIPYRAASEEPIGVHWQRVILVSACGGLVVSFLNAFR
ATLEVSTEESWTSSVKSVLGPVLKTMAACVTLGTGNSLGPEGPSVEIGTS
VAKGVGALLDKGGRRKLSLKAAGSAAGIASGFNAAVGGCFFAVESVLWPS
PAESSLYLTNTTSMVILSAVIASVVSEIGLGSEPAFAVPGYDFRTPTELP
LYLLLGIFCGLVSVALSSCTSFMLQIVENIQMTSGMPKAAFPVLGGLLVG
LVALAYPEILYQGFENVNILLESRPLVKGLSADLLLQLVAVKIVTTSLCR
ASGLVGGYYAPSLFIGAATGTAYGKIVSYIISHADPIFHLSILEVASPQA
YGLVGMAATLAGVCQVPLTAVLLLFELTQNYRIVLPLLGAVGLSSWVTSG
QTRKSVVKDRERLKDARAHMMQRQGTSFSNISSLTYSSGVKPSQKESNLC
KLESSLCLYESDDEENDLARTILVSQAMRTRYVTVLMSTLLTETISLMLA
EKQSCAIIVDENNFLIGLLTLSDIQNYSKLPRAEGNFQEINLIGTELMKR
VSQADRQVIDLRTEADNWKEQFEGLQLEKEVPAEEKNALEQQMRVIASEL
AVEKASSSQVGKDKYILESSFAEQLSKATEEIRSLKELLNQKEVYARELV
QTLTQVQEDLRASTYKIQFLESSLASLKTAYDASEAEKEELRAEIYQWEK
DYEILEDNLSLDVSWAFLNTRLETLVEANHEGFDLNAEIAKAKEAIDKTQ
QRQIFSSPEDEGPEGDGD

TABLE 1

| | | | | | Original | | Mutant |
|---|---|---|---|---|---|---|---|
| | | Sequence | Sequence | Original | amino | Mutant | amino |
| Gene | Mutation | 5′ of SNP | 3′ of SNP | codon | acid | codon | acid |
| CLCe-S | E21K | ctctgctcgag | aagttatcca | gaa | glu | aaa | lys |
| CLCe-S | L58F | aatacgtgat | tttgttggga | ctt | leu | ttt | phe |
| CLCe-S | P141S | accagaaggc | ctagtgttga | cct | pro | tct | ser |
| CLCe-S | G175E | cagctgctg | aatcgcttct | ctc | leu | ttc | phe |
| CLCe-S | S5N | tgcgacagca | caaagtcgata | agc | ser | aac | asn |
| CLCe-S | A34V | tataatctcgg | ttgctttgtt | gct | ala | gtt | val |
| CLCe-S | M124I | tgaagacaat | gccgcttgtg | atg | met | ata | ile |
| CLCe-S | L40F | gctttgttggc | tcttcaccgg | ctc | leu | ttc | phe |
| CLCe-S | D8N | agatatgtgc | acagcagcaa | gac | asp | aac | asn |
| CLCe-S | C35Y | aatctcggctt | ctttgttggcc | tgc | cys | tac | tyr |
| CLCe-S | A30V | caataataccg | tataatctcgg | gct | ala | gtt | val |
| CLCe-S | A177V | gctggaatcg | ttctggtttgt | gct | ala | gtt | val |
| CLCe-S | G42D | ctcttcaccg | tatcagtgtc | ggt | gly | gat | asp |
| CLCe-S | G88D | ccagcttgtg | cggtttggtag | ggc | gly | gac | asp |
| CLCe-S | G155R | ccaagggagtt | gagctctgct | gga | gly | aga | arg |
| CLCe-S | D158N | agctctgctt | ataaaggtggt | gat | asp | aat | asn |
| CLCe-S | A170V | ctcaaggctg | tggatcagctg | gct | ala | gat | asp |
| CLCe-S | A174V | tggatcagctg | tggaatcgctt | gct | ala | gtt | val |
| CLCe-S | A126V | gacaatggccg | ttgtgtcaca | gct | ala | gtt | val |
| CLCe-S | G131R | gtgtcacatta | gaactgggaa | gga | gly | aga | arg |
| CLCe-T | P184S | ctggtttgttc | ccatattattc | ccc | pro | tcc | ser |
| CLCe-T | G89D | accagcttgtg | cggtttggtag | ggc | gly | gac | asp |
| CLCe-T | K166N | ggtcgtagaaa | ctgtcactcaa | aag | lys | aaa | gln |
| CLCe-T | G18R | gtatccagata | gatctctgct | gga | gly | aga | arg |
| CLCe-T | G76R | ggagcccatt | gagtacattgg | gga | gly | aga | arg |
| CLCe-T | G173R | tcaaggctgct | gatcagctgc | gga | gly | aga | arg |
| CLCe-T | P143L | accagaaggcc | tagtgttgaaat | cct | pro | ctt | leu |
| CLCe-T | M1I | aacaagatat | tgcgacagcag | atg | met | ata | ile |
| CLCe-T | S4N | atgtgcgaca | cagcaaagacga | agc | ser | aac | asn |
| CLCe-T | V154I | cccttggttag | ttcatgaaata | gtt | val | att | ile |
| CLCe-T | G89D | cagcttgtg | cggtttggta | ggc | gly | gac | asp |
| CLCe-T | A128V | gacaatggccg | ttgtgtcacat | gct | ala | gtt | val |
| CLCe-T | S137F | aactgggaatt | cttaggacca | tcc | ser | ttc | phe |
| CLCe-T | G181S | gaatcgcttct | gtttgttccc | ggt | gly | agt | ser |
| CLCNt2-S | G503E | cattgccatgg | atcttataca | gga | gly | gaa | glu |
| CLCNt2-S | G471R | attgcatattg | gactcatcact | gga | gly | aga | arg |
| CLCNt2-S | V659I | ccttcttttg | ttctcaagaaa | gtt | val | att | ile |
| CLCNt2-S | S566N | cttcaacctaa | tatttatgaa | agt | ser | aat | asn |
| CLCNt2-S | P637S | gagtagtgcca | cggtgggtct | ccg | pro | tcg | ser |
| CLCNt2-S | A597T | ctggtgagctt | ctgatgtaaag | gct | ala | act | thr |
| CLCNt2-S | P711L | gatttgcatc | cctgactaac | ccc | pro | ctc | leu |
| CLCNt2-S | G135R | gtaccttatg | gatttgcata | gga | gly | aga | arg |
| CLCNt2-S | A151V | tttgatagctg | ccttctctgcg | gcc | ala | gtc | val |
| CLCNt2-S | G690D | agctgagaggg | cggtaagatc | ggc | gly | gac | asp |
| CLCNt2-S | G737R | tcaggcaggtg | ggctccgcca | ggg | gly | agg | arg |
| CLCNt2-S | G135R | gtaccttatg | gatttgcata | gga | gly | aga | arg |
| CLCNt2-S | G163R | ctactgctgca | ggcctggaatt | ggg | gly | agg | arg |
| CLCNt2-S | P480S | gattgctgtg | catctggtctc | cca | pro | tca | ser |
| CLCNt2-S | S520F | cggagcagctt | ccttatggct | tcc | ser | ttc | phe |
| CLCNt2-S | A514T | cagggctgtat | cggttctcgg | gcg | ala | acg | thr |
| CLCNt2-S | A518V | ggttctcggag | agcttccctta | qca | ala | qta | val |
| CLCNt2-S | G476E | catcacttttg | gattgctg | ggg | gly | gag | glu |
| CLCNt2-S | R739S | gtggggctcc | ccacatgctc | cgc | arg | cac | ser |
| CLCNt2-S | G517E | tgcggttctcg | agcagcttcc | gga | gly | gaa | glu |
| CLCNt2-S | E585K | atgccaacccg | agccatggatg | gag | glu | aag | lys |
| CLCNt2-S | V677I | aggagtgggaa | tgagagagaaa | gta | val | ata | ile |
| CLCNt2-T | A514T | cagggctgtat | ccgttctggga | gcc | ala | acc | thr |
| CLCNt2-T | L537F | gcgtcatattt | ttgagctaaca | ctt | leu | ttt | phe |
| CLCNt2-T | R593I | gccatggatga | aaatatcact | aga | arg | ata | ile |
| CLCNt2-T | A749T | caaataccaa | cagcaggggtg | gca | ala | aca | thr |
| CLCNt2-T | G524D | cttatggctg | ttcaatgagaa | ggt | gly | gat | asp |
| CLCNt2-T | S408F | cacttcaagggt | ttgtcctggca | tct | ser | ttt | phe |
| CLCNt2-T | G503R | cattgccatg | gatcttataca | gga | gly | aga | arg |
| CLCNt2-T | P547S | ttctccttctg | caataacaatgc | cca | pro | tca | ser |
| CLCNt2-T | G691D | gctgagaggg | cggtaagatcga | ggc | gly | gac | asp |
| CLCNt2-T | A478V | tttgggattg | tgtgccatctg | gct | ala | gtt | val |
| CLCNt2-T | A749V | ctccgccacatg | tcattgtacc | gct | ala | gtt | val |
| CLCNt2-T | T713I | gcatcccctga | taacacaaccc | act | thr | att | ile |
| CLCNt2-T | M550I | caataacaat | ctggttcttc | atg | met | ata | ile |
| CLCNt2-T | P586S | gccaacccggag | catggatgaga | cca | pro | tca | ser |
| CLCNt2-T | R670K | ccttaatgaaa | acgaaggaca | aga | arg | aaa | lys |
| CLCNt2-T | R678K | gtgggaagtga | agagaaattc | aga | arg | aaa | lys |
| CLCNt2-T | D631N | tccctgtcgtc | atgaaggagtg | gat | asp | aat | asn |
| CLCNt2-T | L657F | gaactcacctt | ttttggttctc | ctt | leu | ttt | phe |
| CLCNt2-T | G737R | caggcaggtg | ggctccgccac | ggg | gly | agg | arg |
| CLCNt2-T | S525L | atggctggtt | aatgagaatga | tca | ser | tta | leu |
| CLCNt2-T | A597T | tggtgagctt | ctgatgtaaag | gct | ala | act | thr |
| CLCNt2-T | E674K | aaggacagag | agtgggaagtg | gag | glu | aag | lys |

CLC-Nt2-s corresponds to the polypeptide sequence shown in SEQ ID NO. 5 that is encoded by SEQ ID NO: 1
CLC-Nt2-t corresponds to the sequence shown in SEQ ID NO. 6 that is encoded by SEQ ID NO: 2
NtCLCe-s corresponds to the sequence shown in SEQ ID NO. 7 that is encoded by SEQ ID NO: 3
NtCLCe-t corresponds to the sequence shown in SEQ ID NO. 13 that is encoded by SEQ ID NO: 4

TABLE 2

| Target | Forward primer | Reverse primer |
|---|---|---|
| gene | (5′ to 3′) | (5′ to 3′) |
| CLCe-s | TATCTCCTCGCCAT | GTGCAAACACACTT |
| | ATCTGTA | GTATTTAC |
| CLCe-t | ACCATCTCTTCCTC | TATAGGATACTCCT |
| | CGGGA | CTGATAAAT |
| CLCe-t | TTGTACAATTTATC | TTGGTTTGAGTGCA |
| | AGAGGAGTA | AACACA |
| CLCNt2-s | ACTATATCGAGGAT | TATCTATTTATACA |
| | AGAAGGTA | TCTGGTTCG |
| CLCNt2-s | CTTGTGATCCATCA | TATGACTATTTCTG |
| | CTTCCC | TGCATCTTT |
| CLCNt2-s | GCCTTGTGATTCAT | TATGACTATTTCTG |
| | CACTTCAA | TGCATCTTA |
| CLCNt2-t | GGTTCTTCTCGCTC | AACGTAAAATAACT |
| | TGAGC | TTGCCACG |

1. A mutant, non-naturally occurring or transgenic plant cell comprising:
(i) a polynucleotide comprising, consisting or consisting essentially of a sequence encoding a member of the CLC family of chloride channels and having at least 60% sequence identity to SEQ ID NO:1 or SEQ ID NO:2 or SEQ ID NO:3 or SEQ ID NO:4 or SEQ ID NO:10 or SEQ ID NO:11;
(ii) a polypeptide encoded by the polynucleotide set forth in (i);
(iii) a polypeptide comprising, consisting or consisting essentially of a sequence encoding a member of the CLC family of chloride channels and having at least 60% sequence identity to SEQ ID NO:5 or SEQ ID NO:6 or SEQ ID NO:7 or SEQ ID NO:12 or SEQ ID NO:13 or SEQ ID NO:14; or
(iv) a construct, vector or expression vector comprising the isolated polynucleotide set forth in (i),
and wherein the expression or activity of the polynucleotide or the polypeptide is modulated as compared to a control plant containing a control plant cell and wherein the biomass levels in the mutant, non-naturally occurring or transgenic plant containing the mutant, non-naturally occurring or transgenic plant cell are modulated as compared to the control plant containing the control plant cell.

2. The mutant plant cell according to claim 1, wherein said mutant, non-naturally occurring or transgenic plant cell comprises one or more mutations that increase the level of biomass in the mutant, non-naturally occurring or transgenic plant containing the mutant, non-naturally occurring or transgenic plant cell as compared to the control plant containing the control plant cell.

3. The mutant plant cell according to claim 1 or claim 2 wherein the mutation(s) comprise a deletion, insertion, substitution or missense mutation at position P184 of SEQ ID NO:13.

4. The mutant plant cell according to claim 3 wherein the mutation is a substitution mutation.

5. The mutant plant cell according to claim 4 wherein the substitution mutation is P184S, as shown in SEQ ID NO:15.

6. The mutant plant cell according to any of claims 1 to 5 wherein the mutation is heterozygous.

7. The mutant plant cell according to any of claims 1 to 5 wherein the mutation is homozygous.

8. A mutant, non-naturally occurring or transgenic plant or component thereof comprising the plant cell according to any of claims 1 to 7.

9. A mutant, non-naturally occurring or transgenic plant, plant component or plant cell according to any of claims 1 to 8 wherein additionally the nitrate content is modulated.

10. A mutant, non-naturally occurring or transgenic plant, plant component, or plant cell according to any of claims 1 to 9 wherein additionally, the 4-(methylnitrosamino)-1-(3-pyridyl)-1-butanone (NNK) content is modulated.

11. A mutant, non-naturally occurring or transgenic plant, plant component, or plant cell according to claim 9 or 10 wherein the mutation(s) further comprise deletion, insertion, substitution or missense mutation at position G163 of SEQ ID NO:5 and/or at position P143 of SEQ ID NO:13.

12. A mutant, non-naturally occurring or transgenic plant, plant component, or plant cell according to claim 11 wherein the mutation at position G163 of SEQ ID NO:5 is substitution mutation G163R and/or the mutation at position P143 of SEQ ID NO:13 is substitution mutation P143L.

13. A method for modulating at least the biomass content of a plant or a component thereof, comprising the steps of:
(a) modulating the expression or activity of:
(i) a polynucleotide comprising, consisting or consisting essentially of a sequence encoding a member of the CLC family of chloride channels and having at least 60% sequence identity to SEQ ID NO:1 or SEQ ID NO:2 or SEQ ID NO:3 or SEQ ID NO:4 or SEQ ID NO:10 or SEQ ID NO:11;
(ii) a polypeptide encoded by the polynucleotide set forth in (i); or
(iii) a polypeptide comprising, consisting or consisting essentially of a sequence encoding a member of the CLC family of chloride channels and having at least 60% sequence identity to SEQ ID NO:5 or SEQ ID NO:6 or SEQ ID NO:7 or SEQ ID NO:12 or SEQ ID NO:13 or SEQ ID NO:14;
and measuring the biomass yield of the mutant;
(b) optionally measuring at least the nitrate content in at least a part of the mutant, non-naturally occurring or transgenic plant obtained in step (a); and
(c) identifying a mutant, non-naturally occurring or transgenic plant in which at least the biomass has changed in comparison to a control plant in which the expression or activity of the polynucleotide or the polypeptide set forth in (a) has not been modulated.

14. The method according to claim 13, wherein additionally the nitrate content in the plant is modulated.

15. The method according to claim 13 or claim 14, wherein additionally the 4-(methylnitrosamino)-1-(3-pyridyl)-1-butanone (NNK) content is modulated in the plant.

16. The method according to any of claims 13 to 15 wherein additionally the nicotine content is modulated in the plant.

17. The method according to any of claims 13 to 16 wherein at least the N-nitrosonicotine (NNN) content is substantially the same as the control plant.

18. The method according to any of claims 13 to 17, wherein the component of the plant is a leaf, suitably, a cured leaf.

19. A plant or a component thereof that is obtained or obtainable by the method according to any of claims 13 to 18.

20. Plant material including biomass, seed, stem or leaves from the plant of any of claims 8 or 19.

21. A tobacco product comprising the plant cell of claim 1, at least a part of the plant of any one of claims 8 or 19, plant material according to claim 20, or suitably processed plant material according to claim 20.

22. An isolated polynucleotide comprising, consisting, or consisting essentially of a sequence encoding a member of the CLC family of chloride channels and having at least 60% sequence identity to SEQ ID NO:1 or SEQ ID NO:2 or SEQ ID NO:3 or SEQ ID NO:4 or SEQ ID NO:10 or SEQ ID NO:11.

23. A polynucleotide construct, vector, or expression vector comprising a polynucleotide according to claim 22.

24. An isolated polypeptide encoded by a polynucleotide according to claim 22.

25. An isolated polypeptide according to claim 23, comprising, consisting or consisting essentially of a sequence having at least 60% sequence identity to SEQ ID NO:5 or SEQ ID NO:6 or SEQ ID NO:7 or SEQ ID NO:12 or SEQ ID NO:13 or SEQ ID NO:14.

26. A plant or a part thereof comprising a suitably homozygous mutation in a CLC gene as set forth in SEQ ID No. 15.
